SEQUENCE LISTING (114 sequences)

SEQ ID NO: 1 - DNA, 20 bp, Synthetic construct
  source 1..20: mol_type = other DNA
agccacaatgctagggagaa

SEQ ID NO: 2 - DNA, 21 bp, Synthetic construct
  source 1..21: mol_type = other DNA
aatggcattgtttacgacacc

SEQ ID NO: 3 - DNA, 21 bp, Synthetic construct
  source 1..21: mol_type = other DNA
tctccatcgacaccgtttatc

SEQ ID NO: 4 - DNA, 20 bp, Synthetic construct
  source 1..20: mol_type = other DNA
ttcctgtggcttttgtttcc

SEQ ID NO: 5 - DNA, 20 bp, Synthetic construct
  source 1..20: mol_type = other DNA
ttcccaagggcattatggta

SEQ ID NO: 6 - DNA, 20 bp, Synthetic construct
  source 1..20: mol_type = other DNA
aacaacccacgttctcaagg

SEQ ID NO: 7 - DNA, 564 bp, Cannabis sativa
  source 1..564: mol_type = genomic DNA
atgttgcagacgatttattcattgccattagtaaatgtggcgacgtgtctccatcgacaccgtttatcttcatcatcagtgactaatttcacatgtgatggtggtagtactactcttaatgtgaaattgaatagtagtaaagatgatgatgatgatgatgatgataattataataaccaaaaaattgtgaagagagatgggtataataatagaagaagaatgaagatgaatgatgaatattttgaaattgagctcaaagttcgtgattatgagctcgatcaatttggtgtcgtaaacaatgccatttatgcaagttattgccaacatggtactcaagaatttatggaaagtgtaattggtataagctgtgatgcaattgcccgcaatggagaaacattagccacctcagaattatcaatcaaattcatttcacctttaagaagtggagataaatttgtggtgaaggtaagggtctctagattttcagctgctcgtgcatactttgagcacaaaattttcaagcttcccaattatgagcctattttggaaacaaaagccacagga

SEQ ID NO: 8 - DNA, 555 bp, Cannabis sativa
  source 1..555: mol_type = genomic DNA
atgttgcagacgatttatccattgccattagtaaatgtggcgacgtgtctccatcgacaccgtttatcttcgtcatcagtgactaatttcacatgtgatggtggtagtactactcttaatgtgaaattgaatagtagtaaagatgatgatgatgataattataataaccaaaaaattgtgaagagagatgggcataaaaatagaagaagaatgaagatgaatgatgaatattttgatattgagctcgaagttcgtgattatgagctcgatcaatttggtgtcgtaaacaatgccatttatgcaagttattgccaacatggtactcatgagtttatggcaagtgtaattggtataagctgtgattcagttgctcgcaatggagaaacattagccacctcagatttgtcaatcaaattcatttcacctttaagaagtggagataaatttgtggtgaaggtaagggtctctagaatttcagctgctcgtgtatactttgagcacaaaattgtcaagcttcccaattatgagcctattttggaaacaaaagccacagga

SEQ ID NO: 9 - DNA, 630 bp, Cannabis sativa
  source 1..630: mol_type = genomic DNA
atgttgcagacgatttatccattgccattagtaaatgtggcgacgtgtctccatcgacaccgtttatcttcatcatcagtgactaatttcacatgtgatggtggtagtactactcttaatgtgaaattgaatagtagtaaagatgatgatgatgatgatgataattataataaccgaaaaattgtgaagagagatgagcataataatagaagaagaatgaagatgaatgatgaatattttgaaattgagctcaaagttcgtgattatgagctcgatcaatttggtgtcgtaaacaatgccatttatgcaagttattgccaacatggtactcaagaatttatggaaagtgtaattggtataagctgtgatgcaattgcccgcaatggagaaacattagccacctcagaattatcaatcaaattcatttcacctttaagaagtggagataaatttgtggtgaaggtaagggtctctagattttcagctgctcgtgcatactttgagcacaaaattttcaagcttcccaattatgagcctattttggaaacaaaagccacaggaatttggttggataagaagaataacccaattcgtgtaccgcaagagataaagaacacgtttattatttga

SEQ ID NO: 10 - protein, 188 aa, Cannabis sativa
MLQTIYSLPLVNVATCLHRHRLSSSSVTNFTCDGGSTTLNVKLNSSKDDDDDDDDNYNNQKIVKRDGYNNRRRMKMNDEYFEIELKVRDYELDQFGVVNNAIYASYCQHGTQEFMESVIGISCDAIARNGETLATSELSIKFISPLRSGDKFVVKVRVSRFSAARAYFEHKIFKLPNYEPILETKATG

SEQ ID NO: 11 - protein, 185 aa, Cannabis sativa
MLQTIYPLPLVNVATCLHRHRLSSSSVTNFTCDGGSTTLNVKLNSSKDDDDDNYNNQKIVKRDGHKNRRRMKMNDEYFDIELEVRDYELDQFGVVNNAIYASYCQHGTHEFMASVIGISCDSVARNGETLATSDLSIKFISPLRSGDKFVVKVRVSRISAARVYFEHKIVKLPNYEPILETKATG

SEQ ID NO: 12 - protein, 209 aa, Cannabis sativa
MLQTIYPLPLVNVATCLHRHRLSSSSVTNFTCDGGSTTLNVKLNSSKDDDDDDDNYNNRKIVKRDEHNNRRRMKMNDEYFEIELKVRDYELDQFGVVNNAIYASYCQHGTQEFMESVIGISCDAIARNGETLATSELSIKFISPLRSGDKFVVKVRVSRFSAARAYFEHKIFKLPNYEPILETKATGIWLDKKNNPIRVPQEIKNTFII

SEQ ID NO: 13 - DNA, 101 bp, Cannabis sativa
  source 1..101: mol_type = genomic DNA
attgaatagtagtaaagatgatgatgatgatgatgataattataataaccgaaaaattgtgaagagagatgagcataataatagaagaagaatgaagatga

SEQ ID NO: 14 - DNA, 101 bp, Cannabis sativa
  source 1..101: mol_type = genomic DNA
tgatgatgatgatgataattataataaccgaaaaattgtgaagagagatgagcataataatagaagaagaatgaagatgaatgatgaatattttgaaattg

SEQ ID NO: 15 - DNA, 100 bp, Cannabis sativa
  source 1..100: mol_type = genomic DNA
catgtgatggtggtagtactactcttaatgtgaaattgaatagtagtaaagatgatgatgatgatgatgataattataataaccgaaaaattgtgaagag

SEQ ID NO: 16 - DNA, 101 bp, Cannabis sativa
  source 1..101: mol_type = genomic DNA
atgatgatgatgataattataataaccgaaaaattgtgaagagagatgagcataataatagaagaagaatgaagatgaatgatgaatattttgaaattgag

SEQ ID NO: 17 - DNA, 106 bp, Cannabis sativa
  source 1..106: mol_type = genomic DNA
catgtgatggtggtagtactactcttaatgtgaaattgaatagtagtaaagatgatgatgatgatgatgataattataataaccgaaaaattgtgaagagagatga

SEQ ID NO: 18 - DNA, 101 bp, Cannabis sativa
  source 1..101: mol_type = genomic DNA
gatgatgataattataataaccgaaaaattgtgaagagagatgagcataataatagaagaagaatgaagatgaatgatgaatattttgaaattgagctcaa

SEQ ID NO: 19 - DNA, 101 bp, Cannabis sativa
  source 1..101: mol_type = genomic DNA
gatgagcataataatagaagaagaatgaagatgaatgatgaatattttgaaattgagctcaaagttcgtgattatgagctcgatcaatttggtgtcgtaaa

SEQ ID NO: 20 - DNA, 101 bp, Cannabis sativa
  source 1..101: mol_type = genomic DNA
ataatagaagaagaatgaagatgaatgatgaatattttgaaattgagctcaaagttcgtgattatgagctcgatcaatttggtgtcgtaaacaatgccatt

SEQ ID NO: 21 - DNA, 101 bp, Cannabis sativa
  source 1..101: mol_type = genomic DNA
ggtgtcgtaaacaatgccatttatgcaagttattgccaacatggtactcaagaatttatggaaagtgtaattggtataagctgtgatgcaattgcccgcaa

SEQ ID NO: 22 - DNA, 101 bp, Cannabis sativa
  source 1..101: mol_type = genomic DNA
caatgccatttatgcaagttattgccaacatggtactcaagaatttatggaaagtgtaattggtataagctgtgatgcaattgcccgcaatggagaaacat

SEQ ID NO: 23 - DNA, 101 bp, Cannabis sativa
  source 1..101: mol_type = genomic DNA
aacatggtactcaagaatttatggaaagtgtaattggtataagctgtgatgcaattgcccgcaatggagaaacattagccacctcagaattatcaatcaaa

SEQ ID NO: 24 - DNA, 101 bp, Cannabis sativa
  source 1..101: mol_type = genomic DNA
atggtactcaagaatttatggaaagtgtaattggtataagctgtgatgcaattgcccgcaatggagaaacattagccacctcagaattatcaatcaaattc

SEQ ID NO: 25 - DNA, 101 bp, Cannabis sativa
  source 1..101: mol_type = genomic DNA
ataagctgtgatgcaattgcccgcaatggagaaacattagccacctcagaattatcaatcaaattcatttcacctttaagaagtggagataaatttgtggt

SEQ ID NO: 26 - DNA, 101 bp, Cannabis sativa
  source 1..101: mol_type = genomic DNA
cacctttaagaagtggagataaatttgtggtgaaggtaagggtctctagattttcagctgctcgtgcatactttgagcacaaaattttcaagcttcccaat

SEQ ID NO: 27 - DNA, 101 bp, Cannabis sativa
  source 1..101: mol_type = genomic DNA
agataaatttgtggtgaaggtaagggtctctagattttcagctgctcgtgcatactttgagcacaaaattttcaagcttcccaattatgagcctattttgg

SEQ ID NO: 28 - DNA, 101 bp, Cannabis sativa
  source 1..101: mol_type = genomic DNA
taagggtctctagattttcagctgctcgtgcatactttgagcacaaaattttcaagcttcccaattatgagcctattttggaaacaaaagccacaggaatt

SEQ ID NO: 29 - DNA, 179 bp, Cannabis sativa
  source 1..179: mol_type = genomic DNA
ttcccaagggcattatggtaatctcacttctttcttccctaaaacgctgaacatagagagagagagagaggagagagagagagagttgaattatcttctccgacgttggtattgctcatcatcagatcaacaacttcgttttctaccgtttcatgttttaggttttccttttaaaaagt

SEQ ID NO: 30 - DNA, 512 bp, Cannabis sativa
  source 1..512: mol_type = genomic DNA
ttcccaagggcattatggtaatctcacttctttcttccctaaaacgctgaacatagagagagagagagagagagagagagagagagagagagttgaattatcttctccgacgttggtattgctcatcatcagatcaacaacttcgttttctaccgtttcatgttttaggttttccttttaaaaagtggagctccattgtttgactattgcggaaaaagtgggtaattttatgggttcccatacgtgagaggactgatgaggaggcgtgttattgtgttaagctgccaattgcttcgcatgatgatgttgtgactctatgacaaaaaagccaccctcttttggatttttatctacttgttttggggctttgggttaggtgaaaaaaggtgatttttttatgtttaaaacaagtacttggccaagccttgagaacgtgggttgttgttggattagactcacagccgaaataagttcacaaagctccaatttctctgttgacttacgaggccaac

SEQ ID NO: 31 - DNA, 101 bp, Cannabis sativa
  source 1..101: mol_type = genomic DNA
cttctttcttccctaaaacgctgaacatagagagagagagagagagagagagagagagagagagagttgaattatcttctccgacgttggtattgctcatc

SEQ ID NO: 32 - DNA, 20 bp, Synthetic construct
  source 1..20: mol_type = other DNA
aaattcaggtccagccacaa

SEQ ID NO: 33 - DNA, 19 bp, Synthetic construct
  source 1..19: mol_type = other DNA
cacattttcggcagtggtc

SEQ ID NO: 34 - DNA, 20 bp, Synthetic construct
  source 1..20: mol_type = other DNA
ttccactagcccagcaactt

SEQ ID NO: 35 - DNA, 21 bp, Synthetic construct
  source 1..21: mol_type = other DNA
tttccagctcattctttgacc

SEQ ID NO: 36 - DNA, 20 bp, Synthetic construct
  source 1..20: mol_type = other DNA
ccctccatcaatggtgagaa

SEQ ID NO: 37 - DNA, 20 bp, Synthetic construct
  source 1..20: mol_type = other DNA
tctcccttcaatgcaaatcc

SEQ ID NO: 38 - DNA, 20 bp, Synthetic construct
  source 1..20: mol_type = other DNA
tcctgcttttgcagcactac

SEQ ID NO: 39 - DNA, 27 bp, Synthetic construct
  source 1..27: mol_type = other DNA
ctctagaggtattagaaaagctactgc

SEQ ID NO: 40 - DNA, 20 bp, Synthetic construct
  source 1..20: mol_type = other DNA
tgccaaaaattccactagcc

SEQ ID NO: 41 - DNA, 21 bp, Synthetic construct
  source 1..21: mol_type = other DNA
tcatgagtgatggaatggtga

SEQ ID NO: 42 - DNA, 20 bp, Synthetic construct
  source 1..20: mol_type = other DNA
gaccgaatcgaaggagatga

SEQ ID NO: 43 - DNA, 23 bp, Synthetic construct
  source 1..23: mol_type = other DNA
gtgggagattgttagcgtttatg

SEQ ID NO: 44 - DNA, 21 bp, Synthetic construct
  source 1..21: mol_type = other DNA
gcctcagttggatgaaatgaa

SEQ ID NO: 45 - DNA, 101 bp, Cannabis sativa
  source 1..101: mol_type = genomic DNA
tagggagaaagcattattattataattcaaacatgttgcagacgatttatccattgccattagtaaatgtggcgacgtgtctccatcgacaccgtttatct

SEQ ID NO: 46 - DNA, 285 bp, Cannabis sativa
  source 1..285: mol_type = genomic DNA
atgttgcagacgatttatccattgccattagtaaatgtggcgacgtgtctccatcgacaccgtttatcttcatcatcagtgactaatttcacatgtgatggtggtagtactactcttaatgtgaaattgaatagtagtaaagatgatgatgatgataattataataaccaaaaaattgtgaagagagatgggcataataatagaagaagaatgaagatgaatgatgaatattttgatattgagctcgaagttcgtgattatgagctcgatcagtttggtgtcgta

SEQ ID NO: 47 - protein, 95 aa, Cannabis sativa
MLQTIYPLPLVNVATCLHRHRLSSSSVTNFTCDGGSTTLNVKLNSSKDDDDDNYNNQKIVKRDGHNNRRRMKMNDEYFDIELEVRDYELDQFGVV

SEQ ID NO: 48 - DNA, 101 bp, Cannabis sativa
  source 1..101: mol_type = genomic DNA
ccacaggaatttggttggataagaagaataacccaattcgtgtaccgcaagagataaagaacacgtttattatttgattaatattatttactcttttttat

SEQ ID NO: 49 - DNA, 101 bp, Cannabis sativa
  source 1..101: mol_type = genomic DNA
ggttggataagaagaataacccaattcgtgtaccgcaagagataaagaacacgtttattatttgattaatattatttactcttttttatactaaataaagg

SEQ ID NO: 50 - DNA, 813 bp, Cannabis sativa
  source 1..813: mol_type = genomic DNA
cattttcggcagtggtcccccatttccggtggccaatcactccgccactccatctcccttcagtgcaaatccaagagttcctccggtggtgttgttaaagctcagggagcaagtcttgaagaaacaagcgccggagtaggccaaaatgtggaggctccggtggtcattgtgactggtgcctctagaggtattggaaaagctactgcattggctttgggaaaagctggttgtaaggtccttgttaattatgctagatcatcaaaggaggctgaggaagtttccaaagagatcgaagcatctggtggccaagctattacttttggaggagatgtttcaaaagaagctgatgttgaattaatgatcaaaacagcagttgatgcttggggaactattgatgtattagtaaataatgcaggaatcacaagggacaacttaattatgagaatgaagaagtcccagtggcaggatgtcattgatttgaatcttactggtgtatttctatgtacacaggctgcagccaaaattatgatgaagaagaaaaagggaaggataatcaatgtagcatcagtcttcggtctagtcggcaatgccgggcaagccaattatagtgctgcaaaagcaggagtaattggcttcacaaaagctattgctaaggagtattccagcagaaacatcactgttaatgctattgctccaggctttattgcatctgacatgactgccaagctaggagatgatattgagaagaaaaacttggagggcatccccttaggaagatatggtcaaccagaagaagttgctgggctagtggaa

SEQ ID NO: 51 - DNA, 813 bp, Cannabis sativa
  source 1..813: mol_type = genomic DNA
cattttcggcagtggtcccccatttccggtggccaatcactccgccactccatctcccttcagtgcaaatccaagagttcctccggtggtgttgttaaagctcagggagcaagtcttgaagaaacaagcgccggagtaggccaaaatgtggaggctccggtggtcattgtgactggtgcctctagaggtattggaaaagctactgcattggctttgggaaaagctggttgtaaggtccttgttaattatgctagatcatcaaaggaggctgaggaagtttccaaagagatcgaagcatctggtggccaagctattacttttggaggagatgtttcaaaagaagctgatgttgaattaatgatcaaaacagcagttgatgcttggggaactgttgatgtattagtaaataatgcaggaatcacaagggacaacttaattatgagaatgaagaaatcccagtggcaggatgtcattgatttgaatcttactggtgtatttctatgtacacaggctgcagccaaaattatgatgaagaagaaaaagggaaggataatcaatgtagcatcagtcttcggtctagtcggcaatgccgggcaagccaattatagtgctgcaaaagcaggagtaattggcttcacaaaagctattgctaaggagtattccagcagaaacatcactgttaatgctattgctccaggctttattgcatctgacatgactgccaagctaggagatgatattgagaagaaaaacttggagggcatccccttaggaagatatggtcaaccagaagaagttgctgggctagtggaa

SEQ ID NO: 52 - DNA, 570 bp, Cannabis sativa
  source 1..570: mol_type = genomic DNA
tcccttcaatgcaaatccaagagttcctccggtggtgttgtgaaagctcaggttgaaactcttgaagaaacaagcgccggagtaggccaaaatgtggaggctccggtggtcattgtgactggtgcccctagaggtattggaaaagctactgcattggctttgggaaaagctggttgtaaggtccttgttaattatgctagatcatcaaaggaggctgaggaagtttccaaagaaattgaagcatctggtggccaagctattacttttggaggagatatttcaaaagaagctgatgttgaattaatgaccaaaatagcagttgatgcttggggaactgttgatgtattagtaaataatgcaggaatcactagggacaacttaatgatgagaatgaagaaatcccaatggcaggatgtcattgatttgaatcttactggtgtatttctatgtacacaggctgcagcgaaaattatgatgaagaagaaaaggggaaggataatcaatgtagcatcaatcttcggtttagtcggcaatgctgggcaagccaattgtagtgctgcaaaagcagga

SEQ ID NO: 53 - DNA, 813 bp, Cannabis sativa
  source 1..813: mol_type = genomic DNA
cattttcggcagtggtcccccatttccagtggccaatcactccgtcactccatctcccttcaatgcaaatccaagagttcctccggtggtgttgtgaaagctcaggttgaaactcttgaagaaacaagcgccggagtaggccaaaatgtggaggctccggtggtcattgtgactggtgcctctagaggtattggaaaagctactgcattggctttgggaaaggctggttgtaaggtccttgttaattatgctagatcatcaaaggaggctgaggaagtttccaaagaaattgaagcatctggtggccaagctattacttttggaggagatatttcaaaagaagctgatgttgaattaatgaccaaaatagcagttgatgcttggggaactgttgatgtattagtaaataatgcaggaatcactagggacaacttaatgatgagaatgaagaaatcccaatggcaggatgtcattgatttgaatcttactggtgtatctctatgtacacaggctgcagcgaaaattatgatgaagaagaaaaagggaaggataatcaatgtagcatcaatcttcggtttagtcggcaatgctgggcaagccaattatagtgctgcaaaagcaggagtaattggcttcacaaaagctgttgctaaggagtattccagcagaaacatcactgttaatgctattgctccaggctttattgcatctgacatgactgccaagctaggagatgatattgagaagaaaaacttggagggcatccccttaggaagatatggtcaaccagaagaagttgctgggctagtggaa

SEQ ID NO: 54 - DNA, 813 bp, Cannabis sativa
  source 1..813: mol_type = genomic DNA
cattttcggcagtggtcccccatttccagtggccaatcactccgtcactccatctcccttcaatgcaaatccaagagttcctccggtggtgttgtgaaagctcaggttgaaactcttgaagaaacaagcgccggagtaggccaaaatgtggaggctccggtggtcattgtgactggtgcctctagaggtattggaaaagctactgcattggctttgggaaaagctggttgtaaggtccttgttaattatgctagatcatcaaaggaggctgaggaagtttccaaagaaattggagcatctggtggccaagctattacttttggaggagatatttcaaaagaagctgatgttgaattaatgaccaaaatagcagttgatgcttggggaactgttgatgtattagtaaataatgcaggaatcactagggacaacttaatgatgagaatgaagaaatcccaatggcaggatgtcattgatttgaatcttactggtgtatttctatgtacacaggctgcagcgaaaattatgatgaagaagaaaaagggaaggataatcaatgtagcatcaatcttcggtttagtcggcaatgctgggcaagccaattatagtgctgcaaaagcaggagtaattggcttcacaaaagctgttgctaaggagtattccagcagaaacatcactgttaatgctattgctccaggctttattgcatctgacatgactgccaagctaggagatgatattgagaagaagaacttggagggcatccccttaggaagatatggtcaaccagaagaagttgctgggctagtggaa

SEQ ID NO: 55 - DNA, 813 bp, Cannabis sativa
  source 1..813: mol_type = genomic DNA
cattttcggcagtggtcccccatttccagtggccgatcactccgtcactccatctcccttcaatgcaaatccaagagttcctccggtggtgttgtgaaagctcaggttgaaactcttgaagaaacaaacgccggagtaggccaaaatgtggaggctccggtggtcattgtgactggtgcctctagaggtattggaaaagctactgcattggctttgggaaaagctggttgtaaggtccttgttaattatgctagatcatcaaaggaggctgaggaagtttccaaagaaattgaagcatctggtggccaagctattacttttggaggagatatttcaaaagaagctgatgttgaattaatgaccaaaatagcagttgatgcttggggaactgttgatgtattagtaaataatgcaggaatcactagggacaacttaatgatgagaatgaagaaatcccaatggcaggatgtcattgatttgaatcttactggtgtatttctatgtacacaggctgcagcgaaaattatgatgaagaagaaaaagggaaggataatcaatgtagcatcaatcttcggtttagtcggcaatgctgggcaagccaattatagtgctgcaaaagcaggagtaattggcttcacaaaagctgttgctaaggagtattccagcagaaacatcactgttaatgctattgctccaggctttattgcatctgacatgactgccaagctaggagatgatattgagaagaaaaacttggagggcatccccttaggaagatatggtcaaccagaagaagttgctgggctagtggaa

SEQ ID NO: 56 - DNA, 813 bp, Cannabis sativa
  source 1..813: mol_type = genomic DNA
cattttcggcagtggtcccccatttccagtggccaatcactccgtcactccatctcccttcaatgcaaatccaagagttcctccggtggtgttgtgaaagctcaggttgaaactcttgaagaaacaagcgccggagtaggccaaaatgtggaggctccggtggtcattgtgactggtgcctctagaggtattggaaaagctactgcattggctttgggaaaagctggttgtaaggtccttgttaattatgctagatcatcaaaggaggctgaggaagtttccgaagaaattgaagcatctggtggccaagctattacttttggaggagatatttcaaaagaagctgatgttgaattaatgaccaaaatagcagttgatgcttggggaactgttgatgtattagtaaataatgcaggaatcactagggacaacttaatgatgagaatgaagaaatcccaatggcaggatgtcattgatttgaatcttactggtgtatttctatgtacacaggctgcagcgaaaattatgatgaagaagaaaaagggaaggataatcaatgtagcatcaatcttcggtttagtcggcaatgctgggcaagccaattatagtgctgcaaaagcaggagtaattggcttcacaaaagctgttgctaaggagtattccagcagaaacatcactgttaatgctattgctccaggctttattgcatctgacatgactgccaagctaggagatgatattgagaagaaaaacttggagggcatccccttaggaagatatggtcaaccagaagaagttgctgggctagtggaa

SEQ ID NO: 57 - DNA, 813 bp, Cannabis sativa
  source 1..813: mol_type = genomic DNA
cattttcggcagtggtcccccatttccagtggccaatcactccgtcactccatctcccttcaatgcaaatccaagagttcctccggtggtgttgtgaaagctcaggttgaaactcttgaagaaacaagcgccggagtaggccaaaatgtggaggctccggtggtcattgtgactggtgcctctagaggtattggaaaagctactgcattggctttgggaaaagctggttgtaaggtccttgttaattatgctagatcatcaaaggaggctgaggaagtttccaaagaaattgaagcatctggtggccaagctattacttttggaggagatatttcaaaagaagctgatgttgaattaatgaccaaaatagcagttgatgcttggggaactgttgatgtattagtaaataatgcaggaatcactagggacaacttaatgatgagaatgaagaaatcccaatggcaggatgtcattgatttgaatcttactggtgtatttctatgtacacaggctgcagcgaaaattatgatgaagaagaaaaagggaaggataatcaatgtagcatcaatctccggtttagtcggcaatgctgggcaagccaattatagtgctgcaaaagcaggagtaattggcttcacaaaagctgttgctaaggagtattccagcagaaacatcactgttaatgctattgctccaggctttattgcatctgacatgactgccaagctaggagatgatattgagaagaaaaacttggagggcatccccttaggaagatatggtcaaccagaagaagttgctgggctagtggaa

SEQ ID NO: 58 - DNA, 813 bp, Cannabis sativa
  source 1..813: mol_type = genomic DNA
cattttcggcagtggtcccccatttccagtggccaatcactccgtcactccatctcccttcaatgcaaatccaagagttcctccggtggtgttgtgaaagctcaggttgaaactcttgaagaaacaagcgccggagtaggccaaaatgtggaggctccggtggtcattgtgactggtgcctctagaggtattggaaaagctactgcattggctttgggaaaagctggttgtaaggtccttgttaattatgctagatcatcaaaggaggctgaggaagtttccaaagggattgaagcatctggtggccaagctattacttttggaggagatatttcaaaagaagctgatgttgaattaatgaccaaaatagcagttgatgcttggggaactgttgatgtattagtaaataatgcaggaatcactagggacaacttaatgatgagaatgaagaaatcccaatggcaggatgtcattgatttgaatcttactggtgtatttttatgtacacaggctgcagccaaaattatgatgaagaagaaaaagggaaggataatcaatgtagcatcaatcttcggtttagtcggcaatgctgggcaagccaattatagtgctgcaaaagcaggagtaattggcttcacaaaagctgttgctaaggagtattccagcagaaacatcactgttaatgctattgctccaggctttattgcatctgacatgactgccaagctaggagatgatattgagaagaaaaacttggagggcatccccttaggaagatatggtcaaccagaagaagttgctgggctagtggaa

SEQ ID NO: 59 - DNA, 813 bp, Cannabis sativa
  source 1..813: mol_type = genomic DNA
cattttcggcagtggtcccccatttccagtggccaatcactccgtcactccatctcccttcaatgcaaatccaagagttcctccggtggtgttgtgaaagctcaggttgaaactcttgaagaaacaagcgccggagtaggccaaaatgtggaggctccggtggtcattgtgactggtgcctctagaggtattggaaaagctactgcattggctttgggaaaagctggttgtaaggtccttgttaattatgctagatcatcaaaggaggctgaggaagtttccaaagagatcgaagcatctggtggccaagctattacttttggaggagatatttcaaaagaagctgatgttgaattaatgaccaaaatagcagttgatgcttggggaactgttgatgtattagtaaataatgcaggaatcactagggacaacttaatgatgagaatgaagaaatcccaatggcaggatgtcattgatttgaatcttactggtgtatttttatgtacacaggctgcagccaaaattatgatgaggaagaaaaagggaaggataatcaatgtagcatcaatcttcggtttagtcggcaatgctgggcaagccaattatagtgctgcaaaagcaggagtaattggcttcacaaaagctgttgctaaggagtattccagcagaaacatcactgttaatgctattgctccaggctttattgcatctgacatgactgccaagctaggagatgatattgagaagaaaaacttggagggcatccccttaggaagatatggtcaaccagaagaagttgctgggctagtggaa

SEQ ID NO: 60 - DNA, 813 bp, Cannabis sativa
  source 1..813: mol_type = genomic DNA
cattttcggcagtggtcccccatttccagtggccaatcactccgtcactccatctcccttcaatgcaaatccaagagttcctccggtggtgttgtgaaagctcaggttgaaactcttgaagaaacaagcgccggagtaggccaaaatatggaggctccggtggtcattgtgactggtgcctctagaggtattggaaaagctactgcattggctctgggaaaagctggttgtaaggtccttgttaattatgctagatcatcaaaggaggctgaggaagtttccaaagagattgaagcatctggtggccaagctattacttttggaggagatatttcaaaagaagctgatgttgaattaatgaccaaaatagcagttgatgcttggggaactgttgatgtattagtaaataatgcaggaatcactagggacaacttaatgatgagaatgaagaaatcccaatggcaggatgtcattgatttgaatcttactggtgtatttttatgtacacaggctgcagccaaaattatgatgaagaagaaaaagggaaggataatcaatgtagcatcaatcttcggtttagtcggcaatgctgggcaagccaattatagtgctgcaaaagcaggagtaattggcttcacaaaagctgttgctaaggagtattccagcagaaacatcactgttaatgctattgctccaggctttattgcatctgacatgactgccaagctaggagatgatattgagaagaaaaacttggagggcatccccttaggaagatatggtcaaccagaagaagttgctgggctagtggaa

SEQ ID NO: 61 - DNA, 813 bp, Cannabis sativa
  source 1..813: mol_type = genomic DNA
cattttcggcagtggtcccccatttccagtggccaatcactccgtcactccatctcccttcaatgcaaatccaagagttcctccggtggtgttgtgaaagctcaggttgaaactcttgaagaaacaagcgccggagtaggccaaaatgtggaggctccggtggtcattgtgactggtgcctctagaggtattggaaaagctactgcattggctttgggaaaagctggttgtaaggtccttgttaattatgctagatcatcaaaggaggctgaggaagtttccaaagagattgaagcatctggtggccaagctattacttttggaggagatattccaaaagaagctgatgttgaattaatgaccaaaatagcagttgatgcttggggaactgttgatgtattagtaaataatgcaggaatcactagggacaacttaatgatgggaatgaagaaatcccaatggcaggatgtcattgatttgaatcttactggtgtatttttatgtacacaggctgcagccaaaattatgatgaagaagaaaaagggaaggataatcaatgtagcatcaatcttcggtttagtcggcaatgctgggcaagccaattatagtgctgcaaaagcaggagtaattggcttcacaaaagctgttgctaaggagtattccagcagaaacatcactgttaatgctattgctccaggctttattgcatctgacatgactgccaagctaggagatgatattgagaagaaaaacttggagggcatccccttaggaagatatggtcaaccagaagaagttgctgggctagtggaa

SEQ ID NO: 62 - DNA, 813 bp, Cannabis sativa
  source 1..813: mol_type = genomic DNA
cattttcggcagtggtcccccatttccagtggccaatcactccgtcactccatctcccttcaatgcaaatccaagagttcctccggtggtgttgtgaaagctcaggttgaaactcttgaagaaacaagcgccggagtaggccaaaatgtggaggctccggtggtcattgtgactggtgcctctagaggtattggaaaagctactgcattggctttgggaaaagctggttgtaaggtccttgttaattatgctagatcatcaaaggaggctgaggaagtttccaaagagattgaagcatctggtggccaagctattacttttggaggagatatttcaaaagaagctgatgttgaattaatgaccaaaatagcagttgatgcttggggaactgttgatgtattagtaaataatgcaggaatcactagggacaacttaatgatgagaatgaagaaatcccaatggcaggatgtcattgatttgaatcttactggtgtatttttatgtacacaggctgcagccaaaattatgatgaagaagaaaaagggaaggataatcaatgtagcatcaatcttcggtttagtcggcaatgctgggcaagccaattatagtgctgcaaaagcaggagtaattggcttcacaaaagctgttgctaaggagtattccagcagaagcatcactgttaatgctattgctccaggctttattgcatctgacatgactgccaagctaggagatgatattgagaagaaaaacttggagggcatccccttaggaagatatggtcaaccagaagaagttgctgggctagtggaa

SEQ ID NO: 63 - DNA, 813 bp, Cannabis sativa
  source 1..813: mol_type = genomic DNA
cattttcggcagtggtcccccatttccagtggccaatcactccgtcactccatctcccttcaatgcaaatccaagagttcctccggtggtgttgtgaaagctcaggttgaaactcttgaagaaacaagcgccggagtaggccaaaatgtggaggctccggtggtcattgtgactggtgcccctagaggtattggaaaagctactgcattggctttgggaaaagctggttgtaaggtccttgttaattatgctagatcatcaaaggaggctgaggaagtttccaaagagattgaagcatctggtggccaagctattacttttggaggagatatttcaaaagaagctgatgttgaattaatgaccaaaatagcagttgatgcttggggaactgttgatgtattagtaaataatgcaggaatcactagggacaacttaatgatgagaatgaagaaatcccaatggcaggatgtcattgatttgaatcttactggtgtatttttatgtacacaggctgcagccaaaattatgatgaagaagaaaaagggaaggataatcaatgtagcatcaatcttcggtttagtcggcaatgccgggcaagccaattatagtgctgcaaaagcaggagtaattggcttcacaaaagctgttgctaaggagtattccagcagaaacatcactgttaatgctattgctccaggctttattgcatctgacatgactgccaagctaggagatgatattgagaagaaaaacttggagggcatccccttaggaagatatggtcaaccagaagaagttgctgggctagtggaa

SEQ ID NO: 64 - DNA, 642 bp, Cannabis sativa
  source 1..642: mol_type = genomic DNA
tctagaggtattagaaaagctactgcattggctttgggaaaagctggttgtaaggtacttgttaattatgctagatcatcaaaggaggctgaggaagtttccaaagagattgaagcatctggtggccaagctattacttttggaggagatgtttcaaaagaagctgatgttgaattaatgatcaaaatagcagttgatgcttggggaactgttgatgtattagtaaataatgcaggaatcacaagggacaacttaattatgagaatgaagaaatcccagtggcaggatgtcattgatttgaatcttactggtgtatttctatgtacacaggctgcagccaaaattatgatgaagaagaaaaagggaaggataatcaatgtagcatcagtcttcggtctagtcggcaatgctgggcaagccaactacagtgctgcaaaagcaggagtaattggcttcacaaaagctattgctaaggagtattccagcagaaacatcactgttaatgctattgctccaggctttattgcatctgacatgactgccaagctaggagatgatattgagaagaaaaacttggagggcatccccttaggaagatatggtcaaccagaagaagttgctgggctagtggaatttttggca

SEQ ID NO: 65 - DNA, 642 bp, Cannabis sativa
  source 1..642: mol_type = genomic DNA
tctagaggtattagaaaagctactgcattggctttgggaaaagctggttgtaaggtacttgttaattatgctagatcatcaaaggaggctgaggaagtttccaaagagattgaagcatctggtggccaagctattacttttggaggagatgtttcaaaagaagctgatgttgaattaatgatcaaaatagcagttgatgcttggggaactgttgatgtattagtaaataatgcaggaatcacaagggacaacttaattatgagaatgaagaaatcccagtggcaggatgtcattgatttgaatcttactggtgtatttctatgtacacaggctgcagccaaaattatgatgaagaagaaaaagggaaggataatcaatgtagcatcagtcttcggtctagtcggcaatgctgggcaagccaattacagtgctgcaaaagcaggagtaattggcttcacaaaagctattgctaaggagtattccagcagaaacatcactgttaatgctattgctccaggctttattgcatctgacatgactgccaagctaggagatgatattgagaagaaaaacttggagggcatccccttaggaagatatggtcaaccagaagaaattgctgggctagtggaatttttggca

SEQ ID NO: 66 - protein, 271 aa, Cannabis sativa
HFRQWSPISGGQSLRHSISLQCKSKSSSGGVVKAQGASLEETSAGVGQNVEAPVVIVTGASRGIGKATALALGKAGCKVLVNYARSSKEAEEVSKEIEASGGQAITFGGDVSKEADVELMIKTAVDAWGTVDVLVNNAGITRDNLIMRMKKSQWQDVIDLNLTGVFLCTQAAAKIMMKKKKGRIINVASVFGLVGNAGQANYSAAKAGVIGFTKAIAKEYSSRNITVNAIAPGFIASDMTAKLGDDIEKKNLEGIPLGRYGQPEEVAGLVE

SEQ ID NO: 67 - protein, 271 aa, Cannabis sativa; identical to SEQ ID NO: 66

SEQ ID NO: 68 - protein, 190 aa, Cannabis sativa
SLQCKSKSSSGGVVKAQVETLEETSAGVGQNVEAPVVIVTGAPRGIGKATALALGKAGCKVLVNYARSSKEAEEVSKEIEASGGQAITFGGDISKEADVELMTKIAVDAWGTVDVLVNNAGITRDNLMMRMKKSQWQDVIDLNLTGVFLCTQAAAKIMMKKKRGRIINVASIFGLVGNAGQANCSAAKAG

SEQ ID NO: 69 - protein, 271 aa, Cannabis sativa
HFRQWSPISSGQSLRHSISLQCKSKSSSGGVVKAQVETLEETSAGVGQNVEAPVVIVTGASRGIGKATALALGKAGCKVLVNYARSSKEAEEVSKEIEASGGQAITFGGDISKEADVELMTKIAVDAWGTVDVLVNNAGITRDNLMMRMKKSQWQDVIDLNLTGVSLCTQAAAKIMMKKKKGRIINVASIFGLVGNAGQANYSAAKAGVIGFTKAVAKEYSSRNITVNAIAPGFIASDMTAKLGDDIEKKNLEGIPLGRYGQPEEVAGLVE

SEQ ID NO: 70 - protein, 271 aa, Cannabis sativa
HFRQWSPISSGQSLRHSISLQCKSKSSSGGVVKAQVETLEETSAGVGQNVEAPVVIVTGASRGIGKATALALGKAGCKVLVNYARSSKEAEEVSKEIGASGGQAITFGGDISKEADVELMTKIAVDAWGTVDVLVNNAGITRDNLMMRMKKSQWQDVIDLNLTGVFLCTQAAAKIMMKKKKGRIINVASIFGLVGNAGQANYSAAKAGVIGFTKAVAKEYSSRNITVNAIAPGFIASDMTAKLGDDIEKKNLEGIPLGRYGQPEEVAGLVE

SEQ ID NO: 71 - protein, 271 aa, Cannabis sativa
HFRQWSPISSGRSLRHSISLQCKSKSSSGGVVKAQVETLEETNAGVGQNVEAPVVIVTGASRGIGKATALALGKAGCKVLVNYARSSKEAEEVSKEIEASGGQAITFGGDISKEADVELMTKIAVDAWGTVDVLVNNAGITRDNLMMRMKKSQWQDVIDLNLTGVFLCTQAAAKIMMKKKKGRIINVASIFGLVGNAGQANYSAAKAGVIGFTKAVAKEYSSRNITVNAIAPGFIASDMTAKLGDDIEKKNLEGIPLGRYGQPEEVAGLVE

SEQ ID NO: 72 - protein, 271 aa, Cannabis sativa
HFRQWSPISSGQSLRHSISLQCKSKSSSGGVVKAQVETLEETSAGVGQNVEAPVVIVTGASRGIGKATALALGKAGCKVLVNYARSSKEAEEVSEEIEASGGQAITFGGDISKEADVELMTKIAVDAWGTVDVLVNNAGITRDNLMMRMKKSQWQDVIDLNLTGVFLCTQAAAKIMMKKKKGRIINVASIFGLVGNAGQANYSAAKAGVIGFTKAVAKEYSSRNITVNAIAPGFIASDMTAKLGDDIEKKNLEGIPLGRYGQPEEVAGLVE

SEQ ID NO: 73 - protein, 271 aa, Cannabis sativa
HFRQWSPISSGQSLRHSISLQCKSKSSSGGVVKAQVETLEETSAGVGQNVEAPVVIVTGASRGIGKATALALGKAGCKVLVNYARSSKEAEEVSKEIEASGGQAITFGGDISKEADVELMTKIAVDAWGTVDVLVNNAGITRDNLMMRMKKSQWQDVIDLNLTGVFLCTQAAAKIMMKKKKGRIINVASISGLVGNAGQANYSAAKAGVIGFTKAVAKEYSSRNITVNAIAPGFIASDMTAKLGDDIEKKNLEGIPLGRYGQPEEVAGLVE

SEQ ID NO: 74 - protein, 271 aa, Cannabis sativa
HFRQWSPISSGQSLRHSISLQCKSKSSSGGVVKAQVETLEETSAGVGQNVEAPVVIVTGASRGIGKATALALGKAGCKVLVNYARSSKEAEEVSKGIEASGGQAITFGGDISKEADVELMTKIAVDAWGTVDVLVNNAGITRDNLMMRMKKSQWQDVIDLNLTGVFLCTQAAAKIMMKKKKGRIINVASIFGLVGNAGQANYSAAKAGVIGFTKAVAKEYSSRNITVNAIAPGFIASDMTAKLGDDIEKKNLEGIPLGRYGQPEEVAGLVE

SEQ ID NO: 75 - protein, 271 aa, Cannabis sativa
HFRQWSPISSGQSLRHSISLQCKSKSSSGGVVKAQVETLEETSAGVGQNVEAPVVIVTGASRGIGKATALALGKAGCKVLVNYARSSKEAEEVSKEIEASGGQAITFGGDISKEADVELMTKIAVDAWGTVDVLVNNAGITRDNLMMRMKKSQWQDVIDLNLTGVFLCTQAAAKIMMRKKKGRIINVASIFGLVGNAGQANYSAAKAGVIGFTKAVAKEYSSRNITVNAIAPGFIASDMTAKLGDDIEKKNLEGIPLGRYGQPEEVAGLVE

SEQ ID NO: 76 - protein, 271 aa, Cannabis sativa
HFRQWSPISSGQSLRHSISLQCKSKSSSGGVVKAQVETLEETSAGVGQNMEAPVVIVTGASRGIGKATALALGKAGCKVLVNYARSSKEAEEVSKEIEASGGQAITFGGDISKEADVELMTKIAVDAWGTVDVLVNNAGITRDNLMMRMKKSQWQDVIDLNLTGVFLCTQAAAKIMMKKKKGRIINVASIFGLVGNAGQANYSAAKAGVIGFTKAVAKEYSSRNITVNAIAPGFIASDMTAKLGDDIEKKNLEGIPLGRYGQPEEVAGLVE

SEQ ID NO: 77 - protein, 271 aa, Cannabis sativa
HFRQWSPISSGQSLRHSISLQCKSKSSSGGVVKAQVETLEETSAGVGQNVEAPVVIVTGASRGIGKATALALGKAGCKVLVNYARSSKEAEEVSKEIEASGGQAITFGGDIPKEADVELMTKIAVDAWGTVDVLVNNAGITRDNLMMGMKKSQWQDVIDLNLTGVFLCTQAAAKIMMKKKKGRIINVASIFGLVGNAGQANYSAAKAGVIGFTKAVAKEYSSRNITVNAIAPGFIASDMTAKLGDDIEKKNLEGIPLGRYGQPEEVAGLVE

SEQ ID NO: 78 - protein, 271 aa, Cannabis sativa
HFRQWSPISSGQSLRHSISLQCKSKSSSGGVVKAQVETLEETSAGVGQNVEAPVVIVTGASRGIGKATALALGKAGCKVLVNYARSSKEAEEVSKEIEASGGQAITFGGDISKEADVELMTKIAVDAWGTVDVLVNNAGITRDNLMMRMKKSQWQDVIDLNLTGVFLCTQAAAKIMMKKKKGRIINVASIFGLVGNAGQANYSAAKAGVIGFTKAVAKEYSSRSITVNAIAPGFIASDMTAKLGDDIEKKNLEGIPLGRYGQPEEVAGLVE

SEQ ID NO: 79 - protein, 271 aa, Cannabis sativa
HFRQWSPISSGQSLRHSISLQCKSKSSSGGVVKAQVETLEETSAGVGQNVEAPVVIVTGAPRGIGKATALALGKAGCKVLVNYARSSKEAEEVSKEIEASGGQAITFGGDISKEADVELMTKIAVDAWGTVDVLVNNAGITRDNLMMRMKKSQWQDVIDLNLTGVFLCTQAAAKIMMKKKKGRIINVASIFGLVGNAGQANYSAAKAGVIGFTKAVAKEYSSRNITVNAIAPGFIASDMTAKLGDDIEKKNLEGIPLGRYGQPEEVAGLVE

SEQ ID NO: 80 - protein, 214 aa, Cannabis sativa
SRGIRKATALALGKAGCKVLVNYARSSKEAEEVSKEIEASGGQAITFGGDVSKEADVELMIKIAVDAWGTVDVLVNNAGITRDNLIMRMKKSQWQDVIDLNLTGVFLCTQAAAKIMMKKKKGRIINVASVFGLVGNAGQANYSAAKAGVIGFTKAIAKEYSSRNITVNAIAPGFIASDMTAKLGDDIEKKNLEGIPLGRYGQPEEVAGLVEFLA

SEQ ID NO: 81 - protein, 214 aa, Cannabis sativa
SRGIRKATALALGKAGCKVLVNYARSSKEAEEVSKEIEASGGQAITFGGDVSKEADVELMIKIAVDAWGTVDVLVNNAGITRDNLIMRMKKSQWQDVIDLNLTGVFLCTQAAAKIMMKKKKGRIINVASVFGLVGNAGQANYSAAKAGVIGFTKAIAKEYSSRNITVNAIAPGFIASDMTAKLGDDIEKKNLEGIPLGRYGQPEEIAGLVEFLA

SEQ ID NO: 82 - DNA, 101 bp, Cannabis sativa
  source 1..101: mol_type = genomic DNA
tgaccggaaagccccacattttcggcagtggtcccccatttccggtggccaatcactccgccactccatctcccttcagtgcaaatccaagagttcctccg

SEQ ID NO: 83 - DNA, 101 bp, Cannabis sativa
  source 1..101: mol_type = genomic DNA
ccggagtaggccaaaatgtggaggctccggtggtcattgtgactggtgcctctagaggtattggaaaagctactgcattggctttgggaaaagctggttgt

SEQ ID NO: 84 - DNA, 101 bp, Cannabis sativa
  source 1..101: mol_type = genomic DNA
aggtccttgttaattatgctagatcatcaaaggaggctgaggaagtttccaaagaggtgagttccatcaagttctattcatgtttgtgttatccctttcaa

SEQ ID NO: 85 - DNA, 101 bp, Cannabis sativa
  source 1..101: mol_type = genomic DNA
tctttatgtagttcaaccaccatcaaaaataattttgactgagttcttacagatcgaagcatctggtggccaagctattacttttggaggagatgtttcaa

SEQ ID NO: 86 - DNA, 101 bp, Cannabis sativa
  source 1..101: mol_type = genomic DNA
tgtagttcaaccaccatcaaaaataattttgactgagttcttacagatcgaagcatctggtggccaagctattacttttggaggagatgtttcaaaagaag

SEQ ID NO: 87 - DNA, 101 bp, Cannabis sativa
  source 1..101: mol_type = genomic DNA
tacagatcgaagcatctggtggccaagctattacttttggaggagatgtttcaaaagaagctgatgttgaattaatgatcaaaacagtgagaaaatgttct

SEQ ID NO: 88 - DNA, 101 bp, Cannabis sativa
  source 1..101: mol_type = genomic DNA
ttgtaatattccatctcataaacctgcaggcagttgatgcttggggaactattgatgtattagtaaataatgcaggttgtttgaaactttcattttcttat

SEQ ID NO: 89 - DNA, 101 bp, Cannabis sativa
  source 1..101: mol_type = genomic DNA
ctgatgctttttcggtgttttcaggaatcacaagggacaacttaattatgagaatgaagaaatcccagtggcaggatgtcattgatttgaatcttactggt

SEQ ID NO: 90 - DNA, 101 bp, Cannabis sativa
  source 1..101: mol_type = genomic DNA
gaagaaatcccagtggcaggatgtcattgatttgaatcttactggtgtatttctatgtacacaggtgtgcatctaaagctttcacttcttaaggtccttca

SEQ ID NO: 91 - DNA, 101 bp, Cannabis sativa
  source 1..101: mol_type = genomic DNA
gaaactaaaattaattctttatttgcaggctgcagccaaaattatgatgaagaagaaaaaggtatatacaccgttttatctctccatttacagtcaaaact

SEQ ID NO: 92 - DNA, 101 bp, Cannabis sativa
  source 1..101: mol_type = genomic DNA
attaattctttatttgcaggctgcagccaaaattatgatgaagaagaaaaaggtatatacaccgttttatctctccatttacagtcaaaactatttttata

SEQ ID NO: 93 - DNA, 101 bp, Cannabis sativa
  source 1..101: mol_type = genomic DNA
ttagtccccaaataataattttgtagggaaggataatcaatgtagcatcagtcttcggtctagtcggcaatgccgggcaagccaattatagtgctgcaaaa

SEQ ID NO: 94 - DNA, 101 bp, Cannabis sativa
  source 1..101: mol_type = genomic DNA
tccccaaataataattttgtagggaaggataatcaatgtagcatcagtcttcggtctagtcggcaatgccgggcaagccaattatagtgctgcaaaagcag

SEQ ID NO: 95 - DNA, 101 bp, Cannabis sativa
  source 1..101: mol_type = genomic DNA
aggagtaattggcttcacaaaagctattgctaaggagtattccagcagaaacatcactgtgagttattccaaagttttttttttcaaataattagatattt

SEQ ID NO: 96 - DNA, 101 bp, Cannabis sativa
  source 1..101: mol_type = genomic DNA
attagtgtgatttctattgttcttcaggaagatatggtcaaccagaagaagttgctgggctagtggaatttttggcacttggtcctgctgcaagttacata

SEQ ID NO: 97 - DNA, 607 bp, Cannabis sativa
  source 1..607: mol_type = genomic DNA
atctggggagagagagtggaaatccatacatggattggagcatcagggaacaatggagtgcaaagagattggctaattcaaaatcaagacaccggccatattttagttcgtggaactagtacatgggtgatgatgaatcgagaaacaaggcgactctcgaagattccagaagaggtgagggatgagatttcgccttggttcatagagaagaaagctatcaaagaagatgtcattgacaaaattgtcaaattggatgataaagctaagtacatgaacaccaacttgaagnnnnnnnnnnnnnnnnnnnnnnnnnnnnnnnnnnnnnnnnnnnnnnnnnnnnnnnnnnnnnnnnnnnnnntcataccggatgccatttcagagtcttaccaattgtctaaaataaccctagaatacaaaagggaatgtgggagtactagggacatagtccaattcctttgtcaacctaatgaagatggaagagggttacttaaacatggagtaaatcaagataataatcataacaatctactaaatactttggctattgaatttctcaaaaacaatggattcacggggtcctttgagatagggccatttaggtacactc

SEQ ID NO: 98 - DNA, 1176 bp, Cannabis sativa
  source 1..1176: mol_type = genomic DNA
atgtctgctttgtcatttccaccattcaatgccataagatgttgttctaatagggacaatgatcaaaacccaagtgttcaaaagtttaatagcattaattttaatggaagttcaagctctcaaactgatcataaacttagcctaaaaattggtcctttaccaagctccttttctttgacttttgtcactaaaaaagaatccttcaatgatgagaaaattcaagagaataccccaacaaagaagcatgtagttgacccttttcgccaagagctcataattgaaggaggagtagggtacagacagactgttgttattagatcttacgaagaagtagcattaaatcatgtagggatgtcaggattcatgagtgatggaatggtgagaaacaatctcatatgggttgtttcaagaatgcaagttcaaattgaacaatatccaatctggggagagagagtggaaatcgatacatggattggagcatcagggaacaatggagtgcaaagagattggctaattcgaaatcaagacaccggccatattttagttcgtggaactagtacatgggtgatgatgaatcgagaaacaaggggactctcgaagatgcaagaagaggtgagggaggagatttcgccttggttcatagagaaaaaagctatcaaagaagatgttattgacaaaattgtcaaattggatgataaagctaagtacatgaacaccaacttaaagcccaagagaagtgatttggacatgaaccaccatgtgaacaatgtcaagtatgtaagatggatgctcgagatcataccggatgccatttcagagtcttaccaattgtctaaaataaccctagaatacaaaagggaatgtgggagtactagggacatagtccaattcctttgtcaacctaatgaagatggaagagggttacttaaacatggagtaaatcaagataataatcataacaatctactaaatactttggctattgaatttctcaaaaacaatggattcacggggtcctttgagatagggccatttaggtacactcatcttccccaaatcgatcatatagagccttctaagtgtactcatctccttcgattcggtcaacttctgcttcaaattctcttcttctttgatcatgttcttcgccacttcttcaatggagtttggtattag

SEQ ID NO: 99 - protein, 201 aa, Cannabis sativa
WGERVEIHTWIGASGNNGVQRDWLIQNQDTGHILVRGTSTWVMMNRETRRLSKIPEEVRDEISPWFIEKKAIKEDVIDKIVKLDDKAKYMNTNLKXXXXXXXXXXXXXXXXXXXXXXXXIPDAISESYQLSKITLEYKRECGSTRDIVQFLCQPNEDGRGLLKHGVNQDNNHNNLLNTLAIEFLKNNGFTGSFEIGPFRYT

SEQ ID NO: 100 - protein, 391 aa, Cannabis sativa
MSALSFPPFNAIRCCSNRDNDQNPSVQKFNSINFNGSSSSQTDHKLSLKIGPLPSSFSLTFVTKKESFNDEKIQENTPTKKHVVDPFRQELIIEGGVGYRQTVVIRSYEEVALNHVGMSGFMSDGMVRNNLIWVVSRMQVQIEQYPIWGERVEIDTWIGASGNNGVQRDWLIRNQDTGHILVRGTSTWVMMNRETRGLSKMQEEVREEISPWFIEKKAIKEDVIDKIVKLDDKAKYMNTNLKPKRSDLDMNHHVNNVKYVRWMLEIIPDAISESYQLSKITLEYKRECGSTRDIVQFLCQPNEDGRGLLKHGVNQDNNHNNLLNTLAIEFLKNNGFTGSFEIGPFRYTHLPQIDHIEPSKCTHLLRFGQLLLQILFFFDHVLRHFFNGVWY

SEQ ID NO: 101 - DNA, 101 bp, Cannabis sativa
  source 1..101: mol_type = genomic DNA
acttttaacacatttatgtataattccttaggggagagagagtggaaatcgatacatggattggagcatcagggaacaatggagtgcaaagagattggcta

SEQ ID NO: 102 - DNA, 101 bp, Cannabis sativa
  source 1..101: mol_type = genomic DNA
atggattggagcatcagggaacaatggagtgcaaagagattggctaattcgaaatcaagacaccggccatattttagttcgtggaactaggtaagaatatc

SEQ ID NO: 103 - DNA, 101 bp, Cannabis sativa
  source 1..101: mol_type = genomic DNA
ttttagttcgtggaactagtacatgggtgatgatgaatcgagaaacaaggggactctcgaagatgcaagaagaggtgagggaggagatttcgccttggttc

SEQ ID NO: 104 - DNA, 101 bp, Cannabis sativa
  source 1..101: mol_type = genomic DNA
aacagtacatgggtgatgatgaatcgagaaacaaggggactctcgaagatgcaagaagaggtgagggaggagatttcgccttggttcatagagaaaaaagc

SEQ ID NO: 105 - DNA, 101 bp, Cannabis sativa
  source 1..101: mol_type = genomic DNA
cagtacatgggtgatgatgaatcgagaaacaaggggactctcgaagatgcaagaagaggtgagggaggagatttcgccttggttcatagagaaaaaagcta

SEQ ID NO: 106 - DNA, 101 bp, Cannabis sativa
  source 1..101: mol_type = genomic DNA
atgaatcgagaaacaaggggactctcgaagatgcaagaagaggtgagggaggagatttcgccttggttcatagagaaaaaagctatcaaagaagatgttat

SEQ ID NO: 107 - DNA, 895 bp, Cannabis sativa
  source 1..895: mol_type = genomic DNA
acgattattagttatgttaatgcaaaccctaaagtttcttgctttttcatatttggagattctttggctgataatggcaacaacaataatctcaacactttggctaaagccaattataagccttatgggattgatttcattgattctcaaccaacaggaagattcaccaatggaagaaccactgttgatattattggtgagtttttgggttttgagaagttaattccagcctttgctagtcttgataataatagcttcaatctattgagtggtgctaattatgcatctgggggagctgggattctcaggcaaacaggaacacatatgggtgaaaatatttgcttgaagaaacaaataaagcatcaccaaattatggtatcaagaataagtgacaaattagggagcaaaaaattagcacaagagctattaaacaagtgcttatattgggttgaaattggaaataatgattacattaacaactacttcatgccttggatttattcttctggaaatctttatacacctcaacaatttgctcttattctcatccaaaaatatcgacttcatatcatggaattgtacaatttgggggcaagaatgatcactttagttggattgggacaaatcggatgcactccgaatgcaattcaaatatatgggacaaacaacgattcttcttcatgtataagttacatgaatgacgctgttcaattattcaataataaacttacaatgctcgtagatgaacttaacagtaattttaccgatgcaaaatttatttacgtcaattcttatgagataggattgtctgaagatctcatcactcttgggtttaaggttttgaatgttggttgttgtggtggagatgagaatggacaatgtaagcctttggaga

SEQ ID NO: 108 - DNA, 1089 bp, Cannabis sativa
  source 1..1089: mol_type = genomic DNA
atggagtcaacattatttgatcagataaaaagtgggagattgttagcgtttatgatcgtgataataacgattattagttatgttaatgcaaaccctaaagtttcttgctttttcatatttggagattctttggctgataatgggaataacaataatctcaacactttggctaaagtcaattataagccttatgggattgacttcattgattctcaaccaacaggaagattcaccaatggaagaaccactgttgatattattggtgagtttttgggttttgagaagttaattccagcctttgctagtcttgataataatagcttcaatctattgagtggtgctaattatgcatctgggggagctgggattctcaggcaaacaggaacacatatgggtgaaaatatttgcttgaagaaacaaataaagcatcaccaaattatggtatcaagaataagtgacaaattagggagcaaaaaattagcacaagagctattaaacaagtgcttatattgggttgaaattggaaataatgattacattaacaactacttcatgccttggatttattcttctggaaatctttatacacctcaacaatttgctcttattctcatccaaaaatatcgacttcatatcttggaattgtacaatttgggggcaagaatgatcactttagttggattgggacaaatcggatgcactccgaatgcaattcaaatatatgggacgaacaacgattcttcttcatgtataagttacatgaatgacgctgttcagttattcaataataaacttacaacgctcgtagatgaacttaacagtgattttaccgatgcaaaatttatttacgtcaattcttatgagataggattgtctgaagatctcatcgctcttgggtttaaggttttgaatgttggttgttgtggtggagatgagaatggacaatgtaagcctttggagattccatgtgagaatagaagagattatgtgttttgggattcatttcatccaactgaggcttacaacttaatcactgcaactaaaatatatcaagcttattataacatatcatatatagatacccaataa

SEQ ID NO: 109 - protein, 298 aa, Cannabis sativa
TIISYVNANPKVSCFFIFGDSLADNGNNNNLNTLAKANYKPYGIDFIDSQPTGRFTNGRTTVDIIGEFLGFEKLIPAFASLDNNSFNLLSGANYASGGAGILRQTGTHMGENICLKKQIKHHQIMVSRISDKLGSKKLAQELLNKCLYWVEIGNNDYINNYFMPWIYSSGNLYTPQQFALILIQKYRLHIMELYNLGARMITLVGLGQIGCTPNAIQIYGTNNDSSSCISYMNDAVQLFNNKLTMLVDELNSNFTDAKFIYVNSYEIGLSEDLITLGFKVLNVGCCGGDENGQCKPLE

SEQ ID NO: 110 - protein, 362 aa, Cannabis sativa
MESTLFDQIKSGRLLAFMIVIITIISYVNANPKVSCFFIFGDSLADNGNNNNLNTLAKVNYKPYGIDFIDSQPTGRFTNGRTTVDIIGEFLGFEKLIPAFASLDNNSFNLLSGANYASGGAGILRQTGTHMGENICLKKQIKHHQIMVSRISDKLGSKKLAQELLNKCLYWVEIGNNDYINNYFMPWIYSSGNLYTPQQFALILIQKYRLHILELYNLGARMITLVGLGQIGCTPNAIQIYGTNNDSSSCISYMNDAVQLFNNKLTTLVDELNSDFTDAKFIYVNSYEIGLSEDLIALGFKVLNVGCCGGDENGQCKPLEIPCENRRDYVFWDSFHPTEAYNLITATKIYQAYYNISYIDTQ

SEQ ID NO: 111 - DNA, 101 bp, Cannabis sativa
  source 1..101: mol_type = genomic DNA
ttctttggctgataatgggaataacaataatctcaacactttggctaaagtcaattataagccttatgggattgacttcattgattctcaaccaacaggaa

SEQ ID NO: 112 - DNA, 101 bp, Cannabis sativa
  source 1..101: mol_type = genomic DNA
ttcagttattcaataataaacttacaacgctcgtagatgaacttaacagtgattttaccgatgcaaaatttatttacgtcaattcttatgagataggattg

SEQ ID NO: 113 - DNA, 101 bp, Cannabis sativa
  source 1..101: mol_type = genomic DNA
aatttatttacgtcaattcttatgagataggattgtctgaagatctcatcgctcttggtaagaattattaccaataatacttttaattttataatttcata

SEQ ID NO: 114 - DNA, 101 bp, Cannabis sativa
  source 1..101: mol_type = genomic DNA
cacctcaacaatttgctcttattctcatccaaaaatatcgacttcatatcttggtaattaattcttaattaatacttctaataattacattattatgcttg

(19) United States
(12) Patent Application Publication
(10) Pub. No.: US 2026/0231886 A1
(43) Pub. Date: Aug. 13, 2026

(54) GENES AND GENETIC MARKERS ASSOCIATED WITH HIGH VARIN PRODUCTION

(71) Applicant: Phylos Bioscience, Inc., Portland, OR (US)

(72) Inventors: Erica Bakker, Portland, OR (US); Alisha Holloway, Portland, OR (US)

(73) Assignee: Phylos Bioscience, Inc., Portland, OR (US)

(21) Appl. No.: 19/161,181

(22) PCT Filed: Feb. 29, 2024
(86) PCT No.: PCT/US2024/017898

§ 371 Date: Aug. 29, 2025

Related U.S. Application Data
(60) Provisional application No. 63/449,248, filed on Mar. 1, 2023.

(51) Int. Cl. A01H 1/04; A01H 1/00; C12Q 1/686; C12Q 1/6869; C12Q 1/6895

(52) U.S. Cl. CPC A01H 1/045; A01H 1/101; C12Q 1/686; C12Q 1/6869; C12Q 1/6895; C12Q 2600/13; C12Q 2600/156

(57) ABSTRACT

Genes associated with varin production in Cannabis plants are provided. Plants that produce varins, or that have increased varin production, and methods of identifying or generating such plants, are also provided.

### CROSS REFERENCE TO RELATED APPLICATIONS

This application claims priority to U.S. Provisional Application No. 63/449,248, filed Mar. 1, 2023, which is incorporated by reference in its entirety.

### FIELD

The present disclosure relates to genes associated with varin production in Cannabis, and methods of producing Cannabis varieties having high varin production.

### SEQUENCE LISTING REFERENCE

The Sequence Listing is submitted as an XML file named “Sequence.xml,” created on Feb. 21, 2024, (128,667 bytes), which is incorporated herein by reference.

### BACKGROUND

Cannabis plants contain over a hundred known cannabinoids, which bind to endogenous endocannabinoid receptors. Varinolic cannabinoids, also known as varins, are a type of cannabinoid compounds having three carbon atoms in their alkyl side chain instead of the five carbon atom alkyl side chains more commonly associated with cannabinoids. Two such varins are tetrahydrocannabivarin (THCV) and cannabidivarin (CBDV), which are homologues of tetrahydrocannabinol (THC) and cannabidiol (CBD), respectively. Each varin has a unique pharmacological profile and distinct molecular targets.

THCV and CBDV have potential benefits across a broad set of applications. Cannabis strains or extracts with high THCV levels, for example, can be used as an agent for anticonvulsant activity, obesity-associated glucose intolerance, appetite suppression, anxiety management for PTSD, diabetic neuropathy, and major neuropathic and pain related pathologies. Another THCV application is for treatment of Parkinson's Disease progression and symptoms. CBDV has been shown to have anti-epileptic and anticonvulsant activity. Cannabichromevarin (CBCV) is a non-psychoactive cannabinoid that is a homolog of CBC and may have anticonvulsant activity. Cannabigerivarin (CBGV) is a non-psychoactive cannabinoid that is a homolog of CBG, and may have analgesic and anti-inflammatory properties.

Research and development as well as the sale of varin products has been limited due to low commonly occurring levels of varins, such as THCV, CBDV, CBCV, or CBGV, in Cannabis flower. The ability to produce Cannabis with high varin levels will create a platform for a new cannabinoid category with differentiated, high margin products in both medical and recreational markets.

The most common way to create Cannabis varieties having modified varin activity is the use of traditional methods of breeding that select for segregated traits over multiple generations. However, traditional breeding methods are laborious and time-consuming. Thus, it is desirable to identify markers and genes for selecting varin attributes.

### SUMMARY

Disclosed herein are methods for identifying Cannabis plants that produce varins, such as THCV, CBDV, CBCV, and/or CBGV, or have increased varin production. In some implementations, the methods include obtaining a nucleic acid sample from the plant or its germplasm, detecting one or more nucleic acid polymorphisms in an ALT4, KR, FATB, GDSL1, or ISS1 (for example, one or more SNPs disclosed herein) gene. In some examples, the polymorphisms are associated with increased varin production. A plant identified as producing varins, or having increased varin production, can be further selected, for example, for further analysis, propagation, breeding, or to make a product. In some examples, the methods include detecting at least two nucleic acid polymorphisms. In some examples, the methods include detecting one or more nucleic acid polymorphisms in at least two of: ALT4, KR, FATB, GDSL1, and ISS1. In some examples, the methods include detecting one or more nucleic acid polymorphisms in ISS1 and KR. In some examples, the methods include detecting one or more nucleic acid polymorphisms in ISS1, KR, and ALT4.

Methods of breeding Cannabis plants that produce varins, such as THCV, CBDV, CBCV, and/or CBGV, or have increased varin production are also disclosed herein. In some implementations, the method includes crossing a plant having one or more nucleic acid polymorphisms in ALT4, KR, FATB, GDSL1, or ISS1 that are associated with varin production or increased varin production (for example, one or more SNPs disclosed herein), to obtain seed and/or progeny plants. In some examples, the progeny plants (e.g., F1) are further screened for the presence of one or more nucleic acid polymorphisms in ALT4, KR, FATB, GDSL1, or ISS1. In some implementations, the progeny plants have increased varin production. Crossing includes, for example, selfing, sibling crossing, outcrossing, or backcrossing. The disclosed breeding methods solve the laborious and time-consuming issues of traditional breeding methods.

Methods of producing modified Cannabis plants are also disclosed. In some implementations, the methods include introducing a genetic modification in an endogenous ALT4, KR, FATB, GDSL1, and/or ISS1 gene (including the promoter region). In some examples, the genetic modification increases ALT4 activity against C4 fatty acids or decreases expression of ISS1 relative to a suitable reference (e.g., relative to the Cannabis plant in an unmodified state). The genetic modification can increase production of varins relative to a suitable reference (e.g., a Cannabis plant in an unmodified state). Exemplary genetic modifications include nucleic acid substitutions, insertions, or deletions. Genetic modifications can be introduced, for example, by mutagenesis or gene editing technique (e.g., RNAi, CRISPR/Cas9, ZFN, or TALEN). Also disclosed are methods of producing a modified Cannabis plant having increased varin production, including introducing a heterologous allele of ALT4, KR, FATB, GDSL1, or ISS1 associated with increased varin production into the plant. Alleles of ALT4, KR, FATB, GDSL1, or ISS1 associated with increased varin production (or “beneficial alleles”) are disclosed herein.

Also disclosed are Cannabis plants identified or produced by any of the methods disclosed herein, including any seed, tissue, cells, or products produced therefrom. In some examples, the Cannabis plant identified or produced by any of the methods disclosed herein includes a total varin content of 4.0% to 35%, for example, 4.0% to 20%, 4.0% to 15%, 4.0% to 10%, 4.0% to 7.0%, 15% to 35%, or 15% to 20%, in at least one plant part. In some examples, the Cannabis plant identified or produced by any of the methods disclosed herein includes a varin ratio of 0.33 to 20, for example, 0.33 to 15, 0.33 to 10, 0.33 to 7, 0.33 to 5, 0.33 to 3, 0.33 to 1, 0.33 to 0.5, 1 to 15, 1 to 10, 1 to 7, 7 to 20, or 7 to 10, in at least one plant part. In some examples, the plant part is a flower, leaf, or trichome.

Modified or transgenic Cannabis plants that include a genetic modification in ALT4, KR, FATB, GDSL1, or ISS1, or include a heterologous beneficial allele of ALT4, KR, FATB, GDSL1, or ISS1, are also disclosed herein. The modified or transgenic Cannabis plants are not, or otherwise exclude, naturally occurring plants.

The foregoing and other objects, features, and advantages of the disclosure will become more apparent from the following detailed description, which proceeds with reference to the accompanying figures.

### SEQUENCES

The nucleic and amino acid sequences listed herein are shown using standard letter abbreviations for nucleotide bases and amino acids. Only one strand of each nucleic acid sequence is shown, but the complementary strand is understood as included by any reference to the displayed strand.

In the accompanying sequence listing:

SEQ ID NO: 1 is an exemplary primer. AGCCACAATGCTAGGGAGAA.
SEQ ID NO: 2 is an exemplary primer. AATGGCATTGTTTACGACACC.
SEQ ID NO: 3 is an exemplary primer. TCTCCATCGACACCGTTTATC.
SEQ ID NO: 4 is an exemplary primer. TTCCTGTGGCTTTTGTTTCC.
SEQ ID NO: 5 is an exemplary primer. TTCCCAAGGGCATTATGGTA.
SEQ ID NO: 6 is an exemplary primer. AACAACCCACGTTCTCAAGG.
SEQ ID NO: 7 is 21VLP5-1-101 ALT4 CDS (1-564 bp).
ATGTTGCAGACGATTTATTCATTGCCATTAGTAAATGTGGCGACGTGTCTCCATCGACACCGTT
TATCTTCATCATCAGTGACTAATTTCACATGTGATGGTGGTAGTACTACTCTTAATGTGAAATTG
AATAGTAGTAAAGATGATGATGATGATGATGATGATAATTATAATAACCAAAAAATTGTGAAG
AGAGATGGGTATAATAATAGAAGAAGAATGAAGATGAATGATGAATATTTTGAAATTGAGCTC
AAAGTTCGTGATTATGAGCTCGATCAATTTGGTGTCGTAAACAATGCCATTTATGCAAGTTATT
GCCAACATGGTACTCAAGAATTTATGGAAAGTGTAATTGGTATAAGCTGTGATGCAATTGCCC
GCAATGGAGAAACATTAGCCACCTCAGAATTATCAATCAAATTCATTTCACCTTTAAGAAGTGG
AGATAAATTTGTGGTGAAGGTAAGGGTCTCTAGATTTTCAGCTGCTCGTGCATACTTTGAGCAC
AAAATTTTCAAGCTTCCCAATTATGAGCCTATTTTGGAAACAAAAGCCACAGGA
SEQ ID NO: 8 is 21TX1-60 ALT4 CDS (1-555 bp).
ATGTTGCAGACGATTTATCCATTGCCATTAGTAAATGTGGCGACGTGTCTCCATCGACACCGTT
TATCTTCGTCATCAGTGACTAATTTCACATGTGATGGTGGTAGTACTACTCTTAATGTGAAATTG
AATAGTAGTAAAGATGATGATGATGATAATTATAATAACCAAAAAATTGTGAAGAGAGATGGG
CATAAAAATAGAAGAAGAATGAAGATGAATGATGAATATTTTGATATTGAGCTCGAAGTTCGT
GATTATGAGCTCGATCAATTTGGTGTCGTAAACAATGCCATTTATGCAAGTTATTGCCAACATG
GTACTCATGAGTTTATGGCAAGTGTAATTGGTATAAGCTGTGATTCAGTTGCTCGCAATGGAGA
AACATTAGCCACCTCAGATTTGTCAATCAAATTCATTTCACCTTTAAGAAGTGGAGATAAATTT
GTGGTGAAGGTAAGGGTCTCTAGAATTTCAGCTGCTCGTGTATACTTTGAGCACAAAATTGTCA
AGCTTCCCAATTATGAGCCTATTTTGGAAACAAAAGCCACAGGA
SEQ ID NO: 9 is Abacus ALT4 CDS (1-630 bp).
ATGTTGCAGACGATTTATCCATTGCCATTAGTAAATGTGGCGACGTGTCTCCATCGACACCGTT
TATCTTCATCATCAGTGACTAATTTCACATGTGATGGTGGTAGTACTACTCTTAATGTGAAATTG
AATAGTAGTAAAGATGATGATGATGATGATGATAATTATAATAACCGAAAAATTGTGAAGAGA
GATGAGCATAATAATAGAAGAAGAATGAAGATGAATGATGAATATTTTGAAATTGAGCTCAAA
GTTCGTGATTATGAGCTCGATCAATTTGGTGTCGTAAACAATGCCATTTATGCAAGTTATTGCC
AACATGGTACTCAAGAATTTATGGAAAGTGTAATTGGTATAAGCTGTGATGCAATTGCCCGCA
ATGGAGAAACATTAGCCACCTCAGAATTATCAATCAAATTCATTTCACCTTTAAGAAGTGGAG
ATAAATTTGTGGTGAAGGTAAGGGTCTCTAGATTTTCAGCTGCTCGTGCATACTTTGAGCACAA
AATTTTCAAGCTTCCCAATTATGAGCCTATTTTGGAAACAAAAGCCACAGGAATTTGGTTGGAT
AAGAAGAATAACCCAATTCGTGTACCGCAAGAGATAAAGAACACGTTTATTATTTGA
SEQ ID NO: 10 is 21VLP5-1-101 ALT4 protein sequence (1-188 AA).
MLQTIYSLPLVNVATCLHRHRLSSSSVTNFTCDGGSTTLNVKLNSSKDDDDDDDDNYNNQKIVKR
DGYNNRRRMKMNDEYFEIELKVRDYELDQFGVVNNAIYASYCQHGTQEFMESVIGISCDAIARNG
ETLATSELSIKFISPLRSGDKFVVKVRVSRFSAARAYFEHKIFKLPNYEPILETKATG
SEQ ID NO: 11 is 21TX1-60 ALT4 protein sequence (1-185 AA).
MLQTIYPLPLVNVATCLHRHRLSSSSVTNFTCDGGSTTLNVKLNSSKDDDDDNYNNQKIVKRDGH
KNRRRMKMNDEYFDIELEVRDYELDQFGVVNNAIYASYCQHGTHEFMASVIGISCDSVARNGETL
ATSDLSIKFISPLRSGDKFVVKVRVSRISAARVYFEHKIVKLPNYEPILETKATG
SEQ ID NO: 12 is Abacus ALT4 protein sequence (1-209 AA).
MLQTIYPLPLVNVATCLHRHRLSSSSVTNFTCDGGSTTLNVKLNSSKDDDDDDDNYNNRKIVKRDE
HNNRRRMKMNDEYFEIELKVRDYELDQFGVVNNAIYASYCQHGTQEFMESVIGISCDAIARNGETL
ATSELSIKFISPLRSGDKFVVKVRVSRFSAARAYFEHKIFKLPNYEPILETKATGIWLDKKNNPIRVPQ
EIKNTFII
SEQ ID NO: 13 is Abacus ALT4 genomic DNA sequence flanking causative SNP (G/A substitution
located at 176 bp in Abacus CDS) located at position 51 bp (Abacus AA substitution R59Q).
ATTGAATAGTAGTAAAGATGATGATGATGATGATGATAATTATAATAACCGAAAAATTGTGAA
GAGAGATGAGCATAATAATAGAAGAAGAATGAAGATGA
SEQ ID NO: 14 is Abacus ALT4 genomic DNA sequence flanking causative SNP (A/G substitution
located at 197 bp in Abacus CDS) located at position 51 bp (Abacus AA substitution E66G).
TGATGATGATGATGATAATTATAATAACCGAAAAATTGTGAAGAGAGATGAGCATAATAATAG
AAGAAGAATGAAGATGAATGATGAATATTTTGAAATTG
SEQ ID NO: 15 is Abacus ALT4 genomic DNA sequence flanking causative indel (---/GAT indel
between positions 141-142 bp in Abacus CDS) located at position 50-52 bp (Abacus AA delction
of a D).
CATGTGATGGTGGTAGTACTACTCTTAATGTGAAATTGAATAGTAGTAAA-
GATGATGATGATGATGATGATAATTATAATAACCGAAAAATTGTGAAGAG
SEQ ID NO: 16 is Abacus ALT4 genomic DNA sequence flanking causative SNP (C/T substitution
located at 199 bp in Abacus CDS) located at position 51 bp (Abacus AA substitution H67Y).
ATGATGATGATGATAATTATAATAACCGAAAAATTGTGAAGAGAGATGAGCATAATAATAGAA
GAAGAATGAAGATGAATGATGAATATTTTGAAATTGAG
SEQ ID NO: 17 is Abacus ALT4 genomic DNA sequence flanking causative indel (GATGAT/------
indel between positions 142-145 bp in Abacus CDS) located at position 51-56 bp (Abacus AA
insertions D48del and D49del).
CATGTGATGGTGGTAGTACTACTCTTAATGTGAAATTGAATAGTAGTAAAGATGATGATGATG
ATGATGATAATTATAATAACCGAAAAATTGTGAAGAGAGATGA
SEQ ID NO: 18 is Abacus ALT4 genomic DNA sequence flanking causative SNP (T/A substitution at
204 bp in Abacus CDS) located at position 51 bp (Abacus AA substitution N68K).
GATGATGATAATTATAATAACCGAAAAATTGTGAAGAGAGATGAGCATAATAATAGAAGAAG
AATGAAGATGAATGATGAATATTTTGAAATTGAGCTCAA
SEQ ID NO: 19 is Abacus ALT4 genomic DNA sequence flanking causative SNP (A/T substitution at
243 bp in Abacus CDS) located at position 51 bp (Abacus AA substitution E81D).
GATGAGCATAATAATAGAAGAAGAATGAAGATGAATGATGAATATTTTGAAATTGAGCTCAAA
GTTCGTGATTATGAGCTCGATCAATTTGGTGTCGTAAA
SEQ ID NO: 20 is Abacus ALT4 genomic DNA sequence flanking causative SNP (A/G substitution at
253 bp in Abacus CDS) located at position 51 bp (Abacus AA substitution K85E).
ATAATAGAAGAAGAATGAAGATGAATGATGAATATTTTGAAATTGAGCTCAAAGTTCGTGATT
ATGAGCTCGATCAATTTGGTGTCGTAAACAATGCCATT
SEQ ID NO: 21 is Abacus ALT4 genomic DNA sequence flanking causative SNP (A/T substitution
located at position 333 bp in Abacus CDS) located at position 51 bp (Abacus AA substitution
Q111H).
GGTGTCGTAAACAATGCCATTTATGCAAGTTATTGCCAACATGGTACTCAAGAATTTATGGAAA
GTGTAATTGGTATAAGCTGTGATGCAATTGCCCGCAA
SEQ ID NO: 22 is Abacus ALT4 genomic DNA sequence flanking causative SNP (A/C substitution
located at position 244 bp in Abacus CDS) located at position 51 bp (Abacus AA substitution
E115A).
CAATGCCATTTATGCAAGTTATTGCCAACATGGTACTCAAGAATTTATGGAAAGTGTAATTGGT
ATAAGCTGTGATGCAATTGCCCGCAATGGAGAAACAT
SEQ ID NO: 23 is Abacus ALT4 genomic DNA sequence flanking causative SNP (G/T substitution
located at 370 bp in Abacus CDS) located at position 51 bp (Abacus AA substitution A124S).
AACATGGTACTCAAGAATTTATGGAAAGTGTAATTGGTATAAGCTGTGATGCAATTGCCCGCA
ATGGAGAAACATTAGCCACCTCAGAATTATCAATCAAA
SEQ ID NO: 24 is Abacus ALT4 genomic DNA sequence flanking causative SNP (A/G substitution
located at 374 bp in Abacus CDS) located at position 51 bp (Abacus AA substitution I125V).
ATGGTACTCAAGAATTTATGGAAAGTGTAATTGGTATAAGCTGTGATGCAATTGCCCGCAATG
GAGAAACATTAGCCACCTCAGAATTATCAATCAAATTC
SEQ ID NO: 25 is Abacus ALT4 genomic DNA sequence flanking causative SNP (A/T substitution
located at 408 bp in Abacus CDS) located at position 51 bp (Abacus AA substitution E136D).
ATAAGCTGTGATGCAATTGCCCGCAATGGAGAAACATTAGCCACCTCAGAATTATCAATCAAA
TTCATTTCACCTTTAAGAAGTGGAGATAAATTTGTGGT
SEQ ID NO: 26 is Abacus ALT4 genomic DNA sequence flanking causative SNP (T/A substitution
located at 478 bp in Abacus CDS) located at position 51 bp (Abacus AA substitution F160I).
CACCTTTAAGAAGTGGAGATAAATTTGTGGTGAAGGTAAGGGTCTCTAGATTTTCAGCTGCTCG
TGCATACTTTGAGCACAAAATTTTCAAGCTTCCCAAT
SEQ ID NO: 27 is Abacus ALT4 genomic DNA sequence flanking causative SNP (C/T substitution
located at 494 bp in Abacus CDS) located at position 51 bp (Abacus AA substitution A165V).
AGATAAATTTGTGGTGAAGGTAAGGGTCTCTAGATTTTCAGCTGCTCGTGCATACTTTGAGCAC
AAAATTTTCAAGCTTCCCAATTATGAGCCTATTTTGG
SEQ ID NO: 28 is Abacus ALT4 genomic DNA sequence flanking causative SNP (T/G substitution
located at 514 bp in Abacus CDS) located at position 51 bp (Abacus AA substitution F172V).
TAAGGGTCTCTAGATTTTCAGCTGCTCGTGCATACTTTGAGCACAAAATTTTCAAGCTTCCCAA
TTATGAGCCTATTTTGGAAACAAAAGCCACAGGAATT
SEQ ID NO: 29 is 22VLV2-1-52 ISS1 upstream region genomic DNA sequence (−512-−333 bp).
TTCCCAAGGGCATTATGGTAATCTCACTTCTTTCTTCCCTAAAACGCTGAACATAGAGAGAGAG
AGAGAGGAGAGAGAGAGAGAGTTGAATTATCTTCTCCGACGTTGGTATTGCTCATCATCAGAT
CAACAACTTCGTTTTCTACCGTTTCATGTTTTAGGTTTTCCTTTTAAAAAGT
SEQ ID NO: 30 is Abacus ISS1 upstream region genomic DNA sequence (−512-−1 bp).
TTCCCAAGGGCATTATGGTAATCTCACTTCTTTCTTCCCTAAAACGCTGAACATAGAGAGAGAG
AGAGAGAGAGAGAGAGAGAGAGAGAGAGTTGAATTATCTTCTCCGACGTTGGTATTGCTCATC
ATCAGATCAACAACTTCGTTTTCTACCGTTTCATGTTTTAGGTTTTCCTTTTAAAAAGTGGAGCT
CCATTGTTTGACTATTGCGGAAAAAGTGGGTAATTTTATGGGTTCCCATACGTGAGAGGACTGA
TGAGGAGGCGTGTTATTGTGTTAAGCTGCCAATTGCTTCGCATGATGATGTTGTGACTCTATGA
CAAAAAAGCCACCCTCTTTTGGATTTTTATCTACTTGTTTTGGGGCTTTGGGTTAGGTGAAAAA
AGGTGATTTTTTTATGTTTAAAACAAGTACTTGGCCAAGCCTTGAGAACGTGGGTTGTTGTTGG
ATTAGACTCACAGCCGAAATAAGTTCACAAAGCTCCAATTTCTCTGTTGACTTACGAGGCCAAC
SEQ ID NO: 31 is Abacus ISS1 genomic DNA sequence flanking causative SNP located at position
51 bp.
CTTCTTTCTTCCCTAAAACGCTGAACATAGAGAGAGAGAGAGAGAGAGAGAGAGAGAGAGAG
AGAGTTGAATTATCTTCTCCGACGTTGGTATTGCTCATC
SEQ ID NO: 32 is an exemplary primer. AAATTCAGGTCCAGCCACAA
SEQ ID NO: 33 is an exemplary primer CACATTTTCGGCAGTGGTC
SEQ ID NO: 34 is an exemplary primer TTCCACTAGCCCAGCAACTT
SEQ ID NO: 35 is an exemplary primer TTTCCAGCTCATTCTTTGACC
SEQ ID NO: 36 is an exemplary primer CCCTCCATCAATGGTGAGAA
SEQ ID NO: 37 is an exemplary primer TCTCCCTTCAATGCAAATCC
SEQ ID NO: 38 is an exemplary primer TCCTGCTTTTGCAGCACTAC
SEQ ID NO: 39 is an exemplary primer CTCTAGAGGTATTAGAAAAGCTACTGC
SEQ ID NO: 40 is an exemplary primer TGCCAAAAATTCCACTAGCC
SEQ ID NO: 41 is an exemplary primer TCATGAGTGATGGAATGGTGA
SEQ ID NO: 42 is an exemplary primer GACCGAATCGAAGGAGATGA
SEQ ID NO: 43 is an exemplary primer GTGGGAGATTGTTAGCGTTTATG
SEQ ID NO: 44 is an exemplary primer GCCTCAGTTGGATGAAATGAA
SEQ ID NO: 45 is Abacus ALT4 genomic DNA sequence flanking causative SNP (C/T substitution at
19 bp in Abacus CDS) located at position 51 bp (Abacus AA substitution P7S)
TAGGGAGAAAGCATTATTATTATAATTCAAACATGTTGCAGACGATTTATCCATTGCCATTAGT
AAATGTGGCGACGTGTCTCCATCGACACCGTTTATCT
SEQ ID NO: 46 is 95-831 ALT4 CDS (1-285 bp)
ATGTTGCAGACGATTTATCCATTGCCATTAGTAAATGTGGCGACGTGTCTCCATCGACACCGTT
TATCTTCATCATCAGTGACTAATTTCACATGTGATGGTGGTAGTACTACTCTTAATGTGAAATTG
AATAGTAGTAAAGATGATGATGATGATAATTATAATAACCAAAAAATTGTGAAGAGAGATGGG
CATAATAATAGAAGAAGAATGAAGATGAATGATGAATATTTTGATATTGAGCTCGAAGTTCGT
GATTATGAGCTCGATCAGTTTGGTGTCGTA
SEQ ID NO: 47 is 95-831 ALT4 protein sequence (1-95 AA)
MLQTIYPLPLVNVATCLHRHRLSSSSVTNFTCDGGSTTLNVKLNSSKDDDDDNYNNQKIVKRDGH
NNRRRMKMNDEYFDIELEVRDYELDQFGVV
SEQ ID NO: 48 is Abacus ALT4 genomic DNA sequence flanking causative SNP (A/G substitution at
604 bp in Abacus CDS) located at position 51 bp (Abacus AA substitution E202Q)
CCACAGGAATTTGGTTGGATAAGAAGAATAACCCAATTCGTGTACCGCAAGAGATAAAGAACA
CGTTTATTATTTGATTAATATTATTTACTCTTTTTTAT
SEQ ID NO: 49 is Abacus ALT4 genomic DNA sequence flanking causative SNP (A/G substitution at
616 bp in Abacus CDS) located at position 51 bp (Abacus AA substitution T206A)
GGTTGGATAAGAAGAATAACCCAATTCGTGTACCGCAAGAGATAAAGAACACGTTTATTATTT
GATTAATATTATTTACTCTTTTTTATACTAAATAAAGG
SEQ ID NO: 50 is Abacus KR CDS (82-801 bp)
CATTTTCGGCAGTGGTCCCCCATTTCCGGTGGCCAATCACTCCGCCACTCCATCTCCCTTCAGTG
CAAATCCAAGAGTTCCTCCGGTGGTGTTGTTAAAGCTCAGGGAGCAAGTCTTGAAGAAACAAG
CGCCGGAGTAGGCCAAAATGTGGAGGCTCCGGTGGTCATTGTGACTGGTGCCTCTAGAGGTAT
TGGAAAAGCTACTGCATTGGCTTTGGGAAAAGCTGGTTGTAAGGTCCTTGTTAATTATGCTAGA
TCATCAAAGGAGGCTGAGGAAGTTTCCAAAGAGATCGAAGCATCTGGTGGCCAAGCTATTACT
TTTGGAGGAGATGTTTCAAAAGAAGCTGATGTTGAATTAATGATCAAAACAGCAGTTGATGCT
TGGGGAACTATTGATGTATTAGTAAATAATGCAGGAATCACAAGGGACAACTTAATTATGAGA
ATGAAGAAGTCCCAGTGGCAGGATGTCATTGATTTGAATCTTACTGGTGTATTTCTATGTACAC
AGGCTGCAGCCAAAATTATGATGAAGAAGAAAAAGGGAAGGATAATCAATGTAGCATCAGTC
TTCGGTCTAGTCGGCAATGCCGGGCAAGCCAATTATAGTGCTGCAAAAGCAGGAGTAATTGGC
TTCACAAAAGCTATTGCTAAGGAGTATTCCAGCAGAAACATCACTGTTAATGCTATTGCTCCAG
GCTTTATTGCATCTGACATGACTGCCAAGCTAGGAGATGATATTGAGAAGAAAAACTTGGAGG
GCATCCCCTTAGGAAGATATGGTCAACCAGAAGAAGTTGCTGGGCTAGTGGAA
SEQ ID NO: 51 is 21TX1-60 KR CDS (82-801 bp)
CATTTTCGGCAGTGGTCCCCCATTTCCGGTGGCCAATCACTCCGCCACTCCATCTCCCTTCAGTG
CAAATCCAAGAGTTCCTCCGGTGGTGTTGTTAAAGCTCAGGGAGCAAGTCTTGAAGAAACAAG
CGCCGGAGTAGGCCAAAATGTGGAGGCTCCGGTGGTCATTGTGACTGGTGCCTCTAGAGGTAT
TGGAAAAGCTACTGCATTGGCTTTGGGAAAAGCTGGTTGTAAGGTCCTTGTTAATTATGCTAGA
TCATCAAAGGAGGCTGAGGAAGTTTCCAAAGAGATCGAAGCATCTGGTGGCCAAGCTATTACT
TTTGGAGGAGATGTTTCAAAAGAAGCTGATGTTGAATTAATGATCAAAACAGCAGTTGATGCT
TGGGGAACTGTTGATGTATTAGTAAATAATGCAGGAATCACAAGGGACAACTTAATTATGAGA
ATGAAGAAATCCCAGTGGCAGGATGTCATTGATTTGAATCTTACTGGTGTATTTCTATGTACAC
AGGCTGCAGCCAAAATTATGATGAAGAAGAAAAAGGGAAGGATAATCAATGTAGCATCAGTC
TTCGGTCTAGTCGGCAATGCCGGGCAAGCCAATTATAGTGCTGCAAAAGCAGGAGTAATTGGC
TTCACAAAAGCTATTGCTAAGGAGTATTCCAGCAGAAACATCACTGTTAATGCTATTGCTCCAG
GCTTTATTGCATCTGACATGACTGCCAAGCTAGGAGATGATATTGAGAAGAAAAACTTGGAGG
GCATCCCCTTAGGAAGATATGGTCAACCAGAAGAAGTTGCTGGGCTAGTGGAA
SEQ ID NO: 52 is 22VLV2-1-52,cpy2AFR,c KR CDS (136-705 bp)
TCCCTTCAATGCAAATCCAAGAGTTCCTCCGGTGGTGTTGTGAAAGCTCAGGTTGAAACTCTTG
AAGAAACAAGCGCCGGAGTAGGCCAAAATGTGGAGGCTCCGGTGGTCATTGTGACTGGTGCCC
CTAGAGGTATTGGAAAAGCTACTGCATTGGCTTTGGGAAAAGCTGGTTGTAAGGTCCTTGTTAA
TTATGCTAGATCATCAAAGGAGGCTGAGGAAGTTTCCAAAGAAATTGAAGCATCTGGTGGCCA
AGCTATTACTTTTGGAGGAGATATTTCAAAAGAAGCTGATGTTGAATTAATGACCAAAATAGC
AGTTGATGCTTGGGGAACTGTTGATGTATTAGTAAATAATGCAGGAATCACTAGGGACAACTT
AATGATGAGAATGAAGAAATCCCAATGGCAGGATGTCATTGATTTGAATCTTACTGGTGTATTT
CTATGTACACAGGCTGCAGCGAAAATTATGATGAAGAAGAAAAGGGGAAGGATAATCAATGT
AGCATCAATCTTCGGTTTAGTCGGCAATGCTGGGCAAGCCAATTGTAGTGCTGCAAAAGCAGG
A
SEQ ID NO: 53 is 22VLV2-1-52,AFR,e KR CDS (82-801 bp)
CATTTTCGGCAGTGGTCCCCCATTTCCAGTGGCCAATCACTCCGTCACTCCATCTCCCTTCAATG
CAAATCCAAGAGTTCCTCCGGTGGTGTTGTGAAAGCTCAGGTTGAAACTCTTGAAGAAACAAG
CGCCGGAGTAGGCCAAAATGTGGAGGCTCCGGTGGTCATTGTGACTGGTGCCTCTAGAGGTAT
TGGAAAAGCTACTGCATTGGCTTTGGGAAAGGCTGGTTGTAAGGTCCTTGTTAATTATGCTAGA
TCATCAAAGGAGGCTGAGGAAGTTTCCAAAGAAATTGAAGCATCTGGTGGCCAAGCTATTACT
TTTGGAGGAGATATTTCAAAAGAAGCTGATGTTGAATTAATGACCAAAATAGCAGTTGATGCT
TGGGGAACTGTTGATGTATTAGTAAATAATGCAGGAATCACTAGGGACAACTTAATGATGAGA
ATGAAGAAATCCCAATGGCAGGATGTCATTGATTTGAATCTTACTGGTGTATCTCTATGTACAC
AGGCTGCAGCGAAAATTATGATGAAGAAGAAAAAGGGAAGGATAATCAATGTAGCATCAATC
TTCGGTTTAGTCGGCAATGCTGGGCAAGCCAATTATAGTGCTGCAAAAGCAGGAGTAATTGGC
TTCACAAAAGCTGTTGCTAAGGAGTATTCCAGCAGAAACATCACTGTTAATGCTATTGCTCCAG
GCTTTATTGCATCTGACATGACTGCCAAGCTAGGAGATGATATTGAGAAGAAAAACTTGGAGG
GCATCCCCTTAGGAAGATATGGTCAACCAGAAGAAGTTGCTGGGCTAGTGGAA
SEQ ID NO: 54 is 22VLV2-1-59,AFR,e KR CDS (82-801 bp)
CATTTTCGGCAGTGGTCCCCCATTTCCAGTGGCCAATCACTCCGTCACTCCATCTCCCTTCAATG
CAAATCCAAGAGTTCCTCCGGTGGTGTTGTGAAAGCTCAGGTTGAAACTCTTGAAGAAACAAG
CGCCGGAGTAGGCCAAAATGTGGAGGCTCCGGTGGTCATTGTGACTGGTGCCTCTAGAGGTAT
TGGAAAAGCTACTGCATTGGCTTTGGGAAAAGCTGGTTGTAAGGTCCTTGTTAATTATGCTAGA
TCATCAAAGGAGGCTGAGGAAGTTTCCAAAGAAATTGGAGCATCTGGTGGCCAAGCTATTACT
TTTGGAGGAGATATTTCAAAAGAAGCTGATGTTGAATTAATGACCAAAATAGCAGTTGATGCT
TGGGGAACTGTTGATGTATTAGTAAATAATGCAGGAATCACTAGGGACAACTTAATGATGAGA
ATGAAGAAATCCCAATGGCAGGATGTCATTGATTTGAATCTTACTGGTGTATTTCTATGTACAC
AGGCTGCAGCGAAAATTATGATGAAGAAGAAAAAGGGAAGGATAATCAATGTAGCATCAATC
TTCGGTTTAGTCGGCAATGCTGGGCAAGCCAATTATAGTGCTGCAAAAGCAGGAGTAATTGGC
TTCACAAAAGCTGTTGCTAAGGAGTATTCCAGCAGAAACATCACTGTTAATGCTATTGCTCCAG
GCTTTATTGCATCTGACATGACTGCCAAGCTAGGAGATGATATTGAGAAGAAGAACTTGGAGG
GCATCCCCTTAGGAAGATATGGTCAACCAGAAGAAGTTGCTGGGCTAGTGGAA
SEQ ID NO: 55 is 22VLV2-1-59,AFR,h KR CDS (82-801 bp)
CATTTTCGGCAGTGGTCCCCCATTTCCAGTGGCCGATCACTCCGTCACTCCATCTCCCTTCAATG
CAAATCCAAGAGTTCCTCCGGTGGTGTTGTGAAAGCTCAGGTTGAAACTCTTGAAGAAACAAA
CGCCGGAGTAGGCCAAAATGTGGAGGCTCCGGTGGTCATTGTGACTGGTGCCTCTAGAGGTAT
TGGAAAAGCTACTGCATTGGCTTTGGGAAAAGCTGGTTGTAAGGTCCTTGTTAATTATGCTAGA
TCATCAAAGGAGGCTGAGGAAGTTTCCAAAGAAATTGAAGCATCTGGTGGCCAAGCTATTACT
TTTGGAGGAGATATTTCAAAAGAAGCTGATGTTGAATTAATGACCAAAATAGCAGTTGATGCT
TGGGGAACTGTTGATGTATTAGTAAATAATGCAGGAATCACTAGGGACAACTTAATGATGAGA
ATGAAGAAATCCCAATGGCAGGATGTCATTGATTTGAATCTTACTGGTGTATTTCTATGTACAC
AGGCTGCAGCGAAAATTATGATGAAGAAGAAAAAGGGAAGGATAATCAATGTAGCATCAATC
TTCGGTTTAGTCGGCAATGCTGGGCAAGCCAATTATAGTGCTGCAAAAGCAGGAGTAATTGGC
TTCACAAAAGCTGTTGCTAAGGAGTATTCCAGCAGAAACATCACTGTTAATGCTATTGCTCCAG
GCTTTATTGCATCTGACATGACTGCCAAGCTAGGAGATGATATTGAGAAGAAAAACTTGGAGG
GCATCCCCTTAGGAAGATATGGTCAACCAGAAGAAGTTGCTGGGCTAGTGGAA
SEQ ID NO: 56 is 22VLV2-1-59,AFR,j KR CDS (82-801 bp)
CATTTTCGGCAGTGGTCCCCCATTTCCAGTGGCCAATCACTCCGTCACTCCATCTCCCTTCAATG
CAAATCCAAGAGTTCCTCCGGTGGTGTTGTGAAAGCTCAGGTTGAAACTCTTGAAGAAACAAG
CGCCGGAGTAGGCCAAAATGTGGAGGCTCCGGTGGTCATTGTGACTGGTGCCTCTAGAGGTAT
TGGAAAAGCTACTGCATTGGCTTTGGGAAAAGCTGGTTGTAAGGTCCTTGTTAATTATGCTAGA
TCATCAAAGGAGGCTGAGGAAGTTTCCGAAGAAATTGAAGCATCTGGTGGCCAAGCTATTACT
TTTGGAGGAGATATTTCAAAAGAAGCTGATGTTGAATTAATGACCAAAATAGCAGTTGATGCT
TGGGGAACTGTTGATGTATTAGTAAATAATGCAGGAATCACTAGGGACAACTTAATGATGAGA
ATGAAGAAATCCCAATGGCAGGATGTCATTGATTTGAATCTTACTGGTGTATTTCTATGTACAC
AGGCTGCAGCGAAAATTATGATGAAGAAGAAAAAGGGAAGGATAATCAATGTAGCATCAATC
TTCGGTTTAGTCGGCAATGCTGGGCAAGCCAATTATAGTGCTGCAAAAGCAGGAGTAATTGGC
TTCACAAAAGCTGTTGCTAAGGAGTATTCCAGCAGAAACATCACTGTTAATGCTATTGCTCCAG
GCTTTATTGCATCTGACATGACTGCCAAGCTAGGAGATGATATTGAGAAGAAAAACTTGGAGG
GCATCCCCTTAGGAAGATATGGTCAACCAGAAGAAGTTGCTGGGCTAGTGGAA
SEQ ID NO: 57 is 21VLP5-1-222,ARF,a KR CDS (82-801 bp)
CATTTTCGGCAGTGGTCCCCCATTTCCAGTGGCCAATCACTCCGTCACTCCATCTCCCTTCAATG
CAAATCCAAGAGTTCCTCCGGTGGTGTTGTGAAAGCTCAGGTTGAAACTCTTGAAGAAACAAG
CGCCGGAGTAGGCCAAAATGTGGAGGCTCCGGTGGTCATTGTGACTGGTGCCTCTAGAGGTAT
TGGAAAAGCTACTGCATTGGCTTTGGGAAAAGCTGGTTGTAAGGTCCTTGTTAATTATGCTAGA
TCATCAAAGGAGGCTGAGGAAGTTTCCAAAGAAATTGAAGCATCTGGTGGCCAAGCTATTACT
TTTGGAGGAGATATTTCAAAAGAAGCTGATGTTGAATTAATGACCAAAATAGCAGTTGATGCT
TGGGGAACTGTTGATGTATTAGTAAATAATGCAGGAATCACTAGGGACAACTTAATGATGAGA
ATGAAGAAATCCCAATGGCAGGATGTCATTGATTTGAATCTTACTGGTGTATTTCTATGTACAC
AGGCTGCAGCGAAAATTATGATGAAGAAGAAAAAGGGAAGGATAATCAATGTAGCATCAATC
TCCGGTTTAGTCGGCAATGCTGGGCAAGCCAATTATAGTGCTGCAAAAGCAGGAGTAATTGGC
TTCACAAAAGCTGTTGCTAAGGAGTATTCCAGCAGAAACATCACTGTTAATGCTATTGCTCCAG
GCTTTATTGCATCTGACATGACTGCCAAGCTAGGAGATGATATTGAGAAGAAAAACTTGGAGG
GCATCCCCTTAGGAAGATATGGTCAACCAGAAGAAGTTGCTGGGCTAGTGGAA
SEQ ID NO: 58 is 21VLP5-1-222,AFR,j KR CDS (82-801 bp)
CATTTTCGGCAGTGGTCCCCCATTTCCAGTGGCCAATCACTCCGTCACTCCATCTCCCTTCAATG
CAAATCCAAGAGTTCCTCCGGTGGTGTTGTGAAAGCTCAGGTTGAAACTCTTGAAGAAACAAG
CGCCGGAGTAGGCCAAAATGTGGAGGCTCCGGTGGTCATTGTGACTGGTGCCTCTAGAGGTAT
TGGAAAAGCTACTGCATTGGCTTTGGGAAAAGCTGGTTGTAAGGTCCTTGTTAATTATGCTAGA
TCATCAAAGGAGGCTGAGGAAGTTTCCAAAGGGATTGAAGCATCTGGTGGCCAAGCTATTACT
TTTGGAGGAGATATTTCAAAAGAAGCTGATGTTGAATTAATGACCAAAATAGCAGTTGATGCT
TGGGGAACTGTTGATGTATTAGTAAATAATGCAGGAATCACTAGGGACAACTTAATGATGAGA
ATGAAGAAATCCCAATGGCAGGATGTCATTGATTTGAATCTTACTGGTGTATTTTTATGTACAC
AGGCTGCAGCCAAAATTATGATGAAGAAGAAAAAGGGAAGGATAATCAATGTAGCATCAATC
TTCGGTTTAGTCGGCAATGCTGGGCAAGCCAATTATAGTGCTGCAAAAGCAGGAGTAATTGGC
TTCACAAAAGCTGTTGCTAAGGAGTATTCCAGCAGAAACATCACTGTTAATGCTATTGCTCCAG
GCTTTATTGCATCTGACATGACTGCCAAGCTAGGAGATGATATTGAGAAGAAAAACTTGGAGG
GCATCCCCTTAGGAAGATATGGTCAACCAGAAGAAGTTGCTGGGCTAGTGGAA
SEQ ID NO: 59 is 22TP1C-140-40,AFR,b KR CDS (82-801 bp)
CATTTTCGGCAGTGGTCCCCCATTTCCAGTGGCCAATCACTCCGTCACTCCATCTCCCTTCAATG
CAAATCCAAGAGTTCCTCCGGTGGTGTTGTGAAAGCTCAGGTTGAAACTCTTGAAGAAACAAG
CGCCGGAGTAGGCCAAAATGTGGAGGCTCCGGTGGTCATTGTGACTGGTGCCTCTAGAGGTAT
TGGAAAAGCTACTGCATTGGCTTTGGGAAAAGCTGGTTGTAAGGTCCTTGTTAATTATGCTAGA
TCATCAAAGGAGGCTGAGGAAGTTTCCAAAGAGATCGAAGCATCTGGTGGCCAAGCTATTACT
TTTGGAGGAGATATTTCAAAAGAAGCTGATGTTGAATTAATGACCAAAATAGCAGTTGATGCT
TGGGGAACTGTTGATGTATTAGTAAATAATGCAGGAATCACTAGGGACAACTTAATGATGAGA
ATGAAGAAATCCCAATGGCAGGATGTCATTGATTTGAATCTTACTGGTGTATTTTTATGTACAC
AGGCTGCAGCCAAAATTATGATGAGGAAGAAAAAGGGAAGGATAATCAATGTAGCATCAATC
TTCGGTTTAGTCGGCAATGCTGGGCAAGCCAATTATAGTGCTGCAAAAGCAGGAGTAATTGGC
TTCACAAAAGCTGTTGCTAAGGAGTATTCCAGCAGAAACATCACTGTTAATGCTATTGCTCCAG
GCTTTATTGCATCTGACATGACTGCCAAGCTAGGAGATGATATTGAGAAGAAAAACTTGGAGG
GCATCCCCTTAGGAAGATATGGTCAACCAGAAGAAGTTGCTGGGCTAGTGGAA
SEQ ID NO: 60 is 22TP1C-140-40,AFR,f KR CDS (82-801 bp)
CATTTTCGGCAGTGGTCCCCCATTTCCAGTGGCCAATCACTCCGTCACTCCATCTCCCTTCAATG
CAAATCCAAGAGTTCCTCCGGTGGTGTTGTGAAAGCTCAGGTTGAAACTCTTGAAGAAACAAG
CGCCGGAGTAGGCCAAAATATGGAGGCTCCGGTGGTCATTGTGACTGGTGCCTCTAGAGGTAT
TGGAAAAGCTACTGCATTGGCTCTGGGAAAAGCTGGTTGTAAGGTCCTTGTTAATTATGCTAGA
TCATCAAAGGAGGCTGAGGAAGTTTCCAAAGAGATTGAAGCATCTGGTGGCCAAGCTATTACT
TTTGGAGGAGATATTTCAAAAGAAGCTGATGTTGAATTAATGACCAAAATAGCAGTTGATGCT
TGGGGAACTGTTGATGTATTAGTAAATAATGCAGGAATCACTAGGGACAACTTAATGATGAGA
ATGAAGAAATCCCAATGGCAGGATGTCATTGATTTGAATCTTACTGGTGTATTTTTATGTACAC
AGGCTGCAGCCAAAATTATGATGAAGAAGAAAAAGGGAAGGATAATCAATGTAGCATCAATC
TTCGGTTTAGTCGGCAATGCTGGGCAAGCCAATTATAGTGCTGCAAAAGCAGGAGTAATTGGC
TTCACAAAAGCTGTTGCTAAGGAGTATTCCAGCAGAAACATCACTGTTAATGCTATTGCTCCAG
GCTTTATTGCATCTGACATGACTGCCAAGCTAGGAGATGATATTGAGAAGAAAAACTTGGAGG
GCATCCCCTTAGGAAGATATGGTCAACCAGAAGAAGTTGCTGGGCTAGTGGAA
SEQ ID NO: 61 is 22TP1C-141-25,AFR,a KR CDS (82-801 bp)
CATTTTCGGCAGTGGTCCCCCATTTCCAGTGGCCAATCACTCCGTCACTCCATCTCCCTTCAATG
CAAATCCAAGAGTTCCTCCGGTGGTGTTGTGAAAGCTCAGGTTGAAACTCTTGAAGAAACAAG
CGCCGGAGTAGGCCAAAATGTGGAGGCTCCGGTGGTCATTGTGACTGGTGCCTCTAGAGGTAT
TGGAAAAGCTACTGCATTGGCTTTGGGAAAAGCTGGTTGTAAGGTCCTTGTTAATTATGCTAGA
TCATCAAAGGAGGCTGAGGAAGTTTCCAAAGAGATTGAAGCATCTGGTGGCCAAGCTATTACT
TTTGGAGGAGATATTCCAAAAGAAGCTGATGTTGAATTAATGACCAAAATAGCAGTTGATGCT
TGGGGAACTGTTGATGTATTAGTAAATAATGCAGGAATCACTAGGGACAACTTAATGATGGGA
ATGAAGAAATCCCAATGGCAGGATGTCATTGATTTGAATCTTACTGGTGTATTTTTATGTACAC
AGGCTGCAGCCAAAATTATGATGAAGAAGAAAAAGGGAAGGATAATCAATGTAGCATCAATC
TTCGGTTTAGTCGGCAATGCTGGGCAAGCCAATTATAGTGCTGCAAAAGCAGGAGTAATTGGC
TTCACAAAAGCTGTTGCTAAGGAGTATTCCAGCAGAAACATCACTGTTAATGCTATTGCTCCAG
GCTTTATTGCATCTGACATGACTGCCAAGCTAGGAGATGATATTGAGAAGAAAAACTTGGAGG
GCATCCCCTTAGGAAGATATGGTCAACCAGAAGAAGTTGCTGGGCTAGTGGAA
SEQ ID NO: 62 is 22TP1C-141-25,AFR,b KR CDS (82-801 bp)
CATTTTCGGCAGTGGTCCCCCATTTCCAGTGGCCAATCACTCCGTCACTCCATCTCCCTTCAATG
CAAATCCAAGAGTTCCTCCGGTGGTGTTGTGAAAGCTCAGGTTGAAACTCTTGAAGAAACAAG
CGCCGGAGTAGGCCAAAATGTGGAGGCTCCGGTGGTCATTGTGACTGGTGCCTCTAGAGGTAT
TGGAAAAGCTACTGCATTGGCTTTGGGAAAAGCTGGTTGTAAGGTCCTTGTTAATTATGCTAGA
TCATCAAAGGAGGCTGAGGAAGTTTCCAAAGAGATTGAAGCATCTGGTGGCCAAGCTATTACT
TTTGGAGGAGATATTTCAAAAGAAGCTGATGTTGAATTAATGACCAAAATAGCAGTTGATGCT
TGGGGAACTGTTGATGTATTAGTAAATAATGCAGGAATCACTAGGGACAACTTAATGATGAGA
ATGAAGAAATCCCAATGGCAGGATGTCATTGATTTGAATCTTACTGGTGTATTTTTATGTACAC
AGGCTGCAGCCAAAATTATGATGAAGAAGAAAAAGGGAAGGATAATCAATGTAGCATCAATC
TTCGGTTTAGTCGGCAATGCTGGGCAAGCCAATTATAGTGCTGCAAAAGCAGGAGTAATTGGC
TTCACAAAAGCTGTTGCTAAGGAGTATTCCAGCAGAAGCATCACTGTTAATGCTATTGCTCCAG
GCTTTATTGCATCTGACATGACTGCCAAGCTAGGAGATGATATTGAGAAGAAAAACTTGGAGG
GCATCCCCTTAGGAAGATATGGTCAACCAGAAGAAGTTGCTGGGCTAGTGGAA
SEQ ID NO: 63 is 22TP1C-141-25,AFR,d KR CDS (82-801 bp)
CATTTTCGGCAGTGGTCCCCCATTTCCAGTGGCCAATCACTCCGTCACTCCATCTCCCTTCAATG
CAAATCCAAGAGTTCCTCCGGTGGTGTTGTGAAAGCTCAGGTTGAAACTCTTGAAGAAACAAG
CGCCGGAGTAGGCCAAAATGTGGAGGCTCCGGTGGTCATTGTGACTGGTGCCCCTAGAGGTAT
TGGAAAAGCTACTGCATTGGCTTTGGGAAAAGCTGGTTGTAAGGTCCTTGTTAATTATGCTAGA
TCATCAAAGGAGGCTGAGGAAGTTTCCAAAGAGATTGAAGCATCTGGTGGCCAAGCTATTACT
TTTGGAGGAGATATTTCAAAAGAAGCTGATGTTGAATTAATGACCAAAATAGCAGTTGATGCT
TGGGGAACTGTTGATGTATTAGTAAATAATGCAGGAATCACTAGGGACAACTTAATGATGAGA
ATGAAGAAATCCCAATGGCAGGATGTCATTGATTTGAATCTTACTGGTGTATTTTTATGTACAC
AGGCTGCAGCCAAAATTATGATGAAGAAGAAAAAGGGAAGGATAATCAATGTAGCATCAATC
TTCGGTTTAGTCGGCAATGCCGGGCAAGCCAATTATAGTGCTGCAAAAGCAGGAGTAATTGGC
TTCACAAAAGCTGTTGCTAAGGAGTATTCCAGCAGAAACATCACTGTTAATGCTATTGCTCCAG
GCTTTATTGCATCTGACATGACTGCCAAGCTAGGAGATGATATTGAGAAGAAAAACTTGGAGG
GCATCCCCTTAGGAAGATATGGTCAACCAGAAGAAGTTGCTGGGCTAGTGGAA
SEQ ID NO: 64 is 23PV1-22-110,cpy2BFR,a KR CDS (262-903 bp)
TCTAGAGGTATTAGAAAAGCTACTGCATTGGCTTTGGGAAAAGCTGGTTGTAAGGTACTTGTTA
ATTATGCTAGATCATCAAAGGAGGCTGAGGAAGTTTCCAAAGAGATTGAAGCATCTGGTGGCC
AAGCTATTACTTTTGGAGGAGATGTTTCAAAAGAAGCTGATGTTGAATTAATGATCAAAATAG
CAGTTGATGCTTGGGGAACTGTTGATGTATTAGTAAATAATGCAGGAATCACAAGGGACAACT
TAATTATGAGAATGAAGAAATCCCAGTGGCAGGATGTCATTGATTTGAATCTTACTGGTGTATT
TCTATGTACACAGGCTGCAGCCAAAATTATGATGAAGAAGAAAAAGGGAAGGATAATCAATGT
AGCATCAGTCTTCGGTCTAGTCGGCAATGCTGGGCAAGCCAACTACAGTGCTGCAAAAGCAGG
AGTAATTGGCTTCACAAAAGCTATTGCTAAGGAGTATTCCAGCAGAAACATCACTGTTAATGCT
ATTGCTCCAGGCTTTATTGCATCTGACATGACTGCCAAGCTAGGAGATGATATTGAGAAGAAA
AACTTGGAGGGCATCCCCTTAGGAAGATATGGTCAACCAGAAGAAGTTGCTGGGCTAGTGGAA
TTTTTGGCA
SEQ ID NO: 65 is 23PV1-22-110,cpy2BFR,f KR CDS (262-903 bp bp)
TCTAGAGGTATTAGAAAAGCTACTGCATTGGCTTTGGGAAAAGCTGGTTGTAAGGTACTTGTTA
ATTATGCTAGATCATCAAAGGAGGCTGAGGAAGTTTCCAAAGAGATTGAAGCATCTGGTGGCC
AAGCTATTACTTTTGGAGGAGATGTTTCAAAAGAAGCTGATGTTGAATTAATGATCAAAATAG
CAGTTGATGCTTGGGGAACTGTTGATGTATTAGTAAATAATGCAGGAATCACAAGGGACAACT
TAATTATGAGAATGAAGAAATCCCAGTGGCAGGATGTCATTGATTTGAATCTTACTGGTGTATT
TCTATGTACACAGGCTGCAGCCAAAATTATGATGAAGAAGAAAAAGGGAAGGATAATCAATGT
AGCATCAGTCTTCGGTCTAGTCGGCAATGCTGGGCAAGCCAATTACAGTGCTGCAAAAGCAGG
AGTAATTGGCTTCACAAAAGCTATTGCTAAGGAGTATTCCAGCAGAAACATCACTGTTAATGCT
ATTGCTCCAGGCTTTATTGCATCTGACATGACTGCCAAGCTAGGAGATGATATTGAGAAGAAA
AACTTGGAGGGCATCCCCTTAGGAAGATATGGTCAACCAGAAGAAATTGCTGGGCTAGTGGAA
TTTTTGGCA
SEQ ID NO: 66 is Abacus KR protein sequence (28-267 AA)
HFRQWSPISGGQSLRHSISLQCKSKSSSGGVVKAQGASLEETSAGVGQNVEAPVVIVTGASRGIGKA
TALALGKAGCKVLVNYARSSKEAEEVSKEIEASGGQAITFGGDVSKEADVELMIKTAVDAWGTVD
VLVNNAGITRDNLIMRMKKSQWQDVIDLNLTGVFLCTQAAAKIMMKKKKGRIINVASVFGLVGNA
GQANYSAAKAGVIGFTKAIAKEYSSRNITVNAIAPGFIASDMTAKLGDDIEKKNLEGIPLGRYGQPE
EVAGLVE
SEQ ID NO: 67 is 21TX1-60 KR protein sequence (28-267 AA)
HFRQWSPISGGQSLRHSISLQCKSKSSSGGVVKAQGASLEETSAGVGQNVEAPVVIVTGASRGIGKA
TALALGKAGCKVLVNYARSSKEAEEVSKEIEASGGQAITFGGDVSKEADVELMIKTAVDAWGTVD
VLVNNAGITRDNLIMRMKKSQWQDVIDLNLTGVFLCTQAAAKIMMKKKKGRIINVASVFGLVGNA
GQANYSAAKAGVIGFTKAIAKEYSSRNITVNAIAPGFIASDMTAKLGDDIEKKNLEGIPLGRYGQPE
EVAGLVE
SEQ ID NO: 68 is 22VLV2-1-52,cpy2AFR,c KR protein sequence (46-235 AA)
SLQCKSKSSSGGVVKAQVETLEETSAGVGQNVEAPVVIVTGAPRGIGKATALALGKAGCKVLVNY
ARSSKEAEEVSKEIEASGGQAITFGGDISKEADVELMTKIAVDAWGTVDVLVNNAGITRDNLMMR
MKKSQWQDVIDLNLTGVFLCTQAAAKIMMKKKRGRIINVASIFGLVGNAGQANCSAAKAG
SEQ ID NO: 69 is 22VLV2-1-52,AFR,e KR protein sequence (28-267 AA)
HFRQWSPISSGQSLRHSISLQCKSKSSSGGVVKAQVETLEETSAGVGQNVEAPVVIVTGASRGIGKA
TALALGKAGCKVLVNYARSSKEAEEVSKEIEASGGQAITFGGDISKEADVELMTKIAVDAWGTVD
VLVNNAGITRDNLMMRMKKSQWQDVIDLNLTGVSLCTQAAAKIMMKKKKGRIINVASIFGLVGN
AGQANYSAAKAGVIGFTKAVAKEYSSRNITVNAIAPGFIASDMTAKLGDDIEKKNLEGIPLGRYGQP
EEVAGLVE
SEQ ID NO: 70 is 22VLV2-1-59,AFR,e KR protein sequence (28-267 AA)
HFRQWSPISSGQSLRHSISLQCKSKSSSGGVVKAQVETLEETSAGVGQNVEAPVVIVTGA
SRGIGKATALALGKAGCKVLVNYARSSKEAEEVSKEIGASGGQAITFGGDISKEADVELMTKIAVD
AWGTVDVLVNNAGITRDNLMMRMKKSQWQDVIDLNLTGVFLCTQAAAKIMMKKKKGRIINVASI
FGLVGNAGQANYSAAKAGVIGFTKAVAKEYSSRNITVNAIAPGFIASDMTAKLGDDIEKKNLEGIPL
GRYGQPEEVAGLVE
SEQ ID NO: 71 is 22VLV2-1-59,AFR,h KR protein sequence (28-267 AA)
HFRQWSPISSGRSLRHSISLQCKSKSSSGGVVKAQVETLEETNAGVGQNVEAPVVIVTGASRGIGKA
TALALGKAGCKVLVNYARSSKEAEEVSKEIEASGGQAITFGGDISKEADVELMTKIAVDAWGTVD
VLVNNAGITRDNLMMRMKKSQWQDVIDLNLTGVFLCTQAAAKIMMKKKKGRIINVASIFGLVGN
AGQANYSAAKAGVIGFTKAVAKEYSSRNITVNAIAPGFIASDMTAKLGDDIEKKNLEGIPLGRYGQP
EEVAGLVE
SEQ ID NO: 72 is 22VLV2-1-59,AFR,j KR protein sequence (28-267 AA)
HFRQWSPISSGQSLRHSISLQCKSKSSSGGVVKAQVETLEETSAGVGQNVEAPVVIVTGASRGIGKA
TALALGKAGCKVLVNYARSSKEAEEVSEEIEASGGQAITFGGDISKEADVELMTKIAVDAWGTVDV
LVNNAGITRDNLMMRMKKSQWQDVIDLNLTGVFLCTQAAAKIMMKKKKGRIINVASIFGLVGNA
GQANYSAAKAGVIGFTKAVAKEYSSRNITVNAIAPGFIASDMTAKLGDDIEKKNLEGIPLGRYGQPE
EVAGLVE
SEQ ID NO: 73 is 21VLP5-1-222,ARF,a KR protein sequence (28-267 AA)
HFRQWSPISSGQSLRHSISLQCKSKSSSGGVVKAQVETLEETSAGVGQNVEAPVVIVTGASRGIGKA
TALALGKAGCKVLVNYARSSKEAEEVSKEIEASGGQAITFGGDISKEADVELMTKIAVDAWGTVD
VLVNNAGITRDNLMMRMKKSQWQDVIDLNLTGVFLCTQAAAKIMMKKKKGRIINVASISGLVGN
AGQANYSAAKAGVIGFTKAVAKEYSSRNITVNAIAPGFIASDMTAKLGDDIEKKNLEGIPLGRYGQP
EEVAGLVE
SEQ ID NO: 74 is 21VLP5-1-222,AFR,j KR protein sequence (28-267 AA)
HFRQWSPISSGQSLRHSISLQCKSKSSSGGVVKAQVETLEETSAGVGQNVEAPVVIVTGASRGIGKA
TALALGKAGCKVLVNYARSSKEAEEVSKGIEASGGQAITFGGDISKEADVELMTKIAVDAWGTVD
VLVNNAGITRDNLMMRMKKSQWQDVIDLNLTGVFLCTQAAAKIMMKKKKGRIINVASIFGLVGN
AGQANYSAAKAGVIGFTKAVAKEYSSRNITVNAIAPGFIASDMTAKLGDDIEKKNLEGIPLGRYGQP
EEVAGLVE
SEQ ID NO: 75 is 22TP1C-140-40,AFR,b KR protein sequence (28-267 AA)
HFRQWSPISSGQSLRHSISLQCKSKSSSGGVVKAQVETLEETSAGVGQNVEAPVVIVTGASRGIGKA
TALALGKAGCKVLVNYARSSKEAEEVSKEIEASGGQAITFGGDISKEADVELMTKIAVDAWGTVD
VLVNNAGITRDNLMMRMKKSQWQDVIDLNLTGVFLCTQAAAKIMMRKKKGRIINVASIFGLVGN
AGQANYSAAKAGVIGFTKAVAKEYSSRNITVNAIAPGFIASDMTAKLGDDIEKKNLEGIPLGRYGQP
EEVAGLVE
SEQ ID NO: 76 is 22TP1C-140-40,AFR,f KR protein sequence (28-267 AA)
HFRQWSPISSGQSLRHSISLQCKSKSSSGGVVKAQVETLEETSAGVGQNMEAPVVIVTGASRGIGKA
TALALGKAGCKVLVNYARSSKEAEEVSKEIEASGGQAITFGGDISKEADVELMTKIAVDAWGTVD
VLVNNAGITRDNLMMRMKKSQWQDVIDLNLTGVFLCTQAAAKIMMKKKKGRIINVASIFGLVGN
AGQANYSAAKAGVIGFTKAVAKEYSSRNITVNAIAPGFIASDMTAKLGDDIEKKNLEGIPLGRYGQP
EEVAGLVE
SEQ ID NO: 77 is 22TP1C-141-25,AFR,a KR protein sequence (28-267 AA)
HFRQWSPISSGQSLRHSISLQCKSKSSSGGVVKAQVETLEETSAGVGQNVEAPVVIVTGASRGIGKA
TALALGKAGCKVLVNYARSSKEAEEVSKEIEASGGQAITFGGDIPKEADVELMTKIAVDAWGTVD
VLVNNAGITRDNLMMGMKKSQWQDVIDLNLTGVFLCTQAAAKIMMKKKKGRIINVASIFGLVGN
AGQANYSAAKAGVIGFTKAVAKEYSSRNITVNAIAPGFIASDMTAKLGDDIEKKNLEGIPLGRYGQP
EEVAGLVE
SEQ ID NO: 78 is 22TP1C-141-25,AFR,b KR protein sequence (28-267 AA)
HFRQWSPISSGQSLRHSISLQCKSKSSSGGVVKAQVETLEETSAGVGQNVEAPVVIVTGASRGIGKA
TALALGKAGCKVLVNYARSSKEAEEVSKEIEASGGQAITFGGDISKEADVELMTKIAVDAWGTVD
VLVNNAGITRDNLMMRMKKSQWQDVIDLNLTGVFLCTQAAAKIMMKKKKGRIINVASIFGLVGN
AGQANYSAAKAGVIGFTKAVAKEYSSRSITVNAIAPGFIASDMTAKLGDDIEKKNLEGIPLGRYGQP
EEVAGLVE
SEQ ID NO: 79 is 22TP1C-141-25,AFR,d KR protein sequence (28-267 AA)
HFRQWSPISSGQSLRHSISLQCKSKSSSGGVVKAQVETLEETSAGVGQNVEAPVVIVTGAPRGIGKA
TALALGKAGCKVLVNYARSSKEAEEVSKEIEASGGQAITFGGDISKEADVELMTKIAVDAWGTVD
VLVNNAGITRDNLMMRMKKSQWQDVIDLNLTGVFLCTQAAAKIMMKKKKGRIINVASIFGLVGN
AGQANYSAAKAGVIGFTKAVAKEYSSRNITVNAIAPGFIASDMTAKLGDDIEKKNLEGIPLGRYGQP
EEVAGLVE
SEQ ID NO: 80 is 23PV1-22-110,cpy2BFR,a KR protein sequence (88-301 AA)
SRGIRKATALALGKAGCKVLVNYARSSKEAEEVSKEIEASGGQAITFGGDVSKEADVELMIKIAVD
AWGTVDVLVNNAGITRDNLIMRMKKSQWQDVIDLNLTGVFLCTQAAAKIMMKKKKGRIINVASV
FGLVGNAGQANYSAAKAGVIGFTKAIAKEYSSRNITVNAIAPGFIASDMTAKLGDDIEKKNLEGIPL
GRYGQPEEVAGLVEFLA
SEQ ID NO: 81 is 23PV1-22-110,cpy2BFR,f KR protein sequence (88-301 AA)
SRGIRKATALALGKAGCKVLVNYARSSKEAEEVSKEIEASGGQAITFGGDVSKEADVELMIKIAVD
AWGTVDVLVNNAGITRDNLIMRMKKSQWQDVIDLNLTGVFLCTQAAAKIMMKKKKGRIINVASV
FGLVGNAGQANYSAAKAGVIGFTKAIAKEYSSRNITVNAIAPGFIASDMTAKLGDDIEKKNLEGIPL
GRYGQPEEIAGLVEFLA
SEQ ID NO: 82 is Abacus KR genomic DNA sequence flanking causative SNP (A/G substitution at
116 bp in Abacus CDS) located at position 51 bp (Abacus AA substitution Q39R)
TGACCGGAAAGCCCCACATTTTCGGCAGTGGTCCCCCATTTCCGGTGGCCAATCACTCCGCCAC
TCCATCTCCCTTCAGTGCAAATCCAAGAGTTCCTCCG
SEQ ID NO: 83 is Abacus KR genomic DNA sequence flanking causative SNP (T/C substitution at
262 bp in Abacus CDS) located at position 51 bp (Abacus AA substitution S88P)
CCGGAGTAGGCCAAAATGTGGAGGCTCCGGTGGTCATTGTGACTGGTGCCTCTAGAGGTATTG
GAAAAGCTACTGCATTGGCTTTGGGAAAAGCTGGTTGT
SEQ ID NO: 84 is Abacus KR genomic DNA sequence flanking causative SNP (A/G substitution at
364 bp in Abacus CDS) located at position 51 bp (Abacus AA substitution K122E)
AGGTCCTTGTTAATTATGCTAGATCATCAAAGGAGGCTGAGGAAGTTTCCAAAGAGGTGAGTT
CCATCAAGTTCTATTCATGTTTGTGTTATCCCTTTCAA
SEQ ID NO: 85 is Abacus KR genomic DNA sequence flanking causative SNP (G/A substitution at
368 bp in Abacus CDS) located at position 51 bp (Abacus AA substitution E123G)
TCTTTATGTAGTTCAACCACCATCAAAAATAATTTTGACTGAGTTCTTACAGATCGAAGCATCT
GGTGGCCAAGCTATTACTTTTGGAGGAGATGTTTCAA
SEQ ID NO: 86 is Abacus KR genomic DNA sequence flanking causative SNP (A/G substitution at
374 bp in Abacus CDS) located at position 51 bp (Abacus AA substitution E125G)
TGTAGTTCAACCACCATCAAAAATAATTTTGACTGAGTTCTTACAGATCGAAGCATCTGGTGGC
CAAGCTATTACTTTTGGAGGAGATGTTTCAAAAGAAG
SEQ ID NO: 87 is Abacus KR genomic DNA sequence flanking causative SNP (T/C substitution at
415 bp in Abacus CDS) located at position 51 bp (Abacus AA substitution S139P)
TACAGATCGAAGCATCTGGTGGCCAAGCTATTACTTTTGGAGGAGATGTTTCAAAAGAAGCTG
ATGTTGAATTAATGATCAAAACAGTGAGAAAATGTTCT
SEQ ID NO: 88 is Abacus KR genomic DNA sequence flanking causative SNP (A/G substitution at
472 bp in Abacus CDS) located at position 51 bp (Abacus AA substitution I158V)
TTGTAATATTCCATCTCATAAACCTGCAGGCAGTTGATGCTTGGGGAACTATTGATGTATTAGT
AAATAATGCAGGTTGTTTGAAACTTTCATTTTCTTAT
SEQ ID NO: 89 is Abacus KR genomic DNA sequence flanking causative SNP (A/G substitution at
523 bp in Abacus CDS) located at position 51 bp (Abacus AA substitution R175G)
CTGATGCTTTTTCGGTGTTTTCAGGAATCACAAGGGACAACTTAATTATGAGAATGAAGAAATC
CCAGTGGCAGGATGTCATTGATTTGAATCTTACTGGT
SEQ ID NO: 90 is Abacus KR genomic DNA sequence flanking causative SNP (T/C substitution at
578 bp in Abacus CDS) located at position 51 bp (Abacus AA substitution F193S)
GAAGAAATCCCAGTGGCAGGATGTCATTGATTTGAATCTTACTGGTGTATTTCTATGTACACAG
GTGTGCATCTAAAGCTTTCACTTCTTAAGGTCCTTCA
SEQ ID NO: 91 is Abacus KR genomic DNA sequence flanking causative SNP (A/G substitution at
614 bp in Abacus CDS) located at position 51 bp (Abacus AA substitution K205R)
GAAACTAAAATTAATTCTTTATTTGCAGGCTGCAGCCAAAATTATGATGAAGAAGAAAAAGGT
ATATACACCGTTTTATCTCTCCATTTACAGTCAAAACT
SEQ ID NO: 92 is Abacus KR genomic DNA sequence flanking causative SNP (A/G substitution at
623 bp in Abacus CDS) located at position 51 bp (Abacus AA substitution K208R)
ATTAATTCTTTATTTGCAGGCTGCAGCCAAAATTATGATGAAGAAGAAAAAGGTATATACACC
GTTTTATCTCTCCATTTACAGTCAAAACTATTTTTATA
SEQ ID NO: 93 is Abacus KR genomic DNA sequence flanking causative SNP (G/A substitution at
649 bp in Abacus CDS) located at position 51 bp (Abacus AA substitution V217I)
TTAGTCCCCAAATAATAATTTTGTAGGGAAGGATAATCAATGTAGCATCAGTCTTCGGTCTAGT
CGGCAATGCCGGGCAAGCCAATTATAGTGCTGCAAAA
SEQ ID NO: 94 is Abacus KR genomic DNA sequence flanking causative SNP (T/C substitution at
653 bp in Abacus CDS) located at position 51 bp (Abacus AA substitution F218S)
TCCCCAAATAATAATTTTGTAGGGAAGGATAATCAATGTAGCATCAGTCTTCGGTCTAGTCGGC
AATGCCGGGCAAGCCAATTATAGTGCTGCAAAAGCAG
SEQ ID NO: 95 is Abacus KR genomic DNA sequence flanking causative SNP (A/G substitution at
752 bp in Abacus CDS) located at position 51 bp (Abacus AA substitution N251S)
AGGAGTAATTGGCTTCACAAAAGCTATTGCTAAGGAGTATTCCAGCAGAAACATCACTGTGAG
TTATTCCAAAGTTTTTTTTTTCAAATAATTAGATATTT
SEQ ID NO: 96 is Abacus KR genomic DNA sequence flanking causative SNP (G/A substitution at
877 bp in Abacus CDS) located at position 51 bp (Abacus AA substitution V293I)
ATTAGTGTGATTTCTATTGTTCTTCAGGAAGATATGGTCAACCAGAAGAAGTTGCTGGGCTAGT
GGAATTTTTGGCACTTGGTCCTGCTGCAAGTTACATA
SEQ ID NO: 97 is 22VLV2-1-52 FATB CDS (439-1045 bp; N=missing sequence)
ATCTGGGGAGAGAGAGTGGAAATCCATACATGGATTGGAGCATCAGGGAACAATGGAGTGCA
AAGAGATTGGCTAATTCAAAATCAAGACACCGGCCATATTTTAGTTCGTGGAACTAGTACATG
GGTGATGATGAATCGAGAAACAAGGCGACTCTCGAAGATTCCAGAAGAGGTGAGGGATGAGA
TTTCGCCTTGGTTCATAGAGAAGAAAGCTATCAAAGAAGATGTCATTGACAAAATTGTCAAATT
GGATGATAAAGCTAAGTACATGAACACCAACTTGAAGNNNNNNNNNNNNNNNNNNNNNNNN
NNNNNNNNNNNNNNNNNNNNNNNNNNNNNNNNNNNNNNNNNNNNNNTCATACCGGATGCCA
TTTCAGAGTCTTACCAATTGTCTAAAATAACCCTAGAATACAAAAGGGAATGTGGGAGTACTA
GGGACATAGTCCAATTCCTTTGTCAACCTAATGAAGATGGAAGAGGGTTACTTAAACATGGAG
TAAATCAAGATAATAATCATAACAATCTACTAAATACTTTGGCTATTGAATTTCTCAAAAACAA
TGGATTCACGGGGTCCTTTGAGATAGGGCCATTTAGGTACACTC
SEQ ID NO: 98 is Abacus FATB CDS (1-1176 bp)
ATGTCTGCTTTGTCATTTCCACCATTCAATGCCATAAGATGTTGTTCTAATAGGGACAATGATC
AAAACCCAAGTGTTCAAAAGTTTAATAGCATTAATTTTAATGGAAGTTCAAGCTCTCAAACTGA
TCATAAACTTAGCCTAAAAATTGGTCCTTTACCAAGCTCCTTTTCTTTGACTTTTGTCACTAAAA
AAGAATCCTTCAATGATGAGAAAATTCAAGAGAATACCCCAACAAAGAAGCATGTAGTTGACC
CTTTTCGCCAAGAGCTCATAATTGAAGGAGGAGTAGGGTACAGACAGACTGTTGTTATTAGAT
CTTACGAAGAAGTAGCATTAAATCATGTAGGGATGTCAGGATTCATGAGTGATGGAATGGTGA
GAAACAATCTCATATGGGTTGTTTCAAGAATGCAAGTTCAAATTGAACAATATCCAATCTGGG
GAGAGAGAGTGGAAATCGATACATGGATTGGAGCATCAGGGAACAATGGAGTGCAAAGAGAT
TGGCTAATTCGAAATCAAGACACCGGCCATATTTTAGTTCGTGGAACTAGTACATGGGTGATGA
TGAATCGAGAAACAAGGGGACTCTCGAAGATGCAAGAAGAGGTGAGGGAGGAGATTTCGCCT
TGGTTCATAGAGAAAAAAGCTATCAAAGAAGATGTTATTGACAAAATTGTCAAATTGGATGAT
AAAGCTAAGTACATGAACACCAACTTAAAGCCCAAGAGAAGTGATTTGGACATGAACCACCAT
GTGAACAATGTCAAGTATGTAAGATGGATGCTCGAGATCATACCGGATGCCATTTCAGAGTCTT
ACCAATTGTCTAAAATAACCCTAGAATACAAAAGGGAATGTGGGAGTACTAGGGACATAGTCC
AATTCCTTTGTCAACCTAATGAAGATGGAAGAGGGTTACTTAAACATGGAGTAAATCAAGATA
ATAATCATAACAATCTACTAAATACTTTGGCTATTGAATTTCTCAAAAACAATGGATTCACGGG
GTCCTTTGAGATAGGGCCATTTAGGTACACTCATCTTCCCCAAATCGATCATATAGAGCCTTCT
AAGTGTACTCATCTCCTTCGATTCGGTCAACTTCTGCTTCAAATTCTCTTCTTCTTTGATCATGTT
CTTCGCCACTTCTTCAATGGAGTTTGGTATTAG
SEQ ID NO: 99 is 22VLV2-1-52 FATB protein sequence (147-348 AA; X = missing sequence)
WGERVEIHTWIGASGNNGVQRDWLIQNQDTGHILVRGTSTWVMMNRETRRLSKIPEEVRDEISPW
FIEKKAIKEDVIDKIVKLDDKAKYMNTNLKXXXXXXXXXXXXXXXXXXXXXXXXIPDAISESYQL
SKITLEYKRECGSTRDIVQFLCQPNEDGRGLLKHGVNQDNNHNNLLNTLAIEFLKNNGFTGSFEIGP
FRYT
SEQ ID NO: 100 is Abacus FATB protein sequence (1-391 AA)
MSALSFPPFNAIRCCSNRDNDQNPSVQKFNSINFNGSSSSQTDHKLSLKIGPLPSSFSLTFVTKKESFN
DEKIQENTPTKKHVVDPFRQELIIEGGVGYRQTVVIRSYEEVALNHVGMSGFMSDGMVRNNLIWV
VSRMQVQIEQYPIWGERVEIDTWIGASGNNGVQRDWLIRNQDTGHILVRGTSTWVMMNRETRGLS
KMQEEVREEISPWFIEKKAIKEDVIDKIVKLDDKAKYMNTNLKPKRSDLDMNHHVNNVKYVRWM
LEIIPDAISESYQLSKITLEYKRECGSTRDIVQFLCQPNEDGRGLLKHGVNQDNNHNNLLNTLAIEFL
KNNGFTGSFEIGPFRYTHLPQIDHIEPSKCTHLLRFGQLLLQILFFFDHVLRHFFNGVWY
SEQ ID NO: 101 is Abacus FATB genomic DNA sequence flanking causative SNP (G/C substitution
at 463 bp in Abacus CDS) located at position 51 bp (Abacus AA substitution D155H)
ACTTTTAACACATTTATGTATAATTCCTTAGGGGAGAGAGAGTGGAAATCGATACATGGATTGG
AGCATCAGGGAACAATGGAGTGCAAAGAGATTGGCTA
SEQ ID NO: 102 is Abacus FATB genomic DNA sequence flanking causative SNP (G/A substitution
at 518 bp in Abacus CDS) located at position 51 bp (Abacus AA substitution R173Q)
ATGGATTGGAGCATCAGGGAACAATGGAGTGCAAAGAGATTGGCTAATTCGAAATCAAGACA
CCGGCCATATTTTAGTTCGTGGAACTAGGTAAGAATATC
SEQ ID NO: 103 is Abacus FATB genomic DNA sequence flanking causative SNP (G/C substitution
at 589 bp in Abacus CDS) located at position 51 bp (Abacus AA substitution G197R)
TTTTAGTTCGTGGAACTAGTACATGGGTGATGATGAATCGAGAAACAAGGGGACTCTCGAAGA
TGCAAGAAGAGGTGAGGGAGGAGATTTCGCCTTGGTTC
SEQ ID NO: 104 is Abacus FATB genomic DNA sequence flanking causative SNP (G/T substitution
at 603 bp in Abacus CDS) located at position 51 bp (Abacus AA substitution M201I)
AACAGTACATGGGTGATGATGAATCGAGAAACAAGGGGACTCTCGAAGATGCAAGAAGAGGT
GAGGGAGGAGATTTCGCCTTGGTTCATAGAGAAAAAAGC
SEQ ID NO: 105 is Abacus FATB genomic DNA sequence flanking causative SNP (A/C substitution
at 605 bp in Abacus CDS) located at position 51 bp (Abacus AA substitution Q202P)
CAGTACATGGGTGATGATGAATCGAGAAACAAGGGGACTCTCGAAGATGCAAGAAGAGGTGA
GGGAGGAGATTTCGCCTTGGTTCATAGAGAAAAAAGCTA
SEQ ID NO: 106 is Abacus FATB genomic DNA sequence flanking causative SNP (G/T substitution
at 621 bp in Abacus CDS) located at position 51 bp (Abacus AA substitution E207D)
ATGAATCGAGAAACAAGGGGACTCTCGAAGATGCAAGAAGAGGTGAGGGAGGAGATTTCGCC
TTGGTTCATAGAGAAAAAAGCTATCAAAGAAGATGTTAT
SEQ ID NO: 107 is 22VLV2-1-52 GDSL1 CDS (67-961 bp)
ACGATTATTAGTTATGTTAATGCAAACCCTAAAGTTTCTTGCTTTTTCATATTTGGAGATTCTTT
GGCTGATAATGGCAACAACAATAATCTCAACACTTTGGCTAAAGCCAATTATAAGCCTTATGG
GATTGATTTCATTGATTCTCAACCAACAGGAAGATTCACCAATGGAAGAACCACTGTTGATATT
ATTGGTGAGTTTTTGGGTTTTGAGAAGTTAATTCCAGCCTTTGCTAGTCTTGATAATAATAGCTT
CAATCTATTGAGTGGTGCTAATTATGCATCTGGGGGAGCTGGGATTCTCAGGCAAACAGGAAC
ACATATGGGTGAAAATATTTGCTTGAAGAAACAAATAAAGCATCACCAAATTATGGTATCAAG
AATAAGTGACAAATTAGGGAGCAAAAAATTAGCACAAGAGCTATTAAACAAGTGCTTATATTG
GGTTGAAATTGGAAATAATGATTACATTAACAACTACTTCATGCCTTGGATTTATTCTTCTGGA
AATCTTTATACACCTCAACAATTTGCTCTTATTCTCATCCAAAAATATCGACTTCATATCATGGA
ATTGTACAATTTGGGGGCAAGAATGATCACTTTAGTTGGATTGGGACAAATCGGATGCACTCC
GAATGCAATTCAAATATATGGGACAAACAACGATTCTTCTTCATGTATAAGTTACATGAATGAC
GCTGTTCAATTATTCAATAATAAACTTACAATGCTCGTAGATGAACTTAACAGTAATTTTACCG
ATGCAAAATTTATTTACGTCAATTCTTATGAGATAGGATTGTCTGAAGATCTCATCACTCTTGG
GTTTAAGGTTTTGAATGTTGGTTGTTGTGGTGGAGATGAGAATGGACAATGTAAGCCTTTGGAG
A
SEQ ID NO: 108 is Abacus GDSL1 CDS (1-1089 bp)
ATGGAGTCAACATTATTTGATCAGATAAAAAGTGGGAGATTGTTAGCGTTTATGATCGTGATAA
TAACGATTATTAGTTATGTTAATGCAAACCCTAAAGTTTCTTGCTTTTTCATATTTGGAGATTCT
TTGGCTGATAATGGGAATAACAATAATCTCAACACTTTGGCTAAAGTCAATTATAAGCCTTATG
GGATTGACTTCATTGATTCTCAACCAACAGGAAGATTCACCAATGGAAGAACCACTGTTGATAT
TATTGGTGAGTTTTTGGGTTTTGAGAAGTTAATTCCAGCCTTTGCTAGTCTTGATAATAATAGCT
TCAATCTATTGAGTGGTGCTAATTATGCATCTGGGGGAGCTGGGATTCTCAGGCAAACAGGAA
CACATATGGGTGAAAATATTTGCTTGAAGAAACAAATAAAGCATCACCAAATTATGGTATCAA
GAATAAGTGACAAATTAGGGAGCAAAAAATTAGCACAAGAGCTATTAAACAAGTGCTTATATT
GGGTTGAAATTGGAAATAATGATTACATTAACAACTACTTCATGCCTTGGATTTATTCTTCTGG
AAATCTTTATACACCTCAACAATTTGCTCTTATTCTCATCCAAAAATATCGACTTCATATCTTGG
AATTGTACAATTTGGGGGCAAGAATGATCACTTTAGTTGGATTGGGACAAATCGGATGCACTC
CGAATGCAATTCAAATATATGGGACGAACAACGATTCTTCTTCATGTATAAGTTACATGAATGA
CGCTGTTCAGTTATTCAATAATAAACTTACAACGCTCGTAGATGAACTTAACAGTGATTTTACC
GATGCAAAATTTATTTACGTCAATTCTTATGAGATAGGATTGTCTGAAGATCTCATCGCTCTTG
GGTTTAAGGTTTTGAATGTTGGTTGTTGTGGTGGAGATGAGAATGGACAATGTAAGCCTTTGGA
GATTCCATGTGAGAATAGAAGAGATTATGTGTTTTGGGATTCATTTCATCCAACTGAGGCTTAC
AACTTAATCACTGCAACTAAAATATATCAAGCTTATTATAACATATCATATATAGATACCCAAT
AA
SEQ ID NO: 109 is 22VLV2-1-52 GDSL1 protein sequence (23-320 AA)
TIISYVNANPKVSCFFIFGDSLADNGNNNNLNTLAKANYKPYGIDFIDSQPTGRFTNGRTTVDIIGEFL
GFEKLIPAFASLDNNSFNLLSGANYASGGAGILRQTGTHMGENICLKKQIKHHQIMVSRISDKLGSK
KLAQELLNKCLYWVEIGNNDYINNYFMPWIYSSGNLYTPQQFALILIQKYRLHIMELYNLGARMIT
LVGLGQIGCTPNAIQIYGTNNDSSSCISYMNDAVQLFNNKLTMLVDELNSNFTDAKFIYVNSYEIGL
SEDLITLGFKVLNVGCCGGDENGQCKPLE
SEQ ID NO: 110 is Abacus GDSL1 protein sequence (1 362 AA)
MESTLFDQIKSGRLLAFMIVIITIISYVNANPKVSCFFIFGDSLADNGNNNNLNTLAKVNYKPYGIDFI
DSQPTGRFTNGRTTVDIIGEFLGFEKLIPAFASLDNNSFNLLSGANYASGGAGILRQTGTHMGENICL
KKQIKHHQIMVSRISDKLGSKKLAQELLNKCLYWVEIGNNDYINNYFMPWIYSSGNLYTPQQFALIL
IQKYRLHILELYNLGARMITLVGLGQIGCTPNAIQIYGTNNDSSSCISYMNDAVQLFNNKLTTLVDEL
NSDFTDAKFIYVNSYEIGLSEDLIALGFKVLNVGCCGGDENGQCKPLEIPCENRRDYVFWDSFHPTE
AYNLITATKIYQAYYNISYIDTQ
SEQ ID NO: 111 is Abacus GSDL1 genomic DNA sequence flanking causative SNP (T/C substitution
at 176 bp in Abacus CDS) located at position 51 bp (Abacus AA substitution V59A)
TTCTTTGGCTGATAATGGGAATAACAATAATCTCAACACTTTGGCTAAAGTCAATTATAAGCCT
TATGGGATTGACTTCATTGATTCTCAACCAACAGGAA
SEQ ID NO: 112 is Abacus GSDL1 genomic DNA sequence flanking causative SNP (G/A substitution
at 823 bp in Abacus CDS) located at position 51 bp (Abacus AA substitution D275N)
TTCAGTTATTCAATAATAAACTTACAACGCTCGTAGATGAACTTAACAGTGATTTTACCGATGC
AAAATTTATTTACGTCAATTCTTATGAGATAGGATTG
SEQ ID NO: 113 is Abacus GSDL1 genomic DNA sequence flanking causative SNP (G/A substitution
at 889 bp in Abacus CDS) located at position 51 bp (Abacus AA substitution A297T)
AATTTATTTACGTCAATTCTTATGAGATAGGATTGTCTGAAGATCTCATCGCTCTTGGTAAGAA
TTATTACCAATAATACTTTTAATTTTATAATTTCATA
SEQ ID NO: 114 is Abacus GSDL1 genomic DNA sequence flanking causative SNP (T/A substitution
at 637 bp in Abacus CDS) located at position 51 bp (Abacus AA substitution L213M)
CACCTCAACAATTTGCTCTTATTCTCATCCAAAAATATCGACTTCATATCTTGGTAATTAATTCT
TAATTAATACTTCTAATAATTACATTATTATGCTTG

### DETAILED DESCRIPTION

Unless otherwise noted, technical terms are used according to conventional usage. As used herein, the singular forms “a”, “an” and “the” are intended to include the plural forms as well, unless the context clearly indicates otherwise. The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. The term “about” refers to an amount within a specific range of a value. For example, “about” indicates within 10% the stated amount (unless indicated otherwise).

Although many methods and materials similar or equivalent to those described herein can be used, specific suitable methods and materials are described herein. In case of conflict, the present specification, including explanations of terms, will control. In addition, the materials, methods, and examples are illustrative only and not intended to be limiting. To facilitate review of the various aspects, the following explanations of terms are provided:

### Definitions

The term “Abacus” as used herein refers to the Cannabis sativa reference genome known as the Abacus Cannabis reference genome version Csat_AbacusV2 (NCBI assembly accession GCA_025232715.1, incorporated by reference herein), which is also sometimes referred to as CsaAba2.

The term “about” refers to a range of 5% of the referenced value unless otherwise indicated. For example, about 100 refers to a range of 95 to 105.

The term “alternative nucleotide call” is a nucleotide polymorphism relative to a reference nucleotide for a SNP marker that is significantly associated with the causative SNP(s) that confer(s) a desired phenotype (e.g., increased varin production).

“Backcrossing” is a process in which a breeder repeatedly crosses hybrid progeny, for example a first generation hybrid (F1), back to one of the parents of the hybrid progeny. Backcrossing can be used to introduce one or more single locus conversions from one genetic background into another.

The term “beneficial” as used herein refers to a genetic element (e.g., gene, allele, or polymorphism) conferring or associated with an increased varin production phenotype.

The term “Cannabis” refers to plants of the genus Cannabis, including Cannabis sativa, Cannabis indica, and Cannabis ruderalis.

The term “CBCV” means cannabichromevarin.

The term “CBCVA” means cannabichromevarinic acid.

The term “CBDV” means cannabidivarin.

The term “CBDVA” means cannabidivarinic acid.

The term “CBGV” means cannabigerivarin.

The term “CBGVA” means cannabigerivarinic acid.

The term “cell” refers to a prokaryotic or eukaryotic cell, including plant cells, capable of replicating DNA, transcribing RNA, translating polypeptides, and secreting proteins.

The term “coding sequence” refers to a DNA sequence which codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences.

The term “control” refers to a reference standard. A control can be a negative or positive control. In some examples, the control is a historical control or a known reference value (or range of values). A practitioner can select a suitable control based on the teachings provided herein. Non-limiting examples of suitable controls include a Cannabis plant not including a polymorphism associated with increased varin production as disclosed herein.

The term “cross” or “crossing” refer to the process by which the pollen of one flower on one plant is applied (artificially or naturally) to the ovule (stigma) of a flower on another plant (or the same plant when selfing). Exemplary types of crosses include selfing, backcrossing, outcrossing, and sibling crossing.

The term “cultivar” means a group of similar plants that by structural features and performance (e.g., morphological and physiological characteristics) can be identified from other varieties within the same species. Furthermore, the term “cultivar” variously refers to a variety, strain or race of plant that has been produced by horticultural or agronomic techniques and is not normally found in wild populations. The terms cultivar, variety, strain, plant and race are often used interchangeably by plant breeders, agronomists and farmers.

The term “detect” or “detecting” refers to any method for determining the presence of a nucleic acid. Methods of detecting nucleic acid polymorphisms, for example, have been described and can include amplification of a target polynucleotide (e.g., by PCR) and/or detection by a probe (e.g., hybridization assays). PCR uses a particular amplification primer pair that specifically hybridize to a target polynucleotide and produce an amplification product (the amplicon). Primers can be designed such that the amplicon can contain a nucleic acid polymorphism of interest. Methods for designing PCR primers and PCR conditions have been described, for example, in Sambrook et al. (2014) Molecular Cloning: A Laboratory Manual (Fourth Edition, Cold Spring Harbor Laboratory Press, Plainview, N.Y.).

The term “expression” or “gene expression” relates to the process by which the coded information of a nucleic acid transcriptional unit (including, e.g., genomic DNA) is converted into an operational, non-operational, or structural part of a cell, often including the synthesis of a protein. Gene expression can be influenced by external signals; for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any suitable, known method, including, without limitation, Northern blot, RT-PCR, Western blot, or in vitro, in situ, or in vivo protein activity assay(s). Elevated (increased) levels refer to higher than average levels of gene expression in comparison to a reference, e.g., the Abacus reference genome.

The term “expression cassette” refers to a discrete nucleic acid fragment into which a nucleic acid sequence or fragment can be moved, typically for expression in a host cell.

The term “functional” as used herein refers to DNA or amino acid sequences which are of sufficient size and sequence to have the desired function (i.e. the ability to cause expression of a gene resulting in gene activity expected of the gene found in a reference genome, e.g., the Abacus reference genome).

The term “gene” or “allele” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” or “recombinant expression construct”, which are used interchangeably, refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “heterologous” gene or allele refers to a gene or allele that is not naturally found in the host, but is artificially introduced (e.g., by genetic engineering or selective plant breeding). Heterologous genes can comprise native genes inserted into a non-native host, or chimeric genes.

The term “genetic modification” as used herein refers to a change from the wild-type or reference sequence of one or more nucleic acid molecules. Genetic modifications or alterations include without limitation, base pair substitutions, additions and deletions of at least one nucleotide from a nucleic acid molecule of known sequence.

The term “genome” as it applies to plant cells encompasses not only chromosomal DNA found within the nucleus, but organelle DNA found within subcellular components (e.g., mitochondrial, plastid) of the cell.

The term “genotype” refers to the genetic makeup of an individual cell, cell culture, tissue, organism (e.g., a plant), or group of organisms. A “detrimental genotype” is a genotype that does not produce varins, or is associated with reduced varin production. Conversely, a “beneficial genotype” refers to a genotype that produces varins, or is associated with increased varin production. A genotype may refer to a particular genetic marker (e.g., a polymorphism), such as a marker associated with varin production. Thus, a “detrimental polymorphism” is a polymorphism associated with reduced varin production or plants that do not produce varins, and a “beneficial polymorphism” refers to a polymorphism that is associated with increased varin production or plants that produce varins. In some examples, a beneficial genotype or polymorphism increases varin production relative to a plant that does not contain the beneficial genotype or polymorphism, or relative to a plant that contains a detrimental genotype or polymorphism.

The term “germplasm” refers to genetic material of or from an individual (e.g., a plant), a group of individuals (e.g., a plant line, variety, or family), or a clone derived from a line, variety, species, or culture. The germplasm can be part of an organism or cell, or can be separate from the organism or cell. In general, germplasm provides genetic material with a specific molecular makeup that provides a physical foundation for some or all of the hereditary qualities of an organism or cell culture. As used herein, germplasm includes cells, seed or tissues from which new plants can be grown, as well as plant parts, such as leaves, stems, pollen, or cells that can be cultured into a whole plant.

A plant is “homozygous” if the individual has only one type of allele at a given locus (e.g., a diploid individual has a copy of the same allele at a locus for each of two homologous chromosomes). An individual is “heterozygous” if more than one allele type is present at a given locus (e.g., a diploid individual with one copy each of two different alleles). The term “homogeneity” indicates that members of a group have the same genotype at one or more specific loci. In contrast, the term “heterogeneity” is used to indicate that individuals within the group differ in genotype at one or more specific loci.

The term “hybrid” refers to a variety or cultivar that is the result of a cross of plants of two different varieties. A hybrid, as described here, can refer to plants that are genetically different at any particular loci. A hybrid can further include a plant that is a variety that has been bred to have at least one different characteristic from the parent. “F1 hybrid” refers to the first generation hybrid, “F2 hybrid” the second generation hybrid, “F3 hybrid” the third generation, and so on. A hybrid refers to any progeny that is either produced, or developed using research and development to create a new line having at least one distinct characteristic.

The terms “hybridizing specifically to,” “specific hybridization,” or “selectively hybridize to,” as used herein refer to the binding, duplexing, or hybridizing of a nucleic acid molecule preferentially to a particular nucleotide sequence under stringent conditions. The term “stringent conditions” refers to conditions under which a nucleic acid will hybridize preferentially to a target sequence, and to a lesser extent to, or not at all to, other off-target sequences. A “stringent hybridization” and “stringent hybridization wash conditions” in the context of nucleic acid hybridization (e.g., as in array, Southern or Northern hybridizations) are sequence dependent, and are different under different environmental parameters. An extensive guide to the hybridization of nucleic acids can be found in, e.g., Tijssen (1993) Laboratory Techniques in Biochemistry and Molecular Biology-Hybridization with Nucleic Acid Probes part I, Ch. 2, “Overview of principles of hybridization and the strategy of nucleic acid probe assays,” Elsevier, N.Y. (“Tijssen”). Generally, highly stringent hybridization and wash conditions are selected to be about 5° C. lower than the thermal melting point for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on an array or on a filter in a Southern or northern blot is 42° C. using standard hybridization solutions (see, e.g., Sambrook et al. (2014) Molecular Cloning: A Laboratory Manual (Fourth Edition, Cold Spring Harbor Laboratory Press, Plainview, N.Y.)).

As used herein, the term “inbreeding” refers to the production of offspring via the mating between relatives. The plants resulting from the inbreeding process are referred to herein as “inbred plants” or “inbreds.”

The terms “increase” or “decrease” refer to a positive (increase) or negative (decrease) difference relative to a reference value, such as a control. The difference can be quantitative. In some examples, the difference is an increase relative to a control of at least 5%, such as at least 10%, at least 20%, at least 30%, at least 40%, at least 50%, at least 60%, at least 70%, at least 80%, at least 90%, at least 100%, at least 150%, at least 200%, at least 250%, at least 300%, at least 350%, at least 400%, at least 500%, or greater than 500%. In some examples, the difference is a decrease relative to a control of at least 5%, such as at least 10%, at least 20%, at least 30%, at least 40%, at least 50%, at least 60%, at least 70%, at least 80%, at least 90%, at least 95%, at least 98%, or 100%. In some examples, the difference is statistically significant (e.g., P-value less than 0.05 or 0.01).

“Increased varin production” refers to a relative increase of varin content in a plant relative to a suitable reference. The reference depends on context, however, in some examples the reference plant is a Cannabis plant not including one or more nucleic acid polymorphisms associated with increased varin production, or an unmodified Cannabis plant. In some examples, the reference is Abacus.

The term “introduced” refers to the incorporation of a particular nucleic acid sequence or protein into a cell, for example, via transformation. “Transformation” encompasses all techniques by which a nucleic acid molecule or protein might be introduced into such a cell, including chemical methods (e.g., calcium-phosphate transfection), physical methods (e.g., electroporation, microinjection, particle bombardment), fusion (e.g., liposomes), lipofection, nucleofection, receptor-mediated endocytosis (e.g., DNA-protein complexes, viral envelope/capsid-DNA complexes), agrobacterium-mediated transformation, biolistics (particle gun accelerator or gene gun), or other transduction and/or transfection methods. Transformation can include stable transformation, where a nucleic acid fragment is incorporated into the genome of a host cell (e.g., chromosome, plasmid, plastid or mitochondrial DNA), or transient transformation, e.g., transformation of an autonomous replicon or other transient molecule (e.g., transfected mRNA). In some examples, a genetic modification (e.g., a substitution, insertion, or deletion) is introduced through a gene editing technique, such as an RNAi, CRISPR/Cas9, ZFN, or TALEN based technique. In some examples, a heterologous gene (or vector carrying a gene) is introduced into a cell by transformation, transfection, or transduction.

The terms “isolated” or “purified” in reference to biological components (such as nucleic acids, proteins, or cells) are components that have been substantially separated from other biological components in the environment in which the component occurs, e.g., separated from other chromosomal and extra-chromosomal DNA and RNA, proteins and/or cells. Nucleic acids and proteins that have been “isolated” include nucleic acids and proteins purified by standard purification methods. The term also embraces nucleic acids and proteins prepared by recombinant expression in a host cell as well as chemically synthesized nucleic acids. Absolute purity or isolation is not required, it is intended as a relative term. Thus, for example, a purified/isolated protein, nucleic acid, or cell preparation is one in which the protein, nucleic acid, or cell is more enriched than the protein, nucleic acid, or cell is in its initial environment. In one example, a preparation is purified/isolated such that the protein, nucleic acid, or cell represents at least 50% of the total content of the preparation. A substantially purified protein or nucleic acid is at least 60%, at least 70%, at least 80%, at least 90%, at least 95%, at least 98%, or at least 99% pure. Thus, in one specific, non-limiting example, a substantially purified protein or nucleic acid is 90% free of other components.

The term “line” is used broadly to include, but is not limited to, a group of plants vegetatively propagated from a single parent plant, via tissue culture techniques or a group of inbred plants which are genetically very similar due to descent from a common parent(s). A plant is said to “belong” to a particular line if it (a) is a primary transformant (TO) plant regenerated from material of that line; (b) has a pedigree comprised of a TO plant of that line; or (c) is genetically very similar due to common ancestry (e.g., via inbreeding or selfing). In this context, the term “pedigree” denotes the lineage of a plant, e.g. in terms of the sexual crosses affected such that a gene or a combination of genes, in heterozygous (hemizygous) or homozygous condition, imparts a desired trait to the plant.

The term “marker,” “genetic marker,” or “molecular marker,” refer to a nucleotide sequence or encoded product thereof (e.g., a protein) used as a point of reference for identifying a linked locus. A marker can be derived from genomic nucleotide sequence or from expressed nucleotide sequences (e.g., from a spliced RNA, a cDNA, etc.), or from an encoded polypeptide, and can be represented by one or more particular variant sequences, or by a consensus sequence. A “marker probe” is a nucleic acid molecule that can be used to identify the presence of a marker, e.g., a nucleic acid probe that is complementary to a marker locus sequence. Alternatively, in some aspects, a marker probe refers to a probe of any type that is able to distinguish (i.e., genotype) the particular allele that is present at a marker locus. A “marker locus” is a locus that can be used to track the presence of a second linked locus, e.g., a linked locus that encodes or contributes to expression of a phenotypic trait. For example, a marker locus can be used to monitor segregation of alleles at a locus, such as a QTL, that are genetically or physically linked to the marker locus. Thus, a “marker allele,” alternatively an “allele of a marker locus,” is one of a plurality of polymorphic nucleotide sequences found at a marker locus in a population that is polymorphic for the marker locus. Examples of markers include restriction fragment length polymorphism (RFLP) markers, amplified fragment length polymorphism (AFLP) markers, single nucleotide polymorphisms (SNPs), microsatellite markers (e.g. SSRs), sequence-characterized amplified region (SCAR) markers, cleaved amplified polymorphic sequence (CAPS) markers or isozyme markers or combinations of the markers described herein which defines a specific genetic and chromosomal location.

The term “marker assisted selection” refers to the diagnostic process of identifying, optionally followed by selecting a plant from a group of plants using the presence of a molecular marker as the diagnostic characteristic or selection criterion. The process usually involves detecting the presence of a certain nucleic acid sequence or polymorphism in the genome of a plant.

The term “modified Cannabis plant” or “modified plant” is not a naturally occurring plant.

The term “neutral cannabinoid” refers to a cannabinoid without carboxylic acid functional groups. Examples of neutral cannabinoids include, but are not limited to, THC, THCV, CBD, CBG, CBC, and CBN.

The term “nucleotide” refers to an organic molecule that serves as a monomeric unit of DNA and RNA. The nucleotide position is the position along a reference sequence wherein any particular monomeric unit of DNA or RNA is positioned relative to the other monomeric units of DNA or RNA.

The term “offspring” refers to any progeny from one or more parent plants. For instance, an offspring plant may be obtained by cloning or selfing of a parent plant or by crossing two parent plants. An F1 is a first-generation offspring produced from parents at least one of which is used for the first time as donor of a trait, while offspring of second generation (F2) or subsequent generations (F3, F4, etc.) are specimens produced from selfings of F1's, F2's etc. An F1 may thus be (and usually is) a hybrid resulting from a cross between two true breeding parents (true-breeding is homozygous for a trait), while an F2 may be (and usually is) an offspring resulting from self-pollination.

The term “operably linked” refers to the association of nucleic acid sequences on a single nucleic acid fragment such that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “plant” refers to a whole plant, cell, tissue, or other plant parts. Plant parts include any part(s) of a plant, including, for example and without limitation: seed (including mature seed and immature seed); a plant cutting; a plant cell; a plant cell culture; a plant organ (e.g., pollen, embryos, flowers, trichomes, fruits, shoots, leaves, roots, stems, and explants). Plant tissue refers to any tissue of a plant, including but not limited to, tissue from an embryo, shoot, root, stem, seed, stipule, leaf, trichome, petal, flower bud, flower, ovule, bract, branch, petiole, internode, bark, pubescence, tiller, rhizome, frond, blade, ovule, pollen, stamen. A plant tissue or plant organ may be a seed, protoplast, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. In contrast, some plant cells are not capable of being regenerated to produce plants. Regenerable cells in a plant cell or tissue culture may be embryos, protoplasts, meristematic cells, callus, pollen, leaves, anthers, roots, root tips, silk, flowers, kernels, ears, cobs, husks, or stalks. Plant parts include harvestable parts and parts useful for propagation of progeny plants. Plant parts useful for propagation include, for example and without limitation: seed; fruit; a cutting; a seedling; a tuber; and a rootstock. A harvestable part of a plant may be any useful part of a plant, including, for example and without limitation: flower; pollen; seedling; tuber; leaf; stem; fruit; seed; and root. A plant cell is the structural and physiological unit of the plant, comprising a protoplast and a cell wall. A plant cell may be in the form of an isolated single cell, or an aggregate of cells (e.g., a friable callus and a cultured cell), and may be part of a higher organized unit (e.g., a plant tissue, plant organ, and plant). Thus, a plant cell may be a protoplast, a gamete producing cell, or a cell or collection of cells that can regenerate into a whole plant. As such, a seed, which comprises multiple plant cells and is capable of regenerating into a whole plant, is considered a “plant cell.” Described herein are plants in the genus of Cannabis and plants derived therefrom, which can be produced by asexual or sexual reproduction.

The terms “polynucleotide,” “polynucleotide sequence,” “nucleotide sequence,” “nucleic acid sequence,” and “nucleic acid fragment,” are used interchangeably. These terms encompass polymers composed of nucleotide units (ribonucleotides, deoxyribonucleotides, related naturally occurring structural variants, and synthetic non-naturally occurring analogs thereof). The term “oligonucleotide” typically refers to short polynucleotides, generally no greater than 150 nucleotides, for example, no greater than 125 nucleotides, no greater than 100 nucleotides, no greater than 75 nucleotides, no greater than 50 nucleotides, or no greater than 25 nucleotides. It will be understood that when a nucleic acid sequence is represented as a DNA sequence (i.e., A, T, G, C), this also includes an RNA sequence (i.e., A, U, G, C) in which “U” replaces “T.” Nucleic acids can be single- or double-stranded. Exemplary nucleic acids include cDNA, genomic DNA, synthetic DNA, RNA, or mixtures thereof.

The term “polymorphism” refers to a difference in the nucleotide or amino acid sequence of a given region as compared to a nucleotide or amino acid sequence in a homologous-region of another individual, in particular, a difference in the nucleotide of amino acid sequence of a given region which differs between individuals of the same species. A polymorphism is generally defined in relation to a reference sequence. Unless indicated otherwise, the reference sequence is the Cannabis Abacus reference genome (version Csat_AbacusV2 (NCBI assembly accession GCA_025232715.1) or CDS produced from the Cannabis Abacus reference genome. Polymorphisms include single nucleotide differences, differences in sequence of more than one nucleotide, and single or multiple nucleotide insertions, inversions and deletions; as well as single amino acid differences, differences in sequence of more than one amino acid, and single or multiple amino acid insertions, inversions, and deletions.

The term “polypeptide” or “protein” refers to a polymer of amino acid residues. The terms apply to amino acid polymers in which one or more amino acid residue is an artificial chemical analogue of a corresponding naturally occurring amino acid, as well as to naturally occurring amino acid polymers. The term “amino acid residue” or “amino acid” includes reference to an amino acid that is incorporated into a protein, polypeptide, or peptide. The amino acid can be a naturally occurring amino acid and, unless otherwise limited, can encompass known analogs of natural amino acids that can function in a similar manner as naturally occurring amino acids. As used herein, “recombinant” includes reference to a protein produced using cells that do not have, in their native state, an endogenous copy of the DNA able to express the protein. The cells produce the recombinant protein because they have been genetically altered by the introduction of the appropriate isolated nucleic acid sequence. The term also includes reference to a cell, or nucleic acid, or vector, that has been modified by the introduction of a heterologous nucleic acid or the alteration of a native nucleic acid to a form not native to that cell, or that the cell is derived from a cell so modified.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirements of the application. For example, in diagnostic applications, the oligonucleotide primer is typically 15-25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

The term “probe,” “nucleic acid probe,” or “oligonucleotide probe” as used herein, is one or more synthetic nucleic acid molecules that are complementary to a nucleic acid sequence of interest (target sequence), and hybridize to a sequence of interest when under hybridization conditions. Probes can be used to detect, analyze, and/or visualize the nucleic acid sequence of interest on a molecular level. Specific hybridization of a probe to a nucleic acid sequence of interest can be detected, for example, through a label on the probe. Probes have a length suitable to achieve a desired specificity to the target sequence, however, are generally at least 10 nucleotides long, for example, at least 15 nucleotides, at least 20 nucleotides, or at least 50 nucleotides long. Probes can be immobilized on a solid surface (e.g., nitrocellulose, glass, quartz, fused silica slides), as in an array. The precise sequence of the particular probes described herein can be modified to a certain degree to produce probes that are “substantially identical” to the disclosed probes, but retain the ability to specifically bind to (i.e., hybridize specifically to) the same targets as the probe from which they were derived. Such modifications are specifically covered by reference to the individual probes described herein.

The term “product” as used in reference to a Cannabis product, is a composition including Cannabis (including a Cannabis plant, plant part, or extract). Products include, but are not limited to: a kief, hashish, bubble hash, an edible product, solvent reduced oil, sludge, e-juice, tincture, or other compositions including Cannabis (e.g., a Cannabis plant disclosed herein, or an extract thereof). The product is not, or excludes, any naturally occurring compositions.

The term “progeny” refers to any subsequent generation of a plant. Progeny is measured using the following nomenclature: F1 refers to the first generation progeny, F2 refers to the second generation progeny, F3 refers to the third generation progeny, and so on.

The term “promoter” refers to a nucleic acid control sequence capable of directing transcription of an operably linked nucleic acid. A promoter includes necessary nucleic acid sequences typically near the start site of transcription, and may include distal enhancer or repressor elements. A “constitutive promoter” is a promoter that is continuously active and is not subject to regulation by external signals or molecules. In contrast, the activity of an “inducible promoter” is regulated by an external signal or molecule (for example, a transcription factor). Exemplary promoters include pol III promoters (e.g., U6), pol II promoter, ubiquitin promoter, Cauliflower Mosaic Virus (CaMV) 35S promoter, or RUBISCO promoter. The terms “initiate transcription,” “initiate expression,” “drive transcription,” and “drive expression” are used interchangeably herein and all refer to the primary function of a promoter.

The term “quantitative trait loci” or “QTL” refers to the genetic elements controlling a quantitative trait.

The term “recombinant” refers to a nucleic acid or protein that has a sequence made by an artificial combination of two otherwise separated segments of sequence (e.g., a “chimeric” sequence). This artificial combination can be accomplished by chemical synthesis or by manipulation of isolated segments of nucleic acids, for example, by standard molecular biology techniques (e.g., cloning). The term “recombinant DNA construct” or “recombinant expression construct” is used interchangeably and refers to a discrete polynucleotide into which a nucleic acid sequence or fragment can be moved. Preferably, it is a plasmid vector, or a fragment thereof, comprising a promoter. The choice of plasmid vector is dependent upon the method that will be used to transform host plants. Similarly, genetic elements that must be present on the plasmid vector to successfully transform, select and propagate host cells containing the chimeric gene is dependent on the specific transformation method. Different independent transformation events typically result in different levels and patterns of expression and thus multiple events must be screened to obtain lines displaying the desired expression level and pattern. Such screening may be accomplished by PCR and Southern analysis of DNA, RT-PCR and Northern analysis of mRNA expression, Western analysis of protein expression, or phenotypic analysis

The term “reference plant” or “reference genome” refers to a reference sequence that genetic markers or sequences of a test sample can be compared to in order to detect a modification of the sequence in the test sample. In some embodiments, the reference plant or genome is Abacus (Csat_AbacusV2, NCBI assembly accession GCA_025232715.1).

The term “RNA transcript” refers to a product resulting from RNA polymerase-catalyzed transcription of a DNA sequence. When an RNA transcript is a perfect complementary copy of a DNA sequence, it is referred to as a primary transcript or it may be a RNA sequence derived from post-transcriptional processing of a primary transcript and is referred to as a mature RNA. “Messenger RNA” (“mRNA”) refers to RNA that is without introns and that can be translated into protein by the cell. “cDNA” refers to a DNA that is complementary to and synthesized from an mRNA template using the enzyme reverse transcriptase. The cDNA can be single-stranded or converted into the double-stranded by using the Klenow fragment of DNA polymerase I. “Sense” RNA refers to RNA transcript that includes mRNA and so can be translated into protein within a cell or in vitro. “Antisense RNA” refers to a RNA transcript that is complementary to all or part of a target primary transcript or mRNA and that blocks expression or transcripts accumulation of a target gene. The complementarity of an antisense RNA may be with any part of the specific gene transcript, i.e. at the 5′ non-coding sequence, 3′ non-coding sequence, introns, or the coding sequence. “Functional RNA” refers to antisense RNA, ribozyme RNA, or other RNA that may not be translated but yet has an effect on cellular processes.

The terms “sequence identity” or “percent identity” are used interchangeably to refer to a sequence comparison based on identical matches between correspondingly identical positions in the sequences being compared between two or more amino acid or nucleotide sequences. The percent identity refers to the extent to which two optimally aligned polynucleotide or peptide sequences are invariant throughout a window of alignment of components, e.g., nucleotides or amino acids. Hybridization experiments and mathematical algorithms known in the art may be used to determine percent identity. Many mathematical algorithms exist as sequence alignment computer programs known in the art that calculate percent identity. These programs may be categorized as either global sequence alignment programs or local sequence alignment programs.

The NCBI Basic Local Alignment Search Tool (BLAST) tool is often used and is available from several sources, including the National Center for Biotechnology Information (blast.ncbi.nlm.nih.gov/Blast.cgi). Various types of BLAST are available, for example, blastp, blastn, blastx, tblastn and tblastx. A description of how to determine sequence identity using this program is available on the NCBI website and other resources. In some examples, percent sequence identity is determined by using BLAST with default parameters.

The term “substantially similar” as used herein refers to nucleic acid fragments wherein changes in one or more nucleotide bases do not affect the ability of the nucleic acid fragment to mediate gene expression or produce a certain phenotype. These terms also refer to modifications of nucleic acid fragments, such as deletion or insertion of one or more nucleotides that do not substantially alter the functional properties of the resulting nucleic acid fragment relative to the initial, unmodified fragment. A “substantially homologous sequence” refers to variants of the disclosed sequences such as those that result from site-directed mutagenesis, as well as synthetically derived sequences. A substantially homologous sequence also refers to fragments of a particular promoter nucleotide sequence disclosed herein that operate to promote the constitutive expression of an operably linked heterologous nucleic acid fragment. These promoter fragments will include at least about 20 contiguous nucleotides, for example, at least 50 contiguous nucleotides, at least 75 contiguous nucleotides, or at least 100 contiguous nucleotides of the particular promoter nucleotide sequence disclosed herein. The nucleotides of such fragments will usually comprise the TATA recognition sequence of the particular promoter sequence. Such fragments may be obtained by use of restriction enzymes to cleave the naturally occurring promoter nucleotide sequences disclosed herein; by synthesizing a nucleotide sequence from the naturally occurring promoter DNA sequence; or may be obtained through the use of PCR technology. Functional variants of these promoter fragments, such as those resulting from site-directed mutagenesis, are encompassed by the present disclosure.

The term “single nucleotide polymorphism (SNP)” refers to a change in which a single base in the DNA differs from the base at the corresponding position of a reference genome or sequence. These single base changes are called SNPs.

The term “THCV” means tetrahydrocannabivarin.

The term “THCVA” means tetrahydrocannabivarinic acid.

The term “transformant” refers to a cell, tissue or organism that has undergone transformation. The original transformant is designated as “TO” or “TO.” Selfing the TO produces a first transformed generation designated as “T1” or “T1.”

The term “transgenic” refers to any cell, cell line, callus, tissue, plant part or plant, the genome of which has been altered by the presence of a heterologous nucleic acid, such as a recombinant DNA construct, including those initial transgenic events as well as those created by sexual crosses or asexual propagation from the initial transgenic event. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation. A “transgene” is a gene that has been introduced into the genome by a transformation procedure.

The term “translation leader sequence” refers to a polynucleotide sequence located between the promoter sequence of a gene and the coding sequence. The translation leader sequence is present in the fully processed mRNA upstream of the translation start sequence. The translation leader sequence may affect processing of the primary transcript to mRNA, mRNA stability or translation efficiency.

The term “variety” as used herein has identical meaning to the corresponding definition in the International Convention for the Protection of New Varieties of Plants (UPOV treaty), of Dec. 2, 1961, as Revised at Geneva on Nov. 10, 1972, on Oct. 23, 1978, and on Mar. 19, 1991. Thus, “variety” means a plant grouping within a single botanical taxon of the lowest known rank, which grouping, irrespective of whether the conditions for the grant of a breeder's right are fully met, can be i) defined by the expression of the characteristics resulting from a given genotype or combination of genotypes, ii) distinguished from any other plant grouping by the expression of at least one of the said characteristics and iii) considered as a unit with regard to its suitability for being propagated unchanged.

The term “varin” also known as “varinolic cannabinoid,” is a type of cannabinoid compound that has three carbon atoms in its alkyl side chain. Exemplary varins include tetrahydrocannabivarin (THCV), cannabigerivarin (CBGV), cannabichromevarin (CBCV), or cannabidivarin (CBDV). The term “varin ratio” refers to [total varin/(total THC+total CBD+total CBC+total CBG)]. The term “total varin” refers to the combination of THCV+CBDV+CBCV+CBGV.

The term “vector” refers to a nucleic acid molecule that can be introduced into a host cell (for example, by transformation), thereby producing a transformed host cell. A vector can include nucleic acid sequences that permit it to replicate in a host cell, such as an origin of replication. Recombinant DNA vectors are vectors containing recombinant DNA. A vector can also include one or more selectable marker genes and other genetic elements. Often vectors are DNA plasmids, however, they can also be viral vectors (DNA or RNA), cosmids, or artificial chromosomes.

### Cannabis

Cannabis has long been used for drug and industrial purposes, fiber (hemp), for seed and seed oils, for medicinal purposes, and for recreational purposes. Industrial hemp products are made from Cannabis plants selected to produce an abundance of fiber. Some Cannabis varieties have been bred to produce minimal levels of THC, the principal psychoactive constituent responsible for the psychoactivity associated with marijuana. Marijuana has historically consisted of the dried flowers of Cannabis plants selectively bred to produce high levels of THC and other psychoactive cannabinoids. Various extracts including hashish and hash oil are also produced from the plant.

Cannabis is an annual, dioecious, flowering herb. The leaves are palmately compound or digitate, with serrate leaflets. Cannabis normally has imperfect flowers, with staminate “male” and pistillate “female” flowers occurring on separate plants. It is not unusual, however, for individual plants to separately bear both male and female flowers (i.e., have monoecious plants). Although monoecious plants are often referred to as “hermaphrodites,” true hermaphrodites (which are less common in Cannabis) bear staminate and pistillate structures on individual flowers, whereas monoecious plants bear male and female flowers at different locations on the same plant.

The life cycle of Cannabis varies with each variety but can be generally summarized into germination, vegetative growth, and reproductive stages. Because of heavy breeding and selection by humans, most Cannabis seeds have lost dormancy mechanisms and do not require any pre-treatments or winterization to induce germination. Seed placed in viable growth conditions are expected to germinate in about 3 to 7 days. The first true leaves of a Cannabis plant contain a single leaflet, with subsequent leaves developing in opposite formation with increasing number of leaflets. Leaflets can be narrow or broad depending on the morphology of the plant grown. Cannabis plants are normally allowed to grow vegetatively for the first 4 to 8 weeks. During this period, the plant responds to increasing light with faster and faster growth. Under ideal conditions, Cannabis plants can grow up to 2.5 inches a day and are capable of reaching heights of up to 20 feet. Indoor growth pruning techniques tend to limit Cannabis size through careful pruning of apical or side shoots.

Cannabis is diploid, having a chromosome complement of 2n=20, although polyploid individuals have been artificially produced. The first genome sequence of Cannabis, which is estimated to be 820 Mb in size, was published in 2011 by a team of Canadian scientists (Bakel et al., “The draft genome and transcriptome of Cannabis sativa” Genome Biology 12: R102).

All known varieties of Cannabis are wind-pollinated and the fruit is an achene. Most varieties of Cannabis are short day plants, with the possible exception of C. sativa subsp. Sativa var. spontanea (=C. ruderalis), which is commonly described as “auto-flowering” and may be day-neutral.

The genus Cannabis was formerly placed in the Nettle (Urticaceae) or Mulberry (Moraceae) family, and later, along with the Humulus genus (hops), in a separate family, the Hemp family (Cannabaceae sensu stricto). Recent phylogenetic studies based on cpDNA restriction site analysis and gene sequencing strongly suggest that the Cannabaceae sensu stricto arose from within the former Celtidaceae family, and that the two families should be merged to form a single monophyletic family, the Cannabaceae sensu lato.

Cannabis plants produce a unique family of terpenophenolic compounds called cannabinoids. Cannabinoids, terpenoids, and other compounds are secreted by glandular trichomes that occur most abundantly on the floral calyxes and bracts of female plants. As a drug it usually comes in the form of dried flower buds (marijuana), resin (hashish), or various extracts collectively known as hashish oil. There are at least 483 identifiable chemical constituents known to exist in the Cannabis plant (Rudolf Brenneisen, 2007, Chemistry and Analysis of Phytocannabinoids (cannabinoids produced produced by Cannabis) and other Cannabis Constituents, In Marijuana and the Cannabinoids, ElSohly, ed) and at least 85 different cannabinoids have been isolated from the plant (see, e.g., El-Alfy, Abir T, et al., 2010, “Antidepressant-like effect of delta-9-tetrahydrocannabinol and other cannabinoids isolated from Cannabis sativa L”, Pharmacology Biochemistry and Behavior 95 (4): 434-42). The two cannabinoids usually produced in greatest abundance are cannabidiol (CBD) and/or Δ⁹-tetrahydrocannabinol (THC). THC is psychoactive while CBD is not. See, ElSohly, ed. (Marijuana and the Cannabinoids, Humana Press Inc., 321 papers, 2007), for a detailed description and literature review on the cannabinoids found in marijuana.

Cannabinoids are the most studied group of secondary metabolites in Cannabis. Most exist in two forms, as acids and in neutral (decarboxylated) forms. The acid form is designated by an “A” at the end of its acronym (i.e. THCA). The phytocannabinoids are synthesized in the plant as acid forms, and while some decarboxylation does occur in the plant, it increases significantly post-harvest and the kinetics increase at high temperatures (Sanchez and Verpoorte 2008). The biologically active forms for human consumption are the neutral forms. Decarboxylation is usually achieved by thorough drying of the plant material followed by heating it, often by either combustion, vaporization, or heating or baking in an oven. Unless otherwise noted, references to cannabinoids in a plant include both the acidic and decarboxylated versions (e.g., CBD and CBDA).

Detection of neutral and acidic forms of cannabinoids are dependent on the detection method utilized. Two popular detection methods are high-performance liquid chromatography (HPLC) and gas chromatography (GC). HPLC separates, identifies, and quantifies different components in a mixture, and passes a pressurized liquid solvent containing the sample mixture through a column filled with a solid adsorbent material. Each molecular component in a sample mixture interacts differentially with the adsorbent material, thus causing different flow rates for the different components and therefore leading to separation of the components. In contrast, GC separates components of a sample through vaporization. The vaporization required for such separation occurs at high temperature. Thus, the main difference between GC and HPLC is that GC involves thermal stress and mainly resolves analytes by boiling points while HPLC does not involve heat and mainly resolves analytes by polarity. The consequence of utilizing different methods for cannabinoid detection therefore is that HPLC is more likely to detect acidic cannabinoid precursors, whereas GC is more likely to detect decarboxylated neutral cannabinoids.

The cannabinoids in Cannabis plants include, but are not limited to, Δ9-Tetrahydrocannabinol (49-THC), Δ8-Tetrahydrocannabinol (Δ8-THC), Cannabichromene (CBC), Cannabicyclol (CBL), Cannabidiol (CBD), Cannabielsoin (CBE), Cannabigerol (CBG), Cannabinidiol (CBND), Cannabinol (CBN), Cannabitriol (CBT), and their propyl homologs, including, but are not limited to cannabidivarin (CBDV), 49-Tetrahydrocannabivarin (THCV), cannabichromevarin (CBCV), and cannabigerovarin (CBGV). See, Holley et al. (Constituents of Cannabis sativa L. XI Cannabidiol and cannabichromene in samples of known geographical origin, J. Pharm. Sci. 64:892-894, 1975) and De Zeeuw et al. (Cannabinoids with a propyl side chain in Cannabis, Occurrence and chromatographic behavior, Science 175:778-779). Non-THC cannabinoids can be collectively referred to as “CBs”, wherein CBs can be one of THCV, CBDV, CBGV, CBCV, CBD, CBC, CBE, CBG, CBN, CBND, and CBT cannabinoids.

Varins are a type of cannabinoid compounds having three carbon atoms in their alkyl side chain instead of the five carbon atom alkyl side chains more commonly associated with cannabinoids. Two such varins are tetrahydrocannabivarin (THCV) and cannabidivarin (CBDV), which are homologues of tetrahydrocannabinol (THC) and cannabidiol (CBD), respectively.

Methods of Identifying Plants with Increased Varin Production

Disclosed are methods of identifying a Cannabis plant that produces varins or has increased varin production, including (i) obtaining a nucleic acid sample from the plant or its germplasm, (ii) detecting one or more nucleic acid polymorphisms in: (a) ALT4, (b) ISS1, (c) KR, (d) FATB, or (e) GDSL (including a respective promoter region), for example, one or more nucleic acid polymorphisms disclosed herein. In some examples, the nucleic acid polymorphisms are in the coding sequence of ALT4, ISS1, KR, FATB, or GDSL. In some examples, the nucleic acid polymorphisms are in a regulatory sequence (e.g., promoter) of ALT4, ISS1, KR, FATB, or GDSL. In some implementations, the one or more nucleic acid polymorphisms are associated with increased varin production. The methods can further include identifying a plant having increased varin production, or selecting a plant identified as having increased varin production. Increased varin production can be determined, for example, by comparison to a reference plant, such as a plant not having the one or more nucleic acid polymorphisms in ALT4, ISS1, KR, FATB, or GDSL associated with increased varin production, or having a detrimental polymorphism in ALT4 ISS1, KR, FATB, or GDSL. Varin levels can be measured using standard biochemical analysis techniques. In some implementations, a plant identified as having increased varin production is selected for further analysis, propagation, breeding, or to make a product (e.g., a kief, hashish, bubble hash, an edible product, solvent reduced oil, sludge, e-juice, or tincture). In some examples, the plant identified as producing varins, or having increased varin production, is selected for propagation, breeding, or to make a product. The product is not, or otherwise excludes, any naturally occurring products. In some examples, the plant identified as producing varins, or having increased varin production is selected for breeding. In some examples, increased varin production is relative to a suitable reference (e.g., a plant without the polymorphisms associated with increased varin production, e.g., Abacus). In some implementations, the Cannabis plant is Cannabis sativa, Cannabis indica, or Cannabis ruderalis. In a non-limiting example, the Cannabis plant is Cannabis sativa.

In some implementations, a plant identified by the methods disclosed herein has a total varin content of at least 1%, for example, at least 1.5%, at least 2.0%, at least 2.5%, at least 3%, at least 3.5%, at least 4%, at least 4.5%, at least 5%, at least 5.5%, at least 6%, at least 6.5%, at least 7%, at least 7.5%, at least 8%, at least 8.5%, at least 9%, at least 9.5%, at least 10%, at least 11%, at least 12%, at least 13%, at least 14%, at least 15%, at least 16%, at least 17%, at least 18%, at least 19%, at least 20%, at least 25%, at least 30%, at least 35%, or at least 40% in at least one plant part (e.g., leaves, flowers, or trichomes). In a non-limiting example, the plant has a total varin content of at least 4%. In another non-limiting example, the plant has a total varin content of at least 5%. In a further non-limiting example, the plant has a total varin content of at least 6%. In some examples, the plant has a total varin content of at least 15%. In another non-limiting example, the plant has a total varin content of at least 10%. In another non-limiting example, the plant has a total varin content of at least 16.5%. In some examples, the plant has a total varin content of at least 20%.

In some implementations, a plant identified by the methods disclosed herein has a total varin content of 1% to 40%, for example, 1% to 39%, 1% to 35%, 1% to 30%, 1% to 25%, 1% to 24%, 1% to 23%, 1% to 22%, 1% to 21%, 1% to 20%, 1% to 19%, 1% to 18%, 1% to 17%, 1% to 16%, 1% to 15%, 1% to 14%, 1% to 13%, 1% to 12%, 1% to 11%, 1% to 10%, 1% to 9%, 1% to 8%, 1% to 7%, 1% to 6%, 1% to 5%, 1% to 4%, 1% to 3%, 1% to 2%, 2% to 40%, 2% to 35%, 2% to 30%, 2% to 25%, 2% to 24%, 2% to 23%, 2% to 22%, 2% to 21%, 2% to 20%, 2% to 19%, 2% to 18%, 2% to 17%, 2% to 16%, 2% to 15%, 2% to 14%, 2% to 13%, 2% to 12%, 2% to 11%, 2% to 10%, 2% to 9%, 2% to 8%, 2% to 7%, 2% to 6%, 2% to 5%, 2% to 4%, 2% to 3%, 3% to 40%, 3% to 35%, 3% to 30%, 3% to 25%, 3% to 24%, 3% to 23%, 3% to 22%, 3% to 21%, 3% to 20%, 3% to 19%, 3% to 18%, 3% to 17%, 3% to 16%, 3% to 15%, 3% to 14%, 3% to 13%, 3% to 12%, 3% to 11%, 3% to 10%, 3% to 9%, 3% to 8%, 3% to 7%, 3% to 6%, 3% to 5%, 3% to 4%, 4% to 40%, 4% to 35%, 4% to 30%, 4% to 25%, 4% to 24%, 4% to 23%, 4% to 22%, 4% to 21%, 4% to 20%, 4% to 19%, 4% to 18%, 4% to 17%, 4% to 16%, 4% to 15%, 4% to 14%, 4% to 13%, 4% to 12%, 4% to 11%, 4% to 10%, 4% to 9%, 4% to 8%, 4% to 7%, 4% to 6%, 4% to 5%, 5% to 40%, 5% to 35%, 5% to 30%, 5% to 25%, 5% to 24%, 5% to 23%, 5% to 22%, 5% to 21%, 5% to 20%, 5% to 19%, 5% to 18%, 5% to 17%, 5% to 16%, 5% to 15%, 5% to 14%, 5% to 13%, 5% to 12%, 5% to 11%, 5% to 10%, 5% to 9%, 5% to 8%, 5% to 7%, 5% to 6%, 6% to 40%, 6% to 35%, 6% to 30%, 6% to 25%, 6% to 24%, 6% to 23%, 6% to 22%, 6% to 21%, 6% to 20%, 6% to 19%, 6% to 18%, 6% to 17%, 6% to 16%, 6% to 15%, 6% to 14%, 6% to 13%, 6% to 12%, 6% to 11%, 6% to 10%, 6% to 9%, 6% to 8%, 6% to 7%, 7% to 40%, 7% to 35%, 7% to 30%, 7% to 25%, 7% to 24%, 7% to 23%, 7% to 22%, 7% to 21%, 7% to 20%, 7% to 19%, 7% to 18%, 7% to 17%, 7% to 16%, 7% to 15%, 7% to 14%, 7% to 13%, 7% to 12%, 7% to 11%, 7% to 10%, 7% to 9%, 7% to 8%, 8% to 40%, 8% to 35%, 8% to 30%, 8% to 25%, 8% to 24%, 8% to 23%, 8% to 22%, 8% to 21%, 8% to 20%, 8% to 19%, 8% to 18%, 8% to 17%, 8% to 16%, 8% to 15%, 8% to 14%, 8% to 13%, 8% to 12%, 8% to 11%, 8% to 10%, 8% to 9%, 9% to 40%, 9% to 35%, 9% to 30%, 9% to 25%, 9% to 24%, 9% to 23%, 9% to 22%, 9% to 21%, 9% to 20%, 9% to 19%, 9% to 18%, 9% to 17%, 9% to 16%, 9% to 15%, 9% to 14%, 9% to 13%, 9% to 12%, 9% to 11%, 9% to 10%, 10% to 40%, 10% to 35%, 10% to 30%, 10% to 25%, 10% to 24%, 10% to 23%, 10% to 22%, 10% to 21%, 10% to 20%, 10% to 19%, 10% to 18%, 10% to 17%, 10% to 16%, 10% to 15%, 10% to 14%, 10% to 13%, 10% to 12%, 10% to 11%, 11% to 19%, 11% to 18%, 11% to 17%, 11% to 16%, 11% to 15%, 11% to 14%, 11% to 13%, 11% to 12%, 12% to 19%, 12% to 18%, 12% to 17%, 12% to 16%, 12% to 15%, 12% to 14%, 12% to 13%, 13% to 19%, 13% to 18%, 13% to 17%, 13% to 16%, 13% to 15%, 13% to 14%, 14% to 19%, 14% to 18%, 14% to 17%, 14% to 16%, 14% to 15%, 15% to 40%, 15% to 35%, 15% to 30%, 15% to 25%, 15% to 24%, 15% to 23%, 15% to 22%, 15% to 21%, 15% to 20%, 15% to 19%, 15% to 18%, 15% to 17%, 15% to 16%, 16% to 19%, 16% to 18%, 16% to 17%, 17% to 19%, 17% to 18%, 18% to 19%, 19% to 40%, 19% to 35%, 19% to 30%, 19% to 25%, 19% to 24%, 19% to 23%, 19% to 22%, 19% to 21%, 19% to 20%, 20% to 40%, 20% to 35%, 20% to 30%, 20% to 25%, 20% to 24%, 20% to 23%, 20% to 22%, 20% to 21%, 25% to 40%, 25% to 35%, 25% to 30%, 30% to 40%, 30% to 35%, or 35% to 40% total varin content in at least one plant part (e.g., leaves, flowers, or trichomes). In some examples, the total varin content is 2% to 7%. In some examples, the total varin content is 3% to 7%. In some examples, the total varin content is 4% to 7%. In a further example, the total varin content is 4% to 18%. In another example, the total varin content is 4% to 16.5%. In another example, the total varin content is 7% to 16.5%. In some examples, the total varin content is 15% to 35%. In some examples, the total varin content is 15% to 20%.

In some implementations, a plant identified by the methods disclosed herein has a varin ratio of at least 0.1, for example, at least 0.2, at least 0.3, at least 0.4, at least 0.5, at least 0.6, at least 0.7, at least 0.8, at least 0.9, at least 1, at least 1.5, at least 2.0, at least 2.5, at least 3, at least 3.5, at least 4, at least 4.5, at least 5, at least 5.5, at least 6, at least 6.5, at least 7, at least 7.5, at least 8, at least 8.5, at least 9, at least 9.5, at least 10, at least 15, or at least 20 in at least one plant part (e.g., leaves, flowers, or trichomes). In a non-limiting example, the plant has a varin ratio of at least 0.2. In a non-limiting example, the plant has a varin ratio of at least 1. In a non-limiting example, the plant has a varin ratio of at least 3. In another non-limiting example, the plant has a varin ratio of at least 4. In some examples, the plant has a varin ratio of at least 7.

In some implementations, a plant identified by the methods disclosed herein has a varin ratio of 0.1 to 30, for example, 0.1 to 25, 0.1 to 20, 0.1 to 15, 0.1 to 10, 0.1 to 5, 0.1 to 4, 0.1 to 3, 0.1 to 2, 0.1 to 1, 0.1 to 0.5, 0.25 to 30, 0.25 to 25, 0.25 to 20, 0.25 to 15, 0.25 to 10, 0.25 to 5, 0.25 to 4, 0.25 to 3, 0.25 to 2, 0.25 to 1, 0.25 to 0.5, 0.33 to 25, 0.33 to 20, 0.33 to 15, 0.33 to 10, 0.33 to 7, 0.33 to 5, 0.33 to 3, 0.33 to 1, 0.33 to 0.5, 0.5 to 30, 0.5 to 25, 0.5 to 20, 0.5 to 15, 0.5 to 10, 0.5 to 5, 0.5 to 4, 0.5 to 3, 0.5 to 2, 0.5 to 1, 0.75 to 25, 0.75 to 20, 0.75 to 15, 0.75 to 10, 0.75 to 5, 0.75 to 4, 0.75 to 3, 0.75 to 2, 0.75 to 1, 1 to 30, 1 to 25, 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, 1 to 2, 2 to 30, 2 to 25, 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4, 2 to 3, 3 to 30, 3 to 25, 3 to 20, 3 to 19, 3 to 18, 3 to 17, 3 to 16, 3 to 15, 3 to 14, 3 to 13, 3 to 12, 3 to 11, 3 to 10, 3 to 9, 3 to 8, 3 to 7, 3 to 6, 3 to 5, 3 to 4, 4 to 30, 4 to 25, 4 to 20, 4 to 19, 4 to 18, 4 to 17, 4 to 16, 4 to 15, 4 to 14, 4 to 13, 4 to 12, 4 to 11, 4 to 10, 4 to 9, 4 to 8, 4 to 7, 4 to 6, 4 to 5, 5 to 30, 5 to 25, 5 to 20, 5 to 19, 5 to 18, 5 to 17, 5 to 16, 5 to 15, 5 to 14, 5 to 13, 5 to 12, 5 to 11, 5 to 10, 5 to 9, 5 to 8, 5 to 7, 5 to 6, 6 to 30, 6 to 25, 6 to 20, 6 to 19, 6 to 18, 6 to 17, 6 to 16, 6 to 15, 6 to 14, 6 to 13, 6 to 12, 6 to 11, 6 to 10, 6 to 9, 6 to 8, 6 to 7, 7 to 30, 7 to 25, 7 to 20, 7 to 19, 7 to 18, 7 to 17, 7 to 16, 7 to 15, 7 to 14, 7 to 13, 7 to 12, 7 to 11, 7 to 10, 7 to 9, 7 to 8, 8 to 30, 8 to 25, 8 to 20, 8 to 19, 8 to 18, 8 to 17, 8 to 16, 8 to 15, 8 to 14, 8 to 13, 8 to 12, 8 to 11, 8 to 10, 8 to 9, 9 to 30, 9 to 25, 9 to 20, 9 to 19, 9 to 18, 9 to 17, 9 to 16, 9 to 15, 9 to 14, 9 to 13, 9 to 12, 9 to 11, 9 to 10, 10 to 30, 10 to 25, 10 to 20, 10 to 18, 10 to 16, 10 to 14, 10 to 12, 12 to 30, 12 to 25, 12 to 20, 12 to 18, 12 to 16, 12 to 14, 14 to 30, 14 to 25, 14 to 20, 14 to 18, 14 to 16, 16 to 30, 16 to 25, 16 to 20, 16 to 18, 18 to 30, 18 to 25, 18 to 20, 20 to 30, 20 to 25, or 25 to 30 in at least one plant part (e.g., leaves, flowers, or trichomes). In some examples, the varin ratio is 1 to 8. In some examples, the varin ratio is 0.25 to 5. In some examples, the varin ratio is 1 to 5. In further examples, the varin ratio is 7 to 20. In some examples, the varin ratio is 7 to 10.

The plant part can be any part of the plant identified by the methods disclosed herein. In some examples, the plant part is flower or inflorescence. In some examples, the plant part is a trichome. In some examples, the plant part is a leaf or other vegetative material.

In some implementations, the one or more nucleic acid polymorphisms are detected in ACYL-LIPID THIOESTERASE 4 (ALT4). In some implementations, the one or more nucleic acid polymorphisms detected in ALT4 are beneficial polymorphisms (polymorphisms associated with increased varin production). In some examples, the beneficial polymorphisms include polymorphisms detected in a varin producing Cannabis line, such as 21VLP5-1-101 and/or 21TX1-60. Beneficial polymorphisms in 21VLP5-1-101 and 21TX1-60 are disclosed herein. In some examples, the one or more nucleic acid polymorphisms detected in ALT4 produce (in the corresponding ALT4 protein) (a) a glutamine corresponding to Abacus reference position 59; (b) a glycine corresponding to Abacus reference position 66; (c) an insertion of aspartic acid immediately following Abacus reference position 47; (d) a tyrosine corresponding to Abacus reference position 67; (e) a deletion of aspartic acid corresponding to Abacus reference position 48; (f) a deletion of aspartic acid corresponding to Abacus reference position 49; (g) a lysine corresponding to Abacus reference position 68; (h) an aspartic acid corresponding to Abacus reference position 81; (i) a glutamic acid corresponding to Abacus reference position 85; (j) a histidine corresponding to Abacus reference position 111; (k) an alanine corresponding to Abacus reference position 115; (l) a serine corresponding to Abacus reference position 124; (m) a valine corresponding to Abacus reference position 125; (n) an aspartic acid corresponding to Abacus reference position 136; (o) an isoleucine corresponding to Abacus reference position 160; (p) a valine corresponding to Abacus reference position 165; (q) a valine corresponding to Abacus reference position 172; (r) a serine corresponding to Abacus reference position 7; (s) a glutamine corresponding to Abacus reference position 202; or (t) an alanine corresponding to Abacus reference position 206. The positions are in reference to the Abacus ALT4 protein sequence (SEQ ID NO: 12).

In some implementations, the one or more nucleic acid polymorphisms detected in ALT4 produce a glutamine corresponding to Abacus reference position 59 or a glycine corresponding to Abacus reference position 66. In some implementations, the one or more nucleic acid polymorphisms detected in ALT4 produce a glutamine corresponding to Abacus reference position 59 and a glycine corresponding to Abacus reference position 66. The positions are in reference to the Abacus ALT4 protein sequence (SEQ ID NO: 12). In some implementations, the one or more nucleic acid polymorphisms detected in ALT4 produce a serine corresponding to Abacus reference position 7 or a lysine corresponding to Abacus reference position 68. In some implementations, the one or more nucleic acid polymorphisms detected in ALT4 produce a serine corresponding to Abacus reference position 7 and a lysine corresponding to Abacus reference position 68. The positions are in reference to the Abacus ALT4 protein sequence (SEQ ID NO: 12).

In some implementations, the one or more nucleic acid polymorphisms detected in ALT4 produce (a) a glutamine corresponding to Abacus reference position 59; (b) a glycine corresponding to Abacus reference position 66; (c) an insertion of aspartic acid immediately following Abacus reference position 47; and (d) a tyrosine corresponding to Abacus reference position 67. In some implementations, the one or more nucleic acid polymorphisms detected in ALT4 produce (a) the glutamine corresponding to Abacus reference position 59; (b) the glycine corresponding to Abacus reference position 66; (e) the deletion of aspartic acid corresponding to Abacus reference position 48; (f) the deletion of aspartic acid corresponding to Abacus reference position 49; (g) the lysine corresponding to Abacus reference position 68; (h) the aspartic acid corresponding to Abacus reference position 81; (i) the glutamic acid corresponding to Abacus reference position 85; (j) the histidine corresponding to Abacus reference position 111; (k) the alanine corresponding to Abacus reference position 115; (l) the serine corresponding to Abacus reference position 124; (m) the valine corresponding to Abacus reference position 125; (n) the aspartic acid corresponding to Abacus reference position 136; (o) the isoleucine corresponding to Abacus reference position 160; (p) the valine corresponding to Abacus reference position 165; (q) the valine corresponding to Abacus reference position 172; (r) a serine corresponding to Abacus reference position 7; (s) a glutamine corresponding to Abacus reference position 202; and (t) an alanine corresponding to Abacus reference position 206. The positions are in reference to the Abacus ALT4 protein sequence (SEQ ID NO: 12).

Specific examples of nucleotide sequences that cause the polymorphisms (a)-(t) as discussed above are provided in Example 1. However, the translation of nucleic acids into amino acids is known and determined by the genetic code. Thus, polymorphisms that result in a particular amino acid of a protein sequence can be readily determined by using the genetic code. For non-limiting, exemplary purposes, arginine is encoded by CGT, CGC, CGA, CGG, AGA, and AGG.

In some implementations, the one or more nucleic acid polymorphisms are detected in β ketoacyl-acyl carrier protein (ACP) reductase (KR). In some implementations, the one or more nucleic acid polymorphisms detected in KR are beneficial polymorphisms (polymorphisms associated with increased varin production). Exemplary beneficial polymorphisms are disclosed in the Examples provided herein. In some examples, the one or more nucleic acid polymorphisms detected in KR produce (in the corresponding KR protein) (a) an arginine at position 39; (b) a proline at position 88; (c) a glutamic acid at position 122; (d) a glycine at position 123; (e) a glycine at position 125; (f) a proline at position 139; (g) a valine at position 158; (h) a glycine at position 175; (i) a serine at position 193; (j) a arginine at position 205; (k) a arginine at position 208; (l) an isoleucine at position 217; (m) a serine at position 218; (n) a serine at position 251; or (o) an isoleucine at position 293. The positions are in reference to the Abacus KR protein sequence (SEQ ID NO: 66). In some examples, the one or more nucleic acid polymorphisms include a valine at position 158 corresponding to Abacus KR sequence set forth as SEQ ID NO: 66.

In some implementations, the one or more nucleic acid polymorphisms detected in KR produce a valine at position 158 or an isoleucine at position 217. In some implementations, the one or more nucleic acid polymorphisms detected in KR produce a valine at position 158 and an isoleucine at position 217. The positions are in reference to the Abacus KR protein sequence (SEQ ID NO: 66).

Specific examples of nucleotide sequences that cause the polymorphisms (a)-(o) as discussed above are provided in Example 1. However, the translation of nucleic acids into amino acids is known and determined by the genetic code. Thus, polymorphisms that result in a particular amino acid of a protein sequence can be readily determined by using the genetic code. For non-limiting, exemplary purposes, arginine is encoded by CGT, CGC, CGA, CGG, AGA, and AGG.

In some implementations, the one or more nucleic acid polymorphisms are detected in fatty acyl-ACP thioesterase B (FATB). In some implementations, the one or more nucleic acid polymorphisms detected in FATB are beneficial polymorphisms (polymorphisms associated with increased varin production). Exemplary beneficial polymorphisms are disclosed in the Examples provided herein. In some examples, the one or more nucleic acid polymorphisms detected in FATB produce (in the corresponding FATB protein) (a) a histidine at position 155; (b) a glutamine at position 173; (c) an arginine at position 197; (d) an isoleucine at position 201; (e) a proline at position 202; or (f) an aspartic acid at position 207. The positions are in reference to the Abacus FATB protein sequence (SEQ ID NO: 100). In some examples, the one or more nucleic acid polymorphisms include an isoleucine at position 201 corresponding to Abacus FATB sequence set forth as SEQ ID NO: 100. Specific examples of nucleotide sequences that cause the polymorphisms (a)-(f) as discussed above are provided in Example 1. However, the translation of nucleic acids into amino acids is known and determined by the genetic code. Thus, polymorphisms that result in a particular amino acid of a protein sequence can be readily determined by using the genetic code. For non-limiting, exemplary purposes, arginine is encoded by CGT, CGC, CGA, CGG, AGA, and AGG.

In some implementations, the one or more nucleic acid polymorphisms are detected in Gly-Asp-Ser-Leu-motif lipase esterase/lipase 1 (GDSL1). In some implementations, the one or more nucleic acid polymorphisms detected in GDSL1 are beneficial polymorphisms (polymorphisms associated with increased varin production). Exemplary beneficial polymorphisms are disclosed in the Examples provided herein. In some examples, the one or more nucleic acid polymorphisms detected in GDSL1 produce (in the corresponding GDSL1 protein) (a) an alanine at position 59; (b) an asparagine at position 275; (c) a threonine at position 297; or (d) a methionine at position 213. In some examples, the one or more nucleic acid polymorphisms include a methionine at position 213 corresponding to Abacus GDSL1 sequence set forth as SEQ ID NO: 110. The positions are in reference to the Abacus GDSL1 protein sequence (SEQ ID NO: 110). Specific examples of nucleotide sequences that cause the polymorphisms (a)-(d) as discussed above are provided in Example 1. However, the translation of nucleic acids into amino acids is known and determined by the genetic code. Thus, polymorphisms that result in a particular amino acid of a protein sequence can be readily determined by using the genetic code. For non-limiting, exemplary purposes, arginine is encoded by CGT, CGC, CGA, CGG, AGA, and AGG.

In some implementations, the one or more nucleic acid polymorphisms are detected in INDOLE SEVERE SENSITIVE1 (ISS1). In some implementations, the one or more nucleic acid polymorphisms detected in ISS1 are beneficial polymorphisms (polymorphisms associated with increased varin production). In some examples, the beneficial polymorphisms include polymorphisms detected in a varin producing Cannabis line, such as 22VLV2-1-52. Beneficial polymorphisms in 22VLV2-1-52 are disclosed herein. In some examples, the one or more nucleic acid polymorphisms detected in ISS1 include a deletion of the A corresponding to Abacus reference position −436 bp upstream from the start codon of ISS1. The nucleotide position is in reference to the Abacus reference Csat_AbacusV2 (NCBI assembly accession GCA_025232715.1; see also, SEQ ID NO: 31).

In some implementations, at least two nucleic acid polymorphisms disclosed herein are detected. In some examples, at least three nucleic acid polymorphisms disclosed herein are detected. In some examples, at least four nucleic acid polymorphisms disclosed herein are detected. In some examples, at least five nucleic acid polymorphisms disclosed herein are detected. In some examples, at least six nucleic acid polymorphisms disclosed herein are detected. In some examples, at least eight nucleic acid polymorphisms disclosed herein are detected. In some examples, at least ten nucleic acid polymorphisms disclosed herein are detected. In some examples, at least twelve nucleic acid polymorphisms disclosed herein are detected. In some examples, at least fourteen nucleic acid polymorphisms disclosed herein are detected.

In some implementations, one or more nucleic acid polymorphisms disclosed herein are detected in two or more of ALT4, ISS1, KR, FATB, or GDSL. In some implementations, one or more nucleic acid polymorphisms disclosed herein are detected in ALT4 and one or more of ISS1, KR, FATB, or GDSL. In some implementations, one or more nucleic acid polymorphisms disclosed herein are detected in ISS1 and one or more of ALT4, KR, FATB, or GDSL. In some implementations, one or more nucleic acid polymorphisms disclosed herein are detected in KR and one or more of ALT4, ISS1, FATB, or GDSL. In some implementations, one or more nucleic acid polymorphisms disclosed herein are detected in FATB and one or more of ALT4, ISS1, KR, or GDSL. In some implementations, one or more nucleic acid polymorphisms disclosed herein are detected in GDSL and one or more of ALT4, ISS1, KR, or FATB. In some implementations, one or more nucleic acid polymorphisms disclosed herein are detected in three or more of ALT4, ISS1, KR, FATB, or GDSL. In some implementations, one or more nucleic acid polymorphisms disclosed herein are detected in four or more of ALT4, ISS1, KR, FATB, or GDSL. In some implementations, one or more nucleic acid polymorphisms disclosed herein are detected in all of ALT4, ISS1, KR, FATB, or GDSL. In some implementations, one or more nucleic acid polymorphisms disclosed herein are detected in ALT4 and ISS1.

In some examples, the one or more nucleic acid polymorphisms include a deletion of A at position −436 bp upstream from the start codon of ISS1 according to Abacus reference is Csat_AbacusV2, NCBI assembly accession GCA_025232715.1; a valine at position 158 corresponding to Abacus KR sequence set forth as SEQ ID NO: 66; a serine at position 7 corresponding to Abacus ALT4 sequence set forth as SEQ ID NO: 12; a lysine at position 68 corresponding to Abacus ALT4 sequence set forth as SEQ ID NO: 12; an isoleucine at position 201 corresponding to Abacus FATB sequence set forth as SEQ ID NO: 100; or a methionine at position 213 corresponding to Abacus GDSL1 sequence set forth as SEQ ID NO: 110. In some examples, the one or more nucleic acid polymorphisms include a deletion of A at position −436 bp upstream from the start codon of ISS1 according to Abacus reference is Csat_AbacusV2, NCBI assembly accession GCA_025232715.1; a valine at position 158 corresponding to Abacus KR sequence set forth as SEQ ID NO: 66; a serine at position 7 corresponding to Abacus ALT4 sequence set forth as SEQ ID NO: 12 or a lysine at position 68 corresponding to Abacus ALT4 sequence set forth as SEQ ID NO: 12; an isoleucine at position 201 corresponding to Abacus FATB sequence set forth as SEQ ID NO: 100; and a methionine at position 213 corresponding to Abacus GDSL1 sequence set forth as SEQ ID NO: 110.

In some examples, the one or more nucleic acid polymorphisms include a deletion of A at position −436 bp upstream from the start codon of ISS1 according to Abacus reference is Csat_AbacusV2, NCBI assembly accession GCA_025232715.1; and a valine at position 158 corresponding to Abacus KR sequence set forth as SEQ ID NO: 66. In some examples, the one or more nucleic acid polymorphisms include a deletion of A at position −436 bp upstream from the start codon of ISS1 according to Abacus reference is Csat_AbacusV2, NCBI assembly accession GCA_025232715.1; a valine at position 158 corresponding to Abacus KR sequence set forth as SEQ ID NO: 66; and a serine at position 7 corresponding to Abacus ALT4 sequence set forth as SEQ ID NO: 12 or a lysine at position 68 corresponding to Abacus ALT4 sequence set forth as SEQ ID NO: 12.

Methods of detecting nucleic acid polymorphisms have been described, and can include amplification of a target polynucleotide (e.g., by PCR). PCR uses a particular amplification primer pair that specifically hybridize to a target polynucleotide and produce an amplification product (the amplicon). Primers can be designed such that the amplicon can contain a nucleic acid polymorphism of interest. Methods for designing PCR primers and PCR conditions have been described, for example, in Sambrook et al. (2014) Molecular Cloning: A Laboratory Manual (Fourth Edition, Cold Spring Harbor Laboratory Press, Plainview, N.Y.). It is understood that a number of parameters in a specific PCR protocol may need to be adjusted to specific laboratory conditions and may be slightly modified and yet allow for the collection of similar results. The primers can be radiolabeled, or labeled by any suitable means (e.g., using a non-radioactive fluorescent tag), to allow for rapid visualization of the different size amplicons following an amplification reaction without any additional labeling step or visualization step.

Other examples of nucleic acid amplification methods include, but are not limited to, reverse-transcription PCR (RT-PCR), quantitative real-time PCR (qPCR), quantitative real-time reverse transcriptase PCR (qRT-PCR) (see, e.g., Adams, A beginner's guide to RT-PCR, qPCR and RT-qPCR, Biochemist (Lond) (2020) 42 (3): 48-53), isothermal amplification methods (see, e.g., Zanoli et al., Biosensors (2013) 3 (1): 18-43), nucleic acid sequence-based amplification (NASBA) (see, e.g., Deiman and Sillekens, Mol Biotechnol (2002) 20 (2): 163-79), loop-mediated isothermal amplification (LAMP) (see, e.g., Notomi et al., (2000) Nucleic Acids Res. 28 (12): e63), helicase-dependent amplification (HDA) (see, e.g., Cao et al., Helicase-dependent amplification of nucleic acids, Curr Protoc Mol Biol, 104:15.11.1-15.11.12, 2013), rolling circle amplification (RCA) (see, e.g, Yao et al. Nature Protocols (2021) 16, 5460-5483), multiple displacement amplification (MDA) (see, e.g, Spits et al. Nature Protocols (2006) 1:1965-1970), recombinase polymerase amplification (RPA) (see, e.g., Lobato et al., Trends Analyt Chem (2018) 98:19-35), ligase chain reaction (LCR) (see e.g., Gibriel and Adel, Mutat Res Rev Mutat Res. (2017) 773:66-90), transcription amplification (see e.g., Kwoh et al. (1989) Proc. Natl. Acad. Sci. USA 86:1173), self-sustained sequence replication (see e.g., Guatelli et al. (1990) Proc. Natl. Acad. Sci. USA 87:1874), dot PCR, and linker adapter PCR. Suitable amplification methods are also described, for example, in Sambrook et al. (2014) Molecular Cloning: A Laboratory Manual (Fourth Edition, Cold Spring Harbor Laboratory Press, Plainview, N.Y.).

In some examples, amplification produces an amplicon that is at least 20 nucleotides in length, for example, at least 50 nucleotides in length, or alternatively, at least 100 nucleotides in length, at least 200 nucleotides in length, at least 300, at least 400, at least 500, at least 600, at least 700, at least 800, at least 900, at least 1000, at least 1500, at least 2000, or at least 2500 nucleotides in length. In some examples, the amplicon is no longer than 10000 nucleotides in length, for example, no longer than 3000, no longer than 5000, no longer than 7000, or no longer than 9000 nucleotides in length. In some examples, marker amplification produces an amplicon that is 20 to 10000 nucleotides in length, for example, 20 to 9000 nucleotides, 20 to 8000 nucleotides, 20 to 7000 nucleotides, 20 to 6000 nucleotides, 20 to 5000 nucleotides, 20 to 4000 nucleotides, 20 to 3000 nucleotides, 20 to 2000 nucleotides, 20 to 1500 nucleotides, 20 to 1000 nucleotides, 20 to 500 nucleotides, 20 to 400 nucleotides, 20 to 300 nucleotides, 20 to 200 nucleotides, 20 to 150 nucleotides, 20 to 100 nucleotides, 20 to 50 nucleotides, 50 to 9000 nucleotides, 50 to 8000 nucleotides, 50 to 7000 nucleotides, 50 to 6000 nucleotides, 50 to 5000 nucleotides, 50 to 4000 nucleotides, 50 to 3000 nucleotides, 50 to 2000 nucleotides, 50 to 1000 nucleotides, 50 to 500 nucleotides, 50 to 400 nucleotides, 50 to 300 nucleotides, 50 to 200 nucleotides, 50 to 150 nucleotides, 50 to 100 nucleotides, 100 to 9000 nucleotides, 100 to 8000 nucleotides, 100 to 7000 nucleotides, 100 to 6000 nucleotides, 100 to 5000 nucleotides, 100 to 4000 nucleotides, 100 to 3000 nucleotides, 100 to 2000 nucleotides, 100 to 1000 nucleotides, 100 to 500 nucleotides, 100 to 400 nucleotides, 100 to 300 nucleotides, 100 to 200 nucleotides, 100 to 150 nucleotides, 250 to 9000 nucleotides, 250 to 8000 nucleotides, 250 to 7000 nucleotides, 250 to 6000 nucleotides, 250 to 5000 nucleotides, 250 to 4000 nucleotides, 250 to 3000 nucleotides, 250 to 2000 nucleotides, 250 to 1000 nucleotides, 250 to 500 nucleotides, 250 to 400 nucleotides, or 250 to 300 nucleotides in length. In some examples, the amplicon is 100 to 4000 nucleotides in length. In some examples, the amplicon is 200 to 3000 nucleotides in length. In some examples, the amplicon is at least 51 nucleotides in length. In some examples, the amplicon is at least 101 nucleotides in length.

The presence of a nucleic acid polymorphism in an amplicon can be determined, for example, by directly sequencing the amplicon, performing a restriction enzyme digest (e.g, restriction fragment length polymorphism (RFLP)), or by using a detection probe. In some implementations, detection includes PCR, quantitative PCR (qPCR), reverse-transcription PCR (RT-PCR), quantitative real-time reverse transcriptase PCR (qRT-PCR), and/or sequencing. In some examples, detection includes PCR, quantitative PCR (qPCR), and/or sequencing.

PCR detection and quantification using dual-labeled fluorogenic oligonucleotide probes, commonly referred to as “TaqMan™” probes, can also be performed according to the present disclosure. These probes are composed of short (e.g., 20-25 base) oligodeoxynucleotides that are labeled with two different fluorescent dyes. On the 5′ terminus of each probe is a reporter dye, and on the 3′ terminus of each probe a quenching dye is found. The oligonucleotide probe sequence is complementary to an internal target sequence present in a PCR amplicon. When the probe is intact, energy transfer occurs between the two fluorophores and emission from the reporter is quenched by the quencher by FRET. During the extension phase of PCR, the probe is cleaved by 5′ nuclease activity of the polymerase used in the reaction, thereby releasing the reporter from the oligonucleotide-quencher and producing an increase in reporter emission intensity. TaqMan™ probes are oligonucleotides that have a label and a quencher, where the label is released during amplification by the exonuclease action of the polymerase used in amplification, providing a real time measure of amplification during synthesis. A variety of TaqMan™ reagents are commercially available, e.g., from Applied Biosystems as well as from a variety of specialty vendors such as Biosearch Technologies.

In some implementations, detection of nucleic acid polymorphisms includes use of an oligonucleotide primer or probe. In general, synthetic methods for making oligonucleotides, including probes or primers are known. For example, oligonucleotides can be synthesized chemically according to the solid phase phosphoramidite triester method described. Oligonucleotides, including modified oligonucleotides, can also be ordered from a variety of commercial sources. Nucleic acid probes to the marker loci can be cloned and/or synthesized. Any suitable label can be used with a probe. Detectable labels suitable for use with nucleic acid probes include, for example, any composition detectable by spectroscopic, radioisotopic, photochemical, biochemical, immunochemical, electrical, optical or chemical means. Useful labels include biotin for staining with labeled streptavidin conjugate, magnetic beads, fluorescent dyes, radio labels, enzymes, and colorimetric labels. Other labels include ligands which bind to antibodies labeled with fluorophores, chemiluminescent agents, and enzymes. A probe can also constitute radio labeled PCR primers that are used to generate a radio labeled amplicon. It is not intended that the nucleic acid probes be limited to any particular size, however, nucleic acid probes are typically 20-100 base pairs.

Amplification is not always required for detection of a nucleic acid polymorphism (e.g. Southern blotting or RFLP detection). Separate detection probes can also be omitted in amplification/detection methods, e.g., by performing a real time amplification reaction that detects product formation by modification of the relevant amplification primer upon incorporation into a product, incorporation of labeled nucleotides into an amplicon, or by monitoring changes in molecular rotation properties of amplicons as compared to unamplified precursors (e.g., by fluorescence polarization).

In some implementations, a nucleic acid polymorphism is detected by sequencing a nucleic acid fragment comprising a target sequence of interest (e.g., at least a portion of ALT4 or ISS1), or by whole genome sequencing (or whole transcriptome sequencing). Non-limiting examples of suitable sequencing methods include capillary electrophoresis (e.g., Sanger sequencing) and high-throughput sequencing (e.g., Illumina® or 454 Sequencing®). High-throughput sequencing includes short read or long read techniques. In some implementations, sequencing includes whole genome sequencing (e.g., sequencing the genome of a Cannabis plant of interest). In some examples, sequencing includes targeted sequencing (sequencing of a particular nucleic acid or amplicon of interest). In some examples, sequencing includes sequencing a transcriptome (RNA-Seq) (e.g., sequencing the transcriptome of a Cannabis plant of interest). In some implementations, sequencing does not include sequencing of RNA. In some implementations, the genome is sequenced.

### Methods of Producing Modified Plants

Disclosed herein are methods of producing a modified Cannabis plant including introducing a genetic modification in the Cannabis plant that increases ALT4, KR, GDSL1, and/or FATB activity or decreases expression and/or activity of ISS1 relative to a reference plant (e.g., a Cannabis plant in an unmodified state). In some examples, increasing activity includes increasing activity for a particular substrate. Also disclosed are methods of increasing production of varin precursor molecules (e.g., C4 fatty acid) in a Cannabis plant, including introducing a genetic modification in the Cannabis plant that increases ALT4, KR, GDSL1, or FATB activity; or decreases expression or activity of ISS1 relative to a reference plant (e.g., a Cannabis plant in an unmodified state). In some examples, the genetic modification that increases ALT4, KR, GDSL1, or FATB activity; or decreases expression and/or activity of ISS1, is one or more amino acid substitutions associated with increased varin production disclosed herein, for example, in Example 1. In some examples, the genetic modification is in ALT4, KR, GDSL1, and/or FATB (e.g., in a coding sequence or a regulatory sequence of ALT4, KR, GDSL1, and/or FATB (e.g., a promoter region)) and increases ALT4, KR, GDSL1, and/or FATB activity, respectively. In some examples, increasing activity of ALT4, KR, GDSL1, or FATB, and/or decreasing expression or activity of ISS1, increases the production of varin precursor molecules (e.g., C4 fatty acids). In some examples, the genetic modification is in ALT4 (e.g., a coding or a regulatory sequence of ALT4, e.g., a promoter region) and increases ALT4 activity relative to a reference plant. In some examples, the genetic modification is in KR (e.g., a coding or a regulatory sequence of KR, e.g., a promoter region) and increases KR activity relative to a reference plant. In some examples, the genetic modification is in GDSL1 (e.g., a coding or a regulatory sequence of GDSL1, e.g., a promoter region) and increases GDSL1 activity relative to a reference plant. In some examples, the genetic modification is in FATB (e.g., a coding or a regulatory sequence of FATB, e.g., a promoter region) and increases FATB activity relative to a reference plant. In some examples, the genetic modification is in ISS1 (e.g., a coding or a regulatory sequence of ISS1, e.g., a promoter region) and decreases expression of ISS1 relative to a reference plant. In some examples, the genetic modification increases production of varins relative to a reference plant (e.g., a Cannabis plant in an unmodified state). In some implementations, the modified Cannabis plant is Cannabis sativa, Cannabis indica, or Cannabis ruderalis. In a non-limiting example, the modified Cannabis plant is Cannabis sativa.

Also disclosed are methods of producing modified Cannabis plants that include introducing a genetic modification in ALT4, ISS1, KR, GDSL1, and/or FATB (e.g., a coding sequence or a regulatory sequence thereof); or include introducing a heterologous beneficial allele of ALT4, ISS1, KR, GDSL1, and/or FATB into the Cannabis plant. The beneficial allele of ALT4, ISS1, KR, GDSL1, and/or FATB can include a respective promoter region. In some examples, the modified Cannabis plant includes a genetic modification in ALT4 (e.g., in the coding sequence or a regulatory element of ALT4, e.g., a promoter region). In some examples, the modified Cannabis plant includes a genetic modification in KR (e.g., in the coding sequence or a regulatory element of KR, e.g., a promoter region). In some examples, the modified Cannabis plant includes a genetic modification in GDSL1 (e.g., in the coding sequence or a regulatory element of GDSL1, e.g., a promoter region). In some examples, the modified Cannabis plant includes a genetic modification in FATB (e.g., in the coding sequence or a regulatory element of FATB, e.g., a promoter region). In some examples, the modified Cannabis plant includes a genetic modification in ISS1 (e.g., in the coding sequence or a regulatory element of ISS1, e.g., a promoter region). In some examples, the genetic modification is in the coding sequence of ALT4, ISS1, KR, GDSL1, and/or FATB. In some examples, the genetic modification is in the promoter sequence of ALT4, ISS1, KR, GDSL1, and/or FATB.

In some examples, the modified Cannabis plant includes a genetic modification in two or more of ALT4, KR, GDSL1, FATB, and ISS1. In some examples, the modified Cannabis plant includes a genetic modification in three or more of ALT4, KR, GDSL1, FATB, and ISS1. In some examples, the modified Cannabis plant includes a genetic modification in four or more of ALT4, KR, GDSL1, FATB, and ISS1. In some examples, the modified Cannabis plant includes a genetic modification in all of ALT4, KR, GDSL1, FATB, and ISS1. In some examples, the modified Cannabis plant includes a genetic modification in ALT4 and ISS1.

In some examples the modified Cannabis plant includes a heterologous beneficial allele of ALT4, KR, GDSL1, FATB, and/or ISS1. Modified Cannabis plants including a heterologous beneficial allele are sometimes referred to as transgenic Cannabis plants. In some examples the modified Cannabis plant includes a heterologous beneficial allele of ALT4. In some examples the modified Cannabis plant includes a heterologous beneficial allele of KR. In some examples the modified Cannabis plant includes a heterologous beneficial allele of GDSL1. In some examples the modified Cannabis plant includes a heterologous beneficial allele of FATB. In some examples, the modified Cannabis plant includes a heterologous beneficial allele of ISS1. In some examples, the heterologous beneficial allele comprises or consists of a coding sequence of ALT4, KR, GDSL1, FATB, and/or ISS1. In some examples, the heterologous beneficial allele comprises a coding sequence of ALT4, KR, GDSL1, FATB, and/or ISS1 and a promoter. The promoter can be a promoter native to ALT4, KR, GDSL1, FATB, or ISS1, respectively, or can be a non-native promoter, for example, a 35S or other promoter for constitutive expression.

In some examples, the modified Cannabis plant includes a heterologous beneficial allele of two or more of: ALT4, KR, GDSL1, FATB, and ISS1. In some examples, the modified Cannabis plant includes a heterologous beneficial allele of three or more of: ALT4, KR, GDSL1, FATB, and ISS1. In some examples, the modified Cannabis plant includes a heterologous beneficial allele of four or more of: ALT4, KR, GDSL1, FATB, and ISS1. In some examples, the modified Cannabis plant includes a heterologous beneficial allele of all of ALT4, KR, GDSL1, FATB, and ISS1. In some examples, the modified Cannabis plant includes a heterologous beneficial allele of ALT4 and ISS1. Exemplary beneficial alleles of ALT4, KR, GDSL1, FATB, and ISS1 are disclosed herein (see, e.g., Example 1).

In some aspects, the methods include introducing a genetic modification in ALT4, KR, GDSL1, FATB, and/or ISS1. In some examples, the genetic modification is associated with varin production or increased varin production. In some examples, the genetic modification increases varin production relative to the plant in an unmodified state (or a reference plant that does not include the genetic modification). In some implementations, the genetic modification produces a plant having a total varin content of at least 1%, for example, at least 1.5%, at least 2.0%, at least 2.5%, at least 3%, at least 3.5%, at least 4%, at least 4.5%, at least 5%, at least 5.5%, at least 6%, at least 6.5%, at least 7%, at least 7.5%, at least 8%, at least 8.5%, at least 9%, at least 9.5%, at least 10%, at least 11%, at least 12%, at least 13%, at least 14%, at least 15%, at least 16%, at least 17%, at least 18%, at least 19%, at least 20%, at least 25%, at least 30%, at least 35%, or at least 40% in at least one plant part (e.g., flower, leaf, or trichome). In a non-limiting example, the plant has a total varin content of at least 4%. In a non-limiting example, the plant has a total varin content of at least 2%. In another non-limiting example, the plant has a total varin content of at least 5%. In a further non-limiting example, the plant has a total varin content of at least 6%. In some examples, the plant has a total varin content of at least 15%. In another non-limiting example, the plant has a total varin content of at least 10%. In some examples, the plant has a total varin content of at least 20%. In another non-limiting example, the plant has a total varin content of at least 16.5%.

In some implementations, the genetic modification results in a plant having a total varin content of 1% to 40%, for example, 1% to 39%, 1% to 35%, 1% to 30%, 1% to 25%, 1% to 24%, 1% to 23%, 1% to 22%, 1% to 21%, 1% to 20%, 1% to 19%, 1% to 18%, 1% to 17%, 1% to 16%, 1% to 15%, 1% to 14%, 1% to 13%, 1% to 12%, 1% to 11%, 1% to 10%, 1% to 9%, 1% to 8%, 1% to 7%, 1% to 6%, 1% to 5%, 1% to 4%, 1% to 3%, 1% to 2%, 2% to 40%, 2% to 35%, 2% to 30%, 2% to 25%, 2% to 24%, 2% to 23%, 2% to 22%, 2% to 21%, 2% to 20%, 2% to 19%, 2% to 18%, 2% to 17%, 2% to 16%, 2% to 15%, 2% to 14%, 2% to 13%, 2% to 12%, 2% to 11%, 2% to 10%, 2% to 9%, 2% to 8%, 2% to 7%, 2% to 6%, 2% to 5%, 2% to 4%, 2% to 3%, 3% to 40%, 3% to 35%, 3% to 30%, 3% to 25%, 3% to 24%, 3% to 23%, 3% to 22%, 3% to 21%, 3% to 20%, 3% to 19%, 3% to 18%, 3% to 17%, 3% to 16%, 3% to 15%, 3% to 14%, 3% to 13%, 3% to 12%, 3% to 11%, 3% to 10%, 3% to 9%, 3% to 8%, 3% to 7%, 3% to 6%, 3% to 5%, 3% to 4%, 4% to 40%, 4% to 35%, 4% to 30%, 4% to 25%, 4% to 24%, 4% to 23%, 4% to 22%, 4% to 21%, 4% to 20%, 4% to 19%, 4% to 18%, 4% to 17%, 4% to 16%, 4% to 15%, 4% to 14%, 4% to 13%, 4% to 12%, 4% to 11%, 4% to 10%, 4% to 9%, 4% to 8%, 4% to 7%, 4% to 6%, 4% to 5%, 5% to 40%, 5% to 35%, 5% to 30%, 5% to 25%, 5% to 24%, 5% to 23%, 5% to 22%, 5% to 21%, 5% to 20%, 5% to 19%, 5% to 18%, 5% to 17%, 5% to 16%, 5% to 15%, 5% to 14%, 5% to 13%, 5% to 12%, 5% to 11%, 5% to 10%, 5% to 9%, 5% to 8%, 5% to 7%, 5% to 6%, 6% to 40%, 6% to 35%, 6% to 30%, 6% to 25%, 6% to 24%, 6% to 23%, 6% to 22%, 6% to 21%, 6% to 20%, 6% to 19%, 6% to 18%, 6% to 17%, 6% to 16%, 6% to 15%, 6% to 14%, 6% to 13%, 6% to 12%, 6% to 11%, 6% to 10%, 6% to 9%, 6% to 8%, 6% to 7%, 7% to 40%, 7% to 35%, 7% to 30%, 7% to 25%, 7% to 24%, 7% to 23%, 7% to 22%, 7% to 21%, 7% to 20%, 7% to 19%, 7% to 18%, 7% to 17%, 7% to 16%, 7% to 15%, 7% to 14%, 7% to 13%, 7% to 12%, 7% to 11%, 7% to 10%, 7% to 9%, 7% to 8%, 8% to 40%, 8% to 35%, 8% to 30%, 8% to 25%, 8% to 24%, 8% to 23%, 8% to 22%, 8% to 21%, 8% to 20%, 8% to 19%, 8% to 18%, 8% to 17%, 8% to 16%, 8% to 15%, 8% to 14%, 8% to 13%, 8% to 12%, 8% to 11%, 8% to 10%, 8% to 9%, 9% to 40%, 9% to 35%, 9% to 30%, 9% to 25%, 9% to 24%, 9% to 23%, 9% to 22%, 9% to 21%, 9% to 20%, 9% to 19%, 9% to 18%, 9% to 17%, 9% to 16%, 9% to 15%, 9% to 14%, 9% to 13%, 9% to 12%, 9% to 11%, 9% to 10%, 10% to 40%, 10% to 35%, 10% to 30%, 10% to 25%, 10% to 24%, 10% to 23%, 10% to 22%, 10% to 21%, 10% to 20%, 10% to 19%, 10% to 18%, 10% to 17%, 10% to 16%, 10% to 15%, 10% to 14%, 10% to 13%, 10% to 12%, 10% to 11%, 11% to 19%, 11% to 18%, 11% to 17%, 11% to 16%, 11% to 15%, 11% to 14%, 11% to 13%, 11% to 12%, 12% to 19%, 12% to 18%, 12% to 17%, 12% to 16%, 12% to 15%, 12% to 14%, 12% to 13%, 13% to 19%, 13% to 18%, 13% to 17%, 13% to 16%, 13% to 15%, 13% to 14%, 14% to 19%, 14% to 18%, 14% to 17%, 14% to 16%, 14% to 15%, 15% to 40%, 15% to 35%, 15% to 30%, 15% to 25%, 15% to 24%, 15% to 23%, 15% to 22%, 15% to 21%, 15% to 20%, 15% to 19%, 15% to 18%, 15% to 17%, 15% to 16%, 16% to 19%, 16% to 18%, 16% to 17%, 17% to 19%, 17% to 18%, 18% to 19%, 19% to 40%, 19% to 35%, 19% to 30%, 19% to 25%, 19% to 24%, 19% to 23%, 19% to 22%, 19% to 21%, 19% to 20%, 20% to 40%, 20% to 35%, 20% to 30%, 20% to 25%, 20% to 24%, 20% to 23%, 20% to 22%, 20% to 21%, 25% to 40%, 25% to 35%, 25% to 30%, 30% to 40%, 30% to 35%, or 35% to 40% total varin content in at least one plant part (e.g., flower, leaf, or trichome). In some examples, the total varin content is 2% to 7%. In some examples, the total varin content is 3% to 7%. In some examples, the total varin content is 4% to 7%. In a further example, the total varin content is 4% to 18%. In another example, the total varin content is 4% to 16.5%. In another example, the total varin content is 7% to 16.5%. In some examples, the total varin content is 15% to 35%. In some examples, the total varin content is 15% to 20%.

In some implementations, the genetic modification results in a plant having a varin ratio of at least 0.1, for example, at least 0.2, at least 0.3, at least 0.33, at least 0.4, at least 0.5, at least 0.6, at least 0.7, at least 0.8, at least 0.9, at least 1, at least 1.5, at least 2.0, at least 2.5, at least 3, at least 3.5, at least 4, at least 4.5, at least 5, at least 5.5, at least 6, at least 6.5, at least 7, at least 7.5, at least 8, at least 8.5, at least 9, at least 9.5, at least 10, at least 15, or at least 20 in at least one plant part (e.g., flower, leaf, or trichome). In a non-limiting example, the plant has a varin ratio of at least 0.2. In a non-limiting example, the plant has a varin ratio of at least 1. In a non-limiting example, the plant has a varin ratio of at least 3. In another non-limiting example, the plant has a varin ratio of at least 4. In some examples, the plant has a varin ratio of at least 7.

In some implementations, the genetic modification results in a plant having a varin ratio of 0.1 to 30, for example, 0.1 to 25, 0.1 to 20, 0.1 to 15, 0.1 to 10, 0.1 to 5, 0.1 to 4, 0.1 to 3, 0.1 to 2, 0.1 to 1, 0.1 to 0.5, 0.25 to 30, 0.25 to 25, 0.25 to 20, 0.25 to 15, 0.25 to 10, 0.25 to 5, 0.25 to 4, 0.25 to 3, 0.25 to 2, 0.25 to 1, 0.25 to 0.5, 0.33 to 25, 0.3 to 20, 0.33 to 15, 0.33 to 10, 0.33 to 7, 0.33 to 5, 0.33 to 3, 0.33 to 1, 0.33 to 0.5, 0.5 to 30, 0.5 to 25, 0.5 to 20, 0.5 to 15, 0.5 to 10, 0.5 to 5, 0.5 to 4, 0.5 to 3, 0.5 to 2, 0.5 to 1, 0.75 to 25, 0.75 to 20, 0.75 to 15, 0.75 to 10, 0.75 to 5, 0.75 to 4, 0.75 to 3, 0.75 to 2, 0.75 to 1, 1 to 30, 1 to 25, 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, 1 to 2, 2 to 30, 2 to 25, 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4, 2 to 3, 3 to 30, 3 to 25, 3 to 20, 3 to 19, 3 to 18, 3 to 17, 3 to 16, 3 to 15, 3 to 14, 3 to 13, 3 to 12, 3 to 11, 3 to 10, 3 to 9, 3 to 8, 3 to 7, 3 to 6, 3 to 5, 3 to 4, 4 to 30, 4 to 25, 4 to 20, 4 to 19, 4 to 18, 4 to 17, 4 to 16, 4 to 15, 4 to 14, 4 to 13, 4 to 12, 4 to 11, 4 to 10, 4 to 9, 4 to 8, 4 to 7, 4 to 6, 4 to 5, 5 to 30, 5 to 25, 5 to 20, 5 to 19, 5 to 18, 5 to 17, 5 to 16, 5 to 15, 5 to 14, 5 to 13, 5 to 12, 5 to 11, 5 to 10, 5 to 9, 5 to 8, 5 to 7, 5 to 6, 6 to 30, 6 to 25, 6 to 20, 6 to 19, 6 to 18, 6 to 17, 6 to 16, 6 to 15, 6 to 14, 6 to 13, 6 to 12, 6 to 11, 6 to 10, 6 to 9, 6 to 8, 6 to 7, 7 to 30, 7 to 25, 7 to 20, 7 to 19, 7 to 18, 7 to 17, 7 to 16, 7 to 15, 7 to 14, 7 to 13, 7 to 12, 7 to 11, 7 to 10, 7 to 9, 7 to 8, 8 to 30, 8 to 25, 8 to 20, 8 to 19, 8 to 18, 8 to 17, 8 to 16, 8 to 15, 8 to 14, 8 to 13, 8 to 12, 8 to 11, 8 to 10, 8 to 9, 9 to 30, 9 to 25, 9 to 20, 9 to 19, 9 to 18, 9 to 17, 9 to 16, 9 to 15, 9 to 14, 9 to 13, 9 to 12, 9 to 11, 9 to 10, 10 to 30, 10 to 25, 10 to 20, 10 to 18, 10 to 16, 10 to 14, 10 to 12, 12 to 30, 12 to 25, 12 to 20, 12 to 18, 12 to 16, 12 to 14, 14 to 30, 14 to 25, 14 to 20, 14 to 18, 14 to 16, 16 to 30, 16 to 25, 16 to 20, 16 to 18, 18 to 30, 18 to 25, 18 to 20, 20 to 30, 20 to 25, or 25 to 30 in at least one plant part (e.g., flower, leaf, or trichome). In some examples, the varin ratio is 1 to 8. In some examples, the varin ratio is 0.25 to 5. In some examples, the varin ratio is 1 to 5. In further examples, the varin ratio is 7 to 20. In some examples, the varin ratio is 7 to 10.

A plant part includes any part of the modified Cannabis plant. In some examples, the plant part is a flower or inflorescence of the modified Cannabis plant. In some examples, the plant part is trichomes of the modified Cannabis plant. In further examples, the plant part is leaf or other vegetative tissue of the modified Cannabis plant.

In some examples, the genetic modification is a nucleic acid substitution, insertion, or deletion. In some examples the modification is homozygous or heterozygous in the modified plant. In some examples, the modification is homozygous in the modified plant. In some implementations, the genetic modification is introduced by mutagenesis or a gene editing technique (e.g., RNAi, CRISPR/Cas9, ZFN, or TALEN based systems).

DNAzyme molecules, enzymatic oligonucleotides, and mutagenesis are commonly known methods for introducing genetic modifications. Any available mutagenesis procedure can be used, including but not limited to, site-directed point mutagenesis, random point mutagenesis, in vitro or in vivo homologous recombination (DNA shuffling), uracil-containing templates, oligonucleotide-directed mutagenesis, phosphorothioate-modified DNA mutagenesis, mutagenesis using gapped duplex DNA, point mismatch repair, repair-deficient host strains, restriction-selection and restriction-purification, deletion mutagenesis, total gene synthesis, double-strand break repair, zinc-finger nucleases (ZFN), or transcription activator-like effector nucleases (TALEN).

Clustered regularly interspaced short palindromic repeats (CRISPR)/CRISPR associated protein (Cas) system comprises genome engineering tools based on the bacterial CRISPR/Cas prokaryotic adaptive immune system. This RNA-based technology allows targeted cleavage of genomic DNA guided by a customizable small noncoding RNA, resulting in gene modifications by both non-homologous end joining (NHEJ) and homology-directed repair (HDR) mechanisms (see, e.g., Belhaj K. et al., (2013) Plant Methods, 9:39). A non-limiting example of a CRISPR/Cas system includes a CRISPR/Cas9 system. In some examples, the target cell expresses a Cas nuclease (e.g., Cas9), and a CRISPR RNA is expressed or transformed into the cell. In some examples, a nucleoprotein complex comprising a Cas nuclease (e.g., Cas9) and a CRISPR RNA is transformed into the target cell. CRISPR-based gene editing systems need not be limited to Cas9 systems, as other suitable/analogous editing enzymes have been described, e.g., MAD7. In some implementations, a CRISPR RNA targets ALT4, KR, GDSL1, FATB, or ISS1 (including a promoter region thereof) to introduce a beneficial polymorphism described herein. Design of CRISPR RNA has been described, and CRISPR RNAs (and Cas9) are commercially available.

In some examples, gene expression of ALT4, KR, GDSL1, FATB, or ISS1 is modified using at least one antisense compound targeting a gene of interest (e.g., ALT4, KR, GDSL1, FATB, or ISS1, respectively), including antisense DNA, antisense RNA, ribozymes, DNAzymes, locked nucleic acid (LNA), or aptamer. In some examples, the molecules are chemically modified. In some examples, the antisense molecule is antisense DNA or an antisense DNA analog.

RNA interference (RNAi) can reduce gene function in plants, which is mediated by RNA-induced silencing complex (RISC), a sequence-specific, multicomponent nuclease that destroys messenger RNAs homologous to the silencing trigger. RISC is known to contain short RNAs (approximately 22 nucleotides) derived from the double-stranded RNA trigger. The short-nucleotide RNA sequences are homologous to the target gene that is being suppressed. Thus, the short-nucleotide sequences appear to serve as guide sequences to instruct a multicomponent nuclease, RISC, to destroy the specific mRNAs. The dsRNA used to initiate RNAi, may be isolated from native source or produced by known means, e.g., transcribed from DNA. Plasmids and vectors for generating RNAi molecules against target sequence are readily available from commercial sources.

In some implementations, the method of producing a modified Cannabis plant comprises introducing a heterologous gene, for example, a beneficial allele (an allele associated with varin production or increased varin production) of ALT4, KR, GDSL1, FATB, and/or ISS1. In some examples, the method includes introducing a beneficial allele of ISS1 and KR. In some examples, the method includes introducing a beneficial allele of ISS1, KR, and ALT4. In some examples, the beneficial allele increases varin production relative to a Cannabis plant in an unmodified state. In some implementations, introducing the beneficial allele produces a plant having a total varin content of at least 1%, for example, at least 1.5%, at least 2.0%, at least 2.5%, at least 3%, at least 3.5%, at least 4%, at least 4.5%, at least 5%, at least 5.5%, at least 6%, at least 6.5%, at least 7%, at least 7.5%, at least 8%, at least 8.5%, at least 9%, at least 9.5%, at least 10%, at least 11%, at least 12%, at least 13%, at least 14%, at least 15%, at least 16%, at least 17%, at least 18%, at least 19%, at least 20%, at least 25%, at least 30%, at least 35%, or at least 40% in at least one plant part (e.g., flower, leaf, or trichome). In a non-limiting example, the plant has a total varin content of at least 4%. In a non-limiting example, the plant has a total varin content of at least 2%. In another non-limiting example, the plant has a total varin content of at least 5%. In a further non-limiting example, the plant has a total varin content of at least 6%. In some examples, the plant has a total varin content of at least 15%. In another non-limiting example, the plant has a total varin content of at least 10%. In another non-limiting example, the plant has a total varin content of at least 16.5%. In some examples, the plant has a total varin content of at least 20%.

In some implementations, introducing the beneficial allele produces a plant having a total varin content of 1% to 40%, for example, 1% to 39%, 1% to 35%, 1% to 30%, 1% to 25%, 1% to 24%, 1% to 23%, 1% to 22%, 1% to 21%, 1% to 20%, 1% to 19%, 1% to 18%, 1% to 17%, 1% to 16%, 1% to 15%, 1% to 14%, 1% to 13%, 1% to 12%, 1% to 11%, 1% to 10%, 1% to 9%, 1% to 8%, 1% to 7%, 1% to 6%, 1% to 5%, 1% to 4%, 1% to 3%, 1% to 2%, 2% to 40%, 2% to 35%, 2% to 30%, 2% to 25%, 2% to 24%, 2% to 23%, 2% to 22%, 2% to 21%, 2% to 20%, 2% to 19%, 2% to 18%, 2% to 17%, 2% to 16%, 2% to 15%, 2% to 14%, 2% to 13%, 2% to 12%, 2% to 11%, 2% to 10%, 2% to 9%, 2% to 8%, 2% to 7%, 2% to 6%, 2% to 5%, 2% to 4%, 2% to 3%, 3% to 40%, 3% to 35%, 3% to 30%, 3% to 25%, 3% to 24%, 3% to 23%, 3% to 22%, 3% to 21%, 3% to 20%, 3% to 19%, 3% to 18%, 3% to 17%, 3% to 16%, 3% to 15%, 3% to 14%, 3% to 13%, 3% to 12%, 3% to 11%, 3% to 10%, 3% to 9%, 3% to 8%, 3% to 7%, 3% to 6%, 3% to 5%, 3% to 4%, 4% to 40%, 4% to 35%, 4% to 30%, 4% to 25%, 4% to 24%, 4% to 23%, 4% to 22%, 4% to 21%, 4% to 20%, 4% to 19%, 4% to 18%, 4% to 17%, 4% to 16%, 4% to 15%, 4% to 14%, 4% to 13%, 4% to 12%, 4% to 11%, 4% to 10%, 4% to 9%, 4% to 8%, 4% to 7%, 4% to 6%, 4% to 5%, 5% to 40%, 5% to 35%, 5% to 30%, 5% to 25%, 5% to 24%, 5% to 23%, 5% to 22%, 5% to 21%, 5% to 20%, 5% to 19%, 5% to 18%, 5% to 17%, 5% to 16%, 5% to 15%, 5% to 14%, 5% to 13%, 5% to 12%, 5% to 11%, 5% to 10%, 5% to 9%, 5% to 8%, 5% to 7%, 5% to 6%, 6% to 40%, 6% to 35%, 6% to 30%, 6% to 25%, 6% to 24%, 6% to 23%, 6% to 22%, 6% to 21%, 6% to 20%, 6% to 19%, 6% to 18%, 6% to 17%, 6% to 16%, 6% to 15%, 6% to 14%, 6% to 13%, 6% to 12%, 6% to 11%, 6% to 10%, 6% to 9%, 6% to 8%, 6% to 7%, 7% to 40%, 7% to 35%, 7% to 30%, 7% to 25%, 7% to 24%, 7% to 23%, 7% to 22%, 7% to 21%, 7% to 20%, 7% to 19%, 7% to 18%, 7% to 17%, 7% to 16%, 7% to 15%, 7% to 14%, 7% to 13%, 7% to 12%, 7% to 11%, 7% to 10%, 7% to 9%, 7% to 8%, 8% to 40%, 8% to 35%, 8% to 30%, 8% to 25%, 8% to 24%, 8% to 23%, 8% to 22%, 8% to 21%, 8% to 20%, 8% to 19%, 8% to 18%, 8% to 17%, 8% to 16%, 8% to 15%, 8% to 14%, 8% to 13%, 8% to 12%, 8% to 11%, 8% to 10%, 8% to 9%, 9% to 40%, 9% to 35%, 9% to 30%, 9% to 25%, 9% to 24%, 9% to 23%, 9% to 22%, 9% to 21%, 9% to 20%, 9% to 19%, 9% to 18%, 9% to 17%, 9% to 16%, 9% to 15%, 9% to 14%, 9% to 13%, 9% to 12%, 9% to 11%, 9% to 10%, 10% to 40%, 10% to 35%, 10% to 30%, 10% to 25%, 10% to 24%, 10% to 23%, 10% to 22%, 10% to 21%, 10% to 20%, 10% to 19%, 10% to 18%, 10% to 17%, 10% to 16%, 10% to 15%, 10% to 14%, 10% to 13%, 10% to 12%, 10% to 11%, 11% to 19%, 11% to 18%, 11% to 17%, 11% to 16%, 11% to 15%, 11% to 14%, 11% to 13%, 11% to 12%, 12% to 19%, 12% to 18%, 12% to 17%, 12% to 16%, 12% to 15%, 12% to 14%, 12% to 13%, 13% to 19%, 13% to 18%, 13% to 17%, 13% to 16%, 13% to 15%, 13% to 14%, 14% to 19%, 14% to 18%, 14% to 17%, 14% to 16%, 14% to 15%, 15% to 40%, 15% to 35%, 15% to 30%, 15% to 25%, 15% to 24%, 15% to 23%, 15% to 22%, 15% to 21%, 15% to 20%, 15% to 19%, 15% to 18%, 15% to 17%, 15% to 16%, 16% to 19%, 16% to 18%, 16% to 17%, 17% to 19%, 17% to 18%, 18% to 19%, 19% to 40%, 19% to 35%, 19% to 30%, 19% to 25%, 19% to 24%, 19% to 23%, 19% to 22%, 19% to 21%, 19% to 20%, 20% to 40%, 20% to 35%, 20% to 30%, 20% to 25%, 20% to 24%, 20% to 23%, 20% to 22%, 20% to 21%, 25% to 40%, 25% to 35%, 25% to 30%, 30% to 40%, 30% to 35%, or 35% to 40% total varin content in at least one plant part (e.g., flower, leaf, or trichome). In some examples, the total varin content is 2% to 7%. In some examples, the total varin content is 3% to 7%. In some examples, the total varin content is 4% to 7%. In a further example, the total varin content is 4% to 18%. In another example, the total varin content is 4% to 16.5%. In another example, the total varin content is 7% to 16.5%. In some examples, the total varin content is 15% to 35%. In some examples, the total varin content is 15% to 20%.

In some implementations, introducing the beneficial allele produces a plant having a varin ratio of at least 0.1, for example, at least 0.2, at least 0.3, at least 0.33, at least 0.4, at least 0.5, at least 0.6, at least 0.7, at least 0.8, at least 0.9, at least 1, at least 1.5, at least 2.0, at least 2.5, at least 3, at least 3.5, at least 4, at least 4.5, at least 5, at least 5.5, at least 6, at least 6.5, at least 7, at least 7.5, at least 8, at least 8.5, at least 9, at least 9.5, at least 10, at least 15, or at least 20 in at least one plant part (e.g., flower, leaf, or trichome). In a non-limiting example, the plant has a varin ratio of at least 0.2. In a non-limiting example, the plant has a varin ratio of at least 1. In a non-limiting example, the plant has a varin ratio of at least 3. In another non-limiting example, the plant has a varin ratio of at least 4. In some examples, the varin ratio is at least 7.

In some implementations, introducing the beneficial allele produces a plant having a varin ratio of 0.1 to 30, for example, 0.1 to 25, 0.1 to 20, 0.1 to 15, 0.1 to 10, 0.1 to 5, 0.1 to 4, 0.1 to 3, 0.1 to 2, 0.1 to 1, 0.1 to 0.5, 0.25 to 30, 0.25 to 25, 0.25 to 20, 0.25 to 15, 0.25 to 10, 0.25 to 5, 0.25 to 4, 0.25 to 3, 0.25 to 2, 0.25 to 1, 0.25 to 0.5, 0.33 to 25, 0.33 to 20, 0.33 to 15, 0.33 to 10, 0.33 to 7, 0.33 to 5, 0.33 to 3, 0.33 to 1, 0.33 to 0.5, 0.5 to 30, 0.5 to 25, 0.5 to 20, 0.5 to 15, 0.5 to 10, 0.5 to 5, 0.5 to 4, 0.5 to 3, 0.5 to 2, 0.5 to 1, 0.75 to 25, 0.75 to 20, 0.75 to 15, 0.75 to 10, 0.75 to 5, 0.75 to 4, 0.75 to 3, 0.75 to 2, 0.75 to 1, 1 to 30, 1 to 25, 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, 1 to 2, 2 to 30, 2 to 25, 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4, 2 to 3, 3 to 30, 3 to 25, 3 to 20, 3 to 19, 3 to 18, 3 to 17, 3 to 16, 3 to 15, 3 to 14, 3 to 13, 3 to 12, 3 to 11, 3 to 10, 3 to 9, 3 to 8, 3 to 7, 3 to 6, 3 to 5, 3 to 4, 4 to 30, 4 to 25, 4 to 20, 4 to 19, 4 to 18, 4 to 17, 4 to 16, 4 to 15, 4 to 14, 4 to 13, 4 to 12, 4 to 11, 4 to 10, 4 to 9, 4 to 8, 4 to 7, 4 to 6, 4 to 5, 5 to 30, 5 to 25, 5 to 20, 5 to 19, 5 to 18, 5 to 17, 5 to 16, 5 to 15, 5 to 14, 5 to 13, 5 to 12, 5 to 11, 5 to 10, 5 to 9, 5 to 8, 5 to 7, 5 to 6, 6 to 30, 6 to 25, 6 to 20, 6 to 19, 6 to 18, 6 to 17, 6 to 16, 6 to 15, 6 to 14, 6 to 13, 6 to 12, 6 to 11, 6 to 10, 6 to 9, 6 to 8, 6 to 7, 7 to 30, 7 to 25, 7 to 20, 7 to 19, 7 to 18, 7 to 17, 7 to 16, 7 to 15, 7 to 14, 7 to 13, 7 to 12, 7 to 11, 7 to 10, 7 to 9, 7 to 8, 8 to 30, 8 to 25, 8 to 20, 8 to 19, 8 to 18, 8 to 17, 8 to 16, 8 to 15, 8 to 14, 8 to 13, 8 to 12, 8 to 11, 8 to 10, 8 to 9, 9 to 30, 9 to 25, 9 to 20, 9 to 19, 9 to 18, 9 to 17, 9 to 16, 9 to 15, 9 to 14, 9 to 13, 9 to 12, 9 to 11, 9 to 10, 10 to 30, 10 to 25, 10 to 20, 10 to 18, 10 to 16, 10 to 14, 10 to 12, 12 to 30, 12 to 25, 12 to 20, 12 to 18, 12 to 16, 12 to 14, 14 to 30, 14 to 25, 14 to 20, 14 to 18, 14 to 16, 16 to 30, 16 to 25, 16 to 20, 16 to 18, 18 to 30, 18 to 25, 18 to 20, 20 to 30, 20 to 25, or 25 to 30 in at least one plant part (e.g., flower, leaf, or trichome). In some examples, the varin ratio is 1 to 8. In some examples, the varin ratio is 0.25 to 5. In some examples, the varin ratio is 1 to 5. In further examples, the varin ratio is 7 to 20. In some examples, the varin ratio is 7 to 10.

A plant part includes any part of the modified Cannabis plant (e.g., the plant carrying a beneficial allele of ALT4, KR, GDSL1, FATB, and/or ISS1). In some examples, the plant part is a flower or inflorescence of the modified Cannabis plant. In some examples, the plant part is trichomes of the modified Cannabis plant. In further examples, the plant part is leaf or other vegetative tissue of the modified Cannabis plant.

Examples of beneficial polymorphisms of ALT4 are described herein in the “Methods of Identifying Plants with Increased Varin Production” section and the Examples. In a non-limiting example, the beneficial allele of ALT4 includes a polymorphism that produces a glutamine corresponding to Abacus reference position 59 and/or a glycine corresponding to Abacus reference position 66. In a further example, the one or more nucleic acid polymorphisms detected in ALT4 produce a serine corresponding to Abacus reference position 7 or a lysine corresponding to Abacus reference position 68. The positions are in reference to the Abacus ALT4 protein sequence (SEQ ID NO: 12). In a non-limiting example, the beneficial allele of ISS1 includes a deletion of the A corresponding to Abacus reference position −436 bp upstream from the start codon of ISS1. This position is in reference to the Abacus genome version Csat_Abacus V2 (NCBI assembly accession GCA_025232715.1; see also, SEQ ID NO: 31).

In further examples, a beneficial allele of ALT4 includes a nucleic acid sequence encoding an amino acid sequence having at least 80%, for example, at least 85%, 90%, 95%, 98%, 99%, sequence identity to SEQ ID NO: 10-12 or 47. In some examples, the beneficial allele of ALT4 includes a nucleic acid sequence encoding an amino acid sequence having at least 95% sequence identity to SEQ ID NO: 10-12 or 47. In some examples, the beneficial allele includes one or more of: (a) a glutamine corresponding to Abacus reference position 59; (b) a glycine corresponding to Abacus reference position 66; (c) an insertion of aspartic acid immediately following Abacus reference position 47; (d) a tyrosine corresponding to Abacus reference position 67; (e) a deletion of aspartic acid corresponding to Abacus reference position 48; (f) a deletion of aspartic acid corresponding to Abacus reference position 49; (g) a lysine corresponding to Abacus reference position 68; (h) an aspartic acid corresponding to Abacus reference position 81; (i) a glutamic acid corresponding to Abacus reference position 85; (j) a histidine corresponding to Abacus reference position 111; (k) an alanine corresponding to Abacus reference position 115; (l) a serine corresponding to Abacus reference position 124; (m) a valine corresponding to Abacus reference position 125; (n) an aspartic acid corresponding to Abacus reference position 136; (o) an isoleucine corresponding to Abacus reference position 160; (p) a valine corresponding to Abacus reference position 165; (q) a valine corresponding to Abacus reference position 172; (r) a serine corresponding to Abacus reference position 7; (s) a glutamine corresponding to Abacus reference position 202; or (t) an alanine corresponding to Abacus reference position 206. In some examples, the beneficial allele of ALT4 includes a serine at position 7 corresponding to Abacus ALT4 sequence set forth as SEQ ID NO: 12 or a lysine at position 68 corresponding to Abacus ALT4 sequence set forth as SEQ ID NO: 12.

In some examples, the beneficial allele of ALT4 includes a nucleic acid sequence encoding an amino acid sequence having at least 95% sequence identity to SEQ ID NO: 12 and further includes one or more of: (a) a glutamine corresponding to Abacus reference position 59; (b) a glycine corresponding to Abacus reference position 66; (c) an insertion of aspartic acid immediately following Abacus reference position 47; (d) a tyrosine corresponding to Abacus reference position 67; (e) a deletion of aspartic acid corresponding to Abacus reference position 48; (f) a deletion of aspartic acid corresponding to Abacus reference position 49; (g) a lysine corresponding to Abacus reference position 68; (h) an aspartic acid corresponding to Abacus reference position 81; (i) a glutamic acid corresponding to Abacus reference position 85; (j) a histidine corresponding to Abacus reference position 111; (k) an alanine corresponding to Abacus reference position 115; (l) a serine corresponding to Abacus reference position 124; (m) a valine corresponding to Abacus reference position 125; (n) an aspartic acid corresponding to Abacus reference position 136; (o) an isoleucine corresponding to Abacus reference position 160; (p) a valine corresponding to Abacus reference position 165; (q) a valine corresponding to Abacus reference position 172; (r) a serine corresponding to Abacus reference position 7; (s) a glutamine corresponding to Abacus reference position 202; or (t) an alanine corresponding to Abacus reference position 206. The positions are in reference to the Abacus ALT4 protein sequence (SEQ ID NO: 12). In some examples, the beneficial allele of ALT4 includes a nucleic acid sequence encoding an amino acid sequence having at least 95% sequence identity to SEQ ID NO: 12 and includes a serine at position 7 corresponding to Abacus ALT4 sequence set forth as SEQ ID NO: 12 or a lysine at position 68 corresponding to Abacus ALT4 sequence set forth as SEQ ID NO: 12.

In some examples a beneficial allele of ALT4 includes a nucleic acid sequence encoding an amino acid sequence having at least 95% sequence identity to SEQ ID NO: 10 and includes one or more of: (a) a glutamine corresponding to Abacus reference position 59; (b) a glycine corresponding to Abacus reference position 66; (c) an insertion of aspartic acid immediately following Abacus reference position 47; and (d) a tyrosine corresponding to Abacus reference position 67. In some examples a beneficial allele of ALT4 includes a nucleic acid sequence encoding an amino acid sequence having at least 90% sequence identity to SEQ ID NO: 11 and includes (a) a glutamine corresponding to Abacus reference position 59; (b) a glycine corresponding to Abacus reference position 66; (e) a deletion of aspartic acid corresponding to Abacus reference position 48; (f) a deletion of aspartic acid corresponding to Abacus reference position 49; (g) a lysine corresponding to Abacus reference position 68; (h) an aspartic acid corresponding to Abacus reference position 81; (i) a glutamic acid corresponding to Abacus reference position 85; (j) a histidine corresponding to Abacus reference position 111; (k) an alanine corresponding to Abacus reference position 115; (l) a serine corresponding to Abacus reference position 124; (m) a valine corresponding to Abacus reference position 125; (n) an aspartic acid corresponding to Abacus reference position 136; (o) an isoleucine corresponding to Abacus reference position 160; (p) a valine corresponding to Abacus reference position 165; (q) the valine corresponding to Abacus reference position 172; (r) a serine corresponding to Abacus reference position 7; (s) a glutamine corresponding to Abacus reference position 202; or (t) an alanine corresponding to Abacus reference position 206. The positions are in reference to the Abacus ALT4 protein sequence (SEQ ID NO: 12).

In some examples, a beneficial allele of ALT4 includes a nucleic acid sequence encoding SEQ ID NO: 10 or SEQ ID NO: 11. In some examples, a beneficial allele of ALT4 consists of or essentially consists of a nucleic acid sequence encoding SEQ ID NO: 10 or SEQ ID NO: 11.

In further examples, a beneficial allele of KR includes a nucleic acid sequence encoding an amino acid sequence having at least 80%, for example, at least 85%, 90%, 95%, 98%, 99%, sequence identity to any one of SEQ ID NOs: 66-81. In some examples, the beneficial allele of KR includes a nucleic acid sequence encoding an amino acid sequence having at least 95% sequence identity to any one of SEQ ID NOs: 66-81. In some examples, the beneficial allele of KR includes a nucleic acid sequence encoding an amino acid sequence having at least 95% sequence identity to any one of SEQ ID NOs: 67-81. In some examples, the beneficial allele of KR further includes one or more of: (a) an arginine at position 39; (b) a proline at position 88; (c) a glutamic acid at position 122; (d) a glycine at position 123; (e) a glycine at position 125; (f) a proline at position 139; (g) a valine at position 158; (h) a glycine at position 175; (i) a serine at position 193; (j) a arginine at position 205; (k) a arginine at position 208; (l) an isoleucine at position 217; (m) a serine at position 218; (n) a serine at position 251; or (o) an isoleucine at position 293. The positions are in reference to the Abacus KR protein sequence (SEQ ID NO: 66). In some examples, the beneficial allele of KR includes a valine at position 158 corresponding to Abacus KR sequence set forth as SEQ ID NO: 66.

In some examples a beneficial allele of KR includes a nucleic acid sequence encoding an amino acid sequence having at least 95% sequence identity to SEQ ID NO: 66 and includes one or more of: (a) an arginine at position 39; (b) a proline at position 88; (c) a glutamic acid at position 122; (d) a glycine at position 123; (c) a glycine at position 125; (f) a proline at position 139; (g) a valine at position 158; (h) a glycine at position 175; (i) a serine at position 193; (j) a arginine at position 205; (k) a arginine at position 208; (l) an isoleucine at position 217; (m) a serine at position 218; (n) a serine at position 251; or (o) an isoleucine at position 293. The positions are in reference to the Abacus KR protein sequence (SEQ ID NO: 66). In some examples, the beneficial allele of KR has at least 95% sequence identity to SEQ ID NO: 66 and includes a valine at position 158 corresponding to Abacus KR sequence set forth as SEQ ID NO: 66.

In further examples, a beneficial allele of FATB includes a nucleic acid sequence encoding an amino acid sequence having at least 80%, for example, at least 85%, 90%, 95%, 98%, 99%, sequence identity to SEQ ID NO: 99 or 100. In some examples, the beneficial allele of FATB includes a nucleic acid sequence encoding an amino acid sequence having at least 95% sequence identity to SEQ ID NO: 99 or 100. In some examples, the beneficial allele of FATB further includes one or more of: (a) a histidine at position 155; (b) a glutamine at position 173; (c) an arginine at position 197; (d) an isoleucine at position 201; (e) a proline at position 202; or (f) an aspartic acid at position 207. The positions are in reference to the Abacus FATB protein sequence (SEQ ID NO: 100). In some examples, the beneficial allele of FATB includes an isoleucine at position 201 corresponding to Abacus FATB sequence set forth as SEQ ID NO: 100.

In some examples a beneficial allele of FATB includes a nucleic acid sequence encoding an amino acid sequence having at least 95% sequence identity to SEQ ID NO: 100 and includes one or more of: (a) a histidine at position 155; (b) a glutamine at position 173; (c) an arginine at position 197; (d) an isoleucine at position 201; (e) a proline at position 202; or (f) an aspartic acid at position 207. The positions are in reference to the Abacus FATB protein sequence (SEQ ID NO: 100). In some examples a beneficial allele of FATB includes a nucleic acid sequence encoding an amino acid sequence having at least 95% sequence identity to SEQ ID NO: 100 and includes an isoleucine at position 201 corresponding to Abacus FATB sequence set forth as SEQ ID NO: 100.

In further examples, a beneficial allele of GDSL1 includes a nucleic acid sequence encoding an amino acid sequence having at least 80%, for example, at least 85%, 90%, 95%, 98%, 99%, sequence identity to SEQ ID NO: 109 or 110. In some examples, the beneficial allele of GDSL1 includes a nucleic acid sequence encoding an amino acid sequence having at least 95% sequence identity to SEQ ID NO: 109 or 110. In some examples, the beneficial allele of GDSL1 further includes one or more of: (a) an alanine at position 59; (b) an asparagine at position 275; (c) a threonine at position 297; or (d) a methionine at position 213. The positions are in reference to the Abacus GDSL1 protein sequence (SEQ ID NO: 110). In some examples, the beneficial allele of GDSL1 includes a methionine at position 213 corresponding to Abacus GDSL1 sequence set forth as SEQ ID NO: 110.

In some examples a beneficial allele of GDSL1 includes a nucleic acid sequence encoding an amino acid sequence having at least 95% sequence identity to SEQ ID NO: 110 and includes one or more of: (a) an alanine at position 59; (b) an asparagine at position 275; (c) a threonine at position 297; or (d) a methionine at position 213. The positions are in reference to the Abacus GDSL1 protein sequence (SEQ ID NO: 110). In some examples, In some examples a beneficial allele of GDSL1 includes a nucleic acid sequence encoding an amino acid sequence having at least 95% sequence identity to SEQ ID NO: 110 and includes a methionine at position 213 corresponding to Abacus GDSL1 sequence set forth as SEQ ID NO: 110.

In some examples, a beneficial allele of ISS1 includes a nucleic acid sequence having at least 80%, for example, at least 85%, 90%, 95%, 98%, 99%, sequence identity to SEQ ID NO: 29. In some examples, the beneficial allele of ISS1 includes a nucleic acid sequence having at least 95% sequence identity to SEQ ID NO: 29. In some examples, a beneficial allele of ISS1 includes a nucleic acid sequence having at least 95% sequence identity to SEQ ID NO: 29 and includes a deletion of the A corresponding to Abacus reference position −436 bp upstream from the start codon of ISS1. This position is in reference to the Abacus genome version Csat_AbacusV2 (NCBI assembly accession GCA_025232715.1; see also, SEQ ID NO: 31). In some examples, a beneficial allele of ISS1 comprises SEQ ID NO: 29. In some examples, a beneficial allele of ISS1 consists of or essentially consists of SEQ ID NO: 29.

Methods of producing a modified Cannabis plant can include transformation steps, for example, to introduce nucleic acids or gene editing components into the plant. Methods of transformation are well characterized. Election of the most appropriate transformation technique can be determined by the practitioner. Suitable methods may include electroporation of plant protoplasts, liposome-mediated transformation, polyethylene glycol (PEG) mediated transformation, transformation using viruses, micro-injection of plant cells, micro-projectile bombardment of plant cells, and Agrobacterium tumefaciens mediated transformation. Transformation includes introducing a nucleotide sequence in a plant in a manner to cause stable (e.g., integrated into the genome) or transient expression (e.g., not integrated into the genome) of the sequence.

In planta transformation techniques (e.g., vacuum-infiltration, floral spraying or floral dip procedures) are well known and may be used to introduce expression cassettes and/or vectors containing expression cassettes (typically in an Agrobacterium-mediated transformation) into meristematic or germline cells of a whole plant. Such methods provide a simple and reliable method of obtaining transformants at high efficiency while avoiding the use of tissue culture. (see, e.g., Bechtold et at. 1993 C. R. Acad. Sci. 316:1194-1199; Chung et at. 2000 Transgenic Res. 9:471-476; Clough et al. 1998 Plant J. 16:735-743; and Desfeux et at. 2000 Plant Physiol 123:895-904). In such techniques, seed produced by the plant comprise the expression cassettes encoding the genes of interest. The seed can be selected based on the ability to germinate under conditions that inhibit germination of the untransformed seed.

If transformation techniques require use of tissue culture, transformed cells may be regenerated into plants in accordance with previously described techniques. The regenerated plants may be grown and crossed with the same or different plant varieties using traditional breeding techniques to produce seed, which are then selected under the appropriate conditions.

A genome editing protein or complex (e.g., CRISPR-Cas nucleoprotein) itself may be introduced into the plant cell in a sufficient quantity to modify the cell, but does not persist after a contemplated period of time has passed or after one or more cell divisions. In such examples, no further steps are needed to remove or segregate away the genome editing protein and the modified cell. The genome editing protein can be prepared in vitro prior to introduction to a plant cell using well known recombinant expression systems (bacterial expression, in vitro translation, yeast cells, insect cells and the like). After expression, the protein is isolated, refolded if needed, purified and optionally treated to remove any purification tags, such as a His-tag. Once crude, partially purified, or more completely purified genome editing proteins are obtained, they may be introduced to a plant cell via electroporation, by bombardment with protein coated particles, by chemical transfection or by some other means of transport across a cell membrane.

Genome editing proteins can also be expressed, for example in Agrobacterium, as fusion proteins fused to an appropriate domain of a virulence protein that is translocated into plants (e.g., VirD2, VirE2, VirE2 and VirF). The Vir protein fused with the genome editing protein travels to the plant cell's nucleus, where the genome editing protein would produce the desired double stranded break in the genome of the cell (see, e.g., Vergunst et at. Science 290:979-82, 2000).

### Plant Breeding

Also disclosed are methods of plant breeding. Plant breeding can be used to produce plants with desired traits. In some examples, one or more Cannabis plants having increased varin production are produced by a method including: (i) analyzing one or more genetic markers in a nucleic acid sample from a Cannabis plant or its germplasm; (ii) detecting one or more genetic markers associated with increased varin production (such as one or more markers disclosed herein), (iii) crossing the Cannabis plant comprising the one or more genetic markers associated with increased varin production, and (iv) obtaining one or more progeny plants comprising the one or more genetic markers associated with increased varin production. In some examples, the one or more progeny plants have increased varin content relative to a control. In some examples, a plant disclosed herein, including a plant selected or identified by a method disclosed herein, is used for plant breeding (e.g., crossing). In some examples, a plant disclosed herein is used to develop new, unique, and superior variety or hybrid with a desired phenotype (e.g., varin production, or increased varin production).

Details of existing Cannabis plants varieties and breeding methods are described in Potter et al. (2011, World Wide Weed: Global Trends in Cannabis Cultivation and Its Control), Holland (2010, The Pot Book: A Complete Guide to Cannabis, Inner Traditions/Bear & Co, ISBN1594778981, 9781594778988), Green I (2009, The Cannabis Grow Bible: The Definitive Guide to Growing Marijuana for Recreational and Medical Use, Green Candy Press, 2009, ISBN 1931160589, 9781931160582), Green II (2005, The Cannabis Breeder's Bible: The Definitive Guide to Marijuana Genetics, Cannabis Botany and Creating Strains for the Seed Market, Green Candy Press, 1931160279, 9781931160278), Starks (1990, Marijuana Chemistry: Genetics, Processing & Potency, ISBN 0914171399, 9780914171393), Clarke (1981, Marijuana Botany, an Advanced Study: The Propagation and Breeding of Distinctive Cannabis, Ronin Publishing, ISBN 091417178X, 9780914171782), Short (2004, Cultivating Exceptional Cannabis: An Expert Breeder Shares His Secrets, ISBN 1936807122, 9781936807123), Cervantes (2004, Marijuana Horticulture: The Indoor/Outdoor Medical Grower's Bible, Van Patten Publishing, ISBN 187882323X, 9781878823236), Franck et al. (1990, Marijuana Grower's Guide, Red Eye Press, ISBN 0929349016, 9780929349015), Grotenhermen and Russo (2002, Cannabis and Cannabinoids: Pharmacology, Toxicology, and Therapeutic Potential, Psychology Press, ISBN 0789015080, 9780789015082), Rosenthal (2007, The Big Book of Buds: More Marijuana Varieties from the World's Great Seed Breeders, ISBN 1936807068, 9781936807062), Clarke, RC (Cannabis: Evolution and Ethnobotany 2013 (In press)), King, J (Cannabible Vols 1-3, 2001-2006), and four volumes of Rosenthal's Big Book of Buds series (2001, 2004, 2007, and 2011).

The development of commercial Cannabis cultivars requires the development of Cannabis varieties, the crossing of these varieties, and the evaluation of the crosses. Pedigree breeding and recurrent selection breeding methods may be used to develop cultivars from breeding populations. Breeding programs may combine desirable traits from two or more varieties or various broad-based sources into breeding pools from which cultivars are developed by selfing and selection of desired phenotypes. The new cultivars may be crossed with other varieties and the hybrids from these crosses are evaluated to determine which have commercial potential.

In some implementations, a plant disclosed herein is crossed. Exemplary types of crosses include selfing, sibling crossing, outcrossing, and backcrossing.

Pedigree selection, where both single plant selection and mass selection practices are employed, may be used for the generating new varieties. Pedigree breeding is used commonly for the improvement of self-pollinating crops or inbred lines of cross-pollinating crops. Two parents which possess favorable, complementary traits are crossed to produce an F1. An F2 population is produced by selfing one or several F1's or by intercrossing two F1's (sib mating). Selection of the best individuals usually begins in the F2 population; then, beginning in the F3, the best individuals in the best families are usually selected. Replicated testing of families, or hybrid combinations involving individuals of these families, often follows in the F4 generation to improve the effectiveness of selection for traits with low heritability. At an advanced stage of inbreeding (e.g., F6 and F7), the best lines or mixtures of phenotypically similar lines are tested for potential release as new cultivars.

Choice of breeding or selection methods depends on the mode of plant reproduction, the heritability of the trait(s) being improved, and the type of cultivar used commercially (e.g., F1 hybrid cultivar, pureline cultivar, etc.). For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection.

Mass and recurrent selections can be used to improve populations of either self- or cross-pollinating crops. A genetically variable population of heterozygous individuals may be identified or created by intercrossing several different parents. The best plants may be selected based on individual superiority, outstanding progeny, or excellent combining ability. Preferably, the selected plants are intercrossed to produce a new population in which further cycles of selection are continued.

Backcross breeding has been used to transfer genes for a simply inherited, highly heritable trait into a desirable homozygous cultivar or line that is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent may be selected and repeatedly crossed (backcrossed) to the recurrent parent. The resulting plant is expected to have the attributes of the recurrent parent (e.g., cultivar) and the desirable trait transferred from the donor parent.

A single-seed descent procedure refers to planting a segregating population, harvesting a sample of one seed per plant, and using the one-seed sample to plant the next generation. When the population has advanced from the F2 to the desired level of inbreeding, the plants from which lines are derived will each trace to different F2 individuals. The number of plants in a population declines each generation due to failure of some seeds to germinate or some plants to produce at least one seed. As a result, not all of the F2 plants originally sampled in the population will be represented by a progeny when generation advance is completed.

Mutation breeding is another method of introducing new traits into Cannabis varieties. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogs like 5-bromo-uracil), antibiotics, alkylating agents (such as sulfur mustards, nitrogen mustards, epoxides, ethyleneamines, sulfates, sulfonates, sulfones, or lactones), azide, hydroxylamine, nitrous acid or acridines. Once a desired trait is observed through mutagenesis the trait may then be incorporated into existing germplasm by traditional breeding techniques. Details of mutation breeding can be found, for example, in Principles of Cultivar Development by Fehr, Macmillan Publishing Company, 1993.

The complexity of inheritance also influences the choice of the breeding method. Backcross breeding may be used to transfer one or a few favorable genes for a highly heritable trait into a desirable cultivar. This approach has been used extensively for breeding disease-resistant cultivars. Various recurrent selection techniques are used to improve quantitatively inherited traits controlled by numerous genes. The use of recurrent selection in self-pollinating crops depends on the ease of pollination, the frequency of successful hybrids from each pollination, and the number of hybrid offspring from each successful cross.

Additional breeding methods are available, e.g., methods discussed in Chahal and Gosal (Principles and procedures of plant breeding: biotechnological and conventional approaches, CRC Press, 2002, ISBN 084931321X, 9780849313219), Taji et al. (In vitro plant breeding, Routledge, 2002, ISBN 156022908X, 9781560229087), Richards (Plant breeding systems, Taylor & Francis US, 1997, ISBN 0412574500, 9780412574504), Hayes (Methods of Plant Breeding, Publisher: READ BOOKS, 2007, ISBN1406737062, 9781406737066).

The production of double haploids can also be used for the development of homozygous varieties in a breeding program. Double haploids are produced by the doubling of a set of chromosomes from a heterozygous plant to produce a completely homozygous individual (e.g., see Wan et al., Theor. Appl. Genet., 77:889-892, 1989).

Some implementations of the methods disclosed herein include marker assisted selection (MAS). MAS is a powerful shortcut to selecting for desired phenotypes and for introgressing desired traits into cultivars (e.g., introgressing desired traits into elite lines). MAS is easily adapted to high throughput molecular analysis methods that can quickly screen large numbers of plant or germplasm genetic material for the markers of interest and is much more cost effective than raising and observing plants for visible traits. Thus, MAS can be used in the methods disclosed herein to produce plants with desired traits (e.g., increased varin production).

### Plants and Products

Also disclosed are plants that produce varins or have increased varin production, such as plants identified, selected, or produced by a method disclosed herein, as well as material derived from such plants, including seed, tissue, or cells (including protoplasts); or progeny, such as F1 or F2 progeny. In some implementations, the plant is a Cannabis plant. In some examples, the Cannabis plant is Cannabis sativa, Cannabis indica, or Cannabis ruderalis. In a non-limiting example, the Cannabis plant is Cannabis sativa. In some implementations, the plant includes one or more beneficial polymorphisms (a polymorphism associated with increased varin production). In some examples, the plant includes two or more beneficial polymorphisms (a polymorphism associated with increased varin production). In some examples, the plant includes three or more beneficial polymorphisms (a polymorphism associated with increased varin production). In some examples, the plant includes four or more beneficial polymorphisms (a polymorphism associated with increased varin production). In some examples, the plant includes five or more beneficial polymorphisms (a polymorphism associated with increased varin production). In some examples, the plant includes six or more beneficial polymorphisms (a polymorphism associated with increased varin production). In some examples, the plant includes eight or more beneficial polymorphisms (a polymorphism associated with increased varin production). In some examples, the plant includes ten or more beneficial polymorphisms (a polymorphism associated with increased varin production). In some examples, the plant includes twelve or more beneficial polymorphisms (a polymorphism associated with increased varin production). In some examples, the plant includes fourteen or more beneficial polymorphisms (a polymorphism associated with increased varin production).

In some examples, the beneficial polymorphisms are in one or more of ALT4, KR, FATB, GDSL1, or ISS1. In some examples, the plant includes one or more beneficial polymorphisms (a polymorphism associated with increased varin production) in ALT4. In some examples, the plant includes one or more beneficial polymorphisms (a polymorphism associated with increased varin production) in KR. In some examples, the plant includes one or more beneficial polymorphisms (a polymorphism associated with increased varin production) in FATB. In some examples, the plant includes one or more beneficial polymorphisms (a polymorphism associated with increased varin production) in GDSL1. In some examples, the plant includes one or more beneficial polymorphisms (a polymorphism associated with increased varin production) in ISS1. Additional description regarding specific polymorphisms associated with increased varin production are disclosed herein, for example, in Example 1 and “Methods of Identifying Plants with Increased Varin Production” section.

In some implementations, the plant includes one or more beneficial polymorphisms (a polymorphism associated with increased varin production) in at least two of: ALT4, KR, FATB, GDSL1, and ISS1. In some implementations, the plant includes one or more beneficial polymorphisms (a polymorphism associated with increased varin production) in at least three of: ALT4, KR, FATB, GDSL1, and ISS1. In some implementations, the plant includes one or more beneficial polymorphisms (a polymorphism associated with increased varin production) in at least four of: ALT4, KR, FATB, GDSL1, and ISS1. In some implementations, the plant includes one or more beneficial polymorphisms (a polymorphism associated with increased varin production) in all of: ALT4, KR, FATB, GDSL1, and ISS1. In some implementations, the plant includes one or more beneficial polymorphisms (a polymorphism associated with increased varin production) in ALT4 and in ISS1. In some examples, the plant includes one or more beneficial polymorphisms (a polymorphism associated with increased varin production) in ISS1 and KR. In some examples, the plant includes one or more beneficial polymorphisms (a polymorphism associated with increased varin production) in ISS1, KR, and ALT4. Examples of beneficial polymorphisms of ALT4, KR, FATB, GDSL1, and ISS1 are described herein, for example, in the “Methods of Identifying Plants with Increased Varin Production” section and the Examples.

In some implementations, the plants disclosed herein have a total varin content of at least 1%, for example, at least 1.5%, at least 2.0%, at least 2.5%, at least 3%, at least 3.5%, at least 4%, at least 4.5%, at least 5%, at least 5.5%, at least 6%, at least 6.5%, at least 7%, at least 7.5%, at least 8%, at least 8.5%, at least 9%, at least 9.5%, at least 10%, at least 11%, at least 12%, at least 13%, at least 14%, at least 15%, at least 16%, at least 17%, at least 18%, at least 19%, at least 20%, at least 25%, at least 30%, at least 35%, or at least 40% in at least one plant part (e.g., flower, leaf, or trichome). In a non-limiting example, the plants have a total varin content of at least 4%. In a non-limiting example, the plants have a total varin content of at least 2%. In another non-limiting example, the plants have a total varin content of at least 5%. In a further non-limiting example, the plants have a total varin content of at least 6%. In a non-limiting example, the plants have a total varin content of at least 15%. In another non-limiting example, the plant has a total varin content of at least 10%. In another non-limiting example, the plant has a total varin content of at least 16.5%. In some examples, the plant has a total varin content of at least 20%.

In some implementations, the plant has a total varin content of 1% to 40%, for example, 1% to 39%, 1% to 35%, 1% to 30%, 1% to 25%, 1% to 24%, 1% to 23%, 1% to 22%, 1% to 21%, 1% to 20%, 1% to 19%, 1% to 18%, 1% to 17%, 1% to 16%, 1% to 15%, 1% to 14%, 1% to 13%, 1% to 12%, 1% to 11%, 1% to 10%, 1% to 9%, 1% to 8%, 1% to 7%, 1% to 6%, 1% to 5%, 1% to 4%, 1% to 3%, 1% to 2%, 2% to 40%, 2% to 35%, 2% to 30%, 2% to 25%, 2% to 24%, 2% to 23%, 2% to 22%, 2% to 21%, 2% to 20%, 2% to 19%, 2% to 18%, 2% to 17%, 2% to 16%, 2% to 15%, 2% to 14%, 2% to 13%, 2% to 12%, 2% to 11%, 2% to 10%, 2% to 9%, 2% to 8%, 2% to 7%, 2% to 6%, 2% to 5%, 2% to 4%, 2% to 3%, 3% to 40%, 3% to 35%, 3% to 30%, 3% to 25%, 3% to 24%, 3% to 23%, 3% to 22%, 3% to 21%, 3% to 20%, 3% to 19%, 3% to 18%, 3% to 17%, 3% to 16%, 3% to 15%, 3% to 14%, 3% to 13%, 3% to 12%, 3% to 11%, 3% to 10%, 3% to 9%, 3% to 8%, 3% to 7%, 3% to 6%, 3% to 5%, 3% to 4%, 4% to 40%, 4% to 35%, 4% to 30%, 4% to 25%, 4% to 24%, 4% to 23%, 4% to 22%, 4% to 21%, 4% to 20%, 4% to 19%, 4% to 18%, 4% to 17%, 4% to 16%, 4% to 15%, 4% to 14%, 4% to 13%, 4% to 12%, 4% to 11%, 4% to 10%, 4% to 9%, 4% to 8%, 4% to 7%, 4% to 6%, 4% to 5%, 5% to 40%, 5% to 35%, 5% to 30%, 5% to 25%, 5% to 24%, 5% to 23%, 5% to 22%, 5% to 21%, 5% to 20%, 5% to 19%, 5% to 18%, 5% to 17%, 5% to 16%, 5% to 15%, 5% to 14%, 5% to 13%, 5% to 12%, 5% to 11%, 5% to 10%, 5% to 9%, 5% to 8%, 5% to 7%, 5% to 6%, 6% to 40%, 6% to 35%, 6% to 30%, 6% to 25%, 6% to 24%, 6% to 23%, 6% to 22%, 6% to 21%, 6% to 20%, 6% to 19%, 6% to 18%, 6% to 17%, 6% to 16%, 6% to 15%, 6% to 14%, 6% to 13%, 6% to 12%, 6% to 11%, 6% to 10%, 6% to 9%, 6% to 8%, 6% to 7%, 7% to 40%, 7% to 35%, 7% to 30%, 7% to 25%, 7% to 24%, 7% to 23%, 7% to 22%, 7% to 21%, 7% to 20%, 7% to 19%, 7% to 18%, 7% to 17%, 7% to 16%, 7% to 15%, 7% to 14%, 7% to 13%, 7% to 12%, 7% to 11%, 7% to 10%, 7% to 9%, 7% to 8%, 8% to 40%, 8% to 35%, 8% to 30%, 8% to 25%, 8% to 24%, 8% to 23%, 8% to 22%, 8% to 21%, 8% to 20%, 8% to 19%, 8% to 18%, 8% to 17%, 8% to 16%, 8% to 15%, 8% to 14%, 8% to 13%, 8% to 12%, 8% to 11%, 8% to 10%, 8% to 9%, 9% to 40%, 9% to 35%, 9% to 30%, 9% to 25%, 9% to 24%, 9% to 23%, 9% to 22%, 9% to 21%, 9% to 20%, 9% to 19%, 9% to 18%, 9% to 17%, 9% to 16%, 9% to 15%, 9% to 14%, 9% to 13%, 9% to 12%, 9% to 11%, 9% to 10%, 10% to 40%, 10% to 35%, 10% to 30%, 10% to 25%, 10% to 24%, 10% to 23%, 10% to 22%, 10% to 21%, 10% to 20%, 10% to 19%, 10% to 18%, 10% to 17%, 10% to 16%, 10% to 15%, 10% to 14%, 10% to 13%, 10% to 12%, 10% to 11%, 11% to 19%, 11% to 18%, 11% to 17%, 11% to 16%, 11% to 15%, 11% to 14%, 11% to 13%, 11% to 12%, 12% to 19%, 12% to 18%, 12% to 17%, 12% to 16%, 12% to 15%, 12% to 14%, 12% to 13%, 13% to 19%, 13% to 18%, 13% to 17%, 13% to 16%, 13% to 15%, 13% to 14%, 14% to 19%, 14% to 18%, 14% to 17%, 14% to 16%, 14% to 15%, 15% to 40%, 15% to 35%, 15% to 30%, 15% to 25%, 15% to 24%, 15% to 23%, 15% to 22%, 15% to 21%, 15% to 20%, 15% to 19%, 15% to 18%, 15% to 17%, 15% to 16%, 16% to 19%, 16% to 18%, 16% to 17%, 17% to 19%, 17% to 18%, 18% to 19%, 19% to 40%, 19% to 35%, 19% to 30%, 19% to 25%, 19% to 24%, 19% to 23%, 19% to 22%, 19% to 21%, 19% to 20%, 20% to 40%, 20% to 35%, 20% to 30%, 20% to 25%, 20% to 24%, 20% to 23%, 20% to 22%, 20% to 21%, 25% to 40%, 25% to 35%, 25% to 30%, 30% to 40%, 30% to 35%, or 35% to 40% total varin content in at least one plant part (e.g., flower, leaf, or trichome). In some examples, the total varin content is 2% to 7%. In some examples, the total varin content is 3% to 7%. In some examples, the total varin content is 4% to 7%. In a further example, the total varin content is 4% to 18%. In another example, the total varin content is 4% to 16.5%. In another example, the total varin content is 7% to 16.5%. In some examples, the total varin content is 15% to 35%. In some examples, the total varin content is 15% to 20%.

In some implementations, the plants disclosed herein have a varin ratio of at least 0.1, for example, at least 0.2, at least 0.3, at least 0.33, at least 0.4, at least 0.5, at least 0.6, at least 0.7, at least 0.8, at least 0.9, at least 1, at least 1.5, at least 2.0, at least 2.5, at least 3, at least 3.5, at least 4, at least 4.5, at least 5, at least 5.5, at least 6, at least 6.5, at least 7, at least 7.5, at least 8, at least 8.5, at least 9, at least 9.5, at least 10, at least 15, or at least 20 in at least one plant part (e.g., flower, leaf, or trichome). In a non-limiting example, the plant has a varin ratio of at least 0.2. In a non-limiting example, the plant has a varin ratio of at least 1. In a non-limiting example, the plant has a varin ratio of at least 3. In another non-limiting example, the plant has a varin ratio of at least 4. In some examples, the varin ratio is at least 7.

In some implementations, the plants disclosed herein have a varin ratio of 0.1 to 30, for example, 0.1 to 25, 0.1 to 20, 0.1 to 15, 0.1 to 10, 0.1 to 5, 0.1 to 4, 0.1 to 3, 0.1 to 2, 0.1 to 1, 0.1 to 0.5, 0.25 to 30, 0.25 to 25, 0.25 to 20, 0.25 to 15, 0.25 to 10, 0.25 to 5, 0.25 to 4, 0.25 to 3, 0.25 to 2, 0.25 to 1, 0.25 to 0.5, 0.33 to 25, 0.33 to 20, 0.33 to 15, 0.33 to 10, 0.33 to 7, 0.33 to 5, 0.33 to 3, 0.33 to 1, 0.33 to 0.5, 0.5 to 30, 0.5 to 25, 0.5 to 20, 0.5 to 15, 0.5 to 10, 0.5 to 5, 0.5 to 4, 0.5 to 3, 0.5 to 2, 0.5 to 1, 0.75 to 25, 0.75 to 20, 0.75 to 15, 0.75 to 10, 0.75 to 5, 0.75 to 4, 0.75 to 3, 0.75 to 2, 0.75 to 1, 1 to 30, 1 to 25, 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, 1 to 2, 2 to 30, 2 to 25, 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4, 2 to 3, 3 to 30, 3 to 25, 3 to 20, 3 to 19, 3 to 18, 3 to 17, 3 to 16, 3 to 15, 3 to 14, 3 to 13, 3 to 12, 3 to 11, 3 to 10, 3 to 9, 3 to 8, 3 to 7, 3 to 6, 3 to 5, 3 to 4, 4 to 30, 4 to 25, 4 to 20, 4 to 19, 4 to 18, 4 to 17, 4 to 16, 4 to 15, 4 to 14, 4 to 13, 4 to 12, 4 to 11, 4 to 10, 4 to 9, 4 to 8, 4 to 7, 4 to 6, 4 to 5, 5 to 30, 5 to 25, 5 to 20, 5 to 19, 5 to 18, 5 to 17, 5 to 16, 5 to 15, 5 to 14, 5 to 13, 5 to 12, 5 to 11, 5 to 10, 5 to 9, 5 to 8, 5 to 7, 5 to 6, 6 to 30, 6 to 25, 6 to 20, 6 to 19, 6 to 18, 6 to 17, 6 to 16, 6 to 15, 6 to 14, 6 to 13, 6 to 12, 6 to 11, 6 to 10, 6 to 9, 6 to 8, 6 to 7, 7 to 30, 7 to 25, 7 to 20, 7 to 19, 7 to 18, 7 to 17, 7 to 16, 7 to 15, 7 to 14, 7 to 13, 7 to 12, 7 to 11, 7 to 10, 7 to 9, 7 to 8, 8 to 30, 8 to 25, 8 to 20, 8 to 19, 8 to 18, 8 to 17, 8 to 16, 8 to 15, 8 to 14, 8 to 13, 8 to 12, 8 to 11, 8 to 10, 8 to 9, 9 to 30, 9 to 25, 9 to 20, 9 to 19, 9 to 18, 9 to 17, 9 to 16, 9 to 15, 9 to 14, 9 to 13, 9 to 12, 9 to 11, 9 to 10, 10 to 30, 10 to 25, 10 to 20, 10 to 18, 10 to 16, 10 to 14, 10 to 12, 12 to 30, 12 to 25, 12 to 20, 12 to 18, 12 to 16, 12 to 14, 14 to 30, 14 to 25, 14 to 20, 14 to 18, 14 to 16, 16 to 30, 16 to 25, 16 to 20, 16 to 18, 18 to 30, 18 to 25, 18 to 20, 20 to 30, 20 to 25, or 25 to 30 in at least one plant part (e.g., flower, leaf, or trichome). In some examples, the varin ratio is 1 to 8. In some examples, the varin ratio is 0.25 to 5. In some examples, the varin ratio is 1 to 5. In further examples, the varin ratio is 7 to 20. In some examples, the varin ratio is 7 to 10.

A plant part includes any part of the plant (e.g., a plant that produces varins or has increased varin production). In some examples, the plant part is a flower or inflorescence. In some examples, the plant part is trichomes. In further examples, the plant part is leaf or other vegetative tissue.

In some implementations, the plant is identified or generated by a method disclosed herein. In some implementations, the plant is a Cannabis plant. In some examples, the Cannabis plant is Cannabis sativa, Cannabis indica, or Cannabis ruderalis. In a non-limiting example, the Cannabis plant is Cannabis sativa.

Further disclosed are products made from a Cannabis plant disclosed herein (including a Cannabis plant identified, selected, or produced by a method disclosed herein), for example, a kief, hashish, bubble hash, extract, edible product, oil, solvent reduced oil, sludge, e-juice, or tincture. Kief refers to a composition of concentrated Cannabis trichomes, which are accumulated by being sifted from Cannabis flowers or buds using a mesh screen or sieve. Hashish (or hash) refers to a compressed or purified preparation from Cannabis tissue containing trichomes (e.g., flowers). Bubble hash refers to a solid concentration of Cannabis trichomes made from a solventless extraction method. As used herein, Cannabis sludges are solvent-free Cannabis extracts made via multigas extraction including the refrigerant 134A, butane, iso-butane and propane in a ratio that delivers a very complete and balanced extraction of cannabinoids and essential oils. E-juice (vape juice) refers to a liquid composition for use in an e-cigarette. A tincture refers to an alcohol-based extract, for example, an extract of Cannabis tissue dissolved in an alcohol. A practitioner can readily determine suitable known methods for producing a product disclosed herein.

The product can be formulated for administration to a subject (e.g., a human), such as by an injection (e.g., intravenous, subcutaneous, intramuscular, parenteral), or by topical, oral, or pulmonary administration. In some examples, the product is a recreational product. In some examples, the product is a therapeutic product (e.g., medicament).

For pulmonary administration, the compositions can include, but are not limited to, dry powder compositions consisting of the powder of a Cannabis oil described herein, and the powder of a suitable carrier and/or lubricant. The compositions for pulmonary administration can be inhaled from any suitable dry powder inhaler device. In certain examples, the compositions may be delivered in the form of an aerosol spray from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound(s) and a suitable powder base, for example, lactose or starch.

For oral administration, a composition can take the form of, e.g., a tablet or a capsule prepared by conventional means with a pharmaceutically acceptable excipient. Preferred are tablets and gelatin capsules comprising the active ingredient(s), together with (a) diluents or fillers, e.g., lactose, dextrose, sucrose, mannitol, maltodextrin, lecithin, agarose, xanthan gum, guar gum, sorbitol, cellulose (e.g., ethyl cellulose, microcrystalline cellulose), glycine, pectin, polyacrylates and/or calcium hydrogen phosphate, calcium sulfate, (b) lubricants; e.g., silica, anhydrous colloidal silica, talcum, stearic acid, its magnesium or calcium salt (e.g., magnesium stearate or calcium stearate), metallic stearates, colloidal silicon dioxide, hydrogenated vegetable oil, corn starch, sodium benzoate, sodium acetate and/or polyethyleneglycol; for tablets also (c) binders, e.g., magnesium aluminum silicate, starch paste, gelatin, tragacanth, methylcellulose, sodium carboxymethylcellulose, polyvinylpyrrolidone and/or hydroxypropyl methylcellulose; if desired (d) disintegrants, e.g., starches (e.g., potato starch or sodium starch), glycolate, agar, alginic acid or its sodium or potassium salt, or effervescent mixtures; (e) wetting agents, e.g., sodium lauryl sulfate, and/or (f) absorbents, colorants, flavors and sweeteners. Tablets can be either uncoated or coated according to known methods. The excipients described herein can also be used for preparation of buccal dosage forms and sublingual dosage forms (e.g., films and lozenges) as described, for example, in U.S. Pat. Nos. 5,981,552 and 8,475,832. Formulation in chewing gums as described, for example, in U.S. Pat. No. 8,722,022, is also contemplated.

Further preparations for oral administration can take the form of, for example, solutions, syrups, suspensions, or toothpastes. Liquid preparations for oral administration can be prepared by conventional means with pharmaceutically acceptable additives, for example, suspending agents, for example, sorbitol syrup, cellulose derivatives, or hydrogenated edible fats; emulsifying agents, for example, lecithin, xanthan gum, or acacia; non-aqueous vehicles, for example, almond oil, sesame oil, hemp seed oil, fish oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate.

Typical formulations for topical administration include creams, ointments, sprays, lotions, hydrocolloid dressings, and patches, as well as eye drops, ear drops, and deodorants. Cannabis extracts/oils can be administered via transdermal patches as described, for example, in U.S. Pat. Appl. Pub. No. 2015/0126595 and U.S. Pat. No. 8,449,908. Formulation for rectal or vaginal administration is also contemplated. The Cannabis oils can be formulated, for example, as suppositories containing conventional suppository bases such as cocoa butter and other glycerides as described in U.S. Pat. Nos. 5,508,037 and 4,933,363. Compositions can contain other solidifying agents such as shea butter, beeswax, kokum butter, mango butter, illipe butter, tamanu butter, carnauba wax, emulsifying wax, soy wax, castor wax, rice bran wax, and candelilla wax. Compositions can further include clays (e.g., bentonite, French green clays, Fuller's earth, Rhassoul clay, white kaolin clay) and salts (e.g., sea salt, Himalayan pink salt, and magnesium salts such as Epsom salt).

The compositions disclosed herein can be formulated for administration by injection, for example, by bolus injection or continuous infusion. Formulations for injection can be presented in unit dosage form, for example, in ampoules or in multi-dose containers, optionally with an added preservative. Injectable compositions are preferably aqueous isotonic solutions or suspensions, and suppositories are preferably prepared from fatty emulsions or suspensions. The compositions may be sterilized and/or contain adjuvants, such as preserving, stabilizing, wetting or emulsifying agents, solution promoters, salts for regulating the osmotic pressure, buffers, and/or other ingredients. Alternatively, the compositions can be in powder form for reconstitution with a suitable vehicle, for example, a carrier oil, before use. In addition, the compositions may also contain other therapeutic agents or substances.

The compositions can be prepared according to conventional mixing, granulating, and/or coating methods, and contain from about 0.1 to about 75%, for example from about 1% to about 50%, of a Cannabis extract. In general, subjects receiving a Cannabis composition orally are administered doses ranging from about 1 to about 2000 mg of Cannabis extract. A small dose ranging from about 1 to about 20 mg can typically be administered orally when treatment is initiated, and the dose can be increased (e.g., doubled) over a period of days or weeks until the optimal or maximum dose is reached.

### Nucleic Acids and Kits

Artificial nucleic acids (non-naturally occurring nucleic acids, e.g., cDNA, recombinant nucleic acids, or other synthetic nucleic acids) encoding a beneficial allele of ALT4, KR, FATB, GDSL1, or ISS1 are also disclosed herein.

In some examples, the beneficial allele of ALT4 includes a nucleic acid sequence encoding an amino acid sequence having at least 80%, for example, at least 85%, 90%, 95%, 98%, 99%, sequence identity to any one of SEQ ID NOs: 10-12 or 47. In some examples, the beneficial allele of ALT4 includes a nucleic acid sequence encoding an amino acid sequence having at least 95% sequence identity to any one of SEQ ID NOs: 10-12 or 47. In some examples, the beneficial allele of ALT4 includes a nucleic acid sequence encoding an amino acid sequence having at least 95% sequence identity to SEQ ID NO: 12. In some examples, the beneficial allele of ALT4 includes (in a corresponding ALT4 protein) one or more of: (a) a glutamine corresponding to Abacus reference position 59; (b) a glycine corresponding to Abacus reference position 66; (c) an insertion of aspartic acid immediately following Abacus reference position 47; (d) a tyrosine corresponding to Abacus reference position 67; (e) a deletion of aspartic acid corresponding to Abacus reference position 48; (f) a deletion of aspartic acid corresponding to Abacus reference position 49; (g) a lysine corresponding to Abacus reference position 68; (h) an aspartic acid corresponding to Abacus reference position 81; (i) a glutamic acid corresponding to Abacus reference position 85; (j) a histidine corresponding to Abacus reference position 111; (k) an alanine corresponding to Abacus reference position 115; (l) a serine corresponding to Abacus reference position 124; (m) a valine corresponding to Abacus reference position 125; (n) an aspartic acid corresponding to Abacus reference position 136; (o) an isoleucine corresponding to Abacus reference position 160; (p) a valine corresponding to Abacus reference position 165; (q) a valine corresponding to Abacus reference position 172; (r) a serine corresponding to Abacus reference position 7; (s) a glutamine corresponding to Abacus reference position 202; or (t) an alanine corresponding to Abacus reference position 206. The positions are in reference to the Abacus ALT4 protein sequence (SEQ ID NO: 12).

In some implementations, the artificial nucleic acid includes a nucleic acid sequence encoding an amino acid sequence having at least 80% sequence identity to SEQ ID NO: 10 or SEQ ID NO: 11, respectively, such as at least 85%, at least 90%, at least 93%, at least 95%, at least 98%, or at least 99% sequence identity. In some examples, the artificial nucleic acid includes a nucleic acid sequence encoding an amino acid sequence having at least 90% sequence identity to SEQ ID NO: 10 or SEQ ID NO: 11, respectively. In some implementations, the amino acid sequence having at least 90% sequence identity to SEQ ID NO: 10 or SEQ ID NO: 11 also includes one or more of: (a) a glutamine corresponding to Abacus reference position 59; (b) a glycine corresponding to Abacus reference position 66; (c) an insertion of aspartic acid immediately following Abacus reference position 47; (d) a tyrosine corresponding to Abacus reference position 67; (c) a deletion of aspartic acid corresponding to Abacus reference position 48; (f) a deletion of aspartic acid corresponding to Abacus reference position 49; (g) a lysine corresponding to Abacus reference position 68; (h) an aspartic acid corresponding to Abacus reference position 81; (i) a glutamic acid corresponding to Abacus reference position 85; (j) a histidine corresponding to Abacus reference position 111; (k) an alanine corresponding to Abacus reference position 115; (l) a serine corresponding to Abacus reference position 124; (m) a valine corresponding to Abacus reference position 125; (n) an aspartic acid corresponding to Abacus reference position 136; (o) an isoleucine corresponding to Abacus reference position 160; (p) a valine corresponding to Abacus reference position 165; (q) a valine corresponding to Abacus reference position 172; (r) a serine corresponding to Abacus reference position 7; (s) a glutamine corresponding to Abacus reference position 202; or (t) an alanine corresponding to Abacus reference position 206. The positions are in reference to the Abacus ALT4 protein sequence (SEQ ID NO: 12). In some implementations, the amino acid sequence having at least 90% sequence identity to SEQ ID NO: 10 also includes (a) a glutamine corresponding to Abacus reference position 59; (b) a glycine corresponding to Abacus reference position 66; (c) an insertion of aspartic acid immediately following Abacus reference position 47; and (d) a tyrosine corresponding to Abacus reference position 67. In some implementations, the amino acid sequence having at least 90% sequence identity to SEQ ID NO: 11 also includes (a) a glutamine corresponding to Abacus reference position 59; (b) a glycine corresponding to Abacus reference position 66; (e) a deletion of aspartic acid corresponding to Abacus reference position 48; (f) a deletion of aspartic acid corresponding to Abacus reference position 49; (g) a lysine corresponding to Abacus reference position 68; (h) an aspartic acid corresponding to Abacus reference position 81; (i) a glutamic acid corresponding to Abacus reference position 85; (j) a histidine corresponding to Abacus reference position 111; (k) an alanine corresponding to Abacus reference position 115; (l) a serine corresponding to Abacus reference position 124; (m) a valine corresponding to Abacus reference position 125; (n) an aspartic acid corresponding to Abacus reference position 136; (o) an isoleucine corresponding to Abacus reference position 160; (p) a valine corresponding to Abacus reference position 165; (q) the valine corresponding to Abacus reference position 172; (r) a serine corresponding to Abacus reference position 7; (s) a glutamine corresponding to Abacus reference position 202; and (t) an alanine corresponding to Abacus reference position 206. The positions are in reference to the Abacus ALT4 protein sequence (SEQ ID NO: 12).

In some examples, the beneficial allele of KR includes a nucleic acid sequence encoding an amino acid sequence having at least 80%, for example, at least 85%, 90%, 95%, 98%, 99%, sequence identity to any one of SEQ ID NOs: 66-81. In some examples, the beneficial allele of KR includes a nucleic acid sequence encoding an amino acid sequence having at least 95% sequence identity to any one of SEQ ID NOs: 66-81. In some examples, the beneficial allele of KR includes a nucleic acid sequence encoding an amino acid sequence having at least 95% sequence identity to any one of SEQ ID NOs: 67-81. In some examples, the beneficial allele of KR further includes (in a corresponding KR protein) one or more of: (a) an arginine at position 39; (b) a proline at position 88; (c) a glutamic acid at position 122; (d) a glycine at position 123; (c) a glycine at position 125; (f) a proline at position 139; (g) a valine at position 158; (h) a glycine at position 175; (i) a serine at position 193; (j) a arginine at position 205; (k) a arginine at position 208; (l) an isoleucine at position 217; (m) a serine at position 218; (n) a serine at position 251; or (o) an isoleucine at position 293. The positions are in reference to the Abacus KR protein sequence (SEQ ID NO: 66).

In some examples a beneficial allele of KR includes a nucleic acid sequence encoding an amino acid sequence having at least 95% sequence identity to any one of SEQ ID NOs: 66 and includes (in a corresponding KR protein) one or more of: (a) an arginine at position 39; (b) a proline at position 88; (c) a glutamic acid at position 122; (d) a glycine at position 123; (e) a glycine at position 125; (f) a proline at position 139; (g) a valine at position 158; (h) a glycine at position 175; (i) a serine at position 193; (j) a arginine at position 205; (k) a arginine at position 208; (l) an isoleucine at position 217; (m) a serine at position 218; (n) a serine at position 251; or (o) an isoleucine at position 293. The positions are in reference to the Abacus KR protein sequence (SEQ ID NO: 66).

In further examples, a beneficial allele of FATB includes a nucleic acid sequence encoding an amino acid sequence having at least 80%, for example, at least 85%, 90%, 95%, 98%, 99%, sequence identity to SEQ ID NO: 99 or 100. In some examples, the beneficial allele of FATB includes a nucleic acid sequence encoding an amino acid sequence having at least 95% sequence identity to SEQ ID NO: 99 or 100. In some examples, the beneficial allele of FATB includes a nucleic acid sequence encoding an amino acid sequence having at least 95% sequence identity to SEQ ID NO: 99. In some examples, the beneficial allele of FATB further includes (in a corresponding FATB protein) one or more of: (a) a histidine at position 155; (b) a glutamine at position 173; (c) an arginine at position 197; (d) an isoleucine at position 201; (e) a proline at position 202; or (f) an aspartic acid at position 207. The positions are in reference to the Abacus FATB protein sequence (SEQ ID NO: 100).

In some examples a beneficial allele of FATB includes a nucleic acid sequence encoding an amino acid sequence having at least 95% sequence identity to SEQ ID NO: 100 and includes (in a corresponding FATB protein) one or more of: (a) a histidine at position 155; (b) a glutamine at position 173; (c) an arginine at position 197; (d) an isoleucine at position 201; (e) a proline at position 202; or (f) an aspartic acid at position 207. The positions are in reference to the Abacus FATB protein sequence (SEQ ID NO: 100).

In some examples a beneficial allele of FATB includes a nucleic acid sequence encoding an amino acid sequence having at least 95% sequence identity to SEQ ID NO: 99 and includes (in a corresponding FATB protein) one or more of: (a) a histidine at position 155; (b) a glutamine at position 173; (c) an arginine at position 197; (d) an isoleucine at position 201; (e) a proline at position 202; or (f) an aspartic acid at position 207. The positions are in reference to the Abacus FATB protein sequence (SEQ ID NO: 100).

In further examples, a beneficial allele of GDSL1 includes a nucleic acid sequence encoding an amino acid sequence having at least 80%, for example, at least 85%, 90%, 95%, 98%, 99%, sequence identity to SEQ ID NO: 109 or 110. In some examples, the beneficial allele of GDSL1 includes a nucleic acid sequence encoding an amino acid sequence having at least 95% sequence identity to SEQ ID NO: 109 or 110. In some examples, the beneficial allele of GDSL1 includes a nucleic acid sequence encoding an amino acid sequence having at least 95% sequence identity to SEQ ID NO: 109. In some examples, the beneficial allele of GDSL1 further includes (in a corresponding GDSL1 protein) one or more of: (a) an alanine at position 59; (b) an asparagine at position 275; (c) a threonine at position 297; or (d) a methionine at position 213. The positions are in reference to the Abacus GDSL1 protein sequence (SEQ ID NO: 110).

In some examples a beneficial allele of GDSL1 includes a nucleic acid sequence encoding an amino acid sequence having at least 95% sequence identity to SEQ ID NO: 110 and includes (in a corresponding GDSL1 protein) one or more of: (a) an alanine at position 59; (b) an asparagine at position 275; (c) a threonine at position 297; or (d) a methionine at position 213. The positions are in reference to the Abacus GDSL1 protein sequence (SEQ ID NO: 110).

In some examples a beneficial allele of GDSL1 includes a nucleic acid sequence encoding an amino acid sequence having at least 95% sequence identity to SEQ ID NO: 109 and includes (in a corresponding GDSL1 protein) one or more of: (a) an alanine at position 59; (b) an asparagine at position 275; (c) a threonine at position 297; or (d) a methionine at position 213. The positions are in reference to the Abacus GDSL1 protein sequence (SEQ ID NO: 110).

In some implementations, the artificial nucleic acid includes a nucleic acid sequence encoding SEQ ID NO: 10, 11, 47, 65-81, 99, or 109. In some implementations, the artificial nucleic acid consists of or essentially consists of a nucleic acid sequence encoding SEQ ID NO: 10, 11, 47, 65-81, 99, or 109.

In some implementations, the artificial nucleic acid includes a nucleic acid sequence having at least 80% sequence identity to SEQ ID NO: 7, 8, 46, 51-65, 97, or 107, respectively, such as at least 85%, at least 90%, at least 93%, at least 95%, at least 98%, or at least 99% sequence identity. In some examples, the artificial nucleic acid includes a nucleic acid sequence having at least 95% sequence identity to SEQ ID NO: 7, 8, 46, 51-65, 97, or 107, respectively. In some implementations, the artificial nucleic acid includes SEQ ID NO: 7, 8, 46, 51-65, 97, or 107. In some implementations, the artificial nucleic acid consists of or essentially consists of SEQ ID NO: 7, 8, 46, 51-65, 97, or 107.

In some implementations, the beneficial allele of ISS1 includes the sequence of ISS1 from 22VLV2-1-52. In some examples, the beneficial allele of ISS1 includes a deletion of the A corresponding to Abacus reference position −436 bp upstream from the start codon of ISS1. The nucleotide position is in reference to the Abacus reference Csat_Abacus V2 (NCBI assembly accession GCA_025232715.1; see also, SEQ ID NO: 31).

In some implementations, the artificial nucleic acid includes a nucleic acid sequence having at least 80% sequence identity to SEQ ID NO: 29, such as at least 85%, at least 90%, at least 93%, at least 95%, at least 98%, or at least 99% sequence identity. In some examples, the artificial nucleic acid includes a nucleic acid sequence having at least 90% sequence identity to SEQ ID NO: 29. In some implementations, the artificial nucleic acid includes a nucleic acid sequence having at least 90% sequence identity to SEQ ID NO: 29 and includes a deletion of the A corresponding to Abacus reference position −436 bp upstream from the start codon of ISS1. This position is in reference to the Abacus genome version Csat_AbacusV2 (NCBI assembly accession GCA_025232715.1; see also, SEQ ID NO: 31). In some implementations, the artificial nucleic acid includes SEQ ID NO: 29. In some implementations, the artificial nucleic acid consists of or essentially consists of SEQ ID NO: 29.

Specific examples of nucleic acid sequences that encode the beneficial alleles as discussed above are provided in Example 1. However, the translation of nucleic acids into amino acids is known and determined by the genetic code. Thus, nucleic acid sequences that result in a particular amino acid of a protein sequence can be readily determined by using the genetic code. For non-limiting, exemplary purposes, arginine is encoded by CGT, CGC, CGA, CGG, AGA, and AGG.

The disclosed nucleic acids can be free nucleic acids or be part of a construct, vector, or host genome. In some examples, the nucleic acids are codon optimized for expression in a particular host, such as E. coli.

Kits for use in diagnostic, research, and prognostic applications are also provided. Such kits may include any or all of the following: assay reagents, buffers, nucleic acids encoding a beneficial allele of ALT4, KR, FATB, GDSL1, and/or ISS1, CRISPR RNA targeting ALT4, KR, FATB, GDSL1, and/or ISS1, a Cas nuclease (e.g., Cas9), and/or nucleic acids for detecting target sequences (e.g, probes and/or primers to detect beneficial alleles of ALT4, KR, FATB, GDSL1, and/or ISS1). In some implementations, the kit components are in separate containers. The kit can be used to practice any of the methods disclosed herein. In some implementations, the kit is for detecting a beneficial polymorphism or gene disclosed herein. In some implementations, the kit is for genetically modifying a Cannabis plant. The kits may include instructional materials containing directions (i.e., protocols) for the practice of the methods of this disclosure. While the instructional materials typically comprise written or printed materials, they are not limited to such. Any medium capable of storing such instructions and communicating them to an end user is contemplated. Such media include, but are not limited to, electronic storage media (e.g., magnetic discs, tapes, cartridges, chips), optical media (e.g., CD ROM), cloud-based media, and the like. Such media may include addresses to internet sites that provide such instructional materials.

### EXAMPLES

### Example 1

### Gene Sequencing and Expression Analysis

Five genes, ALT4, ISS1, KR, FATB, and GDSL1, located near five SNP markers (141840_721728, 140868_21724, 142078_3920202, 141488_308754, and 141840_475273, respectively), which were significantly associated with Total Varin (%; =Total THCV+Total CBDV+Total CBCV+Total CBGV) and Varin Ratio (=Total Varin/(Total THC+Total CBD+Total CBC+Total CBG), were explored for putative causative SNPs responsible for high values of these traits in accessions possessing the beneficial genotype for these markers.

SNP marker 141840_721728 is located at position 62,034,938 bp on chromosome 7 of the Abacus reference genome (version Csat_AbacusV2; NCBI assembly accession GCA_025232715.1); it is located 1.2 Kbp upstream of ALT3/4 (acyl-lipid thioesterase 3/4, AT1G68260/AT1G68280, based on homology with Arabidopsis thaliana). The ALT4 coding sequence (CDS) is located between 62,036,122-62,040,579 bp on the Abacus reference genome. Its CBDRx (version cs10) homolog is LOC115697580 (annotated as acyl-acyl carrier protein thioesterase ALT4) and is located between 69,286,245-69,290,148 bp on chromosome 7 of the CBDRx reference genome.

SNP marker 140868_21724 is located at position 62,979,814 bp on chromosome 7 of the Abacus reference genome; it is located 4.0 Kbp upstream of ISS1 (Indole Severe Sensitive 1, AT1G80360, based on homology with Arabidopsis thaliana). The ISS1 CDS is located between 62,983,821-62,987,235 bp on chromosome 7 of the Abacus reference genome. Its CDBRx homolog is LOC115697430 (annotated as aromatic aminotransferase ISS1), which is located between 70,092,334-70,096,498 bp on chromosome 7 of the CBDRx reference genome.

SNP marker 142078_3920202 is located at position 72,717,623 bp on chromosome 4 of the Abacus reference genome; it is located 20.9 Kbp upstream of mtKR/FabG/FabG1 (β ketoacyl-acyl carrier protein (ACP) reductase, AT1G24360, based on homology with Arabidopsis thaliana). The KR CDS is located between 72,696,599-72,691,998 bp on chromosome 4 of the Abacus reference genome, its CBDRx homolog is LOC115714062 (annotated as 3-oxoacyl-[acyl-carrier-protein] reductase 4), which is located between 6,585,812-6,590,794 bp on chromosome 4 of the CBDRx reference genome.

SNP marker 141488_308754 is located at position 80,072,595 bp on chromosome 4 of the Abacus reference genome; it is located 0.1 Kbp upstream of FATB (fatty acyl-ACP thioesterase B, AT1G08510, based on homology with Arabidopsis thaliana). The FATB CDS is located between 80,072,489-80,070,029 bp on chromosome 4 of the Abacus reference genome. Its CBDRx homolog is LOC115712326 (annotated as palmitoyl-acyl carrier protein thioesterase), which is located between 688,259-691,312 bp on chromosome 4 of the CBDRx reference genome.

SNP marker 141840_475273 is located at position 61,748,400 bp on chromosome 7 of the Abacus reference genome; it is located 0.06 Kbp upstream of GDSL1 (Gly-Asp-Ser-Leu-motif lipase esterase/lipase 1, AT1G29670, based on homology with Arabidopsis thaliana). The GDSL1 CDS is located between 61,748,452-61,750,593 bp on chromosome 7 of the Abacus reference genome. Its CBDRx homolog is LOC115697568 (annotated as GDSL esterase/lipase At1g29670), which is located between 68,968,188-68,970,355 bp on chromosome 7 of the CBDRx reference genome.

ALT4, KR, GDSL1, FATB, and ISS1 were evaluated for DNA sequence variation in Cannabis accessions varying for Total Varin and Varin Ratio (Table 1). Sequences were compared with Abacus reference genome sequences. Abacus is considered non-varin producing (0% Total Varin; Table 1). 21VLP5-1-101 and 21VLP5-1-222 are siblings from the same seed lot, whereas 22VLV2-1-52 and 22VLV2-1-59 are siblings from a selfed sibling of 21VLP5-1-101 and 21VLP5-1-222, and 23PV1-22-110 is the result of two generations of further selfing of a sibling of 22VLV2-1-52 and 22VLV2-1-59. These five related accessions were chosen to capture the allelic variation of the original seed lot, allelic segregation in an F2 population, as well as for the further study of fixed alleles in a more inbred accession.

RNA was extracted from flower tissue from eight varin producing accessions with the beneficial genotype for the five SNP markers and non-varin producing accession Abacus, which has the detrimental genotype for all five SNP makers. Tissue was collected 2-5 weeks after onset of flowering. For varin producing accessions 22TP1C-140-40 and 22TP1C-141-25 RNA was extracted from leaf tissue (Nucleospin RNA Plant and Fungi kit, Macherey-Nagel; Table 1). After concentration adjustment and treatment with DNAse, the RNA was used directly for RT-PCR (OneTaq® One-Step RT-PCR Kit, New England Biolabs). For RT-PCR gene expression analysis, expression levels were compared to ACT2 which was included as a positive control. Sanger sequencing of coding sequence (CDS) was performed based on RT-PCR product (NEB PCR® Cloning Kit; New England Biolabs). Genomic DNA (extracted from leaf tissue with a NucleoMag Plant DNA Kit, Macherey-Nagel) was used to sequence the beginning and end of each gene, as well as the upstream region of ISS1. Sanger sequencing based on cloned RT-PCR or PCR product was performed for areas with high levels of heterozygosity. Sanger sequencing based on RT-PCR or PCR product without cloning was performed for areas with low levels of heterozygosity. Primers for amplification and sequencing of fragments of the five varin genes can be found in Table 5 (SEQ ID NOs: 1-6, and 32-44).

TABLE 1
Varin phenotypes and marker genotypes (A = homozygous reference
allele, X = heterozygous, B = homozygous alternate allele,
based on Abacus as the reference genome) of accessions used for sequencing
of varin genes KR, FATB, GDSL1, ALT4, and ISS1. The beneficial genotype
for high Total Varin and high Varin Ratio is homozygous alternate
allele and heterozygous (B, X; additive effect) for all five markers.

| | | | KR | FATB | GDSL1 | ALT4 | ISS1 |
|---|---|---|---|---|---|---|---|
| | Total | | marker | marker | marker | marker | marker |
| Accession | Varin | Varin | 142078_{—} | 141488_{—} | 141840_{—} | 141840_{—} | 140868_{—} |
| name | (%) | Ratio | 3920202 | 308754 | 475273 | 721728 | 21724 |
| Abacus | 0.00 | 0.00 | A | A | A | A | A |
| 21VLP5-1- | 4.23 | 3.71 | B | X | X | X | X |
| 101 | | | | | | | |
| 21VLP5-1- | 4.14 | 2.98 | B | X | X | X | X |
| 222 | | | | | | | |
| 22VLV2-1- | 6.14 | 4.80 | B | X | B | B | B |
| 52 | | | | | | | |
| 22VLV2-1- | 10.13 | 4.80 | B | A | B | B | B |
| 59 | | | | | | | |
| 23PV1-22- | 13.82 | 7.02 | B | A | B | B | B |
| 110 | | | | | | | |
| 21TX1-60 | 5.40 | 1.00 | B | X | B | B | A |
| 22TP1C- | 20.21 | 4.62 | B | X | B | B | X |
| 140-40 | | | | | | | |
| 22TP1C- | 20.61 | 4.11 | B | X | B | B | X |
| 141-25 | | | | | | | |
| 20VLP4-6- | 2.03 | 0.39 | X | A | B | B | A |
| 15 | | | | | | | |
| 95-831 | 0.91 | 0.04 | B | A | B | B | A |

First column: accession name; Second column: Total Varin (%; =Total THCV + Total CBDV + Total CBCV + Total CBGV) observed for dry flower tissue at maturity. Third column: Varin Ratio (=Total Varin/(Total THC + Total CBD + Total CBC + Total CBG) observed for dry flower tissue at maturity; Fourth column: genotypes observed for SNP marker 142078_3920202 near KR; Fifth column: genotypes observed for SNP marker 141488_308754 near FATB; Sixth column: genotypes observed for SNP marker 141840_475273 near GDSL1; Seventh column: genotypes observed for SNP marker 141840_721728 near ALT4; Eighth column: genotypes observed for SNP marker 140868_21724 located near ISS1.

Alignment of Sanger sequenced fragments was performed per accession for all five genes. The resulting consensus sequences were subsequently aligned per gene. Functional CDS were translated to protein sequences, which were subsequently aligned with Arabidopsis thaliana protein sequence to identify functional domains. Functional domains were explored further in the protein sequence alignments for amino acid substitutions that would alter these domains.

### ALT4

Alignment of ALT4 coding sequence (CDS) and protein sequences of varin (21VLP5-1-101, 21VLP5-1-222, 22VLV2-1-52, 22VLV2-1-59, 23PV1-22-110, 20VLP4-6-15, 21TX1-60, and 95-831) and non-varin (Abacus) producing Cannabis accessions differing for the beneficial genotype of SNP marker 141840_721728 revealed multiple amino acid substitutions, some of those were in common between some of the varin producing accessions with the beneficial genotype for this SNP marker (Table 1).

Two amino acid substitutions were in common between all sequenced varin producing accessions with the beneficial genotype for SNP marker 141840_721728. The first is an R (Arginine, observed in Abacus) to Q (Glutamine, observed in 21VLP5-1-101, 21VLP5-1-222, 22VLV2-1-52, 22VLV2-1-59, 20VLP4-6-15, 95-831, and 21TX1-60) amino acid substitution at position 60 in 21VLP5-1-101 (SEQ ID NO: 10) and position 57 in 21TX1-60 (SEQ ID NO: 11), corresponding with amino acid position 59 in Abacus (R59Q; SEQ ID NO: 12) caused by a G to A nucleotide substitution (Abacus: G; 21VLP5-1-101 and 21TX1-60: A) at CDS position 179 bp of 21VLP5-1-101 (SEQ ID NO: 7), CDS position 170 bp of 21TX1-60 (SEQ ID NO: 8), and CDS position 176 bp of Abacus (SEQ ID NO: 9; located at position 51 bp for SEQ ID NO: 13). The second amino acid substitution in common between varin producing accessions with the beneficial genotype for SNP marker 141840_721728 is an E (Glutamic Acid; E66G; observed in Abacus) to G (Glycine; observed in 21VLP5-1-101 and 21TX1-60) amino acid substitution at position 67 in 21VLP5-1-101 (SEQ ID NO: 10), position 64 in 21TX1-60 (SEQ ID NO: 11), corresponding with position 66 in Abacus (E66G; SEQ ID NO: 12). This amino acid substitution was caused by an A to G nucleotide substitution (Abacus: A; 21VLP5-1-101 and 21TX1-60: G) at CDS position 200 bp for 21VLP5-1-101 (SEQ ID NO: 7), CDS position 191 bp for 21TX1-60 (SEQ ID NO: 8), and CDS position 197 bp for Abacus (SEQ ID NO: 9; located at position 51 bp of SEQ ID NO: 14).

One amino acid substitution and one insertion were in common among the five related varin producing accessions, 21VLP5-1-101, 21VLP5-1-222, 22VLV2-1-52, 22VLV2-1-59, and 23PV1-22-110. The amino acid substitution is a P (Proline; observed in Abacus, 20VLP4-6-15, 95-831, and 21TX1-60) to S (Serine; observed in 21VLP5-1-101, 21VLP5-1-222, 22VLV2-1-52, 22VLV2-1-59, and 23PV1-22-110) at amino acid position 7 in 21VLP5-1-101 (SEQ ID NO: 10), corresponding with amino acid position 7 in Abacus (P7S; SEQ ID NO: 12) caused by a C to T nucleotide substitution (Abacus: C; 21VLP5-1-101: T) at CDS position 19 bp for 21VLP5-1-101 (SEQ ID NO: 7), and CDS position 19 bp for Abacus (SEQ ID NO: 9; located at position 51 bp of SEQ ID NO: 45). The amino acid insertion (D; Aspartic acid, observed in 21VLP5-1-101, 21VLP5-1-222, 22VLV2-1-52, 22VLV2-1-59, and 23PV1-22-110) immediately following position 47 of Abacus (K47D48insD; SEQ ID NO: 10) is caused by a three bp insertion (GAT) at positions 142-144 bp in the CDS (SEQ ID NO: 7). Abacus has a deletion of this amino acid (D; Aspartic acid; SEQ ID NO: 12) corresponding with a deletion of three nucleotides GAT (SEQ ID NO: 9; located at positions 50-52 bp of SEQ ID NO: 15).

One amino acid substitution was specific to varin producing accession 21VLP5-1-101 which is heterozygous for SNP marker 141840_721728. This amino acid substitution specific to 21VLP5-1-101 is an H (histidine; observed in Abacus) to Y (tyrosine; observed in 21VLP5-1-101) amino acid substitution at amino acid position 68 in 21VLP5-1-101 (SEQ ID NO: 10), corresponding with amino acid position 67 in Abacus (H67Y; SEQ ID NO: 12) caused by a C to T nucleotide substitution (Abacus: C; 21VLP5-1-101: T) at CDS position 202 bp for 21VLP5-1-101 (SEQ ID NO: 7), and CDS position 199 bp for Abacus (SEQ ID NO: 9; located at position 51 bp of SEQ ID NO: 16).

Two amino acid deletions were shared between varin producing accessions 21TX1-60 and 95-831. which are homozygous for the beneficial genotype of SNP marker 141840_721728. These two are a deletion of two amino acids (DD; two Aspartic Acid amino acids, observed for 21TX1-60 and 95-831) between positions 47 and 48 (SEQ ID NO: 11) corresponding with a six nucleotide deletion between positions 141 and 142 bp in the CDS (SEQ ID NO: 8). Abacus has a two amino acid (D48del and D49del; two Aspartic acid amino acids) insertion at positions 48-49 (SEQ ID NO: 12) corresponding with a six nucleotide (GATGAT) insertion at positions 142-145 bp (SEQ ID NO: 9; located at position 51 bp of SEQ ID NO: 17).

Two amino acid substitutions were shared between varin producing accessions 21TX1-60, 20VLP4-6-15, and 95-831. The first is an E to D substitution at position 79 in 21TX1-60 (SEQ ID NO: 11), corresponding with amino acid position 81 in Abacus (E81D; SEQ ID NO: 12) caused by an A to T nucleotide substitution (Abacus: A; 21TX1-60: T) located at CDS position 237 bp for 21TX1-60 (SEQ ID NO: 8), and CDS position 243 bp for Abacus (SEQ ID NO: 9; located at position 51 bp for SEQ ID NO: 19).

The second amino acid substitution shared among 21TX1-60, 20VLP4-6-15, and 95-831 is a K (Lysine; observed in Abacus) to E (Glutamic Acid; observed in 21TX1-60) amino acid substitution at position 83 in 21TX1-60 (SEQ ID NO: 11), corresponding with amino acid position 85 in Abacus (K85E; SEQ ID NO: 12) caused by a A to G nucleotide substitution (Abacus: A; 21TX1-60: G) at CDS position 247 bp for 21TX1-60 (SEQ ID NO: 8), and CDS position 253 bp for Abacus (SEQ ID NO: 9; located at position 51 bp for SEQ ID NO: 20).

Nine amino acid substitutions are specific for varin producing accession 21TX1-60 which is homozygous for the beneficial genotype of SNP marker 141840_721728. The first amino acid substitution specific for 21TX1-60 is an N (Asparagine; observed in Abacus) to K (Lysine; observed in 21TX1-60) substitution at position 66 in 21TX1-60 (SEQ ID NO: 11), corresponding with amino acid position 68 in Abacus (N68K; SEQ ID NO: 12) caused by a T to A nucleotide substitution (Abacus: T; 21TX1-60: A) at CDS position 198 bp for 21TX1-60 (SEQ ID NO: 8), and CDS position 204 bp for Abacus (SEQ ID NO: 9; located at position 51 bp for SEQ ID NO: 18).

The second amino acid substitution specific for 21TX1-60 is a Q (Glutamine; observed in Abacus) to H (Histidine; observed in 21TX1-60) amino acid substitution at position 109 in 21TX1-60 (SEQ ID NO: 11) and position 111 in Abacus (Q111H; SEQ ID NO: 12) caused by an A to T nucleotide substitution (Abacus: A; 21TX1-60: T) at CDS position 327 bp in 21TX1-60 (SEQ ID NO: 8) and 333 bp in Abacus (SEQ ID NO: 9; located at position 51 bp for SEQ ID NO: 21).

The third amino acid substitution specific for 21TX1-60 is an E (Glutamic Acid; observed in Abacus) to A (Alanine; observed in 21TX1-60) substitution at position 113 in 21TX1-60 (SEQ ID NO: 11), corresponding with position 115 in Abacus (E115A; SEQ ID NO: 12), caused by a A to C nucleotide substitution (Abacus: A; 21TX1-60: C) at CDS position 338 bp in 21TX1-60 (SEQ ID NO: 8) and CDS position 344 in Abacus (SEQ ID NO: 9; located at position 51 bp for SEQ ID NO: 22).

The fourth amino acid substitution specific for 21TX1-60 is a A (Alanine; observed in Abacus) to S (Serine; observed in 21TX1-60) substitution at position 122 in 21TX1-60 (SEQ ID NO: 11) and position 124 in Abacus (A124S; SEQ ID NO: 12), caused by a G to T nucleotide substitution (Abacus: G; 21TX1-60: T) at CDS position 364 bp in 21TX1-60 (SEQ ID NO: 8) and CDS position 370 bp in Abacus (SEQ ID NO: 9; located at position 51 bp for SEQ ID NO: 23).

The fifth amino acid substitution specific for 21TX1-60 is an I (Isoleucine; observed in Abacus) to V (Valine; observed in 21TX1-60) substitution at position 123 in 21TX1-60 (SEQ ID NO: 11) and position 125 in Abacus (I125V; SEQ ID NO: 12), caused by an A to G nucleotide substitution (Abacus: A; 21TX1-60: G) at CDS position 367 bp in 21TX1-60 (SEQ ID NO: 8) and CDS position 374 bp in Abacus (SEQ ID NO: 9; located at position 51 bp for SEQ ID NO: 24).

The sixth amino acid substitution specific for 21TX1-60 is an E (Glutamic Acid; observed in Abacus) to D (Aspartic Acid; observed in 21TX1-60) substitution at position 134 in 21TX1-60 (SEQ ID NO: 11) and position 136 in Abacus (E136D; SEQ ID NO: 12), caused by an A to T nucleotide substitution (Abacus: A; 21TX1-60: T) at CDS position 402 bp in 21TX1-60 (SEQ ID NO: 8) and CDS position 408 bp in Abacus (SEQ ID NO: 9; located at position 51 bp for SEQ ID NO: 25).

The seventh amino acid substitution specific for 21TX1-60 is an F (Phenylalanine; observed in Abacus) to I (Isoleucine; observed in 21TX1-60) substitution at position 158 in 21TX1-60 (SEQ ID NO: 11) and position 160 in Abacus (F160I; SEQ ID NO: 12), caused by a T to A nucleotide substitution (Abacus: T; 21TX1-60: A) at CDS position 472 bp in 21TX1-60 (SEQ ID NO: 8) and CDS position 478 bp in Abacus (SEQ ID NO: 9; located at position 51 bp for SEQ ID NO: 26).

The eighth amino acid substitution specific for 21TX1-60 is an A (Alanine; observed in Abacus) to V (Valine; observed in 21TX1-60) substitution at position 163 in 21TX1-60 (SEQ ID NO: 11) and position 165 in Abacus (A165V; SEQ ID NO: 12), caused by a C to T nucleotide substitution (Abacus: C; 21TX1-60: T) at CDS position 488 bp in 21TX1-60 (SEQ ID NO: 8) and CDS position 494 bp in Abacus (SEQ ID NO: 9; located at position 51 bp for SEQ ID NO: 27).

The ninth amino acid substitution specific for 21TX1-60 is an F (Phenylalanine; observed in Abacus) to V (Valine; observed in 21TX1-60) substitution at position 170 in 21TX1-60 (SEQ ID NO: 11) and position 172 in Abacus (F172V; SEQ ID NO: 12), caused by a T to G nucleotide substitution (Abacus: T; 21TX1-60: G) at CDS position 508 bp in 21TX1-60 (SEQ ID NO: 8) and CDS position 514 bp in Abacus (SEQ ID NO: 9; located at position 51 bp for SEQ ID NO: 28).

Two amino acid substitutions were specific to varin producing accession 95-831. The first amino acid substitution specific for 95-831 is an E (Glutamic Acid; observed in Abacus) to Q (Glutamine; observed in 95-831) substitution at position 200 in 95-831 (SEQ ID NO: 47) and position 202 in Abacus (E202Q; SEQ ID NO: 12), caused by an G to C nucleotide substitution (Abacus: G; 95-831: C) at CDS position 598 bp in 95-831 (SEQ ID NO: 46) and position 604 bp in Abacus (SEQ ID NO: 9; located at position 51 bp for SEQ ID NO: 48). The second amino acid substitution specific for 95-831 is a T (Threonine; observed in Abacus) to A (Alanine; observed in 95-831) substitution at position 204 in 95-831 (SEQ ID NO: 47) and position 206 in Abacus (T206A; SEQ ID NO: 12), caused by an A to G nucleotide substitution (Abacus: A; 95-831::G) at CDS position 610 bp in 95-831 (SEQ ID NO: 46) and position 616 bp in Abacus (SEQ ID NO: 9; located at position 51 bp for SEQ ID NO: 49).

The thioesterase ALT4 (ACYL-LIPID THIOESTERASE 4; Arabidopsis homolog AT1G68280) is involved in the generation of medium-chain fatty acid precursors through hydrolysis of fatty acyl thioester bonds, releasing linkage to either acyl-carrier protein (ACP) or coenzyme A (CoA; UniProt ID F4HX80). Fatty-acyl thioesterases are important for regulating lipid metabolism and trafficking where they direct newly synthesized fatty acids towards specific metabolic fates. Overexpression of AtALT4 in Nicotiana benthamiana results in accumulation of 6-8 carbon-length fatty acids (Kalinger, et al. “Production of C6-C14 Medium-Chain Fatty Acids in Seeds and Leaves via Overexpression of Single Hotdog-Fold Acyl-Lipid Thioesterases.” Lipids 56.3 (2021): 327-344).

Alignment with Arabidopsis thaliana ALT4 protein sequence revealed the location of the three active sites as described by Pulsifer, Ian P., et al. (“Acyl-lipid thioesterase 1-4 from Arabidopsis thaliana form a novel family of fatty acyl-acyl carrier protein thioesterases with divergent expression patterns and substrate specificities.” Plant molecular biology 84.4 (2014): 549-563.) to be located at amino acid positions 92, 95, and 97 in Abacus (D92, G95, and V97; SEQ ID NO: 12). These amino acids are conserved across Cannabis and Arabidopsis thaliana. In addition, these amino acids are conserved across ALT4 and three additional thioesterases (ALT1, ALT2, and ALT3) in Arabidopsis thaliana. However, when expressed in Escherichia coli these thioesterases generated fatty acids that varied in chain length (C6-C18) with ALT4 showing highest activity against the shortest fatty acid chain length of C6. Without being bound to any particular theory, it is possible that the amino acid substitutions observed in varin producing Cannabis accessions with the beneficial genotype for SNP marker 141840_721728 (21VLP5-1-101, 21VLP5-1-222, 22VLV2-1-52, 22VLV2-1-59, 23PV1-22-110, 20VLP4-6-15, 95-831, and 21TX1-60) alter ALT4 function to have increased activity against C4 fatty acids.

In Cannabis, plastid fatty acid biosynthesis forms the precursor for the acyl chain in THC and THCV (Welling, Matthew T., et al. “Complex patterns of cannabinoid alkyl side-chain inheritance in cannabis.” Scientific reports 9.1 (2019): 1-13.), as well as CBD and CBDV, CBC and CBCV, CBG and CBGV. The allelic variants for ALT4 observed in varin producing Cannabis accessions with the beneficial genotype for SNP marker 141840_721728 (21VLP5-1-101, 21VLP5-1-222, 22VLV2-1-52, 22VLV2-1-59, 23PV1-22-110, 20VLP4-6-15, 95-831 and 21TX1-60) possibly produce ALT4 variants that take C4 fatty acids created by the KR variants present in these accessions as substrate, removing the acyl-carrier protein (ACP) group to release the free 4-carbon chain fatty acid butanoic acid, resulting in a shorter propyl (3-carbon) side chain found in THCV, CBGV, CBCV, and CBDV, instead of a pentyl (5-carbon) group found in THC, CBC, CBG and CBD.

### ISS1

Alignment of ISS1 CDS and protein sequences of varin (22VLV2-1-52) and non-varin (Abacus) producing Cannabis accessions differing for the beneficial genotype of SNP marker 140868_21724 revealed no amino acid substitutions. However, RT-PCR based on young flower of varin producing accession 22VLV2-1-52 and non-varin producing accession Abacus (week 2-3 after daylength flip from 18/6 to 12/12 hours light/dark) differing for the beneficial genotype of SNP marker 140868_21724 revealed higher expression of ISS1 in Abacus as compared to 22VLV2-1-52 (FIG. 1). ISS1 upstream genomic DNA sequence for 22VLV2-1-52 (SEQ ID NO: 29) was compared with Abacus ISS1 upstream genomic DNA sequence (SEQ ID. NO: 30). This upstream sequence contained an A at nucleotide position −436 bp upstream from the start codon in non-varin producing accession Abacus (SEQ ID NO: 30; located at position 51 bp of SEQ ID NO: 31) which was absent (deletion) in varin producing accession 22VLV2-1-52 (SEQ ID NO: 29). The same deletion was observed in one of the cloned 21VLP5-1-101 ISS1 upstream sequences while two other cloned ISS1 upstream sequences of this accession contained an A insertion at this position, indicating that 21VLP5-1-101 is heterozygous for this indel. The observation that this indel was heterozygous in accession 21VLP5-1-101, homozygous for the deletion in 22VLV2-1-52, and homozygous for the insertion (reference allele A) in Abacus reflects the 140868_21724 SNP marker genotypes (Table 1). This deletion resides inside an AG (n) short tandem repeat (STR). Length variation in STRs up to a few hundred base pairs upstream from the start codon of a gene affects gene expression in Arabidopsis, where shorter STRs result in lower gene expression as compared to longer STRs (Reinar, William B., et al. “Length variation in short tandem repeats affects gene expression in natural populations of Arabidopsis thaliana.” The Plant Cell 33.7 (2021): 2221-2234.). The deletion in the STR makes it effectively shorter and therefore potentially results in reduced expression of ISS1.

The aromatic aminotransferase ISS1 (INDOLE SEVERE SENSITIVE 1; Arabidopsis homolog AT1G80360) catalyzes the enzymatic reaction of a 2-oxocarboxylate and L-methionine to 4-methylsulfanyl-2-oxobutanoate and an L-α-amino-acid (UniProt ID Q9C969). In the situation of lower ISS1 expression, as observed for varin producing accession 22VLV1-1-52, there may be larger amounts of 2-oxocarboxylate remaining. 2-oxocarboxylate plus NADPH and H+ converts to a (2R)-2-hydroxy fatty acid. 2,2-dihydroxy-3-methylbutanoic acid (CHEBI: 167881) is a 2-hydroxy fatty acid (CHEBI: 10283). 2-oxobutanoate (CHEBI: 16763) is a 2-oxocarboxylate with a H3C group as the R group (CHEBI: 35179) and converts to 2-oxobutanoic acid (CHEBI: 30831). 2-oxobutanoic acid (CHEBI: 30831) is a conjugate acid of 2-oxobutanoate (CHEBI: 16763). Butanoic acid and 2-oxobutanoic acid and/or 2,2-dihydroxy-3-methylbutanoic acid could be used as additional precursor molecules in varin production (Welling, Matthew T., et al. “Complex patterns of cannabinoid alkyl side-chain inheritance in cannabis.” Scientific reports 9.1 (2019): 1-13.). Therefore, without being bound by any particular theory, lower expression of ISS1 resulting from the deletion of an A in an STR upstream of this gene could result in reduced use of precursor in the enzymatic reaction. As this precursor molecule is a form of butanoic acid, which is a precursor in varin production, this could be a mechanism by which the ISS1 upstream deletion results in increased varin production.

### KR

Alignment of KR coding sequence (CDS) and protein sequences of varin (21VLP5-1-101, 21VLP5-1-222, 22VLV2-1-52, 22VLV2-1-59, 23PV1-22-110, 22TP1C-140-40, 22TP1C-141-25, and 21TX1-60) and non-varin (Abacus) producing Cannabis accessions differing for the beneficial genotype of SNP marker 142078_3920202 revealed fifteen amino acid substitutions, which were not previously described.

The first amino acid substitution is a Q (Glutamine, observed in Abacus) to R (Arginine, observed in 22VLV2-1-59,AFR,h) amino acid substitution at position 39 in 22VLV2-1-59 (SEQ ID NO: 71) and Abacus (Q39R; SEQ ID NO: 66) caused by a A to G nucleotide substitution (Abacus: A; 22VLV2-1-59: G) at CDS position 116 bp of 22VLV2-1-59 (SEQ ID NO: 55) and Abacus (SEQ ID NO: 50; located at position 51 bp for SEQ ID NO: 82).

The second amino acid substitution is a S (Serine, observed in Abacus) to P (Proline, observed in 22VLV2-1-52,cpy2AFR,c) amino acid substitution at position 88 in 22VLV2-1-52 (SEQ ID NO: 68) and Abacus (S88P; SEQ ID NO: 66) caused by a T to C nucleotide substitution (Abacus: T; 22VLV2-1-52: C) at CDS position 262 bp of 22VLV2-1-52 (SEQ ID NO: 52) and Abacus (SEQ ID NO: 50; located at position 51 bp for SEQ ID NO: 83).

The third amino acid substitution is a K (Lysine, observed in Abacus) to E (Glutamic Acid, observed in 22VLV2-1-59,AFR,j) amino acid substitution at position 122 in 22VLV2-1-59 (SEQ ID NO: 72) and Abacus (K122E; SEQ ID NO: 66) caused by an A to G nucleotide substitution (Abacus: A; 22VLV2-1-59: G) at CDS position 364 bp of 22VLV2-1-59 (SEQ ID NO: 56) and Abacus (SEQ ID NO: 50; located at position 51 bp for SEQ ID NO: 84).

The fourth amino acid substitution is a E (Glutamic Acid, observed in Abacus) to G (Glycine, observed in 21VLP5-1-222,AFR,j) amino acid substitution at position 123 in 21VLP5-1-222 (SEQ ID NO: 74) and Abacus (E123G; SEQ ID NO: 66) caused by a G to A nucleotide substitution (Abacus: G; 21VLP5-1-222: A) at CDS position 368 bp of 21VLP5-1-222 (SEQ ID NO: 58) and Abacus (SEQ ID NO: 50; located at position 51 bp for SEQ ID NO: 85).

The fifth amino acid substitution is a E (Glutamic Acid, observed in Abacus) to G (Glycine, observed in 22VLV2-1-59,AFR,e) amino acid substitution at position 125 in 22VLV2-1-59 (SEQ ID NO: 70) and Abacus (E125G; SEQ ID NO: 66) caused by a A to G nucleotide substitution (Abacus: A; 22VLV2-1-59: G) at CDS position 374 bp of 22VLV2-1-59 (SEQ ID NO: 54) and Abacus (SEQ ID NO: 50; located at position 51 bp for SEQ ID NO: 86).

The sixth amino acid substitution is an S (Serine, observed in Abacus) to P (Proline, observed in 22TP1C-141-25,AFR,a) amino acid substitution at position 139 in 22TP1C-141-25 (SEQ ID NO: 77) and Abacus (S139P; SEQ ID NO: new) caused by a T to C nucleotide substitution (Abacus: T; 22TP1C-141-25: C) at CDS position 415 bp of 22TP1C-141-25 (SEQ ID NO: 61) and Abacus (SEQ ID NO: 50; located at position 51 bp for SEQ ID NO: 87).

The seventh amino acid substitution is an I (Isoleucine, observed in Abacus) to V (Valine, observed in 21VLP5-1-101, 21VLP5-1-222, 22VLV2-1-52, 22VLV2-1-59, and 21TX1-60) amino acid substitution at position 158 in 22VLV2-1-59,AFR,e (SEQ ID NO: 70) and Abacus (I158V; SEQ ID NO: 66) caused by a A to G nucleotide substitution (Abacus: A; 21VLP5-1-101, 21VLP5-1-222, 22VLV2-1-52, 22VLV2-1-59, and 21TX1-60: G) at CDS position 472 bp of 22VLV2-1-59 (SEQ ID NO: 54) and Abacus (SEQ ID NO: 50; located at position 51 bp for SEQ ID NO: 88).

The eighth amino acid substitution is an R (Arginine, observed in Abacus) to G (Glycine, observed in 22TP1C-141-25,AFR,a) amino acid substitution at position 175 in 22TP1C-141-25 (SEQ ID NO: 77) and Abacus (R175G; SEQ ID NO: 66) caused by a A to G nucleotide substitution (Abacus: A; 22TP1C-141-25: G) at CDS position 523 bp of 22TP1C-141-25 (SEQ ID NO: 61) and Abacus (SEQ ID NO: 50; located at position 51 bp for SEQ ID NO: 89).

The ninth amino acid substitution is an F (Phenylalanine, observed in Abacus) to S (Serine, observed in 22VLV2-1-52,AFR,e) amino acid substitution at position 193 in 22VLV2-1-52 (SEQ ID NO: 69) and Abacus (F193S; SEQ ID NO: 66) caused by a T to C nucleotide substitution (Abacus: T; 22VLV2-1-52: C) at CDS position 578 bp of 22VLV2-1-52 (SEQ ID NO: 53) and Abacus (SEQ ID NO: 50; located at position 51 bp for SEQ ID NO: 90).

The tenth amino acid substitution is an K (Lysine, observed in Abacus) to R (Arginine, observed in 22TP1C-140-40,AFR,b) amino acid substitution at position 205 in 22TP1C-140-40 (SEQ ID NO: 75) and Abacus (K205R; SEQ ID NO: 66) caused by a A to G nucleotide substitution (Abacus: A; 22TP1C-140-40: G) at CDS position 614 bp of 22TP1C-140-40 (SEQ ID NO: 59) and Abacus (SEQ ID NO: 50; located at position 51 bp for SEQ ID NO: 91).

The eleventh amino acid substitution is a K (Lysine, observed in Abacus) to R (Arginine, observed in 22VLV2-1-52,cpy2AFR,c) amino acid substitution at position 208 in 22VLV2-1-52 (SEQ ID NO: 68) and Abacus (K208R; SEQ ID NO: 66) caused by a A to G nucleotide substitution (Abacus: A; 22VLV2-1-52: G) at CDS position 623 bp of 22VLV2-1-52 (SEQ ID NO: 52) and Abacus (SEQ ID NO: 50; located at position 51 bp for SEQ ID NO: 92).

The twelfth amino acid substitution is an V (Valine, observed in Abacus) to I (Isoleucine, observed in 21VLP5-1-101, 21VLP5-1-222, 22VLV2-1-52, 22VLV2-1-59, 22TP1C-140-40, and 22TP1C-141-25) amino acid substitution at position 217 in 22VLV2-1-52,cpy2AFR,c (SEQ ID NO: 68) and Abacus (V2171; SEQ ID NO: 66) caused by a G to A nucleotide substitution (Abacus: G; 22VLV2-1-52: A) at CDS position 649 bp of 22VLV2-1-52 (SEQ ID NO: 52) and Abacus (SEQ ID NO: 50; located at position 51 bp for SEQ ID NO: 93).

The thirteenth amino acid substitution is an F (Phenylalanine, observed in Abacus) to S (Serine, observed in 21VLP5-1-222,AFR,a) amino acid substitution at position 218 in 21VLP5-1-222 (SEQ ID NO: 73) and Abacus (F218S; SEQ ID NO: 66) caused by a T to C nucleotide substitution (Abacus: T; 21VLP5-1-222: C) at CDS position 653 bp of 21VLP5-1-222 (SEQ ID NO: 57) and Abacus (SEQ ID NO: 50; located at position 51 bp for SEQ ID NO: 94).

The fourteenth amino acid substitution is an N (Asparagine, observed in Abacus) to S (Serine, observed in 22TP1C-141-25,AFR,b) amino acid substitution at position 251 in 22TP1C-141-25 (SEQ ID NO: 78) and Abacus (N251S; SEQ ID NO: 66) caused by a A to G nucleotide substitution (Abacus: A; 22TP1C-141-25: G) at CDS position 752 bp of 22TP1C-141-25 (SEQ ID NO: 62) and Abacus (SEQ ID NO: 50; located at position 51 bp for SEQ ID NO: 95).

The fifteenth amino acid substitution is a V (Valine, observed in Abacus) to I (Isoleucine, observed in 23PV1-22-110,cpy2BFR,f) amino acid substitution at position 293 in 23PV1-22-110 (SEQ ID NO: 81) and Abacus (V293I; SEQ ID NO: 66) caused by a G to A nucleotide substitution (Abacus: G; 23PV1-22-110: A) at CDS position 877 bp of 23PV1-22-110 (SEQ ID NO: 65) and Abacus (SEQ ID NO: 50; located at position 51 bp for SEQ ID NO: 96).

These amino acid substitutions have resulted in the identification of novel alleles for KR in accessions with Total Varin as high as 20% and Varin Ratio as high as 7 (see, Table 1). KR functions to catalyze two of the reactions that constitute the core four-reaction cycle of the fatty acid biosynthesis (FAS) system, which iteratively elongates the acyl-chain by two carbon atoms per cycle (Guan et al. 2020, Plant Physiology 183 (2): 517-529). In Cannabis, plastid fatty acid biosynthesis forms the precursor for the acyl chain in cannabinoids (Welling et al. 2019, Scientific Reports 9 (1): 1-13). Allelic variation of KR likely produces KR variants that result in a shorter propyl (3-carbon) side chain found in THCV, CBDV, CBCV, and CBGV instead of a pentyl (5-carbon) group found in THC, CBD, CBC, and CBG. It was unexpected that substitutions occurring outside conserved domains, such as conserved NADP domains, would have a profound effect on the production of C4 fatty acids, however, without being bound by any particular theory, it may be that each additional amino acid substitution observed in varin producing accessions increases the extent of conformation changes of the KR protein and as a result the quantity of produced C4 relative to longer fatty acids and therefore the quantity of varins as compared to pentyl cannabinoids.

The fact that some accessions have more than two different alleles for KR indicates that this gene has at least one additional related copy in some genetic backgrounds.

TABLE 2
Amino acid substitutions in KR observed in varin producing accessions.

| | 22VLV2-1-52 | | | | | | | | | | | | 23PV1-22-110 | | |
|---|---|---|---|---|---|---|---|---|---|---|---|---|---|---|---|
| | cpy2 | | 22VLV2-1-59 | | | 21VLP5-1-222 | | 22TP1C-140-40 | | 22TP1C-141-25 | | | cpy | cpy2 | |
| | AFR, | AFR, | AFR, | AFR, | AFR, | AFR, | AFR, | AFR, | AFR, | AFR, | AFR, | AFR, | 2BFR, | BFR, | 21TX1-60 |
| Sub. | c | e | e | h | j | a | j | b | f | a | b | d | a | f | BFR |
| Q39R | Q | Q | Q | R | Q | Q | Q | Q | Q | Q | Q | Q | Q | Q | Q |
| S88P | P | S | S | S | S | S | S | S | S | S | S | P | S | S | S |
| K122E | K | K | K | K | E | K | K | K | K | K | K | K | K | K | K |
| E123G | E | E | E | E | E | E | G | E | E | E | E | E | E | E | E |
| E125G | E | E | G | E | E | E | E | E | E | E | E | E | E | E | E |
| S139P | S | S | S | S | S | S | S | S | S | P | S | S | S | S | S |
| I158V | V | V | V | V | V | V | V | V | V | V | V | V | V | V | V |
| R175G | R | R | R | R | R | R | R | R | R | G | R | R | R | R | R |
| F193S | F | S | F | F | F | F | F | F | F | F | F | F | F | F | F |
| K205R | K | K | K | K | K | K | K | R | K | K | K | K | K | K | K |
| K208R | R | K | K | K | K | K | K | K | K | K | K | K | K | K | K |
| V217I | I | I | I | I | I | I | I | I | I | I | I | I | V | V | V |
| F218S | F | F | F | F | F | S | F | F | F | F | F | F | F | F | F |
| N251S | N | N | N | N | N | N | N | N | N | N | S | N | N | N | N |
| V293I | V | V | V | V | V | V | V | V | V | V | V | V | V | I | V |

First column: amino acid substitution as compared to the Abacus reference genome, second through sixteenth column: amino acid observed for varin producing Cannabis accessions. The first row displays accession names; the second row displays abbreviated primer information used to amplify the fragment used for cloning, followed by the letter code for cloned sequences after the comma (the absence of a letter code indicates direct Sanger sequencing of PCR product).

### FATB

Alignment of FATB coding sequence (CDS) and protein sequences of varin (21VLP5-1-222, 22VLV2-1-52, and 21TX1-60) and non-varin (Abacus) producing Cannabis accessions differing for the beneficial genotype of SNP marker 141488_308754 revealed six amino acid substitutions, four of those were in common between varin producing accessions with the beneficial genotype for this SNP marker: 21VLP5-1-222, 22VLV2-1-52, and 21TX1-60 (Table 1; two amino acid substitutions were only observed in 22VLV2-1-52; 21VLP5-1-222 and 21TX1-60 had missing sequence data in this region) and differed from non-varin producing accession Abacus.

The first amino acid substitution is a D (Aspartic Acid, observed in Abacus) to H (Histidine, observed in 22VLV2-1-52) amino acid substitution at position 155 in 2VLV2-1-52 (SEQ ID NO: 99) and Abacus (D155H; SEQ ID NO: 100) caused by a G to C nucleotide substitution (Abacus: G; 22VLV2-1-52: C) at CDS position 463 bp of 22VLV2-1-52 (SEQ ID NO: 97) and Abacus (SEQ ID NO: 98; located at position 51 bp for SEQ ID NO: 101).

The second amino acid substitution is an R (Arginine, observed in Abacus) to Q (Glutamine, observed in 22VLV2-1-52) amino acid substitution at position 173 in 2VLV2-1-52 (SEQ ID NO: 99) and Abacus (R173Q; SEQ ID NO: 100) caused by a G to A nucleotide substitution (Abacus: G; 22VLV2-1-52: A) at CDS position 518 bp of 22VLV2-1-52 (SEQ ID NO: 97) and Abacus (SEQ ID NO: 98; located at position 51 bp for SEQ ID NO: 102).

The third amino acid substitution is an G (Glycine, observed in Abacus) to R (Arginine, observed in 22VLV2-1-52, 21VLP5-1-22, and 21TX1-60) amino acid substitution at position 197 in 2VLV2-1-52 (SEQ ID NO: 99) and Abacus (G197R; SEQ ID NO: 100) caused by a G to C nucleotide substitution (Abacus: G; 22VLV2-1-52, 21VLP5-1-222, and 21TX1-60: C) at CDS position 589 bp of 22VLV2-1-52 (SEQ ID NO: 97) and Abacus (SEQ ID NO: 98; located at position 51 bp for SEQ ID NO: 103).

The fourth amino acid substitution is an M (Methionine, observed in Abacus) to I (Isoleucine, observed in 22VLV2-1-52, 21VLP5-1-22, and 21TX1-60) amino acid substitution at position 201 in 2VLV2-1-52 (SEQ ID NO: 99) and Abacus (M201I; SEQ ID NO: 100) caused by a G to T nucleotide substitution (Abacus: G; 22VLV2-1-52, 21VLP5-1-222, and 21TX1-60: T) at CDS position 603 bp of 22VLV2-1-52 (SEQ ID NO: 97) and Abacus (SEQ ID NO:102; located at position 51 bp for SEQ ID NO: 104).

The fifth amino acid substitution is an Q (Glutamine, observed in Abacus) to P (Proline, observed in 22VLV2-1-52, 21VLP5-1-22, and 21TX1-60) amino acid substitution at position 202 in 2VLV2-1-52 (SEQ ID NO: 99) and Abacus (Q202P; SEQ ID NO: 100) caused by a A to C nucleotide substitution (Abacus: A; 22VLV2-1-52, 21VLP5-1-222, and 21TX1-60: C) at CDS position 605 bp of 22VLV2-1-52 (SEQ ID NO: 97) and Abacus (SEQ ID NO: 98; located at position 51 bp for SEQ ID NO: 105).

The sixth amino acid substitution is an E (Glutamic Acid, observed in Abacus) to D (Aspartic Acid, observed in 22VLV2-1-52, 21VLP5-1-22, and 21TX1-60) amino acid substitution at position 207 in 2VLV2-1-52 (SEQ ID NO: 99) and Abacus (E207D; SEQ ID NO: 100) caused by a G to T nucleotide substitution (Abacus: G; 22VLV2-1-52, 21VLP5-1-222, and 21TX1-60: T) at CDS position 621 bp of 22VLV2-1-52 (SEQ ID NO: 97) and Abacus (SEQ ID NO: 98; located at position 51 bp for SEQ ID NO: 106).

NCBI conserved domain search (Marchler-Bauer, Aron, et al. “CDD/SPARCLE: functional classification of proteins via subfamily domain architectures.” Nucleic acids research 45.D1 (2017): D200-D203.) identified an acyl-ACP thioesterase domain between amino acid positions 83-295 in the Abacus protein sequence of FATB. All six of the amino acid substitutions (D155H, R173Q, G197R, M201I, Q202P, and E207D) are located inside this domain. Amino acid substitutions M201I and Q202P are each located 1 amino acid from two residues that affect substrate specificity (Lys-200 in Abacus corresponding with Lys-175 in Arabidopsis thaliana; Glu-203 in Abacus corresponding with Glu-178 in Arabidopsis thaliana; Mayer, Kimberly M., and John Shanklin. “A structural model of the plant acyl-acyl carrier protein thioesterase FatB comprises two helix/4-stranded sheet domains, the N-terminal domain containing residues that affect specificity and the C-terminal domain containing catalytic residues.” Journal of Biological Chemistry 280.5 (2005): 3621-3627.).

FATB is like ALT4 an acyl-acyl carrier protein thioesterase involved in chain termination during de novo fatty acid synthesis (Kalinger, Rebecca S., et al. “Production of C6-C14 Medium-Chain Fatty Acids in Seeds and Leaves via Overexpression of Single Hotdog-Fold Acyl-Lipid Thioesterases.” Lipids 56.3 (2021): 327-344.). Without being bound to any particular theory, due to the close proximity of two amino acid substitutions to two amino acids involved in substrate specificity it is hypothesized that these amino acid substitutions observed in varin producing accessions have altered FATB substrate specificity, which has shifted from medium size carbon chain fatty acids (14-18 carbons in length) to shorter sized carbon chain fatty acids, including carbon chain fatty acids with chain length of 4, which are precursors to varins.

### GDSL1

Alignment of GDSL1 coding sequence (CDS) and protein sequences of varin (21VLP5-1-101, 21VLP5-1-222, 22VLV2-1-52, 22VLV2-1-29, and 23PV1-22-110) and non-varin (Abacus) producing Cannabis accessions differing for the beneficial genotype of SNP marker 141840_475273 revealed three amino acid substitutions which were in common between varin producing accessions with the beneficial genotype for this SNP marker and differed from non-varin producing accession Abacus (Table 1).

The first amino acid substitution is a V (Valine, observed in Abacus) to an A (Alanine, observed in 22VLV2-1-52, 22VLV2-1-59, and 23PV1-22-110) amino acid substitution at position 59 in 2VLV2-1-52 (SEQ ID NO: 109) and Abacus (V59A; SEQ ID NO: 110) caused by a T to C nucleotide substitution (Abacus: T; 22VLV2-1-52: C) at CDS position 176 bp of 22VLV2-1-52 (SEQ ID NO: 107) and Abacus (SEQ ID NO: 108; located at position 51 bp for SEQ ID NO: 111). Accessions 21VLP5-1-101 and 21VLP5-1-222 which were heterozygous for SNP marker 141840_475273 were also heterozygous for T/C at nucleotide position 176 bp causing heterozygosity for the V and A amino acid positions.

The second amino acid substitution is a D (Aspartic Acid, observed in Abacus) to an N (Asparagine, observed in 22VLV2-1-52, 22VLV2-1-59, and 23PV1-22-110) amino acid substitution at position 275 in 2VLV2-1-52 (SEQ ID NO: 109) and Abacus (D275N; SEQ ID NO: 110) caused by a G to A nucleotide substitution (Abacus: G; 22VLV2-1-52: A) at CDS position 823 bp of 22VLV2-1-52 (SEQ ID NO: 107) and Abacus (SEQ ID NO: 108; located at position 51 bp for SEQ ID NO: 112). Accessions 21VLP5-1-101 and 21VLP5-1-222 which were heterozygous for SNP marker 141840_475273 were also heterozygous for G/A at nucleotide position 823 bp causing heterozygosity for the D and N amino acid positions.

The third amino acid substitution is an A (Alanine, observed in Abacus) to a T (Threonine, observed in 22VLV2-1-52, 22VLV2-1-59, and 23PV1-22-110) substitution at position 297 in 2VLV2-1-52 (SEQ ID NO: 109) and Abacus (A297T; SEQ ID NO: 110) caused by a G to A nucleotide substitution (Abacus: G; 22VLV2-1-52: A) at CDS position 889 bp of 22VLV2-1-52 (SEQ ID NO: 107) and Abacus (SEQ ID NO: 108; located at position 51 bp for SEQ ID NO: 113). Accessions 21VLP5-1-101 and 21VLP5-1-222 which were heterozygous for SNP marker 141840_475273 were also heterozygous for G/A at nucleotide position 889 bp causing heterozygosity for the A and T amino acid positions.

In addition, there was one amino acid substitution in common between varin producing accessions 21VLP5-1-101, 22VLV2-1-52, 22VLV2-1-59, and 23PV1-22-110 which differed from non-varin producing accession Abacus. This amino acid substitution is an L (Leucine, observed in Abacus) to M (Methionine, observed in 21VLP5-1-101, 22VLV2-1-52, 22VLV2-1-59, and 23PV1-22-110 (L213M) substitution at position 213 in 22VLV2-1-52 (SEQ ID NO: 109) and Abacus (L213M; SEQ ID NO: 110) caused by a T to A nucleotide substitution (Abacus: T; 22VLV2-1-52: A) at CDS position 637 bp of 22VLV2-1-52 (SEQ ID NO: 107) and Abacus (SEQ ID NO: 108; located at position 51 bp for SEQ ID NO: 114). Accession 21VLP5-1-101 which was heterozygous for SNP marker 141840_475273 was also heterozygous for T/A at nucleotide position 637 bp causing heterozygosity for the L and M amino acid positions.

NCBI conserved domain search (Marchler-Bauer, Aron, et al. “CDD/SPARCLE: functional classification of proteins via subfamily domain architectures.” Nucleic acids research 45.D1 (2017): D200-D203.) identified an SGNH plant lipase-like domain between amino acid positions 36-353 in the Abacus protein sequence of GDSL1. All four of the amino acid substitutions (V59A, L213M, D275N, and A297T) are located inside this domain. Overexpression of GDSL1 in Arabidopsis thaliana and Brassica napus promoted lipid catabolism, while RNAi-mediated suppression caused changes in the fatty acid composition with an increase in C18:1 and a decrease in C18:2 and C18:3 (Ding, Li-Na, et al. “Improving seed germination and oil contents by regulating the GDSL transcriptional level in Brassica napus.” Plant Cell Reports 38 (2019): 243-253.). Without being bound to any particular theory, it is hypothesized that the amino acid substitutions in varin producing accessions affect lipid catabolism to produce increased amounts of carbon chain fatty acids with chain length of 4 (C4), which are precursors to varins, as compared to fatty acids with longer chain lengths.

### Combination of Genetic Variants for High Total Varin and High Varin Ratio

Provided below are tables showing substitutions that are present in the indicated Cannabis lines.

TABLE 3
Select amino acid substitutions and genotype of the indicated Cannabis accession.

| Accession | Total | Varin | KR | FATB | GDSL1 | ALT4 | ALT4 | ISS1 |
|---|---|---|---|---|---|---|---|---|
| name | Varin (%) | Ratio | I158V | M201I | T267M | P7S | N68K | Del A |
| Abacus | 0.00 | 0.00 | I | M | T | P | N | A |
| 22VLV2-1-52 | 6.14 | 4.80 | V | I | M | S | N | Del A |
| 21TX1-60 | 5.40 | 1.00 | V | I | NA | P | K | A |
| 22TP1C-140-40 | 20.21 | 4.62 | V | I* | NA/M* | P/S* | N/K* | A/Del A* |

*= inferred based on genotypes of sibs of parents.
NA = no data,
/ = heterozygous.

TABLE 4
Select amino acid substitutions and genotype of the indicated Cannabis accession.

| | Total | Varin | KR | FATB | GDSL1 | ALT4 | ALT4 | ISS1 |
|---|---|---|---|---|---|---|---|---|
| Accession name | Varin (%) | Ratio | I158V | M201I | T267M | P7S | N68K | Del A |
| Abacus | 0.00 | 0.00 | I | M | T | P | N | A |
| 21VLP5-1-101 | 4.23 | 3.71 | V | NA | T/M | S | N | A/Del A |
| 21VLP5-1-222 | 4.14 | 2.98 | V | I | T/M | NA | N | NA |
| 22VLV2-1-52 | 6.14 | 4.80 | V | I | M | S | N | Del A |
| 22VLV2-1-59 | 10.13 | 4.80 | V | NA | M | S | N | NA |
| 23PV1-22-110 | 13.82 | 7.02 | V | NA | M | S | N | NA |
| 21TX1-60 | 5.40 | 1.00 | V | I | NA | P | K | A |
| 22TP1C-140-40 | 20.21 | 4.62 | V | I* | NA/M* | P/S* | N/K* | A/Del A* |
| 22TP1C-141-25 | 20.61 | 4.11 | V | I* | NA/M* | P/S* | N/K* | A/Del A* |
| 20VLP4-6-15 | 2.03 | 0.39 | NA | NA | NA | P | N | A |
| 95-831 | 0.91 | 0.04 | NA | NA | NA | P | N | A |

*= inferred based on genotypes of sibs of parents.
NA = no data.

Breeding or selecting Cannabis plants having modified Total Varin and/or a high Varin Ratio can involve selecting for beneficial amino acid substitutions identified herein (e.g., a combination of substitutions listed in Tables 3 and 4), and bringing them together in optimized combinations to fine tune desired varin content.

TABLE 5
Genomic DNA, coding, and protein sequences of genes and primers used for sequencing.

| SEQ ID NO | Description |
|---|---|
| SEQ ID NO: 1 | ALT4S_AF |
| SEQ ID NO: 2 | ALT4S_AR |
| SEQ ID NO: 3 | ALT4C_AF |
| SEQ ID NO: 4 | ALT4C_CR |
| SEQ ID NO: 5 | ISS1U_AF |
| SEQ ID NO: 6 | ISS1U_AR |
| SEQ ID NO: 7 | 21VLP5-1-101 ALT4 CDS (1-564 bp)* |
| SEQ ID NO: 8 | 21TX1-60 ALT4 CDS (1-555 bp)* |
| SEQ ID NO: 9 | Abacus ALT4 CDS (1-630 bp) |
| SEQ ID NO: 10 | 21VLP5-1-101 ALT4 protein sequence (1-188 AA)** |
| SEQ ID NO: 11 | 21TX1-60 ALT4 protein sequence (1-185 AA)** |
| SEQ ID NO: 12 | Abacus ALT4 protein sequence (1-209 AA) |
| SEQ ID NO: 13 | Abacus ALT4 genomic DNA sequence flanking causative SNP (G/A substitution |
| | located at 176 bp in Abacus CDS) located at position 51 bp (Abacus AA |
| | substitution R59Q) |
| SEQ ID NO: 14 | Abacus ALT4 genomic DNA sequence flanking causative SNP (A/G substitution |
| | located at 197 bp in Abacus CDS) located at position 51 bp (Abacus AA |
| | substitution E66G) |
| SEQ ID NO: 15 | Abacus ALT4 genomic DNA sequence flanking causative indel (—/GAT indel |
| | between positions 141-142 bp in Abacus CDS) located at position 50-52 bp |
| | (Abacus AA deletion of a D) |
| SEQ ID NO: 16 | Abacus ALT4 genomic DNA sequence flanking causative SNP (C/T substitution |
| | located at 199 bp in Abacus CDS) located at position 51 bp (Abacus AA |
| | substitution H67Y) |
| SEQ ID NO: 17 | Abacus ALT4 genomic DNA sequence flanking causative indel (GATGAT/— |
| | indel between positions 142-145 bp in Abacus CDS) located at position 51-56 |
| | bp (Abacus AA insertions D48del and D49del) |
| SEQ ID NO: 18 | Abacus ALT4 genomic DNA sequence flanking causative SNP (T/A substitution |
| | at 204 bp in Abacus CDS) located at position 51 bp (Abacus AA substitution |
| | N68K) |
| SEQ ID NO: 19 | Abacus ALT4 genomic DNA sequence flanking causative SNP (A/T substitution |
| | at 243 bp in Abacus CDS) located at position 51 bp (Abacus AA substitution |
| | E81D) |
| SEQ ID NO: 20 | Abacus ALT4 genomic DNA sequence flanking causative SNP (A/G substitution |
| | at 253 bp in Abacus CDS) located at position 51 bp (Abacus AA substitution |
| | K85E) |
| SEQ ID NO: 21 | Abacus ALT4 genomic DNA sequence flanking causative SNP (A/T substitution |
| | located at position 333 bp in Abacus CDS) located at position 51 bp (Abacus AA |
| | substitution Q111H) |
| SEQ ID NO: 22 | Abacus ALT4 genomic DNA sequence flanking causative SNP (A/C substitution |
| | located at position 244 bp in Abacus CDS) located at position 51 bp (Abacus AA |
| | substitution E115A) |
| SEQ ID NO: 23 | Abacus ALT4 genomic DNA sequence flanking causative SNP (G/T substitution |
| | located at 370 bp in Abacus CDS) located at position 51 bp (Abacus AA |
| | substitution A124S) |
| SEQ ID NO: 24 | Abacus ALT4 genomic DNA sequence flanking causative SNP (A/G substitution |
| | located at 374 bp in Abacus CDS) located at position 51 bp (Abacus AA |
| | substitution I125V) |
| SEQ ID NO: 25 | Abacus ALT4 genomic DNA sequence flanking causative SNP (A/T substitution |
| | located at 408 bp in Abacus CDS) located at position 51 bp (Abacus AA |
| | substitution E136D) |
| SEQ ID NO: 26 | Abacus ALT4 genomic DNA sequence flanking causative SNP (T/A substitution |
| | located at 478 bp in Abacus CDS) located at position 51 bp (Abacus AA |
| | substitution F160I) |
| SEQ ID NO: 27 | Abacus ALT4 genomic DNA sequence flanking causative SNP (C/T substitution |
| | located at 494 bp in Abacus CDS) located at position 51 bp (Abacus AA |
| | substitution A165V) |
| SEQ ID NO: 28 | Abacus ALT4 genomic DNA sequence flanking causative SNP (T/G substitution |
| | located at 514 bp in Abacus CDS) located at position 51 bp (Abacus AA |
| | substitution F172V) |
| SEQ ID NO: 29 | 22VLV2-1-52 ISS1 upstream region genomic DNA sequence (−512-−333 bp) |
| SEQ ID NO: 30 | Abacus ISS1 upstream region genomic DNA sequence (−512-−1 bp) |
| SEQ ID NO: 31 | Abacus ISS1 genomic DNA sequence flanking causative SNP located at position |
| | 51 bp |
| SEQ ID NO: 32 | ALT4S_BF |
| SEQ ID NO: 33 | KR1C_AF |
| SEQ ID NO: 34 | KR1C_AR |
| SEQ ID NO: 35 | KR1C_BF |
| SEQ ID NO: 36 | KR1C_BR |
| SEQ ID NO: 37 | KR1C_cpy2_AF |
| SEQ ID NO: 38 | KR1C_cpy2_AR |
| SEQ ID NO: 39 | KR1C_cpy2_BF |
| SEQ ID NO: 40 | KR1C_cpy2_BR |
| SEQ ID NO: 41 | FATBC_1AF |
| SEQ ID NO: 42 | FATBC_1AR |
| SEQ ID NO: 43 | GDSL1C_AF |
| SEQ ID NO: 44 | GDSL1C_AR |
| SEQ ID NO: 45 | Abacus ALT4 genomic DNA sequence flanking causative SNP (C/T substitution |
| | at 19 bp in Abacus CDS) located at position 51 bp (Abacus AA substitution P7S) |
| SEQ ID NO: 46 | 95-831 ALT4 CDS (1-285 bp)** |
| SEQ ID NO: 47 | 95-831 ALT4 protein sequence (1-95 AA)** |
| SEQ ID NO: 48 | Abacus ALT4 genomic DNA sequence flanking causative SNP (A/G substitution |
| | at 604 bp in Abacus CDS) located at position 51 bp (Abacus AA substitution |
| | E202Q) |
| SEQ ID NO: 49 | Abacus ALT4 genomic DNA sequence flanking causative SNP (A/G substitution |
| | at 616 bp in Abacus CDS) located at position 51 bp (Abacus AA substitution |
| | T206A) |
| SEQ ID NO: 50 | Abacus KR CDS (82-801 bp)* |
| SEQ ID NO: 51 | 21TX1-60 KR CDS (82-801 bp)* |
| SEQ ID NO: 52 | 22VLV2-1-52, cpy2AFR, c KR CDS (136-705 bp)* |
| SEQ ID NO: 53 | 22VLV2-1-52, AFR, e KR CDS (82-801 bp)* |
| SEQ ID NO: 54 | 22VLV2-1-59, AFR, e KR CDS (82-801 bp)* |
| SEQ ID NO: 55 | 22VLV2-1-59, AFR, h KR CDS (82-801 bp)* |
| SEQ ID NO: 56 | 22VLV2-1-59, AFR, j KR CDS (82-801 bp)* |
| SEQ ID NO: 57 | 21 VLP5-1-222, ARF, a KR CDS (82-801 bp)* |
| SEQ ID NO: 58 | 21VLP5-1-222, AFR, j KR CDS (82-801 bp)* |
| SEQ ID NO: 59 | 22TP1C-140-40, AFR, b KR CDS (82-801 bp)* |
| SEQ ID NO: 60 | 22TP1C-140-40, AFR, f KR CDS (82-801 bp)* |
| SEQ ID NO: 61 | 22TP1C-141-25, AFR, a KR CDS (82-801 bp)* |
| SEQ ID NO: 62 | 22TP1C-141-25, AFR, b KR CDS (82-801 bp)* |
| SEQ ID NO: 63 | 22TP1C-141-25, AFR, d KR CDS (82-801 bp)* |
| SEQ ID NO: 64 | 23PV1-22-110, cpy2BFR, a KR CDS (262-903 bp)* |
| SEQ ID NO: 65 | 23PV1-22-110, cpy2BFR, f KR CDS (262-903 bp bp)* |
| SEQ ID NO: 66 | Abacus KR protein sequence (28-267 AA)** |
| SEQ ID NO: 67 | 21TX1-60 KR protein sequence (28-267 AA)** |
| SEQ ID NO: 68 | 22VLV2-1-52, cpy2AFR, c KR protein sequence (46-235 AA)** |
| SEQ ID NO: 69 | 22VLV2-1-52, AFR, e KR protein sequence (28-267 AA)** |
| SEQ ID NO: 70 | 22VLV2-1-59, AFR, e KR protein sequence (28-267 AA)** |
| SEQ ID NO: 71 | 22VLV2-1-59, AFR, h KR protein sequence (28-267 AA)** |
| SEQ ID NO: 72 | 22VLV2-1-59, AFR, j KR protein sequence (28-267 AA)** |
| SEQ ID NO: 73 | 21VLP5-1-222, ARF, a KR protein sequence (28-267 AA)** |
| SEQ ID NO: 74 | 21VLP5-1-222, AFR, j KR protein sequence (28-267 AA)** |
| SEQ ID NO: 75 | 22TP1C-140-40, AFR, b KR protein sequence (28-267 AA)** |
| SEQ ID NO: 76 | 22TP1C-140-40, AFR, f KR protein sequence (28-267 AA)** |
| SEQ ID NO: 77 | 22TP1C-141-25, AFR, a KR protein sequence (28-267 AA)** |
| SEQ ID NO: 78 | 22TP1C-141-25, AFR, b KR protein sequence (28-267 AA)** |
| SEQ ID NO: 79 | 22TP1C-141-25, AFR, d KR protein sequence (28-267 AA)** |
| SEQ ID NO: 80 | 23PV1-22-110, cpy2BFR, a KR protein sequence (88-301 AA)** |
| SEQ ID NO: 81 | 23PV1-22-110, cpy2BFR, f KR protein sequence (88-301 AA)** |
| SEQ ID NO: 82 | Abacus KR genomic DNA sequence flanking causative SNP (A/G substitution at |
| | 116 bp in Abacus CDS) located at position 51 bp (Abacus AA substitution Q39R) |
| SEQ ID NO: 83 | Abacus KR genomic DNA sequence flanking causative SNP (T/C substitution at |
| | 262 bp in Abacus CDS) located at position 51 bp (Abacus AA substitution S88P) |
| SEQ ID NO: 84 | Abacus KR genomic DNA sequence flanking causative SNP (A/G substitution at |
| | 364 bp in Abacus CDS) located at position 51 bp (Abacus AA substitution |
| | K122E) |
| SEQ ID NO: 85 | Abacus KR genomic DNA sequence flanking causative SNP (G/A substitution at |
| | 368 bp in Abacus CDS) located at position 51 bp (Abacus AA substitution |
| | E123G) |
| SEQ ID NO: 86 | Abacus KR genomic DNA sequence flanking causative SNP (A/G substitution at |
| | 374 bp in Abacus CDS) located at position 51 bp (Abacus AA substitution |
| | E125G) |
| SEQ ID NO: 87 | Abacus KR genomic DNA sequence flanking causative SNP (T/C substitution at |
| | 415 bp in Abacus CDS) located at position 51 bp (Abacus AA substitution S139P) |
| SEQ ID NO: 88 | Abacus KR genomic DNA sequence flanking causative SNP (A/G substitution at |
| | 472 bp in Abacus CDS) located at position 51 bp (Abacus AA substitution I158V) |
| SEQ ID NO: 89 | Abacus KR genomic DNA sequence flanking causative SNP (A/G substitution at |
| | 523 bp in Abacus CDS) located at position 51 bp (Abacus AA substitution |
| | R175G) |
| SEQ ID NO: 90 | Abacus KR genomic DNA sequence flanking causative SNP (T/C substitution at |
| | 578 bp in Abacus CDS) located at position 51 bp (Abacus AA substitution F193S) |
| SEQ ID NO: 91 | Abacus KR genomic DNA sequence flanking causative SNP (A/G substitution at |
| | 614 bp in Abacus CDS) located at position 51 bp (Abacus AA substitution |
| | K205R) |
| SEQ ID NO: 92 | Abacus KR genomic DNA sequence flanking causative SNP (A/G substitution at |
| | 623 bp in Abacus CDS) located at position 51 bp (Abacus AA substitution |
| | K208R) |
| SEQ ID NO: 93 | Abacus KR genomic DNA sequence flanking causative SNP (G/A substitution at |
| | 649 bp in Abacus CDS) located at position 51 bp (Abacus AA substitution V217I) |
| SEQ ID NO: 94 | Abacus KR genomic DNA sequence flanking causative SNP (T/C substitution at |
| | 653 bp in Abacus CDS) located at position 51 bp (Abacus AA substitution F218S) |
| SEQ ID NO: 95 | Abacus KR genomic DNA sequence flanking causative SNP (A/G substitution at |
| | 752 bp in Abacus CDS) located at position 51 bp (Abacus AA substitution |
| | N251S) |
| SEQ ID NO: 96 | Abacus KR genomic DNA sequence flanking causative SNP (G/A substitution at |
| | 877 bp in Abacus CDS) located at position 51 bp (Abacus AA substitution V293I) |
| SEQ ID NO: 97 | 22VLV2-1-52 FATB CDS (439-1045 bp; N = missing sequence)* |
| SEQ ID NO: 98 | Abacus FATB CDS (1-1176 bp) |
| SEQ ID NO: 99 | 22VLV2-1-52 FATB protein sequence (147-348 AA; X = missing sequence)** |
| SEQ ID NO: 100 | Abacus FATB protein sequence (1-391 AA) |
| SEQ ID NO: 101 | Abacus FATB genomic DNA sequence flanking causative SNP (G/C substitution |
| | at 463 bp in Abacus CDS) located at position 51 bp (Abacus AA substitution |
| | D155H) |
| SEQ ID NO: 102 | Abacus FATB genomic DNA sequence flanking causative SNP (G/A substitution |
| | at 518 bp in Abacus CDS) located at position 51 bp (Abacus AA substitution |
| | R173Q) |
| SEQ ID NO: 103 | Abacus FATB genomic DNA sequence flanking causative SNP (G/C substitution |
| | at 589 bp in Abacus CDS) located at position 51 bp (Abacus AA substitution |
| | G197R) |
| SEQ ID NO: 104 | Abacus FATB genomic DNA sequence flanking causative SNP (G/T substitution |
| | at 603 bp in Abacus CDS) located at position 51 bp (Abacus AA substitution |
| | M201I) |
| SEQ ID NO: 105 | Abacus FATB genomic DNA sequence flanking causative SNP (A/C substitution |
| | at 605 bp in Abacus CDS) located at position 51 bp (Abacus AA substitution |
| | Q202P) |
| SEQ ID NO: 106 | Abacus FATB genomic DNA sequence flanking causative SNP (G/T substitution |
| | at 621 bp in Abacus CDS) located at position 51 bp (Abacus AA substitution |
| | E207D) |
| SEQ ID NO: 107 | 22VLV2-1-52 GDSL1 CDS (67-961 bp)* |
| SEQ ID NO: 108 | Abacus GDSL1 CDS (1-1089 bp) |
| SEQ ID NO: 109 | 22VLV2-1-52 GDSL1 protein sequence (23-320 AA)** |
| SEQ ID NO: 110 | Abacus GDSL1 protein sequence (1 362 AA) |
| SEQ ID NO: 111 | Abacus GSDL1 genomic DNA sequence flanking causative SNP (T/C |
| | substitution at 176 bp in Abacus CDS) located at position 51 bp (Abacus AA |
| | substitution V59A) |
| SEQ ID NO: 112 | Abacus GSDL1 genomic DNA sequence flanking causative SNP (G/A |
| | substitution at 823 bp in Abacus CDS) located at position 51 bp (Abacus AA |
| | substitution D275N) |
| SEQ ID NO: 113 | Abacus GSDL1 genomic DNA sequence flanking causative SNP (G/A |
| | substitution at 889 bp in Abacus CDS) located at position 51 bp (Abacus AA |
| | substitution A297T) |
| SEQ ID NO: 114 | Abacus GSDL1 genomic DNA sequence flanking causative SNP (T/A |
| | substitution at 637 bp in Abacus CDS) located at position 51 bp (Abacus AA |
| | substitution L213M) |

First column: sequence ID;
second column: description of sequence, with in brackets for CDS the start-end positions in bp counted by starting at the start codon,
*CDS coordinates based on start codon identified based on alignment with Abacus,
**protein sequence coordinates based on start codon identified based on alignment with Abacus; for upstream sequences start - end positions in bp to the start codon (for 22VLV2-1-52 the upstream region between −332 and −1 bp from the start codon of ISS1 was not sequenced).

### Example 2

### Gene Expression in Cannabis

Cannabis plants can be modified to express an allele or gene associated with varin production to produce plants with high levels of varins, such as THCV, CBDV, or CBGV. Suitable methods of transgene expression in Cannabis have been described, for example, in Galán-Ávila et al. (A novel and rapid method for Agrobacterium-mediated production of stably transformed Cannabis sativa L. plants, Industrial Crops & Products, 170:113691, 2021). In brief, a varin allele or gene (such as beneficial varin alleles or genes disclosed herein, e.g., beneficial alleles of ISS1, ALT4, KR, GDSL1, and/or FATB) is inserted into a vector, such as a pUC19. The vector is transformed into plant cells using a suitable transformation method, such as Agrobacterium-mediated transformation or protoplast transformation. When Agrobacterium-mediated transformation is used, the vector carrying the allele or gene is first transformed into an Agrobacterium tumefaciens strain, such as LBA4404, and cultured in Luria broth. Explants are placed into Petri dishes with the Agrobacterium inoculant. Hypocotyls, cotyledons or other meristematic tissue from C. sativa seedlings can be used as explants. The explants are then cultured in Petri dishes with a co-culture medium containing Murashige and Skoog medium, sucrose, antibiotics, acetosyringone, and alpha-naphthaleneacetic acid for several days. The explants are then cultured in a regeneration medium containing Murashige and Skoog medium, sucrose, and antibiotics for approximately one month. After root and shoot development, plants are genotyped to identify transgenic plants containing the varin allele or gene and showing varin production.

### Example 3

### CRISPR Editing

One or more nucleotides of a target gene (e.g., an allele or gene disclosed herein, e.g., ISS1, ALT4, KR, GDSL1, or FATB) are altered by a gene editing method, such as CRISPR, to develop plants that produce high levels of THCV, CBDV, or CBGV. A guide RNA (gRNA) is designed to be complementary to the region to be edited in a target gene (e.g., ISS1, ALT4, KR, GDSL1, and/or FATB, or a respective promoter region). Both the gRNA and the Cas9 protein are expressed in Cannabis protoplasts. The gRNA and Cas9 form a complex. The gRNA hybridizes with the complementary DNA sequence and Cas9 introduces a double strand break. The break is repaired via non-homologous end-joining (which can introduce indels) or homology-directed repair with a desired template DNA (such as a template that would introduce a varin allele). Edited cells are regenerated into plants and subsequently screened to identify plants having varin production.

### CLAUSES

Clause 1. A method of identifying a Cannabis plant that produces varins or has increased varin production, comprising: (i) obtaining a nucleic acid sample from the plant or its germplasm; (ii) detecting one or more nucleic acid polymorphisms in: (a) ALT4, or (b) ISS1; wherein the one or more nucleic acid polymorphisms are associated with increased varin production, thereby identifying a plant that produces varins or has increased varin production.

Clause 2. The method of clause 1, wherein the identified plant is selected.

Clause 3. The method of clause 2, further comprising selecting the identified plant for further analysis, propagation, breeding, or to make a product.

Clause 4. The method of any one of the prior clauses, wherein the one or more nucleic acid polymorphisms are detected in ALT4.

Clause 5. The method of clause 4, wherein the one or more nucleic acid polymorphisms detected in ALT4 produce: (a) a glutamine at position 59; (b) a glycine at position 66; (c) an insertion of aspartic acid immediately following position 47; (d) a tyrosine at position 67; (e) a deletion of aspartic acid at position 48; (f) a deletion of aspartic acid at position 49; (g) a lysine at position 68; (h) an aspartic acid at position 81; (i) a glutamic acid at position 85; (j) a histidine at position 111; (k) an alanine at position 115; (l) a serine at position 124; (m) a valine at position 125; (n) an aspartic acid at position 136; (o) an isoleucine at position 160; (p) a valine at position 165; or (q) a valine at position 172; wherein the positions are according to a reference Abacus ALT4 sequence set forth as SEQ ID NO: 12.

Clause 6. The method of clause 4 or 5, wherein the one or more nucleic acid polymorphisms detected in ALT4 produce: a glutamine at position 59 and a glycine at position 66; or a glutamine at position 59 or a glycine at position 66, wherein the positions are according to a reference Abacus ALT4 sequence set forth as SEQ ID NO: 12.

Clause 7. The method of clause 5, wherein the one or more nucleic acid polymorphisms detected in ALT4 produce: (a) the glutamine at position 59; (b) the glycine at position 66; (c) the insertion of aspartic acid immediately following position 47; and (d) the tyrosine at position 67.

Clause 8. The method of clause 5, wherein the one or more nucleic acid polymorphisms detected in ALT4 produce: (a) the glutamine at position 59; (b) the glycine at position 66; (e) the deletion of aspartic acid at position 48; (f) the deletion of aspartic acid at position 49; (g) the lysine at position 68; (h) the aspartic acid at position 81; (i) the glutamic acid at position 85; (j) the histidine at position 111; (k) the alanine at position 115; (l) the serine at position 124; (m) the valine at position 125; (n) the aspartic acid at position 136; (o) the isoleucine at position 160; (p) the valine at position 165; and (q) the valine at position 172.

Clause 9. The method of any one of the prior clauses, wherein the one or more nucleic acid polymorphisms are detected in ISS1.

Clause 10. The method of clause 9, wherein the one or more nucleic acid polymorphisms detected in ISS1 is a deletion of A at position −436 bp upstream from the start codon of ISS1, wherein the position is according to Abacus reference is Csat_AbacusV2, NCBI assembly accession GCA_025232715.1.

Clause 11. The method of any one of the prior clauses, comprising detecting one or more nucleic acid polymorphisms in ALT4 and ISS1.

Clause 12. The method of any one of the prior clauses, wherein the identified plant has a total varin content of 4.0% to 35% in at least one plant part.

Clause 13. The method of any one of the prior clauses, wherein the identified plant has a total varin content of 4.0% to 20% in at least one plant part.

Clause 14. The method of any one of the prior clauses, wherein the identified plant has a total varin content of 4.0% to 7.0% in at least one plant part.

Clause 15. The method of any one of the prior clauses, wherein the identified plant has a varin ratio of 1 to 20 in at least one plant part.

Clause 16. The method of any one of the prior clauses, wherein the identified plant has a varin ratio of 1 to 7 in at least one plant part.

Clause 17. The method of any one of clauses 12-16, wherein the plant part is a flower, leaf, or trichome of the identified plant.

Clause 18. The method of any one of the prior clauses, wherein detecting comprises using PCR, quantitative PCR (qPCR), and/or sequencing.

Clause 19. The method of any one of the prior clauses, wherein detecting comprises using an oligonucleotide primer set or probe.

Clause 20. A method of plant breeding, comprising crossing the plant identified by the method of any one of the prior clauses.

Clause 21. The method of clause 20, wherein crossing comprises selfing, sibling crossing, outcrossing, or backcrossing.

Clause 22. A Cannabis plant identified by the method of any one of the prior clauses.

Clause 23. A seed, tissue, cell, or F1 progeny of the Cannabis plant of clause 22.

Clause 24. A product made from the Cannabis plant of clause 22, or the seed, tissue, or cells of clause 23.

Clause 25. A method of producing a modified Cannabis plant, comprising:
introducing a genetic modification in the Cannabis plant, wherein the genetic modification increases ALT4 activity against C4 fatty acids or decreases expression of ISS1,
wherein the increasing or decreasing is relative to the Cannabis plant in an unmodified state.

Clause 26. A method of producing a modified Cannabis plant, comprising introducing a genetic modification in the Cannabis plant, wherein the genetic modification is in ALT4 or ISS1.

Clause 27. The method of clause 25 or clause 26, wherein the genetic modification increases production of varins relative to the Cannabis plant in an unmodified state.

Clause 28. The method of any one of clauses 25-27, wherein the genetic modification is in ALT4 and/or increases ALT4 activity against C4 fatty acids.

Clause 29. The method of clause 28, wherein the genetic modification produces:
(a) a glutamine at position 59;
(b) a glycine at position 66;
(c) an insertion of aspartic acid immediately following position 47;
(d) a tyrosine at position 67;
(e) a deletion of aspartic acid at position 48;
(f) a deletion of aspartic acid at position 49;
(g) a lysine at position 68;
(h) an aspartic acid at position 81;
(i) a glutamic acid at position 85;
(j) a histidine at position 111;
(k) an alanine at position 115;
(l) a serine at position 124;
(m) a valine at position 125;
(n) an aspartic acid at position 136;
(o) an isoleucine at position 160;
(p) a valine at position 165; or
(q) a valine at position 172;
wherein the positions are according to a reference Abacus ALT4 sequence set forth as SEQ ID NO: 12.

Clause 30. The method of clause 28, wherein the genetic modification produces:
a glutamine at position 59 and a glycine at position 66; or
a glutamine at position 59 or a glycine at position 66,
wherein the positions are according to a reference Abacus ALT4 sequence set forth as SEQ ID NO: 12.

Clause 31. The method of any one of clauses 25-30, wherein the genetic modification is in ISS1 and/or decreases expression of ISS1.

Clause 32. The method of clause 31, wherein the genetic modification is a deletion of A at position −436 bp upstream from the start codon of ISS1, wherein the position is according to Abacus reference Csat_Abacus V2, NCBI assembly accession GCA_025232715.1.

Clause 33. The method of any one of clauses 25-32, wherein the modified plant has a total varin content of 4.0% to 35% in at least one plant part.

Clause 34. The method of any one of clauses 25-33, wherein the modified plant has a total varin content of 4.0% to 20% in at least one plant part.

Clause 35. The method of any one of clauses 25-34, wherein the modified plant has a total varin content of 4.0% to 7.0% in at least one plant part.

Clause 36. The method of any one of clauses 25-35, wherein the modified plant has a varin ratio of 1 to 20 in at least one plant part.

Clause 37. The method of any one of clauses 25-36, wherein the modified plant has a varin ratio of 1 to 7 in at least one plant part.

Clause 38. The method of any one of clauses 33-37, wherein the plant part is a flower, leaf, or trichome of the modified plant.

Clause 39. The method of any one of clauses 25-38, wherein the genetic modification is a nucleic acid substitution, insertion, or deletion.

Clause 40. The method of any one of clauses 25-39, wherein the genetic modification is introduced by mutagenesis or gene editing.

Clause 41. The method of clause 40, wherein gene editing comprises use of an RNA interference (RNAi), clustered regularly interspaced short palindromic repeats/CRISPR associated protein (CRISPR/Cas), zinc-finger nucleases (ZFN), or transcription activator-like effector nucleases (TALEN)-based editing system.

Clause 42. A method of producing a modified Cannabis plant having increased varin production, comprising introducing a heterologous beneficial allele of ALT4 or ISS1 associated with increased varin production.

Clause 43. The method of clause 42, wherein the allele of ALT4 comprises:
a nucleic acid sequence encoding an amino acid sequence having at least 90% sequence identity to SEQ ID NO: 10 or SEQ ID NO: 11, or
a nucleic acid sequence encoding SEQ ID NO: 10 or SEQ ID NO: 11.

Clause 44. The method of clause 42 or clause 43, wherein the allele of ISS1 comprises a nucleic acid sequence having at least 90% sequence identity to SEQ ID NO: 29, or comprises SEQ ID NO: 29.

Clause 45. A modified Cannabis plant produced by the method of any one of clauses 25-44.

Clause 46. A modified Cannabis plant comprising a non-naturally occurring beneficial allele of ALT4 and/or ISS1.

Clause 47. The modified Cannabis plant of clause 46, wherein the beneficial allele of ALT4 and/or ISS1 increases varin production in the modified Cannabis plant relative to the Cannabis plant in an unmodified state.

Clause 48. The modified Cannabis plant of clause 46 or clause 47, wherein the beneficial allele of ALT4 comprises:
a nucleic acid sequence encoding an amino acid sequence having at least 90% sequence identity to SEQ ID NO: 10 or SEQ ID NO: 11, or
a nucleic acid sequence encoding SEQ ID NO: 10 or SEQ ID NO: 11.

Clause 49. The modified Cannabis plant of any one of clauses 46 to 48, wherein the beneficial allele of ISS1 comprises a nucleic acid sequence having at least 90% sequence identity to SEQ ID NO: 29, or comprises SEQ ID NO: 29.

Clause 50. The modified Cannabis plant of any one of clauses 46 to 49, wherein the beneficial allele of ALT4 and/or ISS1 is introduced by genome editing or transformation of a heterologous gene encoding the beneficial allele of ALT4 and/or ISS1.

It will be apparent that the precise details of the methods or compositions described may be varied or modified without departing from the spirit of the described aspects of the disclosure. We claim all such modifications and variations that fall within the scope and spirit of the claims below.

1. A method of producing a Cannabis plant that produces varins or has increased varin production, comprising:
(i) obtaining a nucleic acid sample from a Cannabis plant or its germplasm;
(ii) detecting one or more nucleic acid polymorphisms in:
(a) ISS1,
(b) ALT4,
(c) FATB,
(d) KR, or
(e) GDSL1;
wherein the one or more nucleic acid polymorphisms are associated with increased varin production,
(iii) selecting a Cannabis plant comprising the one or more nucleic acid polymorphisms associated with increased varin production, and
(iv) crossing the selected plant to obtain a progeny plant comprising the one or more nucleic acid polymorphisms associated with increased varin production, thereby producing the Cannabis plant that produces varins or has increased varin production.

2. The method of claim 1, wherein the one or more nucleic acid polymorphisms are detected in ISS1.

3. The method of claim 2, wherein the one or more nucleic acid polymorphisms detected in ISS1 is a deletion of A at position −436 bp upstream from the start codon of ISS1, wherein the position is according to Abacus reference sequence Csat_AbacusV2, NCBI assembly accession GCA_025232715.1.

4. The method of claim 1, wherein:
(i) the one or more nucleic acid polymorphisms are detected in ALT4, and the one or more nucleic acid polymorphisms detected in ALT4 produce:
(a) a glutamine at position 59;
(b) a glycine at position 66;
(c) an insertion of aspartic acid immediately following position 47;
(d) a tyrosine at position 67;
(e) a deletion of aspartic acid at position 48;
(f) a deletion of aspartic acid at position 49;
(g) a lysine at position 68;
(h) an aspartic acid at position 81;
(i) a glutamic acid at position 85;
(i) a histidine at position 111;
(k) an alanine at position 115;
(l) a serine at position 124;
(m) a valine at position 125;
(n) an aspartic acid at position 136;
(o) an isoleucine at position 160;
(p) a valine at position 165;
(q) a valine at position 172;
(r) a serine at position 7;
(s) a glutamine at position 202;
(t) an alanine at position 206;
wherein the positions are according to a reference Abacus ALT4 sequence set forth as SEQ ID NO: 12;
(ii) the one or more nucleic acid polymorphisms are detected in FATB, wherein the one or more nucleic acid polymorphisms detected in FATB produce:
(a) a histidine at position 155;
(b) a glutamine at position 173;
(c) an arginine at position 197;
(d) an isoleucine at position 201;
(e) a proline at position 202; or
(f) an aspartic acid at position 207;
wherein the positions are according to a reference Abacus FATB sequence set forth as SEQ ID NO: 100;
(iii) the one or more nucleic acid polymorphisms are detected in KR, wherein the one or more nucleic acid polymorphisms detected in KR produce:
(a) an arginine at position 39;
(b) a proline at position 88;
(c) a glutamic acid at position 122;
(d) a glycine at position 123;
(e) a glycine at position 125;
(f) a proline at position 139;
(g) a valine at position 158;
(h) a glycine at position 175;
(i) a serine at position 193;
(j) an arginine at position 205;
(k) an arginine at position 208;
(l) an isoleucine at position 217;
(m) a serine at position 218;
(n) a serine at position 251; or
(o) an isoleucine at position 293;
wherein the positions are according to a reference Abacus KR sequence set forth as SEQ ID NO: 66; or
(iv) the one or more nucleic acid polymorphisms are detected in GDSL1, wherein the one or more nucleic acid polymorphisms detected in GDSL1 produce:
(a) an alanine at position 59;
(b) an asparagine at position 275;
(c) a threonine at position 297; or
(d) a methionine at position 213;
wherein the positions are according to a reference Abacus GDSL1 sequence set forth as SEQ ID NO: 110.

5-12. (canceled)

13. The method of claim 4, wherein:
(i) the one or more nucleic acid polymorphisms detected in ALT4 produce:
a serine at position 7 and a lysine at position 68; or
a serine at position 7 or a lysine at position 68,
wherein the positions are according to a reference Abacus ALT4 sequence set forth as SEQ ID NO: 12; or
(ii) the one or more nucleic acid polymorphisms detected in KR produce:
a valine at position 158 and an isoleucine at position 217; or
a valine at position 158 or an isoleucine at position 217,
wherein the positions are according to a reference Abacus KR sequence set forth as SEQ ID NO: 66.

14-15. (canceled)

16. The method of claim 1, comprising detecting one or more nucleic acid polymorphisms in at least two of: ALT4, KR, FATB, GDSL1, and ISS1.

17. The method of claim 1, comprising detecting one or more nucleic acid polymorphisms in:
(i) ISS1 and KR;
(ii) ISS1, KR, and ALT4; or
(iii) ISS1 and GDSL1.

18. The method of claim 1, wherein detecting comprises using PCR, quantitative PCR (qPCR), and/or sequencing.

19. The method of claim 1, wherein detecting comprises using an oligonucleotide primer set or probe.

20-21. (canceled)

22. The method of claim 1, wherein crossing comprises selfing, sibling crossing, outcrossing, or backcrossing.

23. A method of producing a genetically modified Cannabis plant, comprising introducing a genetic modification in the Cannabis plant, wherein the genetic modification is in ALT4, KR, FATB, GDSL1, or ISS1.

24-28. (canceled)

29. A method of producing a modified Cannabis plant having increased varin production, comprising introducing a heterologous beneficial allele of ALT4, KR, FATB, or GDSL1 associated with increased varin production.

30-32. (canceled)

33. The method of claim 1, wherein the Cannabis plant comprising the one or more nucleic acid polymorphisms associated with increased varin production has a total varin content of:
(i) 4.0% to 35%,
(ii) 15% to 35%,
(iii) 15% to 22%,
(iv) 4.0% to 22%, or
(v) 4.0% to 7.0%;
in at least one plant part.

34. The method of claim 1, wherein the identified or modified Cannabis plant comprising the one or more nucleic acid polymorphisms associated with increased varin production has a varin ratio of:
(i) 1 to 20,
(ii) 7 to 20,
(iii) 7 to 10,
(iv) 1 to 10, or
(v) 1 to 7;
in at least one plant part.

35. The method of claim 33, wherein the plant part is a flower, leaf, or trichome.

36. A Cannabis plant produced by the method of claim 1.

37. A seed, tissue, cell, or F1 progeny of the Cannabis plant of claim 36.

38. A product made from the Cannabis plant of claim 36.

39. The product of claim 38, wherein the product is a kief, hashish, bubble hash, an edible product, solvent reduced oil, sludge, e-juice, or tincture.

40. A genetically modified Cannabis plant produced by the method of claim 23.
